(19) United States
(12) Patent Application Publication
(10) Pub. No.: US 2023/0172056 A1
(43) Pub. Date: Jun. 1, 2023

(54) LIGHT-EMITTING DEVICE INCLUDING AMINE-BASED COMPOUND, ELECTRONIC APPARATUS INCLUDING THE LIGHT-EMITTING DEVICE, AND THE AMINE-BASED COMPOUND

(71) Applicant: Samsung Display Co., Ltd., Yongin-si (KR)

(72) Inventors: Dongjun KIM, Yongin-si (KR); Chaeyeong KIM, Yongin-si (KR); Hankyu PAK, Yongin-si (KR); Byeongwook YOO, Yongin-si (KR); Seowon CHO, Yongin-si (KR); Sohee JO, Yongin-si (KR)

(73) Assignee: Samsung Display Co., Ltd., Yongin-si (KR)

(21) Appl. No.: 17/994,825

(22) Filed: Nov. 28, 2022

(30) Foreign Application Priority Data
Nov. 29, 2021 (KR) 10-2021-0167728

(51) Int. Cl. H01L 51/00; C07D 307/91; C07D 209/88; C09K 11/06; C07C 211/61

(52) U.S. Cl. CPC H01L 51/006; C07D 307/91; C07D 209/88; C09K 11/06; H01L 51/0061; C07C 211/61; H01L 51/0056; C09K 2211/1014; H01L 51/0073; H01L 51/0058; H01L 51/5088; C09K 2211/1007; H01L 51/0072; C07C 2603/97; H01L 27/3244; C09K 2211/1018; C07C 2603/18; H01L 51/5056; C09K 2211/1011

(57) ABSTRACT

An amine-based compound is represented by Formula 1. A light-emitting device includes a first electrode, a second electrode facing the first electrode, an interlayer between the first electrode and the second electrode and including an emission layer, and the amine-based compound. An electronic apparatus includes the light-emitting device:

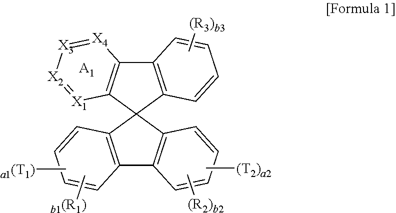

The description of Formula 1 is the same as in the specification.

### CROSS-REFERENCE TO RELATED APPLICATION(S)

This application claims priority to and benefits of Korean Patent Application No. 10-2021-0167728 under 35 U.S.C. § 119, filed on Nov. 29, 2021, in the Korean Intellectual Property Office, the entire contents of which are incorporated herein by reference.

### BACKGROUND

### 1. Technical Field

Embodiments relate to a light-emitting device including an amine-based compound, an electronic apparatus including the light-emitting device, and the amine-based compound.

### 2. Description of the Related Art

Organic light-emitting devices are self-emissive devices that, as compared with devices of the related art, have wide viewing angles, high contrast ratios, short response times, and excellent characteristics in terms of luminance, driving voltage, and response speed, and produce full-color images.

Organic light-emitting devices may include a first electrode located on a substrate, and a hole transport region, an emission layer, an electron transport region, and a second electrode sequentially stacked on the first electrode. Holes provided from the first electrode may move toward the emission layer through the hole transport region, and electrons provided from the second electrode may move toward the emission layer through the electron transport region. Carriers, such as holes and electrons, may recombine in such an emission layer region to produce excitons. These excitons transition from an excited state to a ground state, thereby generating light.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

Provided are a light-emitting device including an amine-based compound, an electronic apparatus including the light-emitting device, and the amine-based compound.

Additional aspects will be set forth in part in the description which follows and, in part, will be apparent from the description, or may be learned by practice of the embodiments of the disclosure.

According to embodiments, a light-emitting device may include a first electrode, a second electrode facing the first electrode, an interlayer between the first electrode and the second electrode and including an emission layer, and an amine-based compound represented by Formula 1.

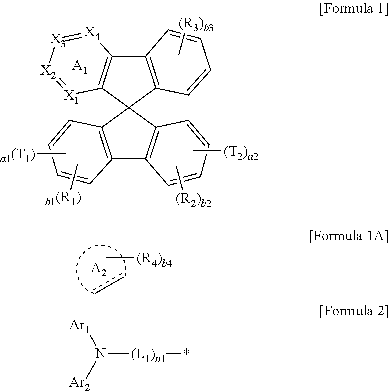

In Formulae 1, 1A, and 2,

X₁ may be C(Z₁), X₂ may be C(Z₂), X₃ may be C(Z₃), and X₄ may be C(Z₄),

wherein in Z₁ to Z₄: Z₁ and Z₂ may be bonded together to form ring A₂ represented by Formula 1A; Z₂ and Z₃ may be bonded together to form ring A₂ represented by Formula 1A; or Z₃ and Z₄ may be bonded together to form ring A₂ represented by Formula 1A,

ring A₁ and ring A₂ represented by Formula 1A may be condensed with each other,

[symbol] in Formula 1A may indicate a single bond or a double bond,

ring A₂ may be a C₃-C₃₀ carbocyclic group,

Z₁ to Z₄ may each independently be a group represented by Formula 2, hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),

at least one of Z₁ to Z₄ may be a group represented by Formula 2,

T₁ and T₂ may each independently be a group represented by Formula 2,

a1 and a2 may each independently be an integer from 0 to 4,

the sum of a1 and a2 may be 1 or more,

R₁ to R₄ may each independently be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),

b1 to b3 may each independently be an integer from 0 to 4,

b4 may be an integer from 0 to 10,

L₁ may be a single bond, a C₃-C₆₀ carbocyclic group that is unsubstituted or substituted with at least one R₁₀ₐ, or a C₁-C₆₀ heterocyclic group that is unsubstituted or substituted with at least one R₁₀ₐ,

n1 may be an integer from 1 to 5,

Ar₁ and Ar₂ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,

* may indicate a binding site to a neighboring atom, and

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, a pyridinyl group, a pyrimidinyl group, a pyridazinyl group, a pyrazinyl group, a triazinyl group, or any combination thereof.

In an embodiment, the interlayer may include the amine-based compound.

In an embodiment, the first electrode may be an anode, the second electrode may be a cathode, the interlayer may further include a hole transport region between the emission layer and the first electrode, and the hole transport region may include the amine-based compound.

In an embodiment, the hole transport region may include at least one of a hole injection layer and a hole transport layer, and at least one of the hole injection layer and the hole transport layer may include the amine-based compound.

In an embodiment, the light-emitting device may further include a capping layer located outside the first electrode or located outside the second electrode, wherein the capping layer may include the amine-based compound.

In an embodiment, the light-emitting device may further include: a first capping layer located outside the first electrode and comprising the amine-based compound; a second capping layer located outside the second electrode and comprising the amine-based compound; or the first capping layer and the second capping layer.

According to embodiments, an electronic apparatus may include the light-emitting device.

In an embodiment, the electronic apparatus may further include a thin-film transistor, wherein the thin-film transistor may include a source electrode and a drain electrode, and the first electrode of the light-emitting device may be electrically connected to the source electrode or the drain electrode.

In an embodiment, the electronic apparatus may further include a color filter, a color conversion layer, a touch screen layer, a polarizing layer, or any combination thereof.

According to embodiments, an amine-based compound may be represented by Formula 1.

In an embodiment, ring A₂ may be a benzene group or a naphthalene group.

In an embodiment, the amine-based compound represented by Formula 1 may be represented by one of Formulae 1-1 to 1-3, which are explained below.

In an embodiment, Z₁ to Z₄ may each independently be: a group represented by Formula 2, hydrogen, or deuterium; a C₁-C₁₀ alkyl group that is unsubstituted or substituted with deuterium, a phenyl group, or any combination thereof; or a phenyl group or a naphthyl group, each unsubstituted or substituted with deuterium, a C₁-C₁₀ alkyl group, a phenyl group, or any combination thereof.

In an embodiment, one of Z₁ to Z₄ may be a group represented by Formula 2.

In an embodiment, the sum of a1 and a2 may be 1.

In an embodiment, L₁ may be: a single bond; or a benzene group or a naphthalene group, each unsubstituted or substituted with at least one R₁₀ₐ.

In an embodiment, Ar₁ and Ar₂ may each independently be a C₆-C₆₀ aryl group that is unsubstituted or substituted with at least one R₁₀ₐ, a C₁-C₆₀ heteroaryl group that is unsubstituted or substituted with at least one R₁₀ₐ, a monovalent non-aromatic condensed polycyclic group that is unsubstituted or substituted with at least one R₁₀ₐ, or a monovalent non-aromatic condensed heteropolycyclic group that is unsubstituted or substituted with at least one R₁₀ₐ.

In an embodiment, Ar₁ and Ar₂ may each independently be a group represented by one of Formulae 2A-1 to 2A-14, which are explained below.

In an embodiment, the amine-based compound represented by Formula 1 may be represented by one of Formulae 1-1-1 to 1-1-16, 1-2-1 to 1-2-16, and 1-3-1 to 1-3-16, which are explained below.

### BRIEF DESCRIPTION OF THE DRAWINGS

The above and other aspects and features of the disclosure will be more apparent by describing in detail embodiments thereof with reference to the accompanying drawings, in which:

FIG. 1 is a schematic cross-sectional view of a structure of a light-emitting device according to an embodiment;

FIG. 2 is a schematic cross-sectional view of a structure of an electronic apparatus according to an embodiment; and

FIG. 3 is a schematic cross-sectional view of a structure of an electronic apparatus according to another embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like numbers refer to like elements throughout.

In the description, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the description, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group of” for the purpose of its meaning and interpretation. For example, “at least one of A and B” may be understood to mean “A, B, or A and B.” When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

A light-emitting device may include a first electrode, a second electrode facing the first electrode, an interlayer between the first electrode and the second electrode and including an emission layer, and an amine-based compound represented by Formula 1:

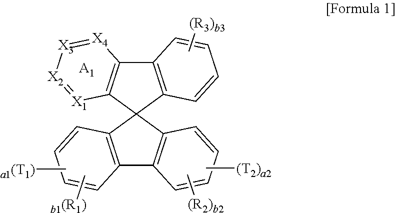

In Formula 1, X₁ may be C(Z₁), X₂ may be C(Z₂), Xₐ may be C(Z₃), and X₄ may be C(Z₄).

In Formula 1, in Z₁ to Z₄: Z₁ and Z₂ may be bonded together to form ring A₂ represented by Formula 1A; Z₂ and Zₐ may be bonded together to form ring A₂ represented by Formula 1A; or Zₐ and Z₄ may be bonded together to form ring A₂ represented by Formula 1A. In Formula 1, ring A₁ and ring A₂ represented by Formula 1A may be condensed with each other. Formula 1A is the same as described in the specification.

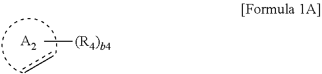

In Formula 1A, [symbol] indicates a single bond or a double bond.

In Formula 1A, ring A₂ may be a C₃-C₃₀ carbocyclic group.

In an embodiment, ring A₂ may be a C₆-C₆₀ aryl group.

In an embodiment, ring A₂ may be a benzene group or a naphthalene group.

In an embodiment, the amine-based compound represented by Formula 1 may be represented by one of Formulae 1-1 to 1-3:

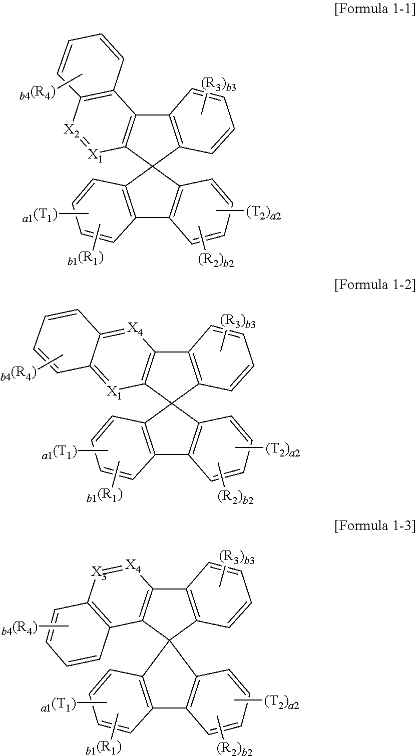

In Formulae 1-1 to 1-3, b4 may be an integer from 0 to 4, and X₁ to X₄, T₁, T₂, a1, a2, R₁ to R₄, and b1 to b3 are respectively the same as described in the specification.

In Formula 1, Z₁ to Z₄ may each independently be a group represented by Formula 2, hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —N(Q₁)(Q₂), —B(Q₁)(Q₂), —C(═O(Q₁), —S(═O)₂(Q₁), or —P(═O)(Q₁)(Q₂).

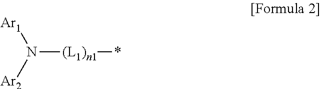

Formula 2 is the same as described in the specification.

In an embodiment, Z₁ to Z₄ may each independently be:

a group represented by Formula 2;

hydrogen, deuterium, —F, —Cl, —Br, —I, a hydroxyl group, a cyano group, a nitro group, a C₁-C₂₀ alkyl group, a C₁-C₂₀ alkoxy group, or a C₁-C₂₀ alkylthio group;

a C₁-C₂₀ alkyl group, a C₁-C₂₀ alkoxy group, or a C₁-C₂₀ alkylthio group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbomanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbornanyl group, a norbomenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a C₁-C₂₀ alkylthio group, a cyclopentyl group, a cyclohexyl group, a cycloheptyl group, a cyclooctyl group, an adamantanyl group, a norbornanyl group, a norbomenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, an azadibenzosilolyl group, —Si(Q₃₁)(Q₃₂)(Q₃₃), —N(Q₃₁)(Q₃₂), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof; or

—Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and

Q₁ to Q₃ and Q₃₁ to Q₃₃ may each independently be: —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or

an n-propyl group, an iso-propyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with at least one of deuterium, a C₁-C₁₀ alkyl group, a phenyl group, a biphenyl group, a pyridinyl group, a pyrimidinyl group, a pyridazinyl group, a pyrazinyl group, and a triazinyl group.

In an embodiment, Z₁ to Z₄ may each independently be:

a group represented by Formula 2, hydrogen, or deuterium;

a C₁-C₁₀ alkyl group that is unsubstituted or substituted with deuterium, a phenyl group, or any combination thereof; or

a phenyl group or a naphthyl group, each unsubstituted or substituted with deuterium, a C₁-C₁₀ alkyl group, a phenyl group, or any combination thereof.

In Formula 1, at least one of Z₁ to Z₄ may be a group represented by Formula 2.

In an embodiment, one of Z₁ to Z₄ may be a group represented by Formula 2.

In Formula 1, T₁ and T₂ may each independently be a group represented by Formula 2.

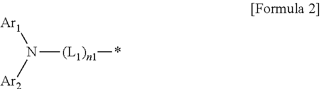

Formula 2 is the same as described in the specification.

In Formula 1, a1 and a2 may each independently be an integer from 0 to 4.

In Formula 1, the sum of a1 and a2 may be 1 or more.

In an embodiment, in Formula 1, the sum of a1 and a2 may be 1.

In Formula 1, R₁ to R₄ may each independently be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₁-C₆₀ aryloxy group that is unsubstituted or substituted with at least one R₁₀ₐ, a C₁-C₆₀ arylthio group that is unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂). R₁₀ₐ and Q₁ to Q₃ are respectively the same as described in the specification.

In an embodiment, R₁ to R₄ may each independently be:

hydrogen, deuterium, —F, —Cl, —Br, —I, a hydroxyl group, a cyano group, a nitro group, a C₁-C₂₀ alkyl group, a C₁-C₂₀ alkoxy group, or a C₁-C₂₀ alkylthio group;

a C₁-C₂₀ alkyl group, a C₁-C₂₀ alkoxy group, or a C₁-C₂₀ alkylthio group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbomanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbomanyl group, a norbomenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a C₁-C₂₀ alkylthio group, a cyclopentyl group, a cyclohexyl group, a cycloheptyl group, a cyclooctyl group, an adamantanyl group, a norbornanyl group, a norbomenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, an azadibenzosilolyl group, —Si(Q₃₁)(Q₃₂)(Q₃₃), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or any combination thereof; or

—Si(Q₁)(Q₂)(Q₃), —B(Q₁)(Q₂), —C(═O)(Q₁), —S(═O)₂(Q₁), or —P(═O)(Q₁)(Q₂), and

Q₁ to Q₃ and Q₃₁ to Q₃₃ may each independently be: —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or

an n-propyl group, an iso-propyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with at least one of deuterium, a C₁-C₁₀ alkyl group, a phenyl group, a biphenyl group, a pyridinyl group, a pyrimidinyl group, a pyridazinyl group, a pyrazinyl group, and a triazinyl group.

In an embodiment, R₁ to R₄ may each independently be: hydrogen or deuterium;

a C₁-C₁₀ alkyl group that is unsubstituted or substituted with deuterium, a phenyl group, or any combination thereof; or

a phenyl group or a naphthyl group, each unsubstituted or substituted with deuterium, a C₁-C₁₀ alkyl group, a phenyl group, or any combination thereof.

In Formula 1, b1 to b3 may each independently be an integer from 0 to 4.

In Formula 1, b4 may be an integer from 0 to 10.

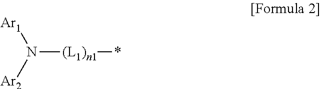

In Formula 2, L₁ may be a single bond, a C₃-C₆₀ carbocyclic group that is unsubstituted or substituted with at least one R₁₀ₐ, or a C₁-C₆₀ heterocyclic group that is unsubstituted or substituted with at least one R₁₀ₐ. R₁₀ₐ may be the same as described in the specification.

In an embodiment, L₁ may be: a single bond; or

a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ. R₁₀ₐ may be the same as described in the specification.

In an embodiment, L₁ may be: a single bond; or a benzene group or a naphthalene group, each unsubstituted or substituted with at least one R₁₀ₐ. R₁₀ₐ may be the same as described in the specification.

In Formula 2, n1 may be an integer from 1 to 5.

In Formula 2, Ar₁ and Ar₂ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ. R₁₀ₐ may be the same as described in the specification.

In an embodiment, Ar₁ and Ar₂ may each independently be a C₆-C₆₀ aryl group that is unsubstituted or substituted with at least one R₁₀ₐ, a C₁-C₆₀ heteroaryl group that is unsubstituted or substituted with at least one R₁₀ₐ, a monovalent non-aromatic condensed polycyclic group that is unsubstituted or substituted with at least one R₁₀ₐ, or a monovalent non-aromatic condensed heteropolycyclic group that is unsubstituted or substituted with at least one R₁₀ₐ.

In an embodiment, Ar₁ and Ar₂ may each independently be a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with at least one R₁₀ₐ. R₁₀ₐ may be the same as described in the specification.

In an embodiment, Ar₁ and Ar₂ may each independently be a group represented by one of Formulae 2A-1 to 2A-14:

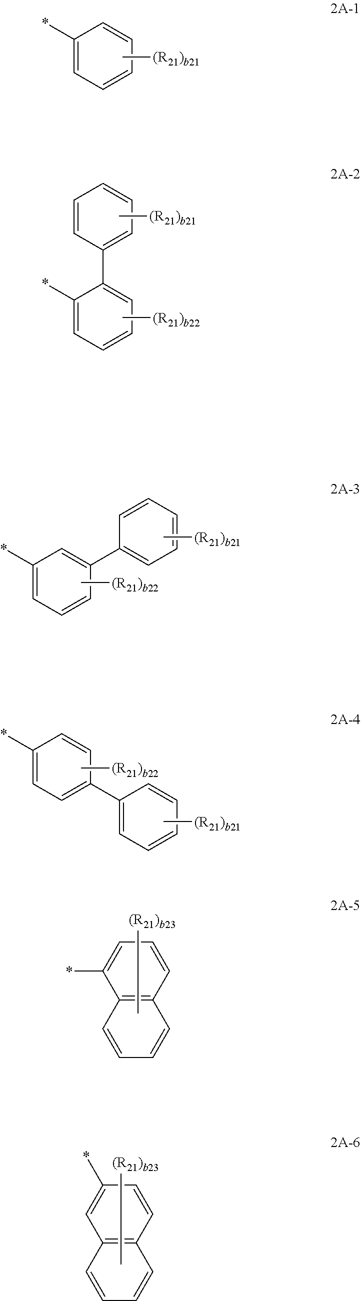

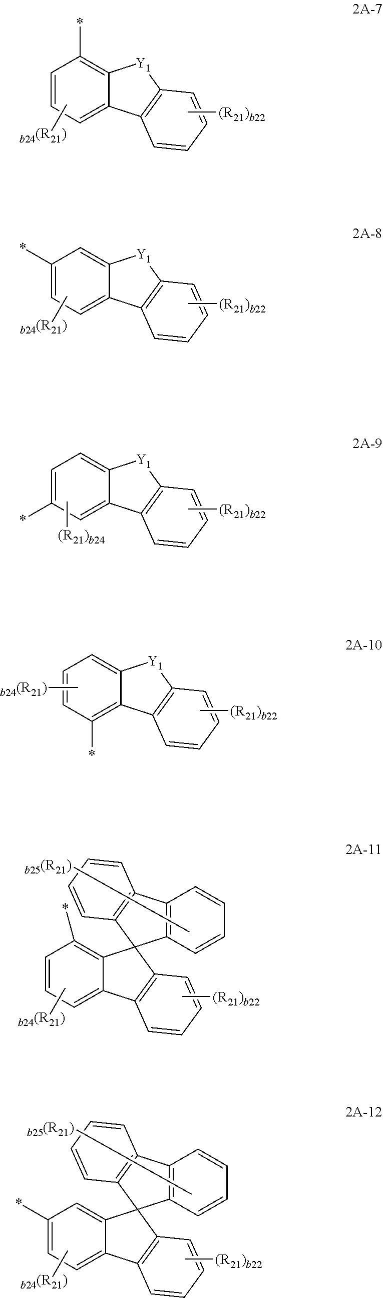

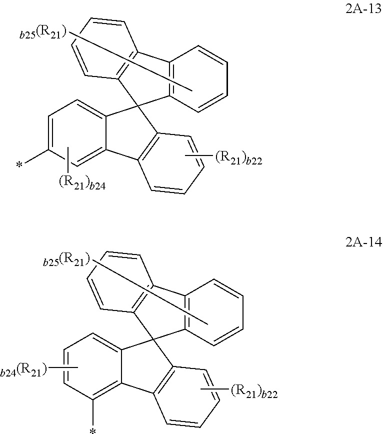

In Formulae 2A-1 to 2A-14,

Y₁ may be O, S, N(R₁ₐ), C(R₁ₐ)(R_{1b}), or Si(R₁ₐ)(R_{1b}),

R₂₁ may be hydrogen, deuterium, —F, —Cl, —Br, —I, a hydroxyl group, a cyano group, a nitro group, a C₁-C₅₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),

R₁ₐ and R_{1b} may each independently be hydrogen, deuterium, a C₁-C₆₀ alkyl group that is unsubstituted or substituted with at least one R₁₀ₐ, a C₃-C₆₀ carbocyclic group that is unsubstituted or substituted with at least one R₁₀ₐ, or a C₁-C₆₀ heterocyclic group that is unsubstituted or substituted with at least one R₁₀ₐ,

b21 may be an integer from 0 to 5,

b22 may be an integer from 0 to 4,

b23 may be an integer from 0 to 7,

b24 may be an integer from 0 to 3,

b25 may be an integer from 0 to 8,

* may indicate a binding site to a neighboring atom, and

R₁₀ₐ and Q₁ to Q₃ are respectively the same as described in the specification.

In an embodiment, R₁ₐ and R_{1b} may each independently be hydrogen, deuterium, a C₁-C₂₀ alkyl group that is unsubstituted or substituted with at least one R₁₀ₐ, or a C₆-C₃₀ aryl group that is unsubstituted or substituted with at least one R₁₀ₐ. R₁₀ₐ may be the same as described in the specification.

In Formula 2, * may indicate a binding site to a neighboring atom.

In an embodiment, the amine-based compound represented by Formula 1 may be represented by one of Formulae 1-1-1 to 1-1-16, 1-2-1 to 1-2-16, and 1-3-1 to 1-3-16:

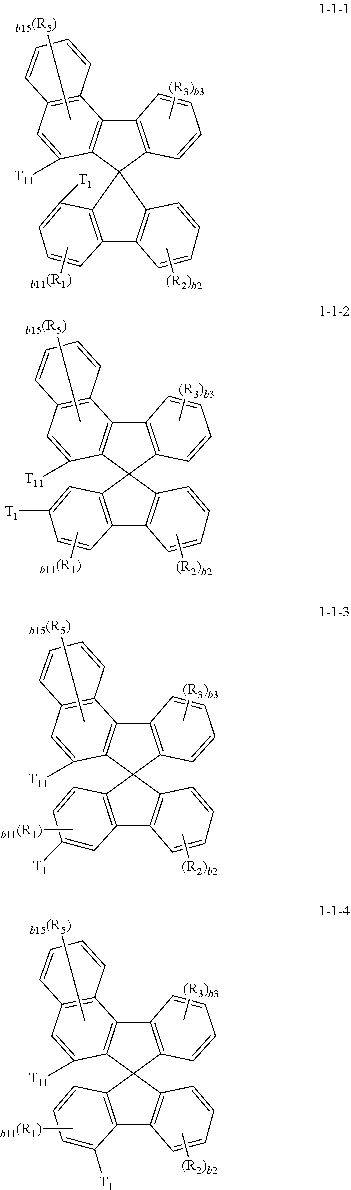

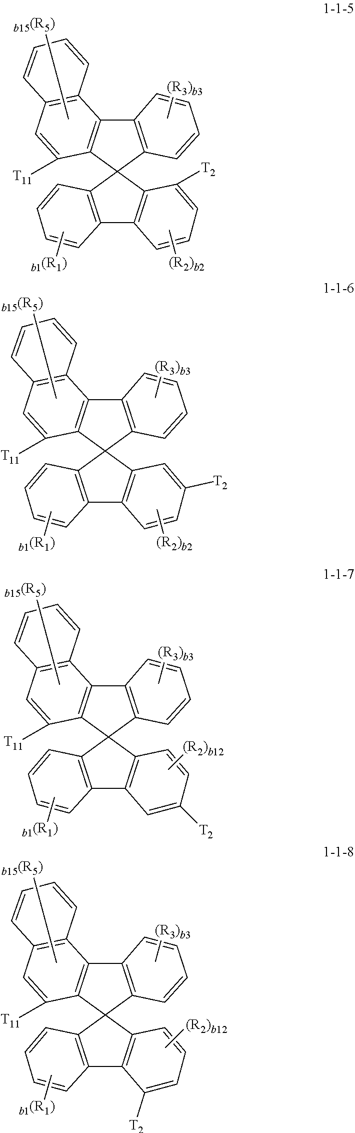

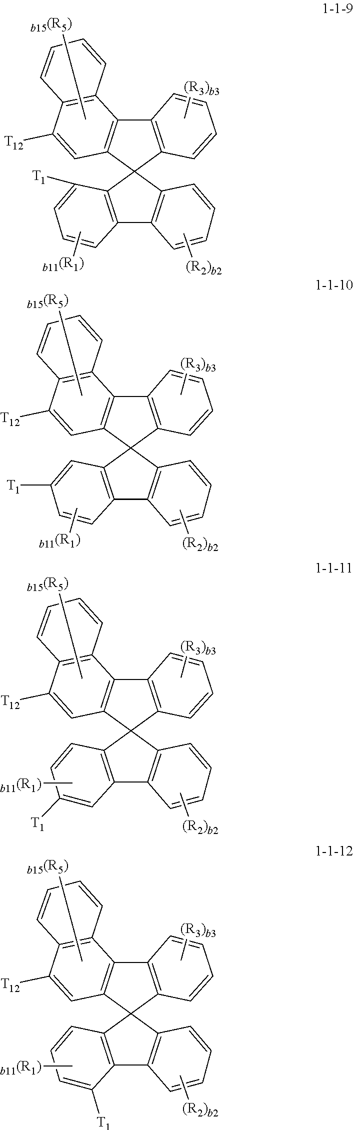

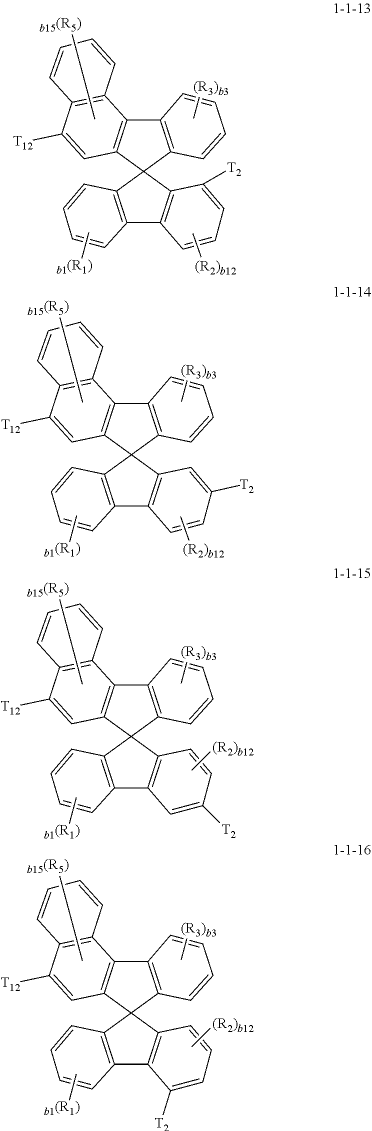

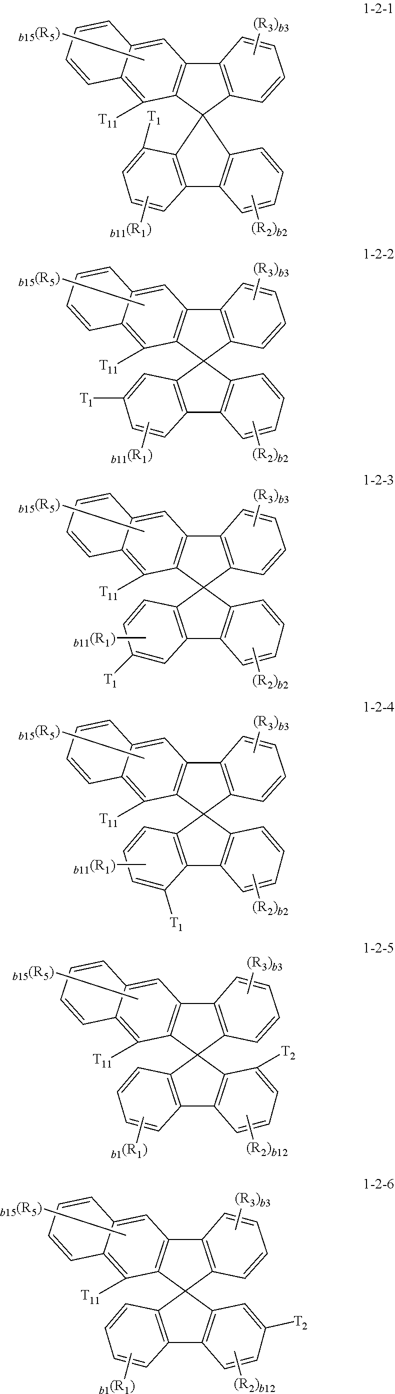

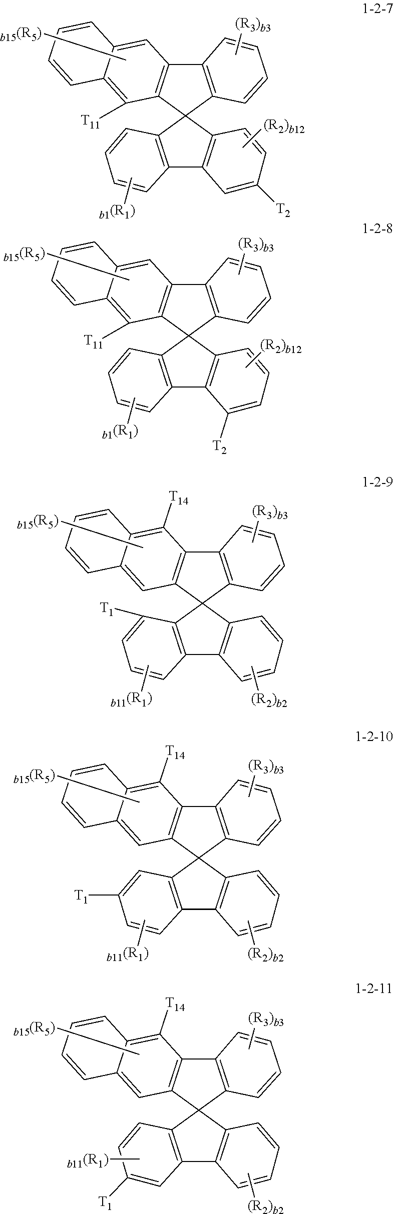

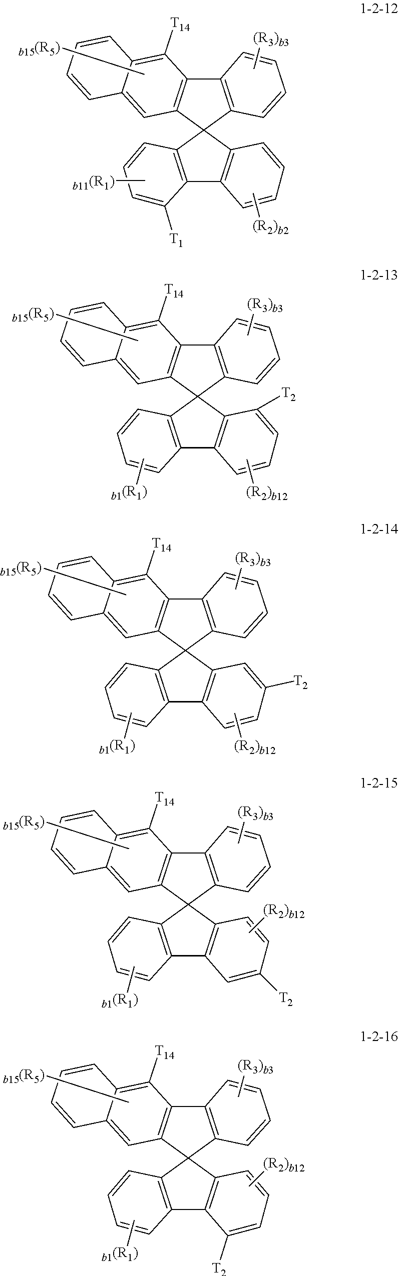

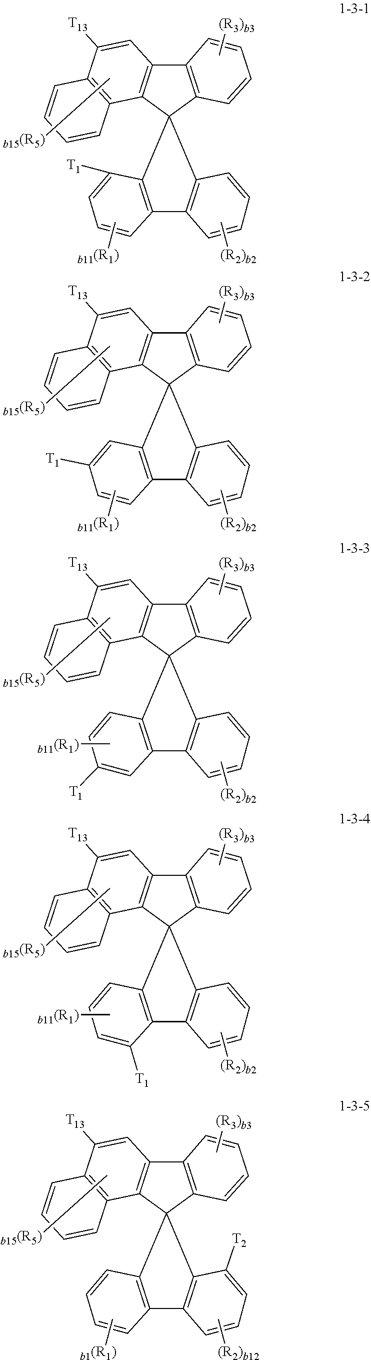

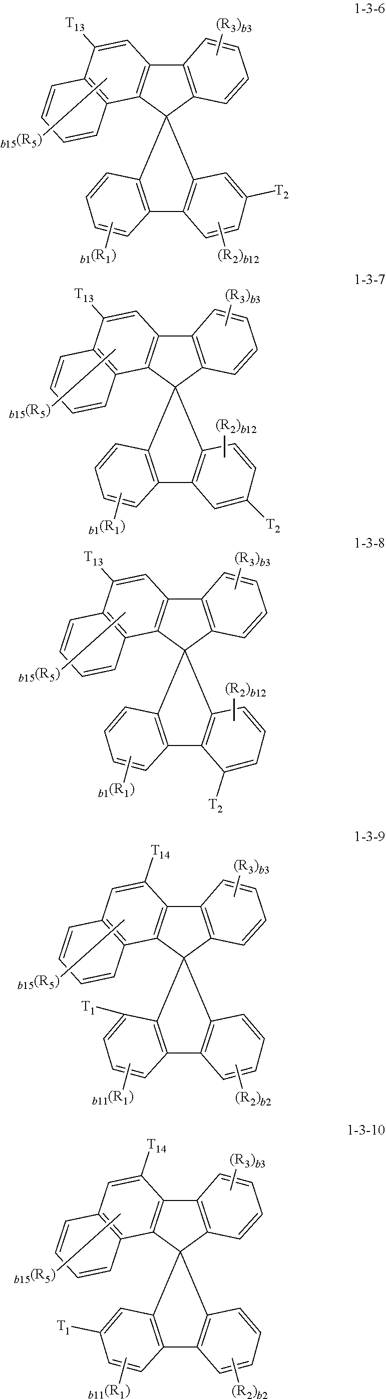

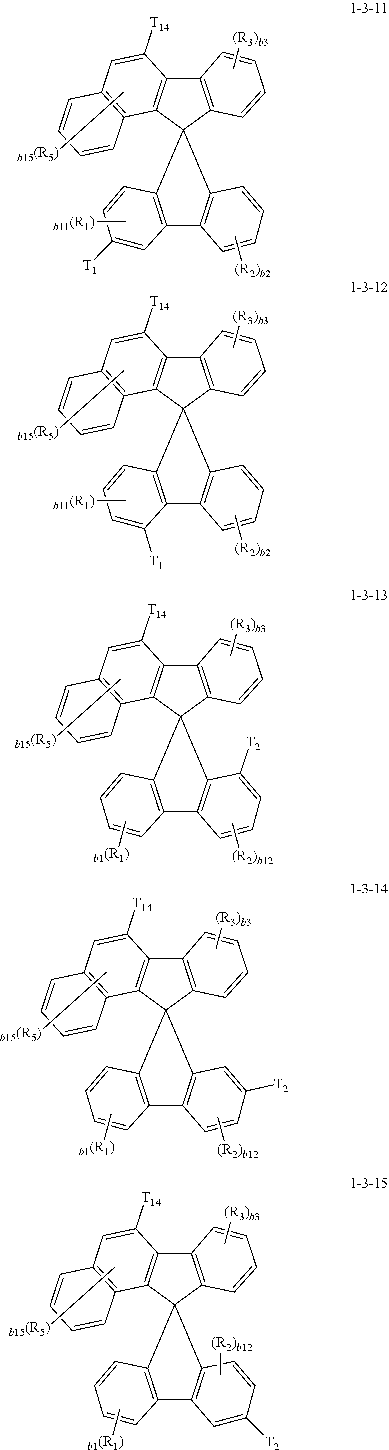

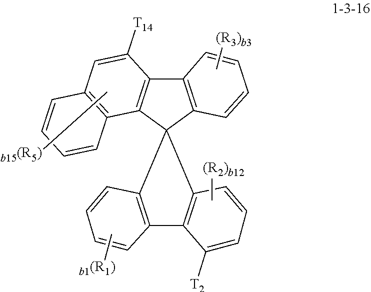

In Formulae 1-1-1 to 1-1-16, 1-2-1 to 1-2-16, and 1-3-1 to 1-3-16,

T₁, T₂, and T₁₁ to T₁₄ may each independently be a group represented by Formula 2,

R₅ may be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),

b1 to b3 may each independently be an integer from 0 to 4,

b11 and b12 may each independently be an integer from 0 to 3,

b15 may be an integer from 0 to 5, and

R₁ to R₃, R₁₀ₐ, and Q₁ to Q₃ are respectively the same as described in the specification.

In an embodiment, the amine-based compound may be one of Compounds 1 to 563:

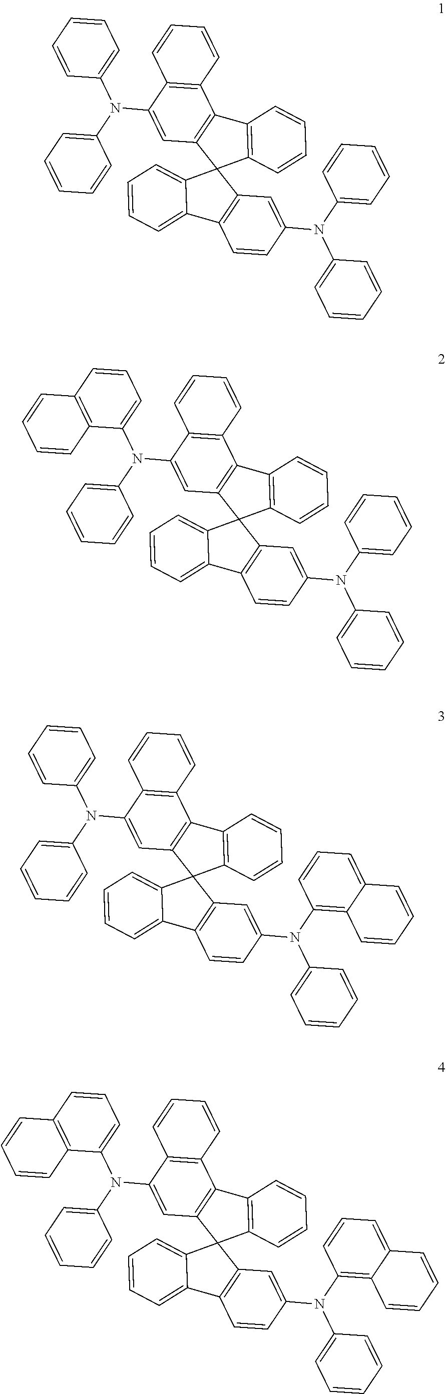 [SMILES: c1ccc(N(c2ccccc2)c2ccc3c(c2)C2(c4ccccc4-3)c3ccccc3-c3c2cc(N(c2ccccc2)c2ccccc2)c2ccccc32)cc1 ; c1ccc(N(c2ccccc2)c2ccc3c(c2)C2(c4ccccc4-3)c3ccccc3-c3c2cc(N(c2ccccc2)c2cccc4ccccc24)c2ccccc32)cc1 ; c1ccc(N(c2ccc3c(c2)C2(c4ccccc4-3)c3ccccc3-c3c2cc(N(c2ccccc2)c2ccccc2)c2ccccc32)c2cccc3ccccc23)cc1 ; c1ccc(N(c2ccc3c(c2)C2(c4ccccc4-3)c3ccccc3-c3c2cc(N(c2ccccc2)c2cccc4ccccc24)c2ccccc32)c2cccc3ccccc23)cc1]

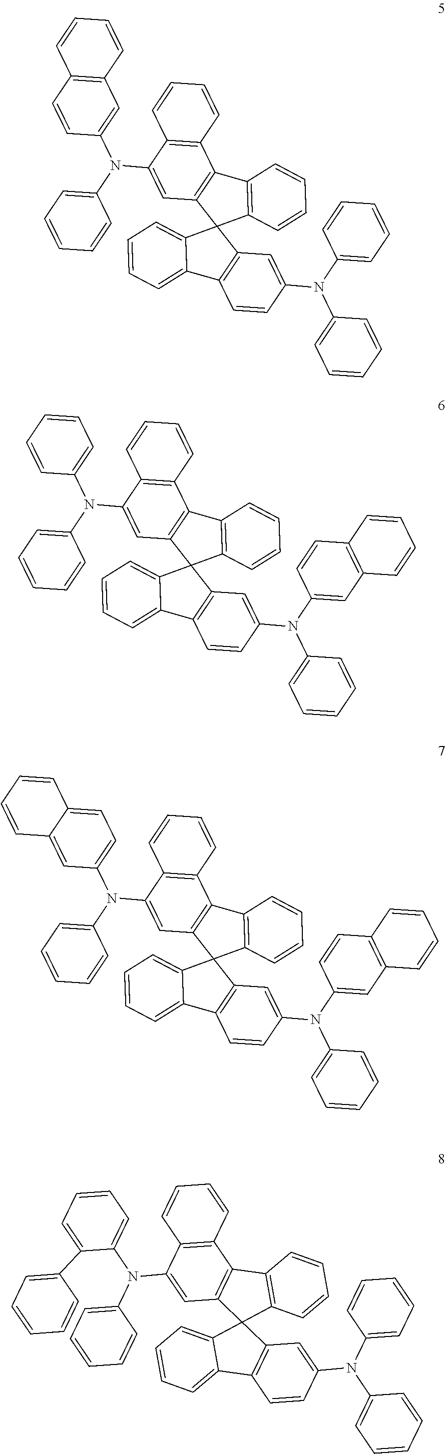 [SMILES: c1ccc(N(c2ccccc2)c2ccc3c(c2)C2(c4ccccc4-3)c3ccccc3-c3c2cc(N(c2ccccc2)c2ccc4ccccc4c2)c2ccccc32)cc1 ; c1ccc(N(c2ccc3c(c2)C2(c4ccccc4-3)c3ccccc3-c3c2cc(N(c2ccccc2)c2ccccc2)c2ccccc32)c2ccc3ccccc3c2)cc1 ; c1ccc(N(c2ccc3c(c2)C2(c4ccccc4-3)c3ccccc3-c3c2cc(N(c2ccccc2)c2ccc4ccccc4c2)c2ccccc32)c2ccc3ccccc3c2)cc1 ; c1ccc(-c2ccccc2N(c2ccccc2)c2cc3c(c4ccccc24)-c2ccccc2C32c3ccccc3-c3ccc(N(c4ccccc4)c4ccccc4)cc32)cc1]

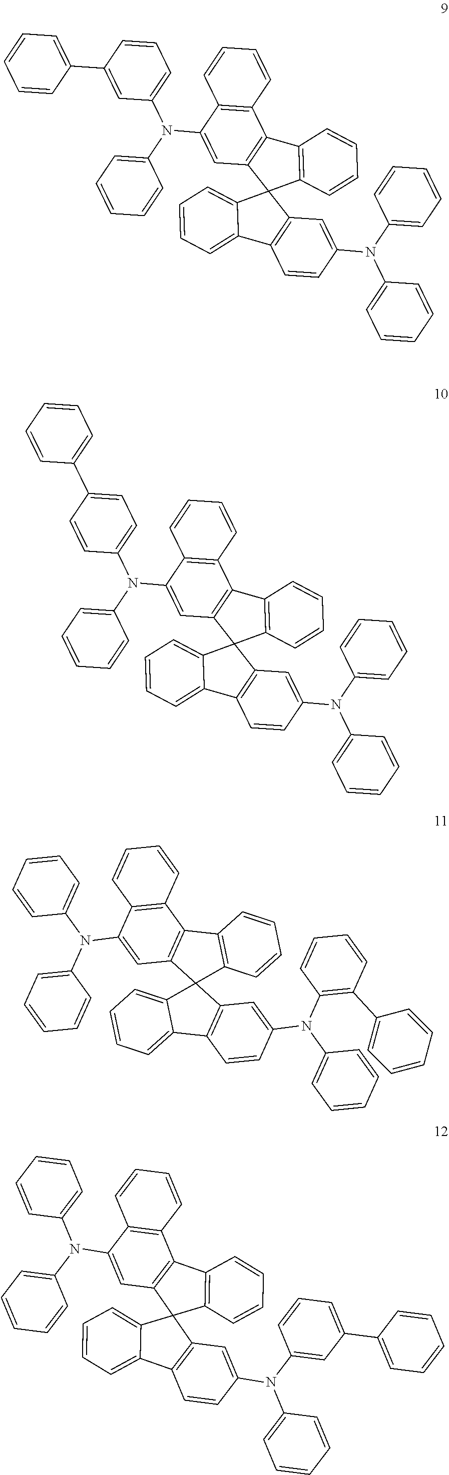 [SMILES: c1ccc(-c2cccc(N(c3ccccc3)c3cc4c(c5ccccc35)-c3ccccc3C43c4ccccc4-c4ccc(N(c5ccccc5)c5ccccc5)cc43)c2)cc1 ; c1ccc(-c2ccc(N(c3ccccc3)c3cc4c(c5ccccc35)-c3ccccc3C43c4ccccc4-c4ccc(N(c5ccccc5)c5ccccc5)cc43)cc2)cc1 ; c1ccc(-c2ccccc2N(c2ccccc2)c2ccc3c(c2)C2(c4ccccc4-3)c3ccccc3-c3c2cc(N(c2ccccc2)c2ccccc2)c2ccccc32)cc1 ; c1ccc(-c2cccc(N(c3ccccc3)c3ccc4c(c3)C3(c5ccccc5-4)c4ccccc4-c4c3cc(N(c3ccccc3)c3ccccc3)c3ccccc43)c2)cc1]

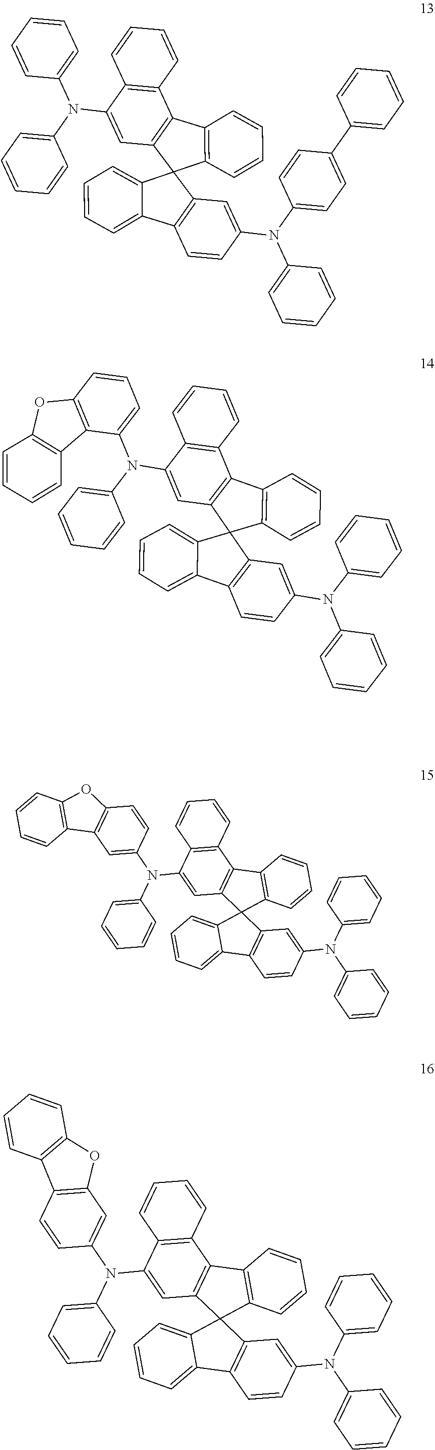 [SMILES: c1ccc(-c2ccc(N(c3ccccc3)c3ccc4c(c3)C3(c5ccccc5-4)c4ccccc4-c4c3cc(N(c3ccccc3)c3ccccc3)c3ccccc43)cc2)cc1 ; c1ccc(N(c2ccccc2)c2ccc3c(c2)C2(c4ccccc4-3)c3ccccc3-c3c2cc(N(c2ccccc2)c2cccc4oc5ccccc5c24)c2ccccc32)cc1 ; c1ccc(N(c2ccccc2)c2ccc3c(c2)C2(c4ccccc4-3)c3ccccc3-c3c2cc(N(c2ccccc2)c2ccc4oc5ccccc5c4c2)c2ccccc32)cc1 ; c1ccc(N(c2ccccc2)c2ccc3c(c2)C2(c4ccccc4-3)c3ccccc3-c3c2cc(N(c2ccccc2)c2ccc4c(c2)oc2ccccc24)c2ccccc32)cc1]

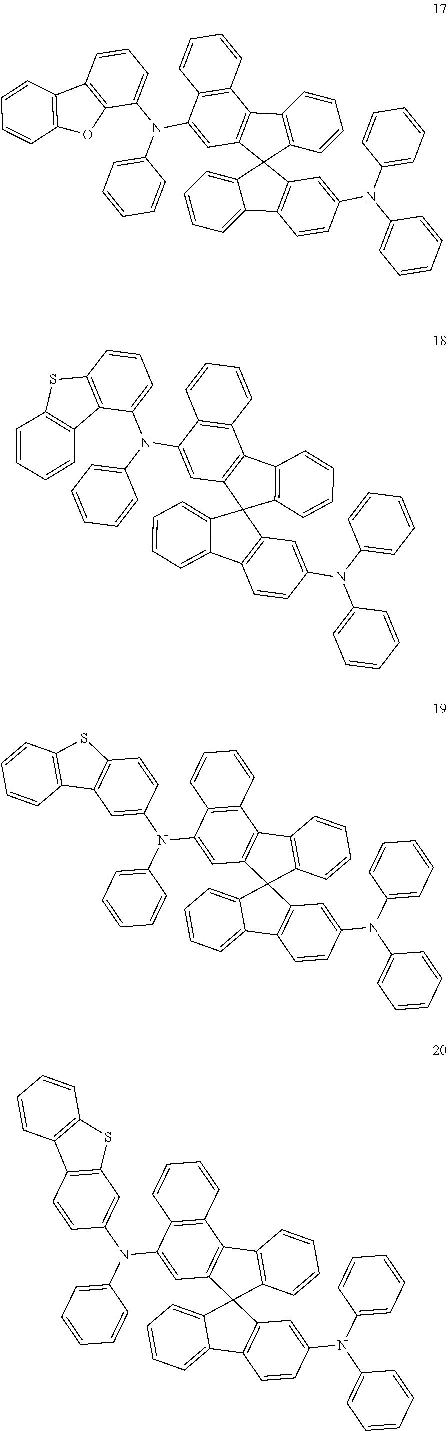 [SMILES: c1ccc(N(c2ccccc2)c2ccc3c(c2)C2(c4ccccc4-3)c3ccccc3-c3c2cc(N(c2ccccc2)c2cccc4c2oc2ccccc24)c2ccccc32)cc1 ; c1ccc(N(c2ccccc2)c2ccc3c(c2)C2(c4ccccc4-3)c3ccccc3-c3c2cc(N(c2ccccc2)c2cccc4sc5ccccc5c24)c2ccccc32)cc1 ; c1ccc(N(c2ccccc2)c2ccc3c(c2)C2(c4ccccc4-3)c3ccccc3-c3c2cc(N(c2ccccc2)c2ccc4sc5ccccc5c4c2)c2ccccc32)cc1 ; c1ccc(N(c2ccccc2)c2ccc3c(c2)C2(c4ccccc4-3)c3ccccc3-c3c2cc(N(c2ccccc2)c2ccc4c(c2)sc2ccccc24)c2ccccc32)cc1]

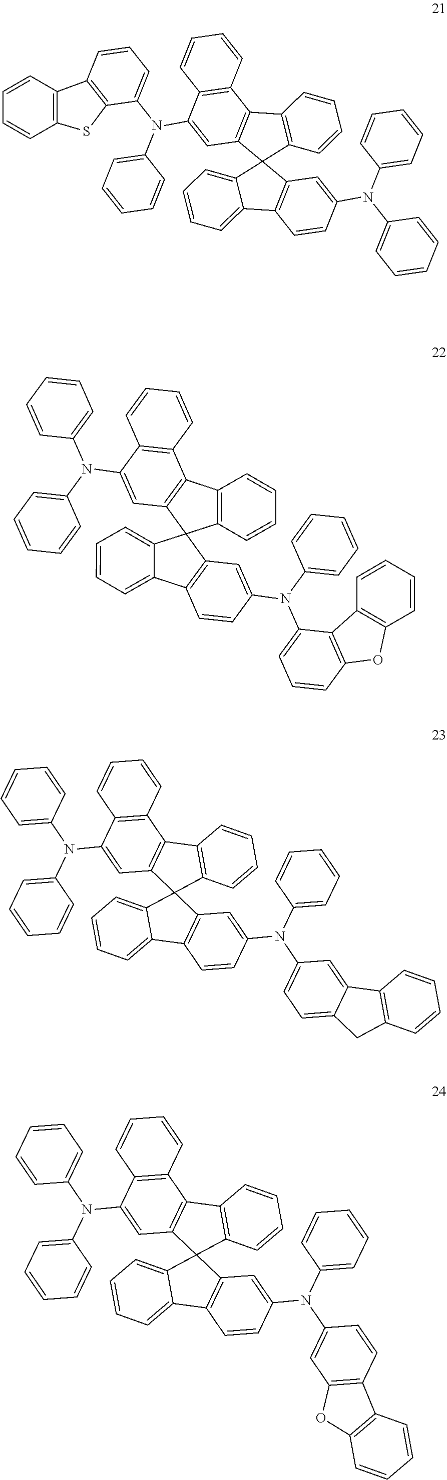 [SMILES: c1ccc(N(c2ccccc2)c2ccc3c(c2)C2(c4ccccc4-3)c3ccccc3-c3c2cc(N(c2ccccc2)c2cccc4c2sc2ccccc24)c2ccccc32)cc1 ; c1ccc(N(c2ccccc2)c2cc3c(c4ccccc24)-c2ccccc2C32c3ccccc3-c3ccc(N(c4ccccc4)c4cccc5oc6ccccc6c45)cc32)cc1 ; c1ccc(N(c2ccc3c(c2)-c2ccccc2C3)c2ccc3c(c2)C2(c4ccccc4-3)c3ccccc3-c3c2cc(N(c2ccccc2)c2ccccc2)c2ccccc32)cc1 ; c1ccc(N(c2ccc3c(c2)C2(c4ccccc4-3)c3ccccc3-c3c2cc(N(c2ccccc2)c2ccccc2)c2ccccc32)c2ccc3c(c2)oc2ccccc23)cc1]

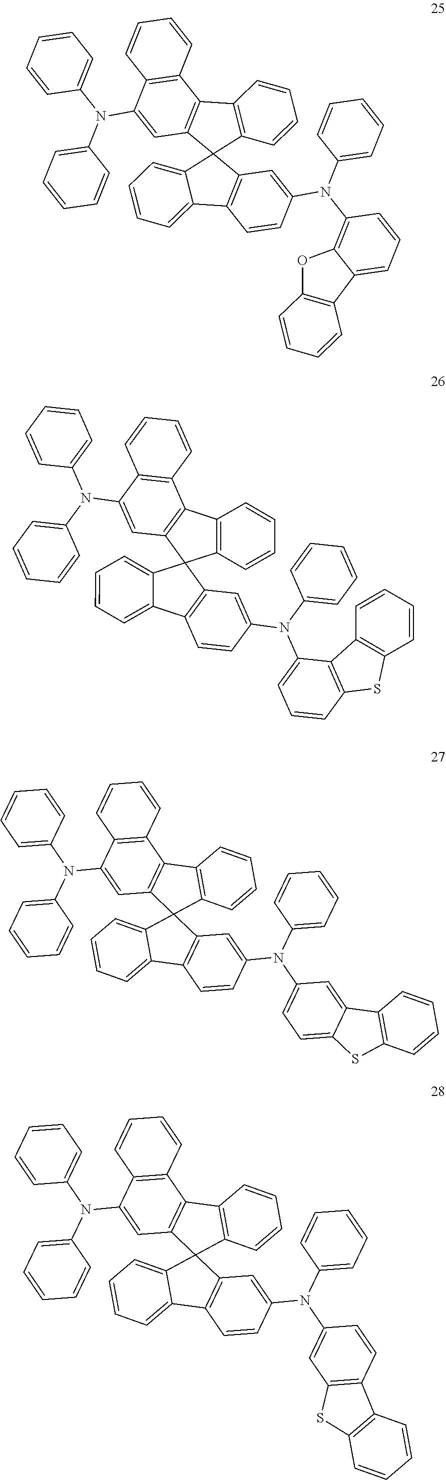 [SMILES: c1ccc(N(c2ccccc2)c2cc3c(c4ccccc24)-c2ccccc2C32c3ccccc3-c3ccc(N(c4ccccc4)c4cccc5c4oc4ccccc45)cc32)cc1 ; c1ccc(N(c2ccccc2)c2cc3c(c4ccccc24)-c2ccccc2C32c3ccccc3-c3ccc(N(c4ccccc4)c4cccc5sc6ccccc6c45)cc32)cc1 ; c1ccc(N(c2ccc3c(c2)C2(c4ccccc4-3)c3ccccc3-c3c2cc(N(c2ccccc2)c2ccccc2)c2ccccc32)c2ccc3sc4ccccc4c3c2)cc1 ; c1ccc(N(c2ccc3c(c2)C2(c4ccccc4-3)c3ccccc3-c3c2cc(N(c2ccccc2)c2ccccc2)c2ccccc32)c2ccc3c(c2)sc2ccccc23)cc1]

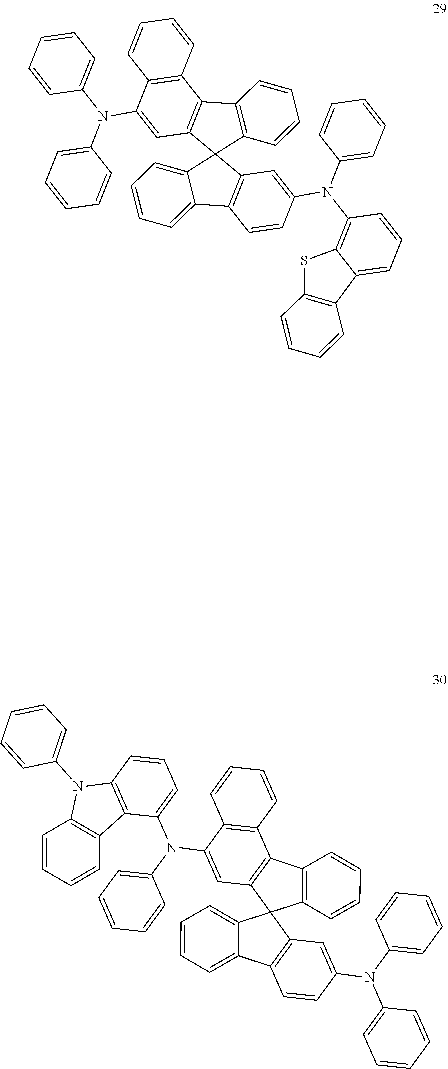 [SMILES: c1ccc(N(c2ccccc2)c2cc3c(c4ccccc24)-c2ccccc2C32c3ccccc3-c3ccc(N(c4ccccc4)c4cccc5c4sc4ccccc45)cc32)cc1 ; c1ccc(N(c2ccccc2)c2ccc3c(c2)C2(c4ccccc4-3)c3ccccc3-c3c2cc(N(c2ccccc2)c2cccc4c2c2ccccc2n4-c2ccccc2)c2ccccc32)cc1]

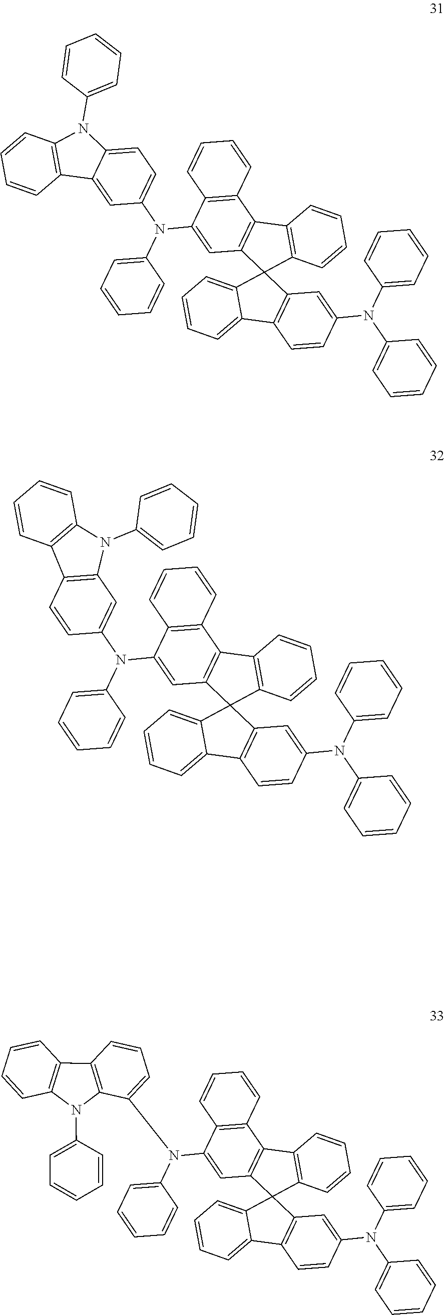 [SMILES: c1ccc(N(c2ccccc2)c2ccc3c(c2)C2(c4ccccc4-3)c3ccccc3-c3c2cc(N(c2ccccc2)c2ccc4c(c2)c2ccccc2n4-c2ccccc2)c2ccccc32)cc1 ; c1ccc(N(c2ccccc2)c2ccc3c(c2)C2(c4ccccc4-3)c3ccccc3-c3c2cc(N(c2ccccc2)c2ccc4c5ccccc5n(-c5ccccc5)c4c2)c2ccccc32)cc1 ; c1ccc(N(c2ccccc2)c2ccc3c(c2)C2(c4ccccc4-3)c3ccccc3-c3c2cc(N(c2ccccc2)c2cccc4c5ccccc5n(-c5ccccc5)c24)c2ccccc32)cc1]

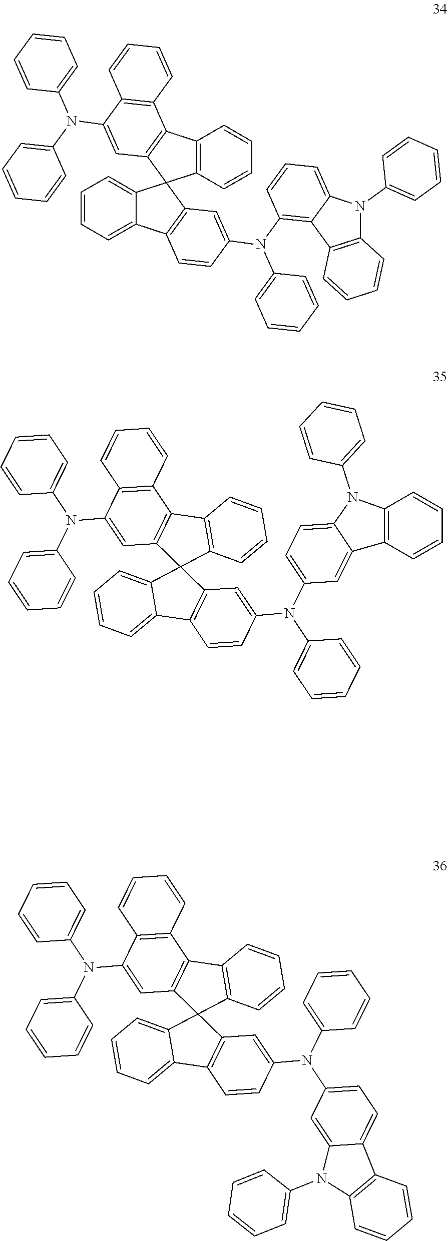 [SMILES: c1ccc(N(c2ccccc2)c2cc3c(c4ccccc24)-c2ccccc2C32c3ccccc3-c3ccc(N(c4ccccc4)c4cccc5c4c4ccccc4n5-c4ccccc4)cc32)cc1 ; c1ccc(N(c2ccc3c(c2)C2(c4ccccc4-3)c3ccccc3-c3c2cc(N(c2ccccc2)c2ccccc2)c2ccccc32)c2ccc3c(c2)c2ccccc2n3-c2ccccc2)cc1 ; c1ccc(N(c2ccc3c(c2)C2(c4ccccc4-3)c3ccccc3-c3c2cc(N(c2ccccc2)c2ccccc2)c2ccccc32)c2ccc3c4ccccc4n(-c4ccccc4)c3c2)cc1]

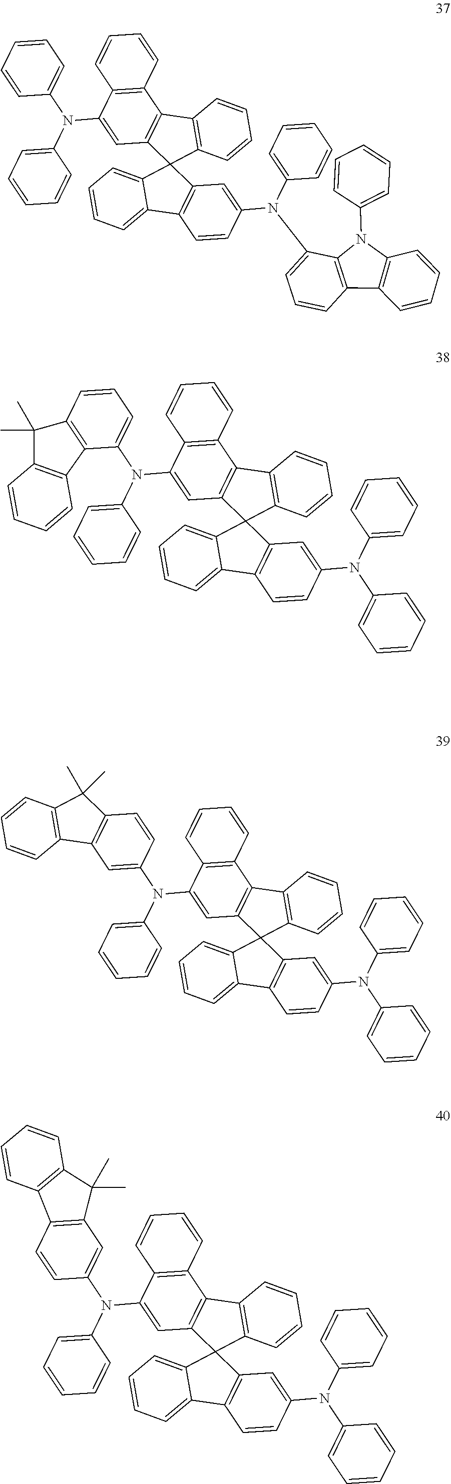 [SMILES: c1ccc(N(c2ccccc2)c2cc3c(c4ccccc24)-c2ccccc2C32c3ccccc3-c3ccc(N(c4ccccc4)c4cccc5c6ccccc6n(-c6ccccc6)c45)cc32)cc1 ; CC1(C)c2ccccc2-c2c(N(c3ccccc3)c3cc4c(c5ccccc35)-c3ccccc3C43c4ccccc4-c4ccc(N(c5ccccc5)c5ccccc5)cc43)cccc21 ; CC1(C)c2ccccc2-c2cc(N(c3ccccc3)c3cc4c(c5ccccc35)-c3ccccc3C43c4ccccc4-c4ccc(N(c5ccccc5)c5ccccc5)cc43)ccc21 ; CC1(C)c2ccccc2-c2ccc(N(c3ccccc3)c3cc4c(c5ccccc35)-c3ccccc3C43c4ccccc4-c4ccc(N(c5ccccc5)c5ccccc5)cc43)cc21]

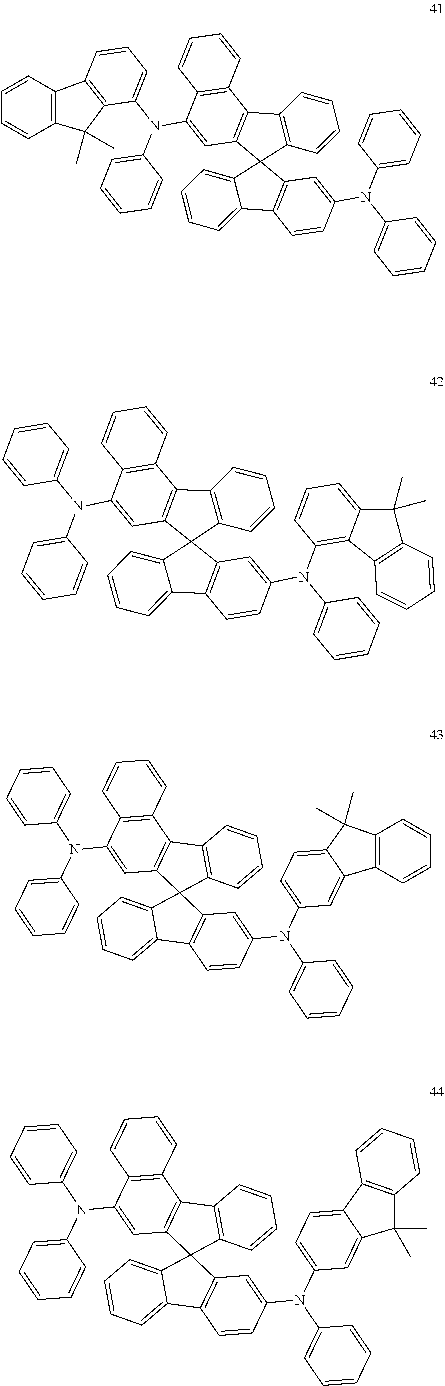 [SMILES: CC1(C)c2ccccc2-c2cccc(N(c3ccccc3)c3cc4c(c5ccccc35)-c3ccccc3C43c4ccccc4-c4ccc(N(c5ccccc5)c5ccccc5)cc43)c21 ; CC1(C)c2ccccc2-c2c(N(c3ccccc3)c3ccc4c(c3)C3(c5ccccc5-4)c4ccccc4-c4c3cc(N(c3ccccc3)c3ccccc3)c3ccccc43)cccc21 ; CC1(C)c2ccccc2-c2cc(N(c3ccccc3)c3ccc4c(c3)C3(c5ccccc5-4)c4ccccc4-c4c3cc(N(c3ccccc3)c3ccccc3)c3ccccc43)ccc21 ; CC1(C)c2ccccc2-c2ccc(N(c3ccccc3)c3ccc4c(c3)C3(c5ccccc5-4)c4ccccc4-c4c3cc(N(c3ccccc3)c3ccccc3)c3ccccc43)cc21]

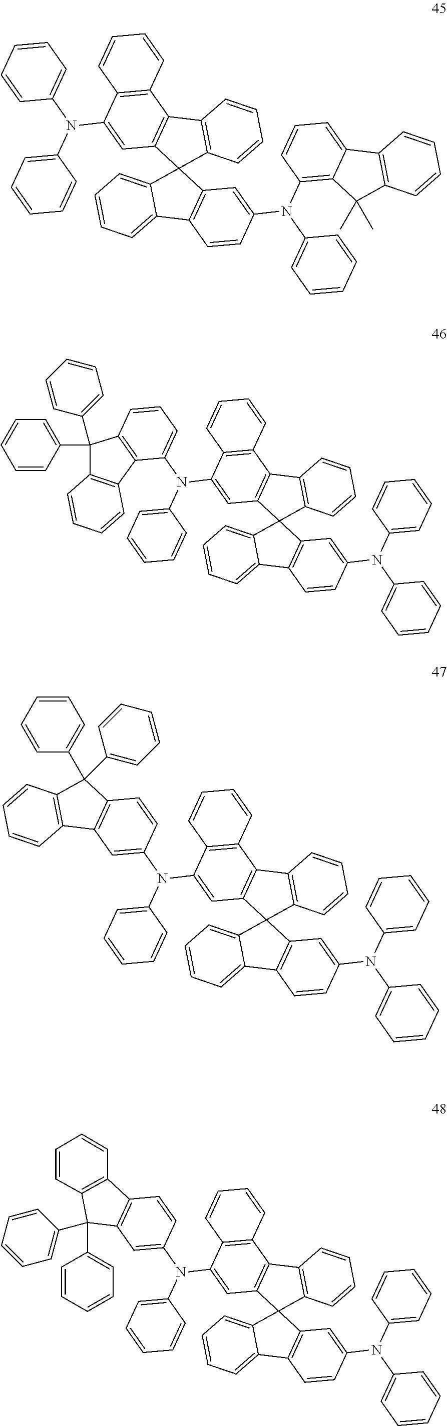 [SMILES: CC1(C)c2ccccc2-c2cccc(N(c3ccccc3)c3ccc4c(c3)C3(c5ccccc5-4)c4ccccc4-c4c3cc(N(c3ccccc3)c3ccccc3)c3ccccc43)c21 ; c1ccc(N(c2ccccc2)c2ccc3c(c2)C2(c4ccccc4-3)c3ccccc3-c3c2cc(N(c2ccccc2)c2cccc4c2-c2ccccc2C4(c2ccccc2)c2ccccc2)c2ccccc32)cc1 ; c1ccc(N(c2ccccc2)c2ccc3c(c2)C2(c4ccccc4-3)c3ccccc3-c3c2cc(N(c2ccccc2)c2ccc4c(c2)-c2ccccc2C4(c2ccccc2)c2ccccc2)c2ccccc32)cc1 ; c1ccc(N(c2ccccc2)c2ccc3c(c2)C2(c4ccccc4-3)c3ccccc3-c3c2cc(N(c2ccccc2)c2ccc4c(c2)C(c2ccccc2)(c2ccccc2)c2ccccc2-4)c2ccccc32)cc1]

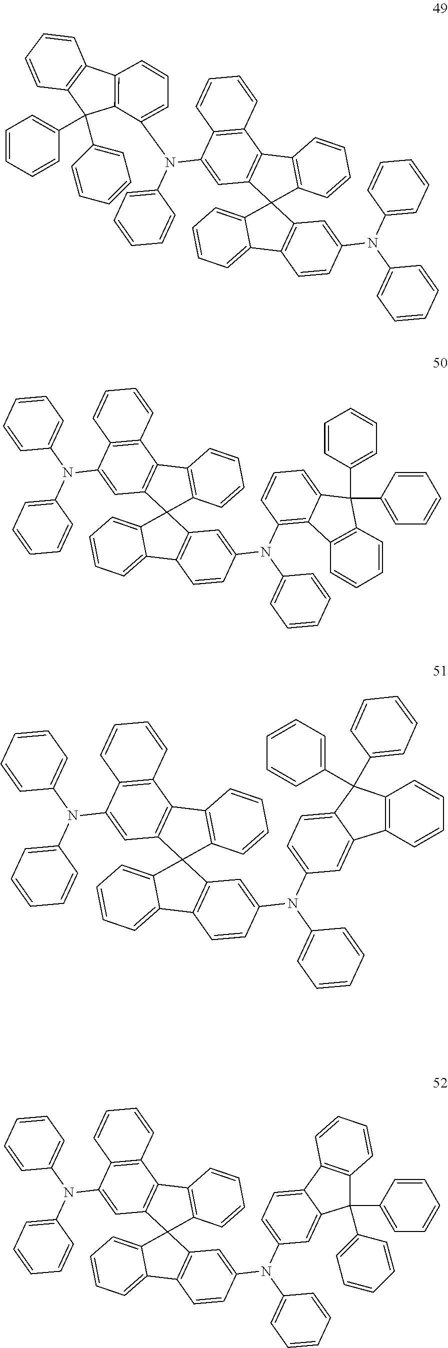

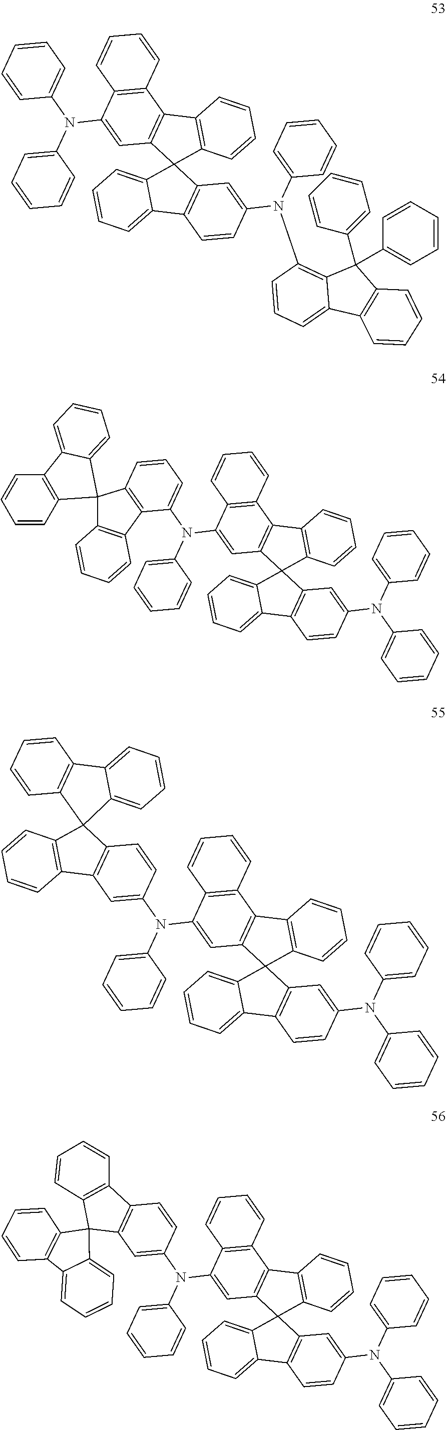

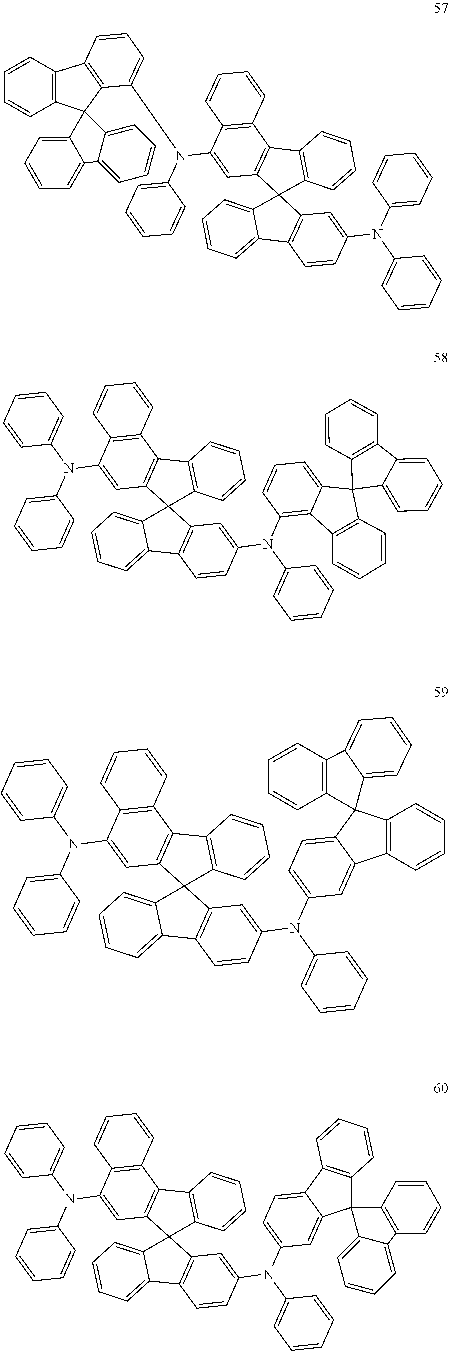

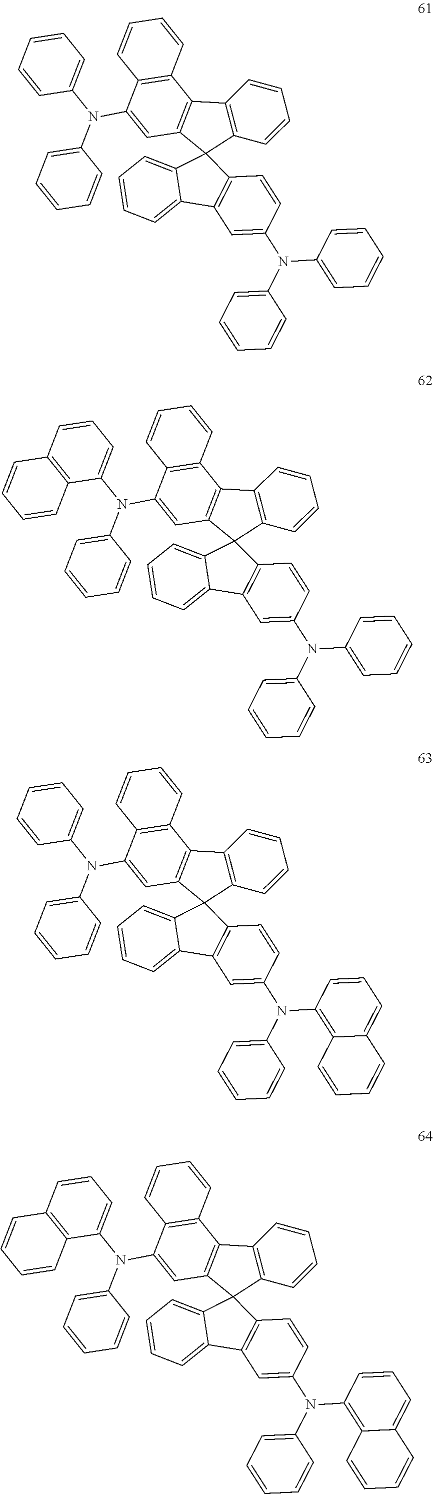 [SMILES: c1ccc(N(c2ccccc2)c2ccc3c(c2)-c2ccccc2C32c3ccccc3-c3c2cc(N(c2ccccc2)c2ccccc2)c2ccccc32)cc1 ; c1ccc(N(c2ccccc2)c2ccc3c(c2)-c2ccccc2C32c3ccccc3-c3c2cc(N(c2ccccc2)c2cccc4ccccc24)c2ccccc32)cc1 ; c1ccc(N(c2ccc3c(c2)-c2ccccc2C32c3ccccc3-c3c2cc(N(c2ccccc2)c2ccccc2)c2ccccc32)c2cccc3ccccc23)cc1 ; c1ccc(N(c2ccc3c(c2)-c2ccccc2C32c3ccccc3-c3c2cc(N(c2ccccc2)c2cccc4ccccc24)c2ccccc32)c2cccc3ccccc23)cc1]

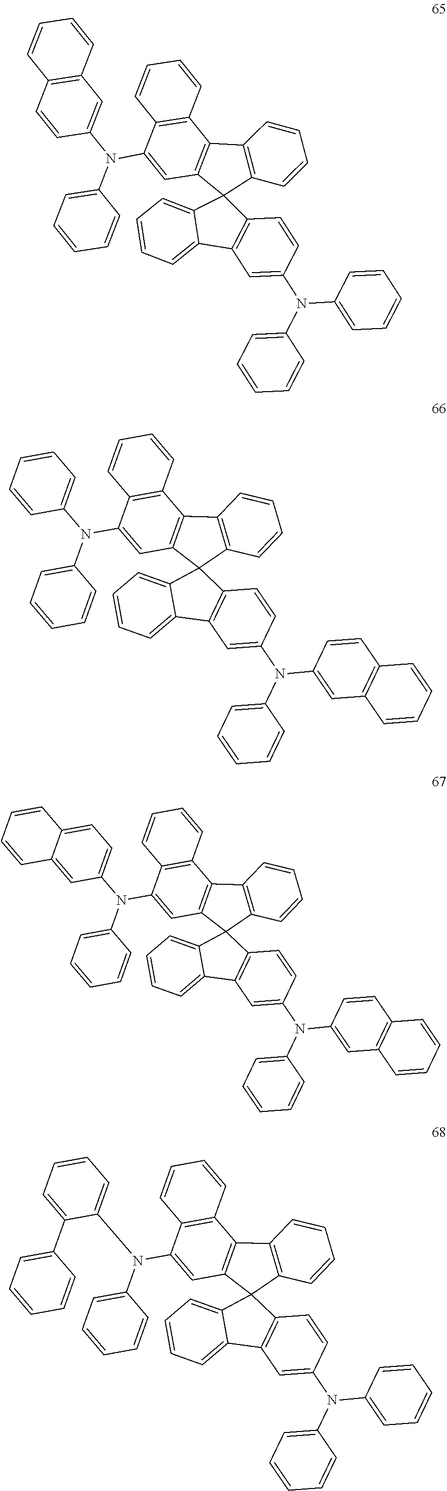 [SMILES: c1ccc(N(c2ccccc2)c2ccc3c(c2)-c2ccccc2C32c3ccccc3-c3c2cc(N(c2ccccc2)c2ccc4ccccc4c2)c2ccccc32)cc1 ; c1ccc(N(c2ccc3c(c2)-c2ccccc2C32c3ccccc3-c3c2cc(N(c2ccccc2)c2ccccc2)c2ccccc32)c2ccc3ccccc3c2)cc1 ; c1ccc(N(c2ccc3c(c2)-c2ccccc2C32c3ccccc3-c3c2cc(N(c2ccccc2)c2ccc4ccccc4c2)c2ccccc32)c2ccc3ccccc3c2)cc1 ; c1ccc(-c2ccccc2N(c2ccccc2)c2cc3c(c4ccccc24)-c2ccccc2C32c3ccccc3-c3cc(N(c4ccccc4)c4ccccc4)ccc32)cc1]

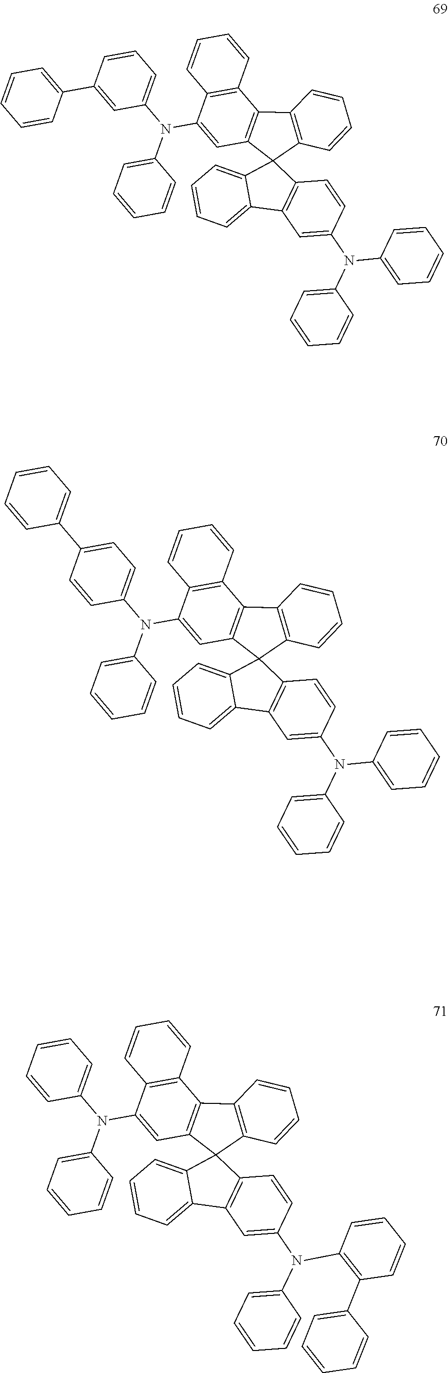 [SMILES: c1ccc(-c2cccc(N(c3ccccc3)c3cc4c(c5ccccc35)-c3ccccc3C43c4ccccc4-c4cc(N(c5ccccc5)c5ccccc5)ccc43)c2)cc1 ; c1ccc(-c2ccc(N(c3ccccc3)c3cc4c(c5ccccc35)-c3ccccc3C43c4ccccc4-c4cc(N(c5ccccc5)c5ccccc5)ccc43)cc2)cc1 ; c1ccc(-c2ccccc2N(c2ccccc2)c2ccc3c(c2)-c2ccccc2C32c3ccccc3-c3c2cc(N(c2ccccc2)c2ccccc2)c2ccccc32)cc1]

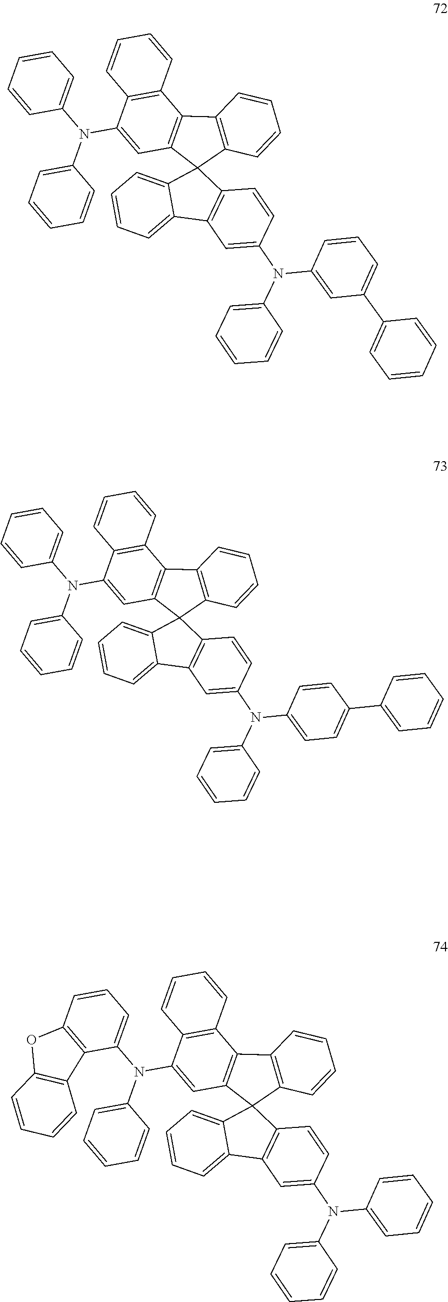 [SMILES: c1ccc(-c2cccc(N(c3ccccc3)c3ccc4c(c3)-c3ccccc3C43c4ccccc4-c4c3cc(N(c3ccccc3)c3ccccc3)c3ccccc43)c2)cc1 ; c1ccc(-c2ccc(N(c3ccccc3)c3ccc4c(c3)-c3ccccc3C43c4ccccc4-c4c3cc(N(c3ccccc3)c3ccccc3)c3ccccc43)cc2)cc1 ; c1ccc(N(c2ccccc2)c2ccc3c(c2)-c2ccccc2C32c3ccccc3-c3c2cc(N(c2ccccc2)c2cccc4oc5ccccc5c24)c2ccccc32)cc1]

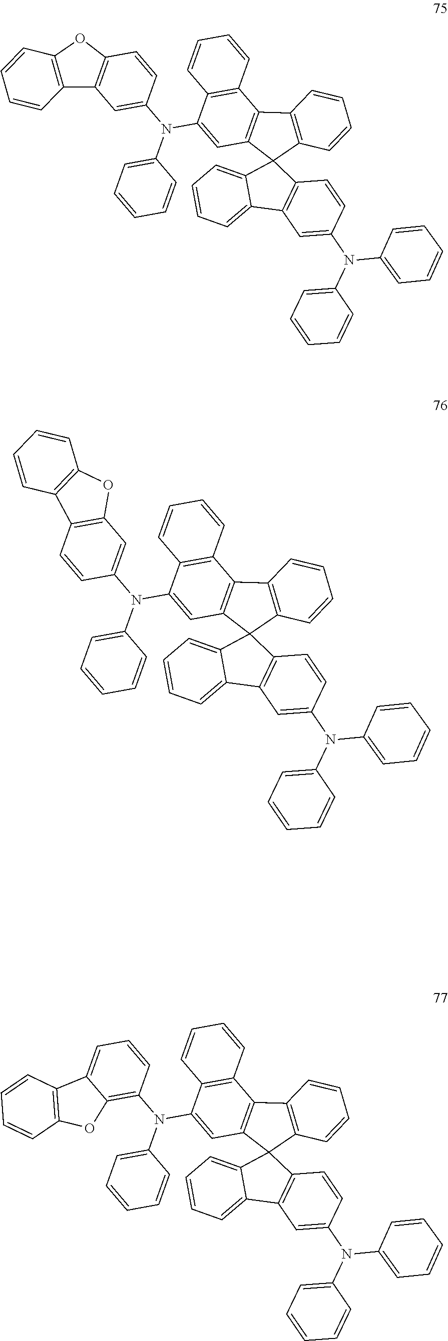 [SMILES: c1ccc(N(c2ccccc2)c2ccc3c(c2)-c2ccccc2C32c3ccccc3-c3c2cc(N(c2ccccc2)c2ccc4oc5ccccc5c4c2)c2ccccc32)cc1 ; c1ccc(N(c2ccccc2)c2ccc3c(c2)-c2ccccc2C32c3ccccc3-c3c2cc(N(c2ccccc2)c2ccc4c(c2)oc2ccccc24)c2ccccc32)cc1 ; c1ccc(N(c2ccccc2)c2ccc3c(c2)-c2ccccc2C32c3ccccc3-c3c2cc(N(c2ccccc2)c2cccc4c2oc2ccccc24)c2ccccc32)cc1]

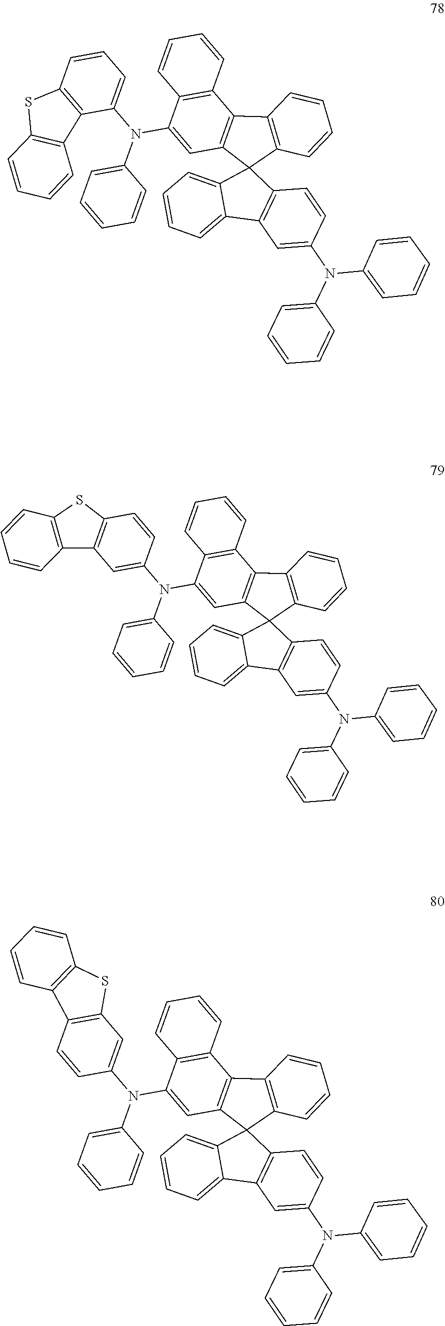 [SMILES: c1ccc(N(c2ccccc2)c2ccc3c(c2)-c2ccccc2C32c3ccccc3-c3c2cc(N(c2ccccc2)c2cccc4sc5ccccc5c24)c2ccccc32)cc1 ; c1ccc(N(c2ccccc2)c2ccc3c(c2)-c2ccccc2C32c3ccccc3-c3c2cc(N(c2ccccc2)c2ccc4sc5ccccc5c4c2)c2ccccc32)cc1 ; c1ccc(N(c2ccccc2)c2ccc3c(c2)-c2ccccc2C32c3ccccc3-c3c2cc(N(c2ccccc2)c2ccc4c(c2)sc2ccccc24)c2ccccc32)cc1]

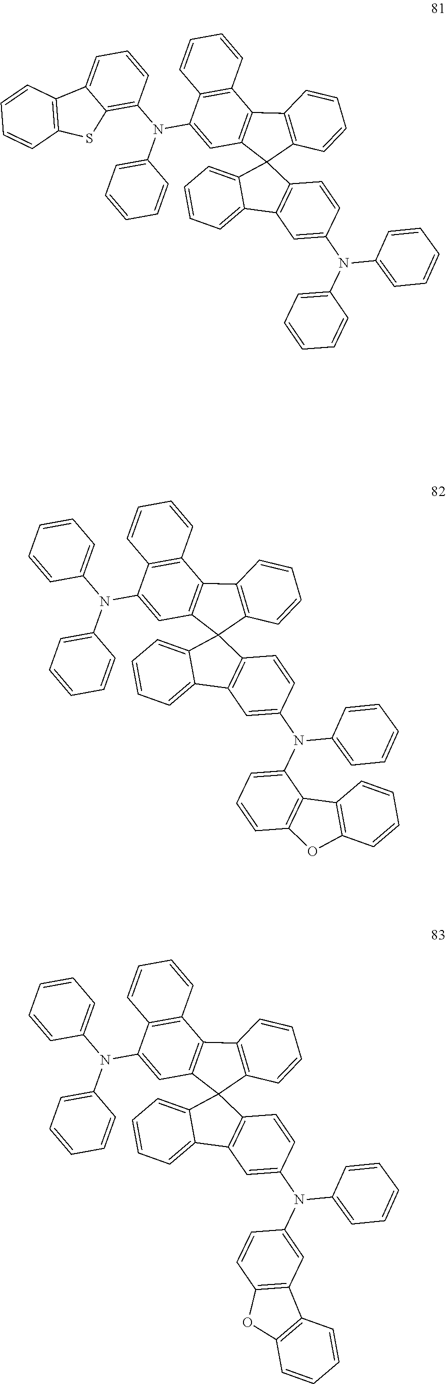 [SMILES: c1ccc(N(c2ccccc2)c2ccc3c(c2)-c2ccccc2C32c3ccccc3-c3c2cc(N(c2ccccc2)c2cccc4c2sc2ccccc24)c2ccccc32)cc1 ; c1ccc(N(c2ccccc2)c2cc3c(c4ccccc24)-c2ccccc2C32c3ccccc3-c3cc(N(c4ccccc4)c4cccc5oc6ccccc6c45)ccc32)cc1 ; c1ccc(N(c2ccc3c(c2)-c2ccccc2C32c3ccccc3-c3c2cc(N(c2ccccc2)c2ccccc2)c2ccccc32)c2ccc3oc4ccccc4c3c2)cc1]

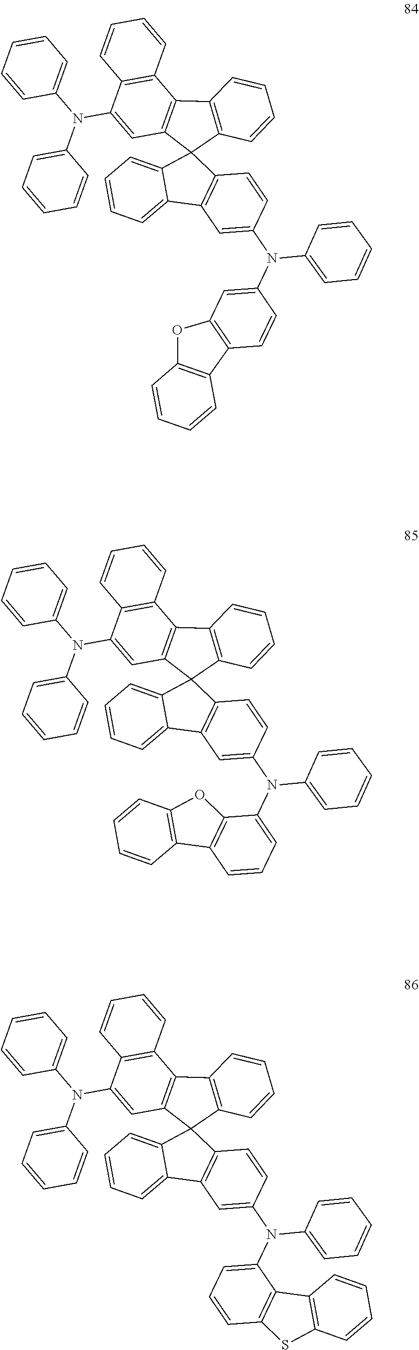 [SMILES: c1ccc(N(c2ccc3c(c2)-c2ccccc2C32c3ccccc3-c3c2cc(N(c2ccccc2)c2ccccc2)c2ccccc32)c2ccc3c(c2)oc2ccccc23)cc1 ; c1ccc(N(c2ccccc2)c2cc3c(c4ccccc24)-c2ccccc2C32c3ccccc3-c3cc(N(c4ccccc4)c4cccc5c4oc4ccccc45)ccc32)cc1 ; c1ccc(N(c2ccccc2)c2cc3c(c4ccccc24)-c2ccccc2C32c3ccccc3-c3cc(N(c4ccccc4)c4cccc5sc6ccccc6c45)ccc32)cc1]

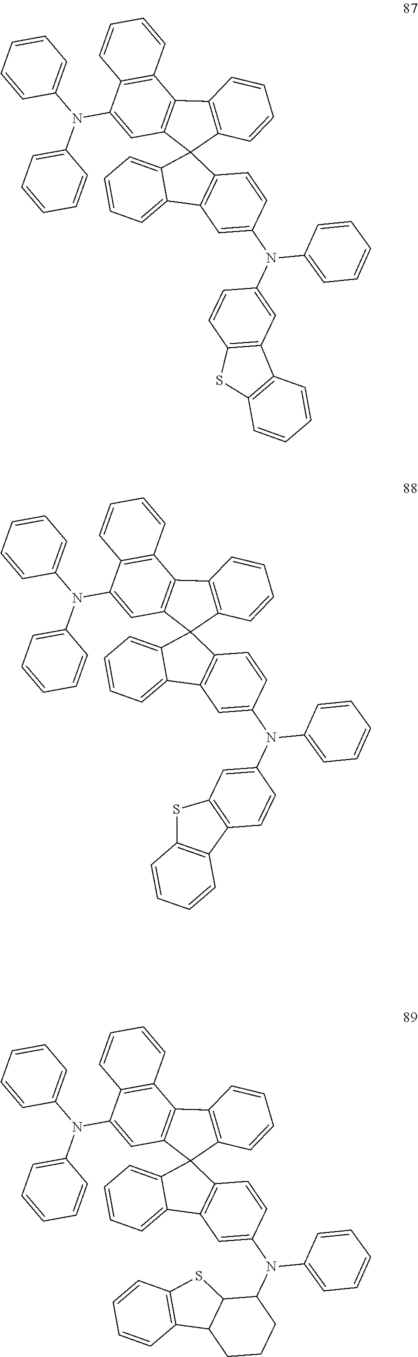 [SMILES: c1ccc(N(c2ccc3c(c2)-c2ccccc2C32c3ccccc3-c3c2cc(N(c2ccccc2)c2ccccc2)c2ccccc32)c2ccc3sc4ccccc4c3c2)cc1 ; c1ccc(N(c2ccc3c(c2)-c2ccccc2C32c3ccccc3-c3c2cc(N(c2ccccc2)c2ccccc2)c2ccccc32)c2ccc3c(c2)sc2ccccc23)cc1 ; c1ccc(N(c2ccccc2)c2cc3c(c4ccccc24)-c2ccccc2C32c3ccccc3-c3cc(N(c4ccccc4)C4CCCC5c6ccccc6SC54)ccc32)cc1]

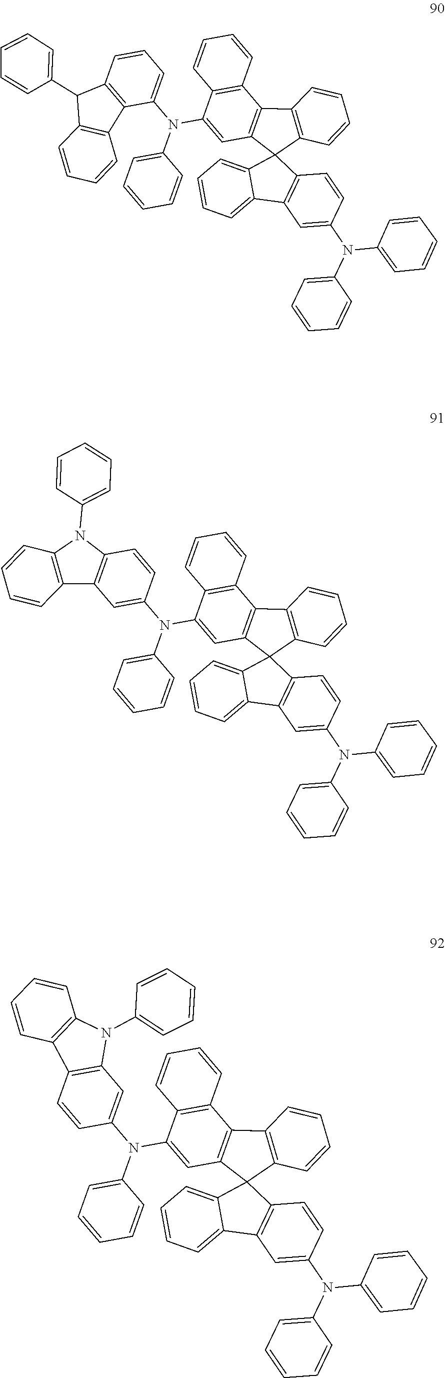 [SMILES: c1ccc(C2c3ccccc3-c3c2cccc3N(c2ccccc2)c2cc3c(c4ccccc24)-c2ccccc2C32c3ccccc3-c3cc(N(c4ccccc4)c4ccccc4)ccc32)cc1 ; c1ccc(N(c2ccccc2)c2ccc3c(c2)-c2ccccc2C32c3ccccc3-c3c2cc(N(c2ccccc2)c2ccc4c(c2)c2ccccc2n4-c2ccccc2)c2ccccc32)cc1 ; c1ccc(N(c2ccccc2)c2ccc3c(c2)-c2ccccc2C32c3ccccc3-c3c2cc(N(c2ccccc2)c2ccc4c5ccccc5n(-c5ccccc5)c4c2)c2ccccc32)cc1]

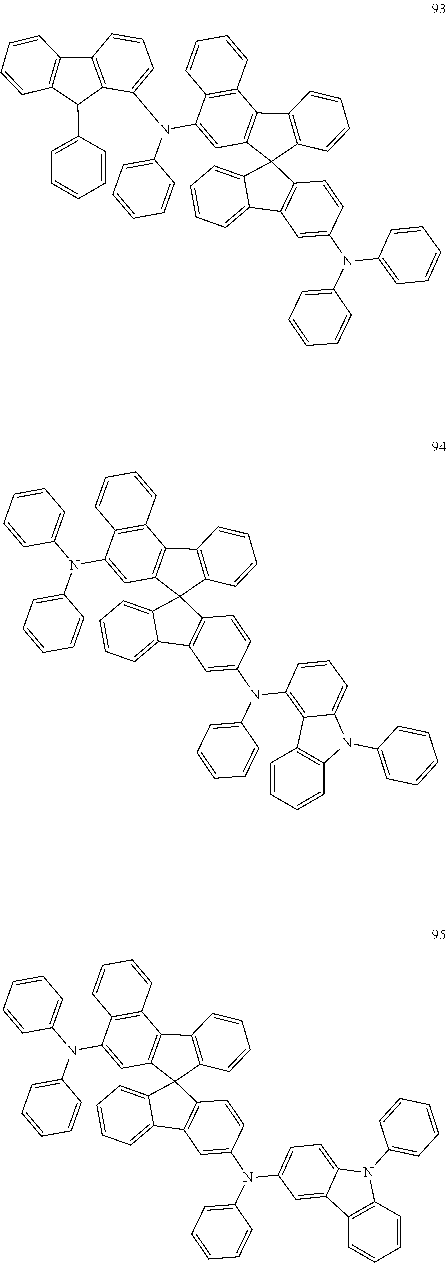 [SMILES: c1ccc(C2c3ccccc3-c3cccc(N(c4ccccc4)c4cc5c(c6ccccc46)-c4ccccc4C54c5ccccc5-c5cc(N(c6ccccc6)c6ccccc6)ccc54)c32)cc1 ; c1ccc(N(c2ccccc2)c2cc3c(c4ccccc24)-c2ccccc2C32c3ccccc3-c3cc(N(c4ccccc4)c4cccc5c4c4ccccc4n5-c4ccccc4)ccc32)cc1 ; c1ccc(N(c2ccc3c(c2)-c2ccccc2C32c3ccccc3-c3c2cc(N(c2ccccc2)c2ccccc2)c2ccccc32)c2ccc3c(c2)c2ccccc2n3-c2ccccc2)cc1]

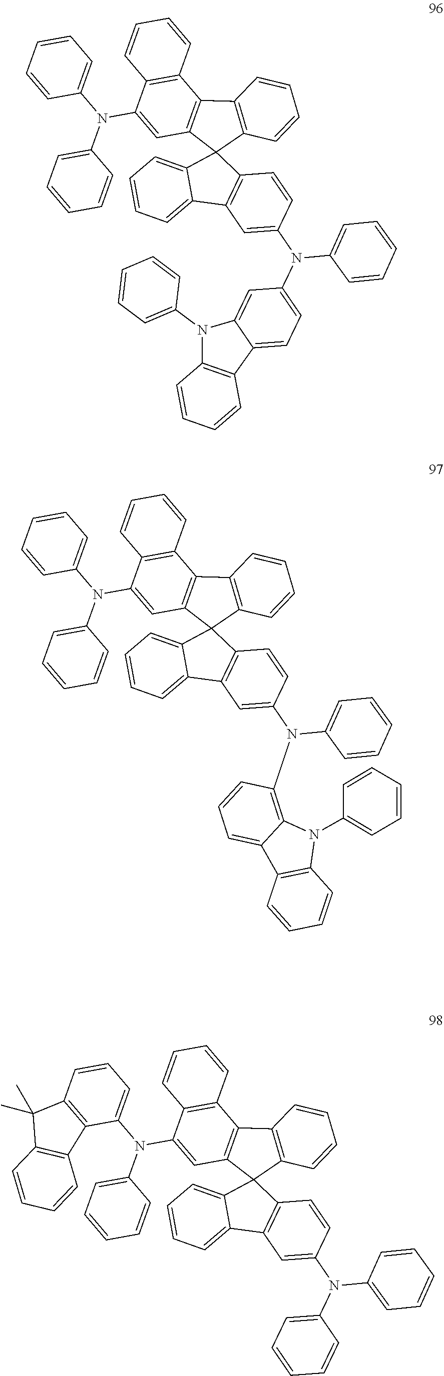 [SMILES: c1ccc(N(c2ccc3c(c2)-c2ccccc2C32c3ccccc3-c3c2cc(N(c2ccccc2)c2ccccc2)c2ccccc32)c2ccc3c4ccccc4n(-c4ccccc4)c3c2)cc1 ; c1ccc(N(c2ccccc2)c2cc3c(c4ccccc24)-c2ccccc2C32c3ccccc3-c3cc(N(c4ccccc4)c4cccc5c6ccccc6n(-c6ccccc6)c45)ccc32)cc1 ; CC1(C)c2ccccc2-c2c(N(c3ccccc3)c3cc4c(c5ccccc35)-c3ccccc3C43c4ccccc4-c4cc(N(c5ccccc5)c5ccccc5)ccc43)cccc21]

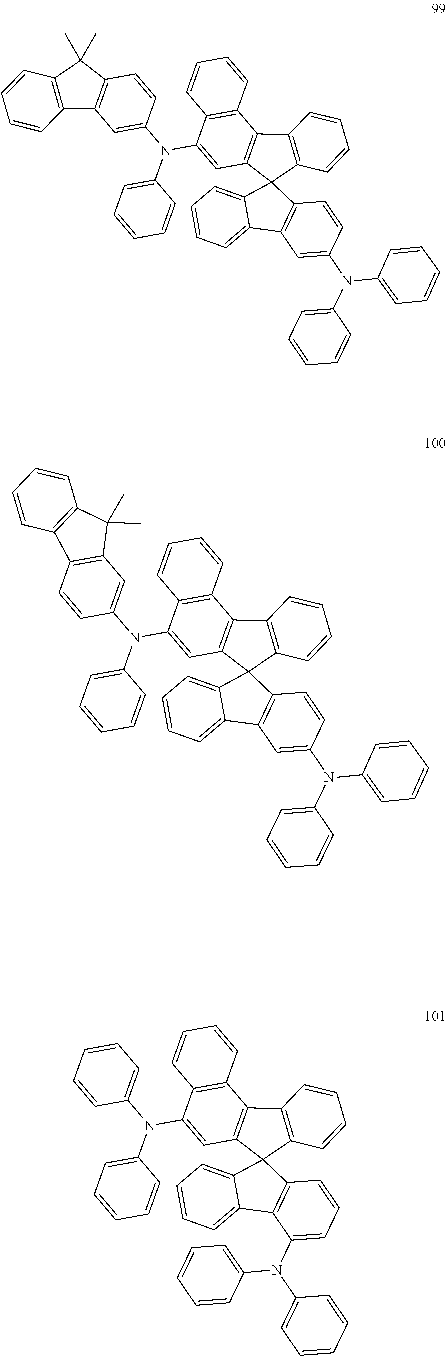

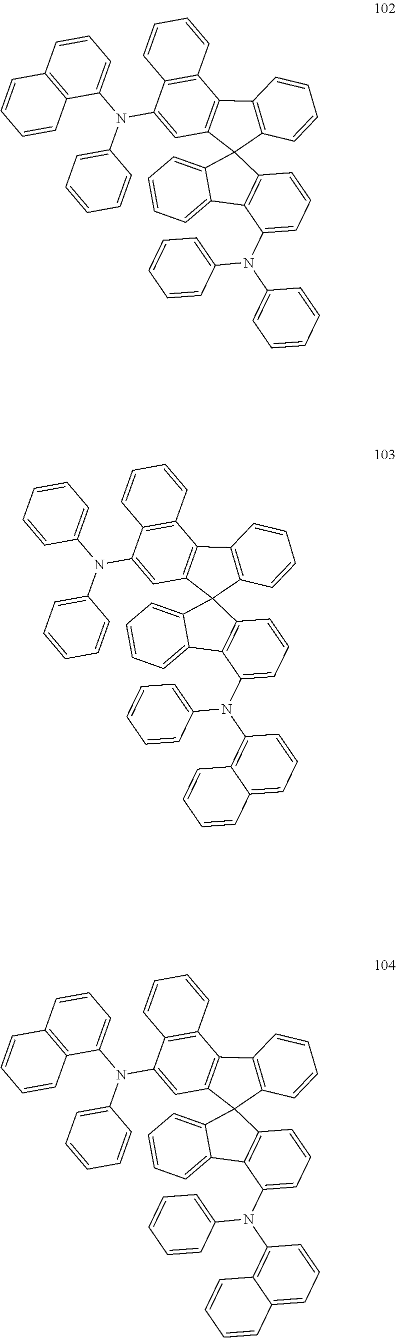

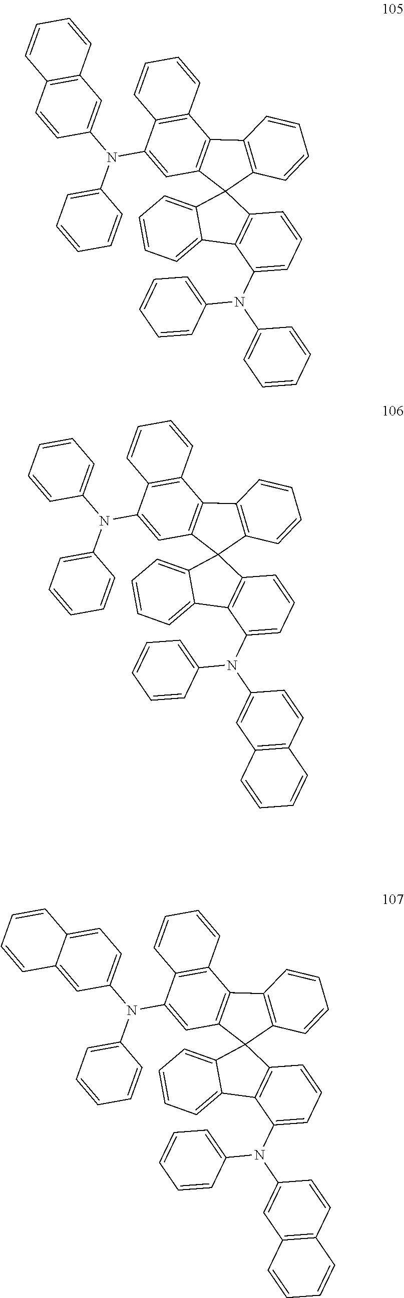 [SMILES: c1ccc(N(c2ccccc2)c2cccc3c2-c2ccccc2C32c3ccccc3-c3c2cc(N(c2ccccc2)c2ccc4ccccc4c2)c2ccccc32)cc1 ; c1ccc(N(c2ccc3ccccc3c2)c2cccc3c2-c2ccccc2C32c3ccccc3-c3c2cc(N(c2ccccc2)c2ccccc2)c2ccccc32)cc1 ; c1ccc(N(c2ccc3ccccc3c2)c2cccc3c2-c2ccccc2C32c3ccccc3-c3c2cc(N(c2ccccc2)c2ccc4ccccc4c2)c2ccccc32)cc1]

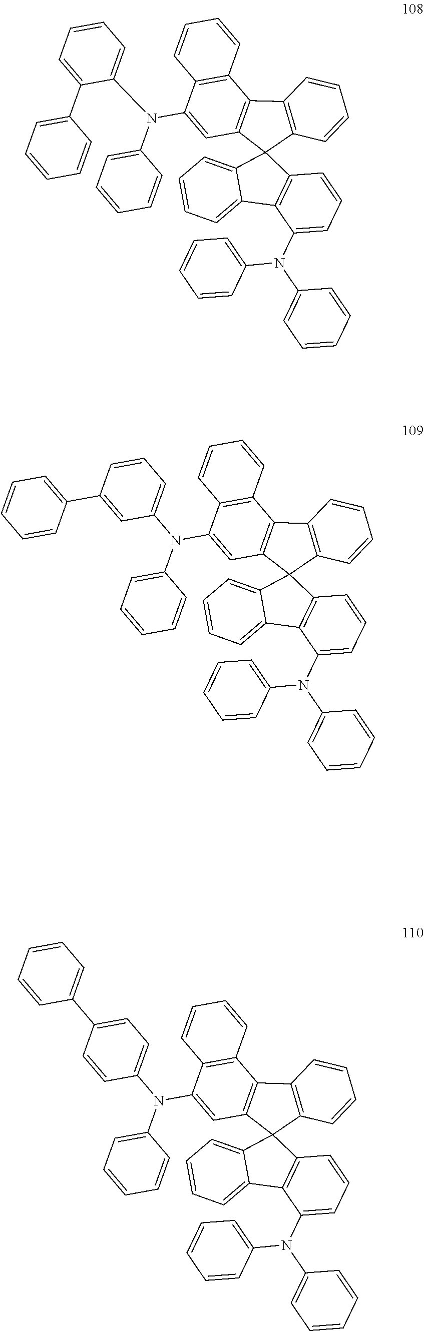 [SMILES: c1ccc(-c2ccccc2N(c2ccccc2)c2cc3c(c4ccccc24)-c2ccccc2C32c3ccccc3-c3c(N(c4ccccc4)c4ccccc4)cccc32)cc1 ; c1ccc(-c2cccc(N(c3ccccc3)c3cc4c(c5ccccc35)-c3ccccc3C43c4ccccc4-c4c(N(c5ccccc5)c5ccccc5)cccc43)c2)cc1 ; c1ccc(-c2ccc(N(c3ccccc3)c3cc4c(c5ccccc35)-c3ccccc3C43c4ccccc4-c4c(N(c5ccccc5)c5ccccc5)cccc43)cc2)cc1]

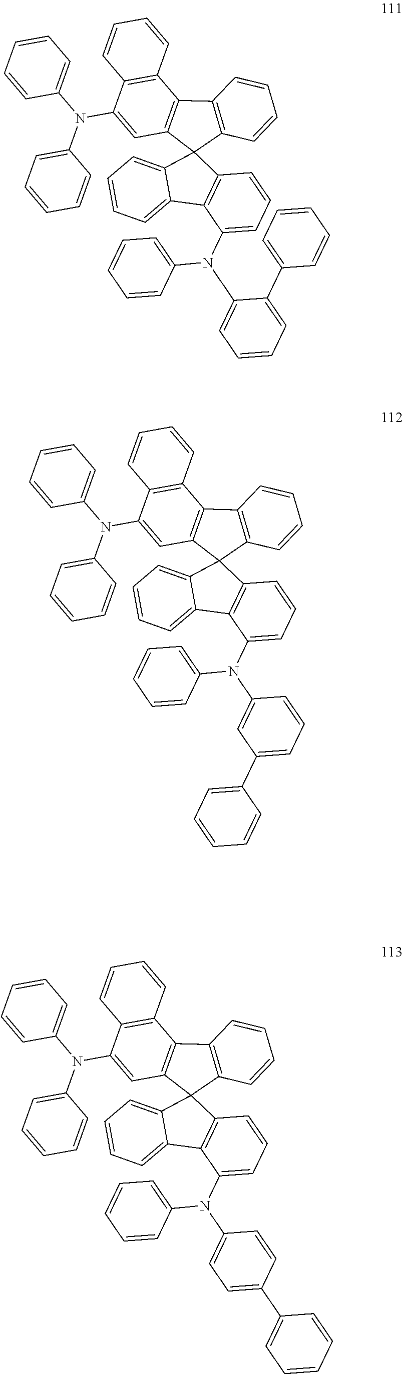 [SMILES: c1ccc(-c2ccccc2N(c2ccccc2)c2cccc3c2-c2ccccc2C32c3ccccc3-c3c2cc(N(c2ccccc2)c2ccccc2)c2ccccc32)cc1 ; c1ccc(-c2cccc(N(c3ccccc3)c3cccc4c3-c3ccccc3C43c4ccccc4-c4c3cc(N(c3ccccc3)c3ccccc3)c3ccccc43)c2)cc1 ; c1ccc(-c2ccc(N(c3ccccc3)c3cccc4c3-c3ccccc3C43c4ccccc4-c4c3cc(N(c3ccccc3)c3ccccc3)c3ccccc43)cc2)cc1]

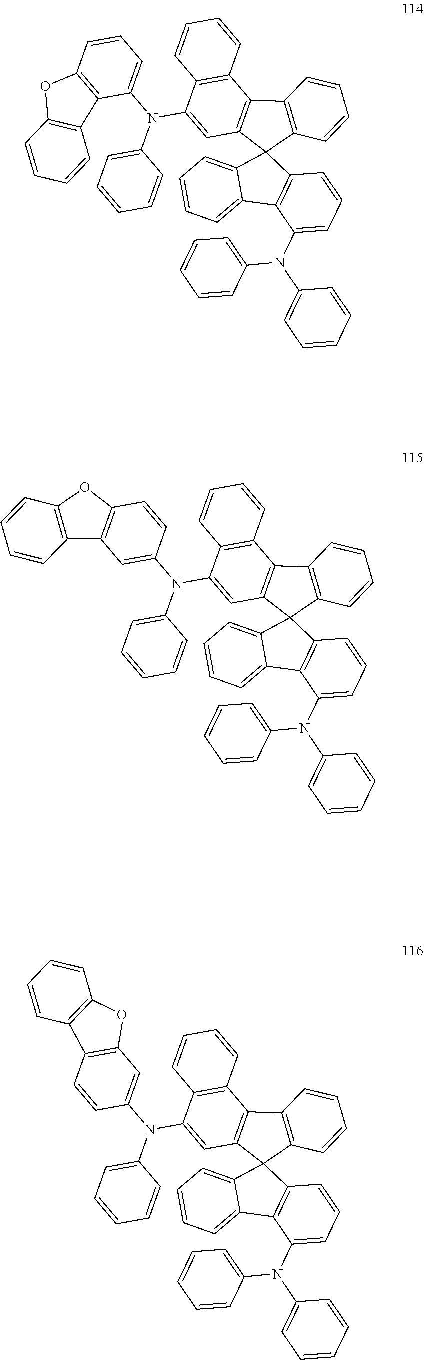 [SMILES: c1ccc(N(c2ccccc2)c2cccc3c2-c2ccccc2C32c3ccccc3-c3c2cc(N(c2ccccc2)c2cccc4oc5ccccc5c24)c2ccccc32)cc1 ; c1ccc(N(c2ccccc2)c2cccc3c2-c2ccccc2C32c3ccccc3-c3c2cc(N(c2ccccc2)c2ccc4oc5ccccc5c4c2)c2ccccc32)cc1 ; c1ccc(N(c2ccccc2)c2cccc3c2-c2ccccc2C32c3ccccc3-c3c2cc(N(c2ccccc2)c2ccc4c(c2)oc2ccccc24)c2ccccc32)cc1]

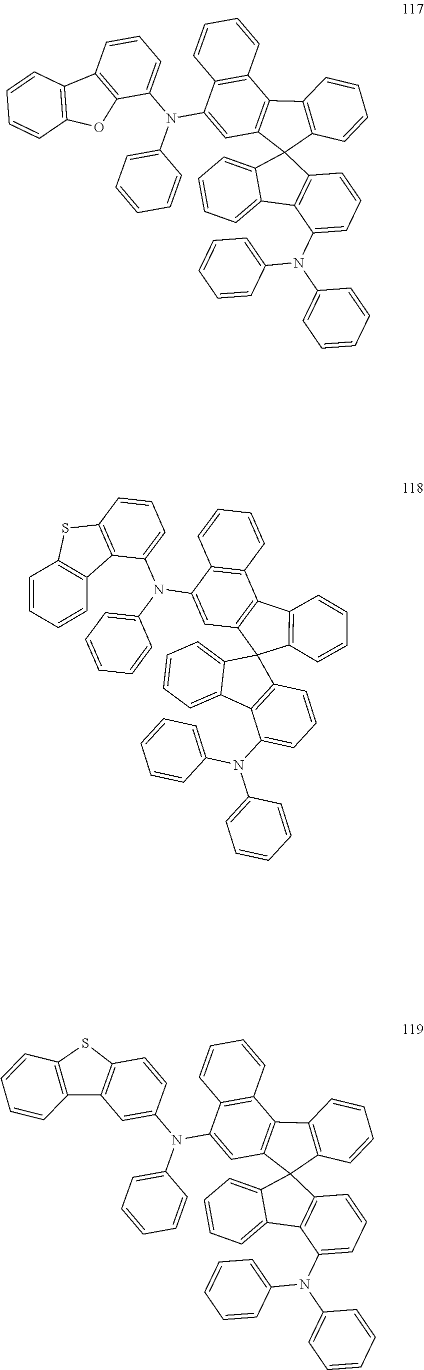 [SMILES: c1ccc(N(c2ccccc2)c2cccc3c2-c2ccccc2C32c3ccccc3-c3c2cc(N(c2ccccc2)c2cccc4c2oc2ccccc24)c2ccccc32)cc1 ; c1ccc(N(c2ccccc2)c2cccc3c2-c2ccccc2C32c3ccccc3-c3c2cc(N(c2ccccc2)c2cccc4sc5ccccc5c24)c2ccccc32)cc1 ; c1ccc(N(c2ccccc2)c2cccc3c2-c2ccccc2C32c3ccccc3-c3c2cc(N(c2ccccc2)c2ccc4sc5ccccc5c4c2)c2ccccc32)cc1]

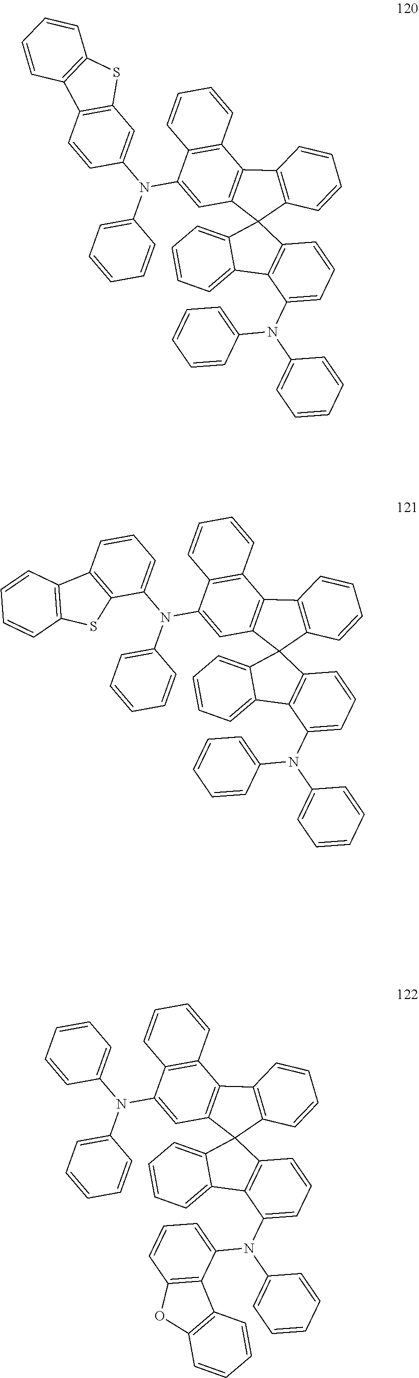 [SMILES: c1ccc(N(c2ccccc2)c2cccc3c2-c2ccccc2C32c3ccccc3-c3c2cc(N(c2ccccc2)c2ccc4c(c2)sc2ccccc24)c2ccccc32)cc1 ; c1ccc(N(c2ccccc2)c2cccc3c2-c2ccccc2C32c3ccccc3-c3c2cc(N(c2ccccc2)c2cccc4c2sc2ccccc24)c2ccccc32)cc1 ; c1ccc(N(c2ccccc2)c2cc3c(c4ccccc24)-c2ccccc2C32c3ccccc3-c3c(N(c4ccccc4)c4cccc5oc6ccccc6c45)cccc32)cc1]

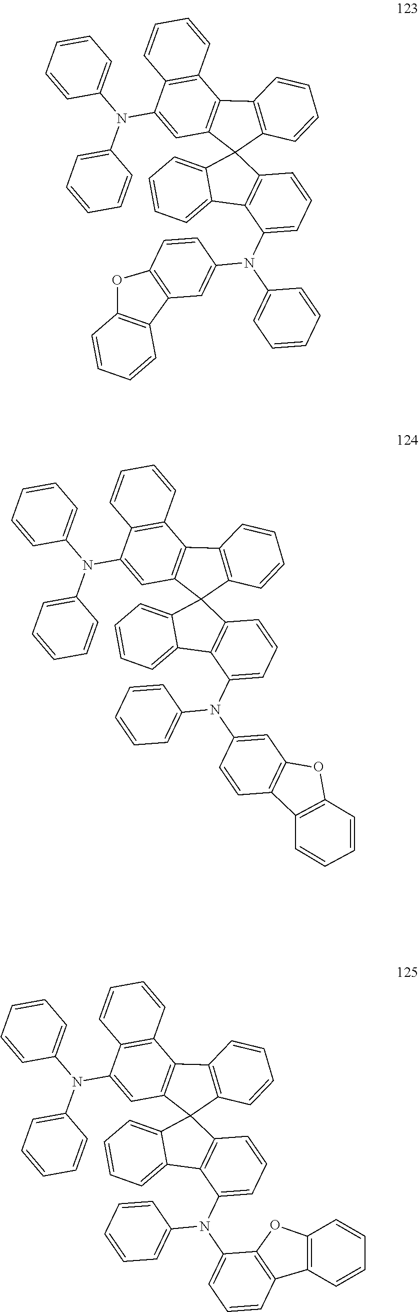 [SMILES: c1ccc(N(c2ccc3oc4ccccc4c3c2)c2cccc3c2-c2ccccc2C32c3ccccc3-c3c2cc(N(c2ccccc2)c2ccccc2)c2ccccc32)cc1 ; c1ccc(N(c2ccc3c(c2)oc2ccccc23)c2cccc3c2-c2ccccc2C32c3ccccc3-c3c2cc(N(c2ccccc2)c2ccccc2)c2ccccc32)cc1 ; c1ccc(N(c2ccccc2)c2cc3c(c4ccccc24)-c2ccccc2C32c3ccccc3-c3c(N(c4ccccc4)c4cccc5c4oc4ccccc45)cccc32)cc1]

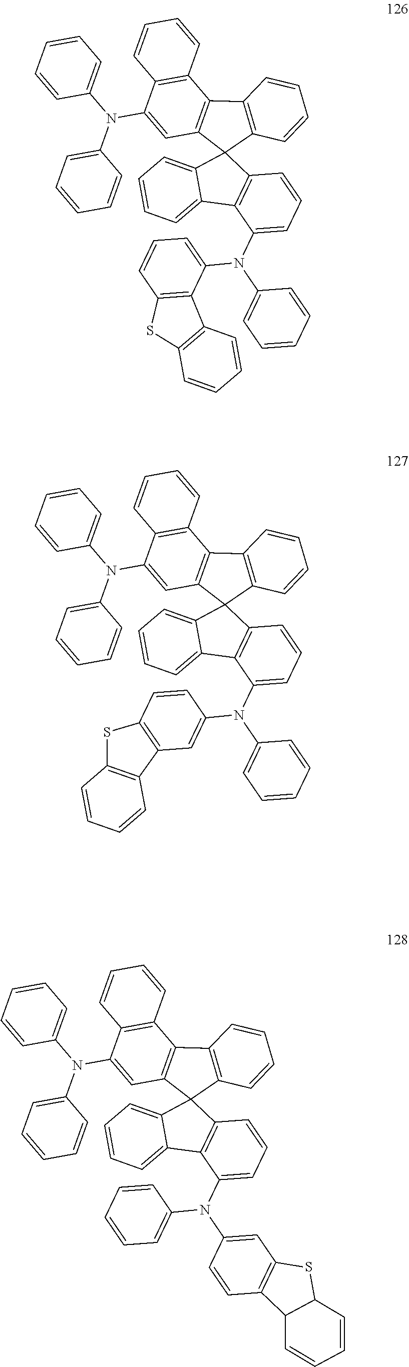 [SMILES: c1ccc(N(c2ccccc2)c2cc3c(c4ccccc24)-c2ccccc2C32c3ccccc3-c3c(N(c4ccccc4)c4cccc5sc6ccccc6c45)cccc32)cc1 ; c1ccc(N(c2ccc3sc4ccccc4c3c2)c2cccc3c2-c2ccccc2C32c3ccccc3-c3c2cc(N(c2ccccc2)c2ccccc2)c2ccccc32)cc1 ; C1=CC2Sc3cc(N(c4ccccc4)c4cccc5c4-c4ccccc4C54c5ccccc5-c5c4cc(N(c4ccccc4)c4ccccc4)c4ccccc54)ccc3C2C=C1]

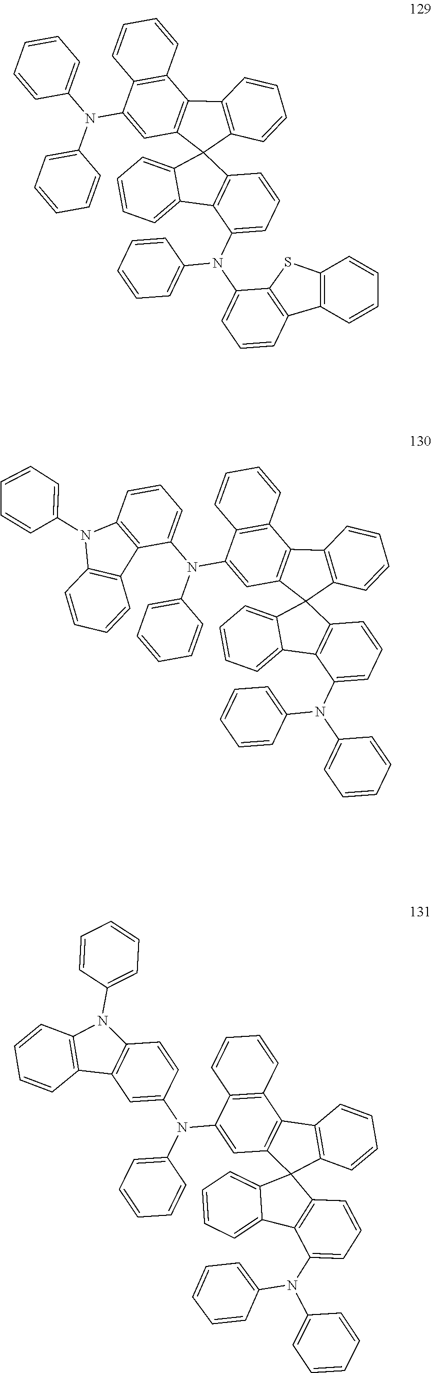 [SMILES: c1ccc(N(c2ccccc2)c2cc3c(c4ccccc24)-c2ccccc2C32c3ccccc3-c3c(N(c4ccccc4)c4cccc5c4sc4ccccc45)cccc32)cc1 ; c1ccc(N(c2ccccc2)c2cccc3c2-c2ccccc2C32c3ccccc3-c3c2cc(N(c2ccccc2)c2cccc4c2c2ccccc2n4-c2ccccc2)c2ccccc32)cc1 ; c1ccc(N(c2ccccc2)c2cccc3c2-c2ccccc2C32c3ccccc3-c3c2cc(N(c2ccccc2)c2ccc4c(c2)c2ccccc2n4-c2ccccc2)c2ccccc32)cc1]

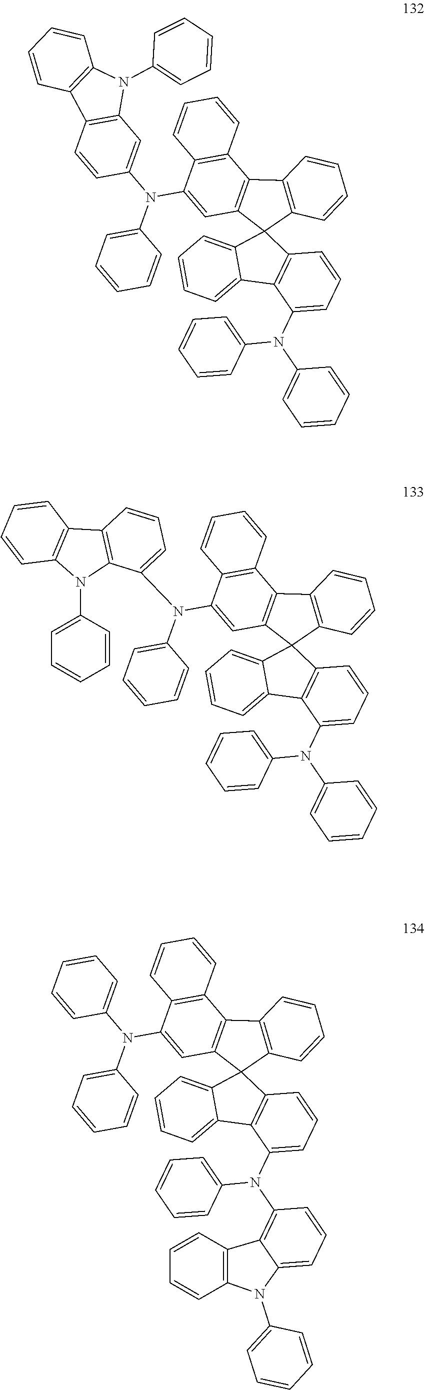 [SMILES: c1ccc(N(c2ccccc2)c2cccc3c2-c2ccccc2C32c3ccccc3-c3c2cc(N(c2ccccc2)c2ccc4c5ccccc5n(-c5ccccc5)c4c2)c2ccccc32)cc1 ; c1ccc(N(c2ccccc2)c2cccc3c2-c2ccccc2C32c3ccccc3-c3c2cc(N(c2ccccc2)c2cccc4c5ccccc5n(-c5ccccc5)c24)c2ccccc32)cc1 ; c1ccc(N(c2ccccc2)c2cc3c(c4ccccc24)-c2ccccc2C32c3ccccc3-c3c(N(c4ccccc4)c4cccc5c4c4ccccc4n5-c4ccccc4)cccc32)cc1]

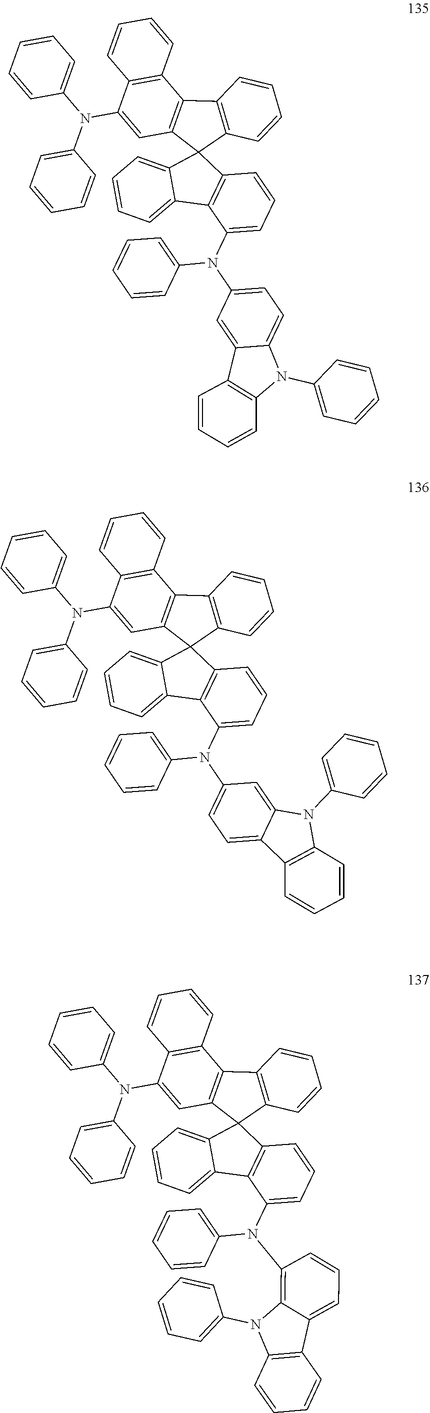 [SMILES: c1ccc(N(c2ccc3c(c2)c2ccccc2n3-c2ccccc2)c2cccc3c2-c2ccccc2C32c3ccccc3-c3c2cc(N(c2ccccc2)c2ccccc2)c2ccccc32)cc1 ; c1ccc(N(c2ccc3c4ccccc4n(-c4ccccc4)c3c2)c2cccc3c2-c2ccccc2C32c3ccccc3-c3c2cc(N(c2ccccc2)c2ccccc2)c2ccccc32)cc1 ; c1ccc(N(c2ccccc2)c2cc3c(c4ccccc24)-c2ccccc2C32c3ccccc3-c3c(N(c4ccccc4)c4cccc5c6ccccc6n(-c6ccccc6)c45)cccc32)cc1]

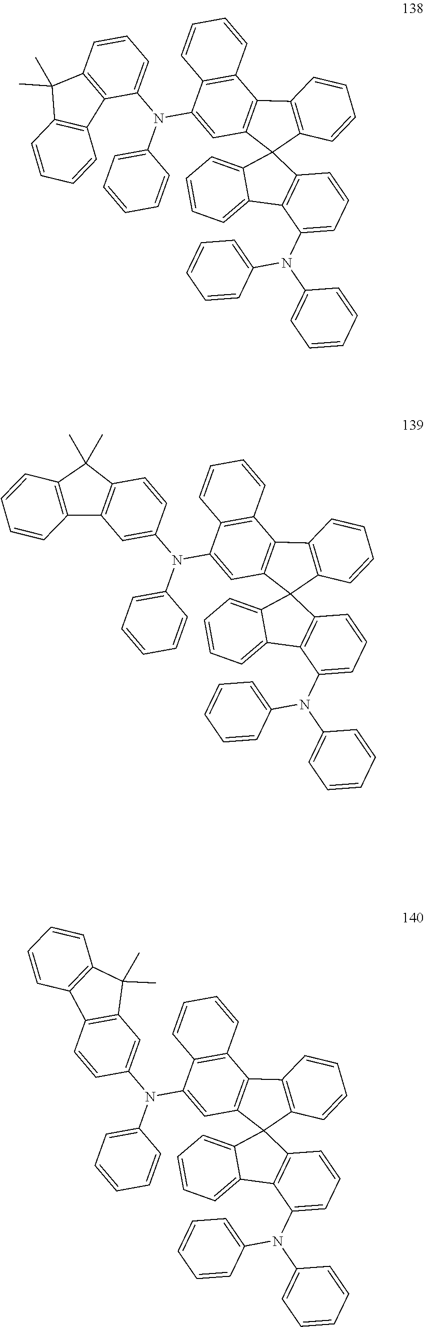 [SMILES: CC1(C)c2ccccc2-c2c(N(c3ccccc3)c3cc4c(c5ccccc35)-c3ccccc3C43c4ccccc4-c4c(N(c5ccccc5)c5ccccc5)cccc43)cccc21 ; CC1(C)c2ccccc2-c2cc(N(c3ccccc3)c3cc4c(c5ccccc35)-c3ccccc3C43c4ccccc4-c4c(N(c5ccccc5)c5ccccc5)cccc43)ccc21 ; CC1(C)c2ccccc2-c2ccc(N(c3ccccc3)c3cc4c(c5ccccc35)-c3ccccc3C43c4ccccc4-c4c(N(c5ccccc5)c5ccccc5)cccc43)cc21]

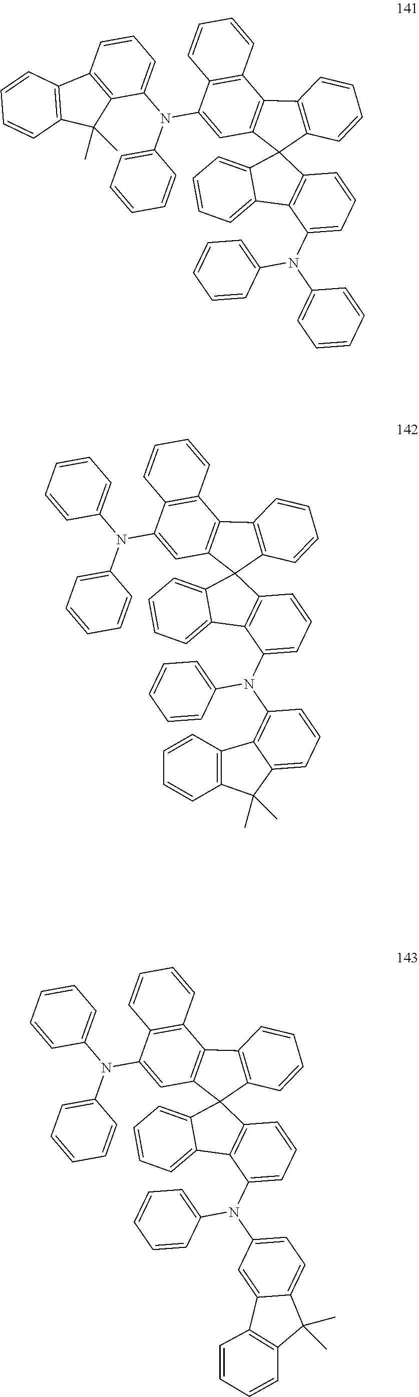 [SMILES: CC1(C)c2ccccc2-c2cccc(N(c3ccccc3)c3cc4c(c5ccccc35)-c3ccccc3C43c4ccccc4-c4c(N(c5ccccc5)c5ccccc5)cccc43)c21 ; CC1(C)c2ccccc2-c2c(N(c3ccccc3)c3cccc4c3-c3ccccc3C43c4ccccc4-c4c3cc(N(c3ccccc3)c3ccccc3)c3ccccc43)cccc21 ; CC1(C)c2ccccc2-c2cc(N(c3ccccc3)c3cccc4c3-c3ccccc3C43c4ccccc4-c4c3cc(N(c3ccccc3)c3ccccc3)c3ccccc43)ccc21]

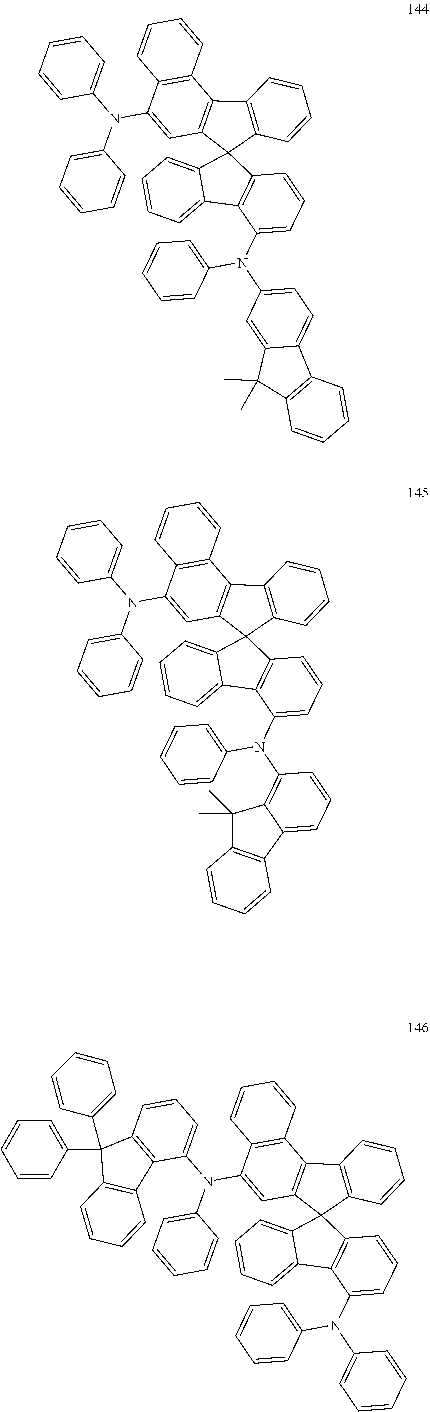 [SMILES: CC1(C)c2ccccc2-c2ccc(N(c3ccccc3)c3cccc4c3-c3ccccc3C43c4ccccc4-c4c3cc(N(c3ccccc3)c3ccccc3)c3ccccc43)cc21 ; CC1(C)c2ccccc2-c2cccc(N(c3ccccc3)c3cccc4c3-c3ccccc3C43c4ccccc4-c4c3cc(N(c3ccccc3)c3ccccc3)c3ccccc43)c21 ; c1ccc(N(c2ccccc2)c2cccc3c2-c2ccccc2C32c3ccccc3-c3c2cc(N(c2ccccc2)c2cccc4c2-c2ccccc2C4(c2ccccc2)c2ccccc2)c2ccccc32)cc1]

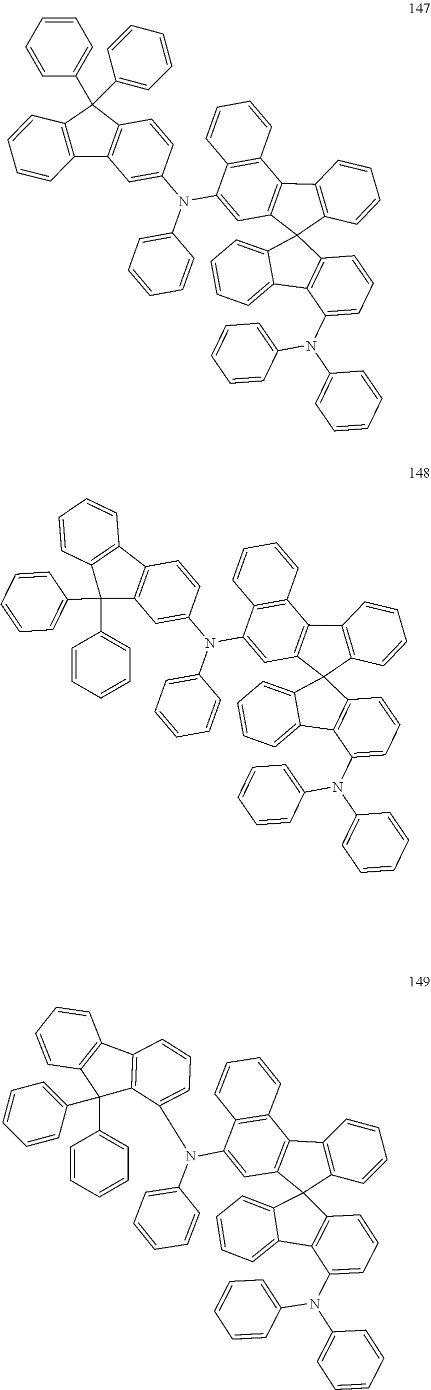 [SMILES: c1ccc(N(c2ccccc2)c2cccc3c2-c2ccccc2C32c3ccccc3-c3c2cc(N(c2ccccc2)c2ccc4c(c2)-c2ccccc2C4(c2ccccc2)c2ccccc2)c2ccccc32)cc1 ; c1ccc(N(c2ccccc2)c2cccc3c2-c2ccccc2C32c3ccccc3-c3c2cc(N(c2ccccc2)c2ccc4c(c2)C(c2ccccc2)(c2ccccc2)c2ccccc2-4)c2ccccc32)cc1 ; c1ccc(N(c2ccccc2)c2cccc3c2-c2ccccc2C32c3ccccc3-c3c2cc(N(c2ccccc2)c2cccc4c2C(c2ccccc2)(c2ccccc2)c2ccccc2-4)c2ccccc32)cc1]

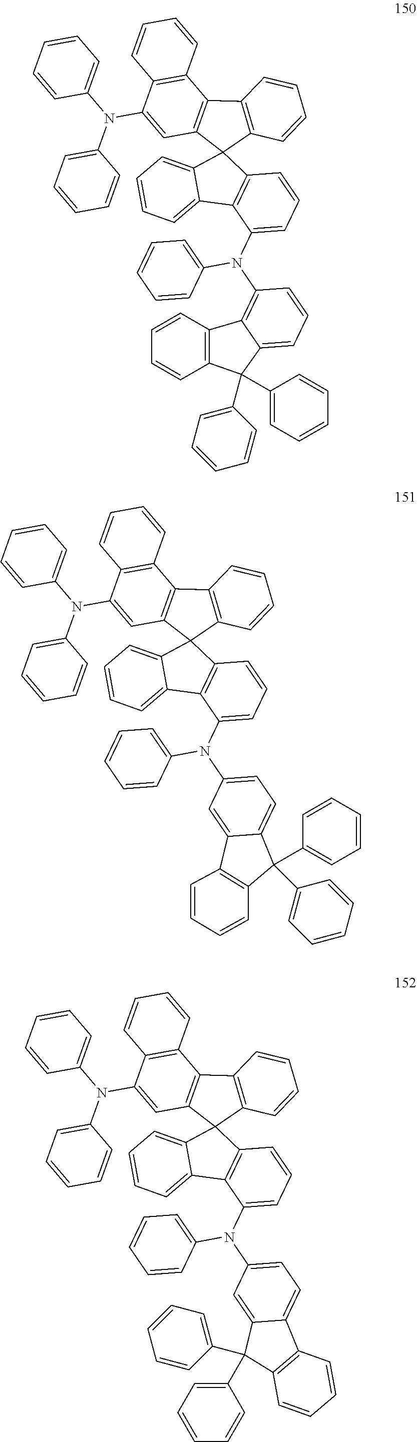 [SMILES: c1ccc(N(c2cccc3c2-c2ccccc2C3(c2ccccc2)c2ccccc2)c2cccc3c2-c2ccccc2C32c3ccccc3-c3c2cc(N(c2ccccc2)c2ccccc2)c2ccccc32)cc1 ; c1ccc(N(c2ccc3c(c2)-c2ccccc2C3(c2ccccc2)c2ccccc2)c2cccc3c2-c2ccccc2C32c3ccccc3-c3c2cc(N(c2ccccc2)c2ccccc2)c2ccccc32)cc1 ; c1ccc(N(c2ccc3c(c2)C(c2ccccc2)(c2ccccc2)c2ccccc2-3)c2cccc3c2-c2ccccc2C32c3ccccc3-c3c2cc(N(c2ccccc2)c2ccccc2)c2ccccc32)cc1]

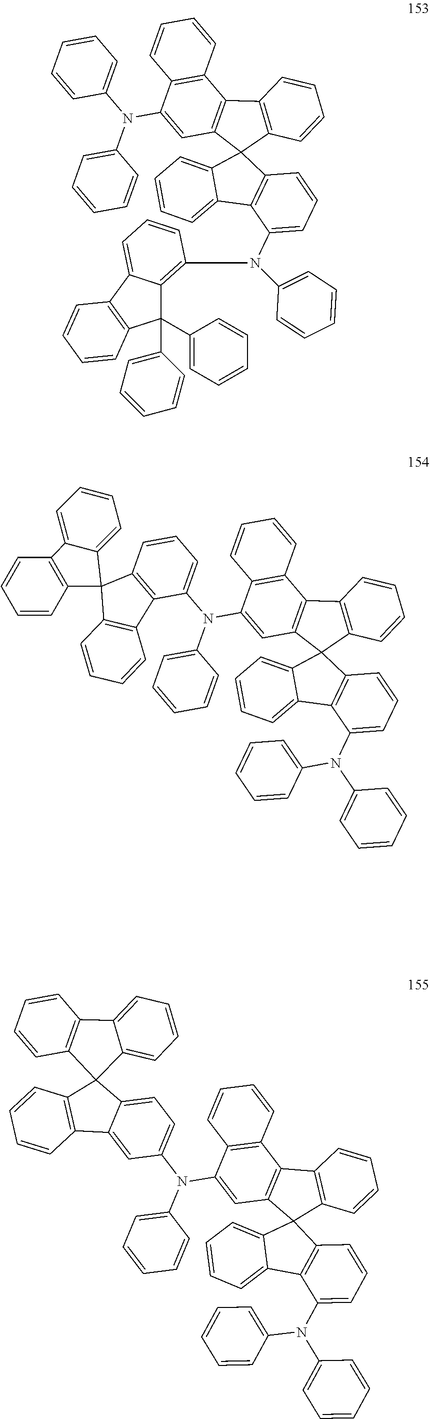 [SMILES: c1ccc(N(c2cccc3c2-c2ccccc2C32c3ccccc3-c3c2cc(N(c2ccccc2)c2ccccc2)c2ccccc32)c2cccc3c2C(c2ccccc2)(c2ccccc2)c2ccccc2-3)cc1 ; c1ccc(N(c2ccccc2)c2cccc3c2-c2ccccc2C32c3ccccc3-c3c2cc(N(c2ccccc2)c2cccc4c2-c2ccccc2C42c4ccccc4-c4ccccc42)c2ccccc32)cc1 ; c1ccc(N(c2ccccc2)c2cccc3c2-c2ccccc2C32c3ccccc3-c3c2cc(N(c2ccccc2)c2ccc4c(c2)-c2ccccc2C42c4ccccc4-c4ccccc42)c2ccccc32)cc1]

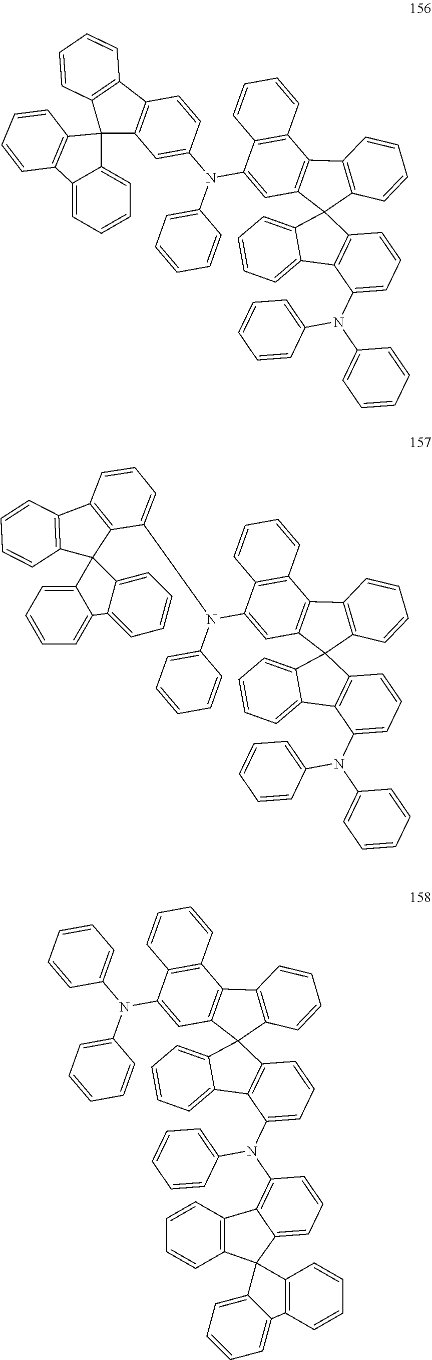 [SMILES: c1ccc(N(c2ccccc2)c2cccc3c2-c2ccccc2C32c3ccccc3-c3c2cc(N(c2ccccc2)c2ccc4c(c2)C2(c5ccccc5-c5ccccc52)c2ccccc2-4)c2ccccc32)cc1 ; c1ccc(N(c2ccccc2)c2cccc3c2-c2ccccc2C32c3ccccc3-c3c2cc(N(c2ccccc2)c2cccc4c2C2(c5ccccc5-c5ccccc52)c2ccccc2-4)c2ccccc32)cc1 ; c1ccc(N(c2cccc3c2-c2ccccc2C32c3ccccc3-c3ccccc32)c2cccc3c2-c2ccccc2C32c3ccccc3-c3c2cc(N(c2ccccc2)c2ccccc2)c2ccccc32)cc1]

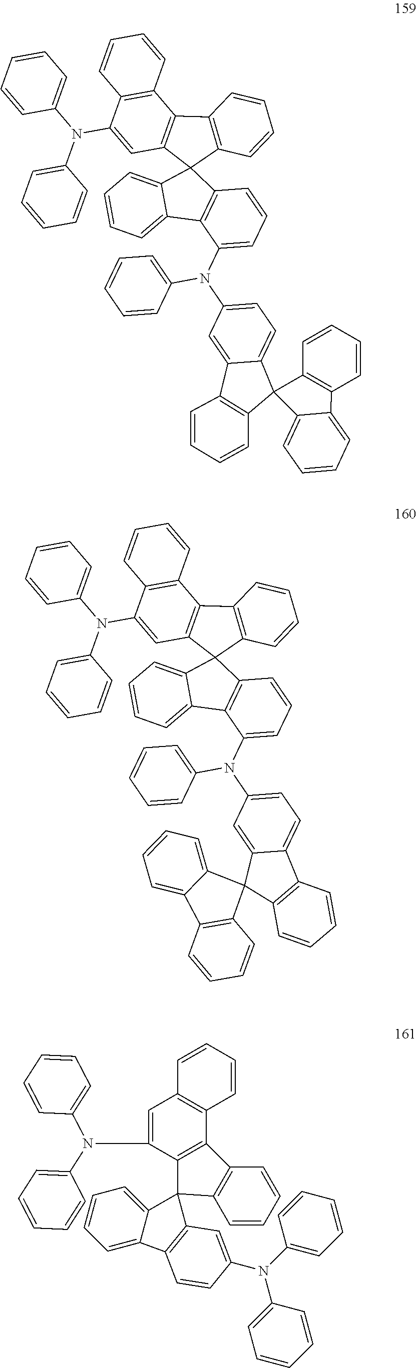 [SMILES: c1ccc(N(c2ccc3c(c2)-c2ccccc2C32c3ccccc3-c3ccccc32)c2cccc3c2-c2ccccc2C32c3ccccc3-c3c2cc(N(c2ccccc2)c2ccccc2)c2ccccc32)cc1 ; c1ccc(N(c2ccc3c(c2)C2(c4ccccc4-c4ccccc42)c2ccccc2-3)c2cccc3c2-c2ccccc2C32c3ccccc3-c3c2cc(N(c2ccccc2)c2ccccc2)c2ccccc32)cc1 ; c1ccc(N(c2ccccc2)c2ccc3c(c2)C2(c4ccccc4-3)c3ccccc3-c3c2c(N(c2ccccc2)c2ccccc2)cc2ccccc32)cc1]

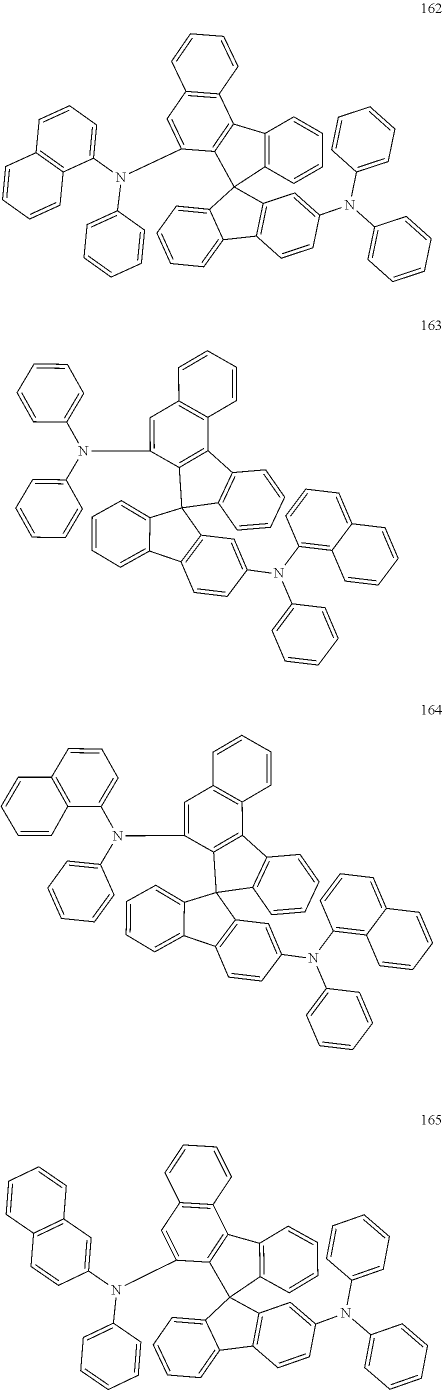 [SMILES: c1ccc(N(c2ccccc2)c2ccc3c(c2)C2(c4ccccc4-3)c3ccccc3-c3c2c(N(c2ccccc2)c2cccc4ccccc24)cc2ccccc32)cc1 ; c1ccc(N(c2ccccc2)c2cc3ccccc3c3c2C2(c4ccccc4-c4ccc(N(c5ccccc5)c5cccc6ccccc56)cc42)c2ccccc2-3)cc1 ; c1ccc(N(c2ccc3c(c2)C2(c4ccccc4-3)c3ccccc3-c3c2c(N(c2ccccc2)c2cccc4ccccc24)cc2ccccc32)c2cccc3ccccc23)cc1 ; c1ccc(N(c2ccccc2)c2ccc3c(c2)C2(c4ccccc4-3)c3ccccc3-c3c2c(N(c2ccccc2)c2ccc4ccccc4c2)cc2ccccc32)cc1]

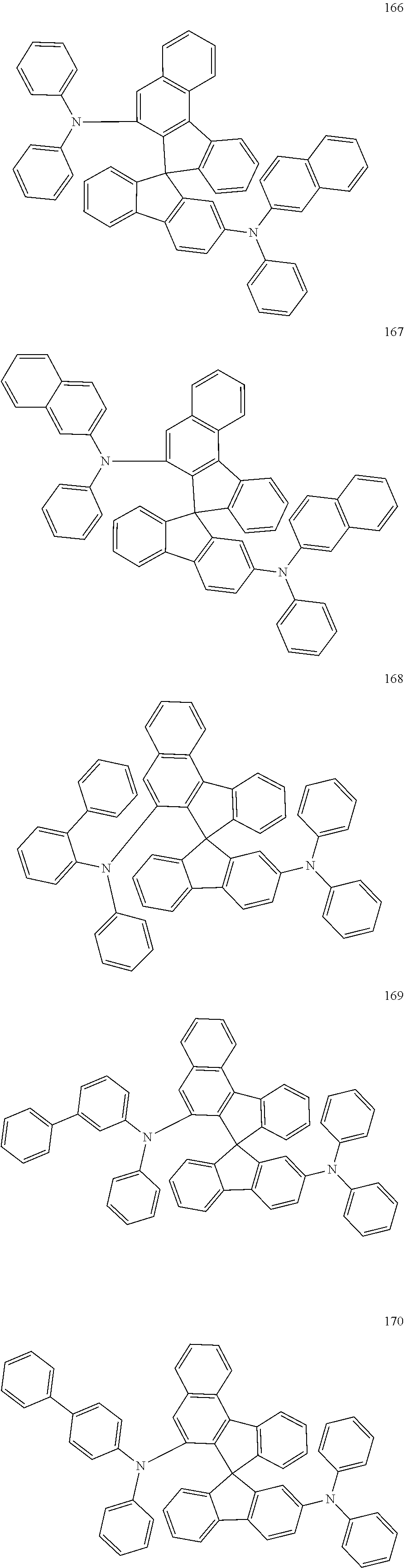

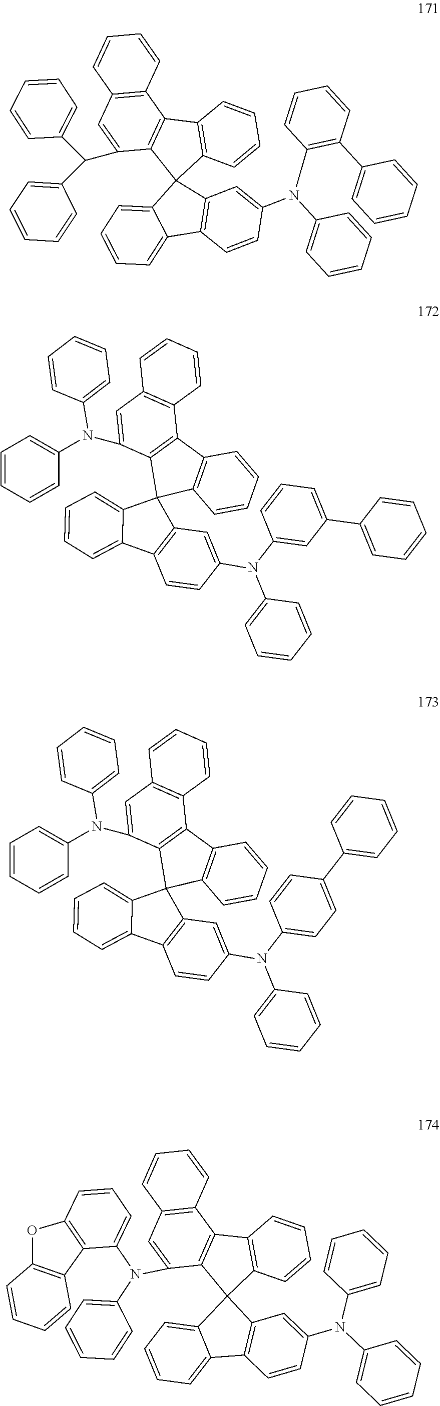 [SMILES: c1ccc(-c2ccccc2N(c2ccccc2)c2ccc3c(c2)C2(c4ccccc4-3)c3ccccc3-c3c2c(C(c2ccccc2)c2ccccc2)cc2ccccc32)cc1 ; c1ccc(-c2cccc(N(c3ccccc3)c3ccc4c(c3)C3(c5ccccc5-4)c4ccccc4-c4c3c(N(c3ccccc3)c3ccccc3)cc3ccccc43)c2)cc1 ; c1ccc(-c2ccc(N(c3ccccc3)c3ccc4c(c3)C3(c5ccccc5-4)c4ccccc4-c4c3c(N(c3ccccc3)c3ccccc3)cc3ccccc43)cc2)cc1 ; c1ccc(N(c2ccccc2)c2ccc3c(c2)C2(c4ccccc4-3)c3ccccc3-c3c2c(N(c2ccccc2)c2cccc4oc5ccccc5c24)cc2ccccc32)cc1]

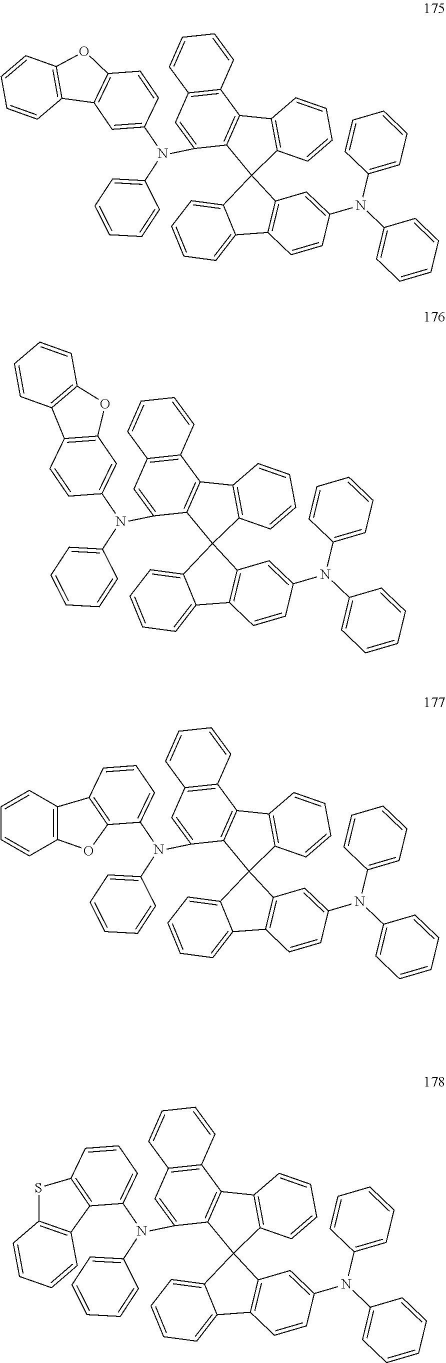 [SMILES: c1ccc(N(c2ccccc2)c2ccc3c(c2)C2(c4ccccc4-3)c3ccccc3-c3c2c(N(c2ccccc2)c2ccc4oc5ccccc5c4c2)cc2ccccc32)cc1 ; c1ccc(N(c2ccccc2)c2ccc3c(c2)C2(c4ccccc4-3)c3ccccc3-c3c2c(N(c2ccccc2)c2ccc4c(c2)oc2ccccc24)cc2ccccc32)cc1 ; c1ccc(N(c2ccccc2)c2ccc3c(c2)C2(c4ccccc4-3)c3ccccc3-c3c2c(N(c2ccccc2)c2cccc4c2oc2ccccc24)cc2ccccc32)cc1 ; c1ccc(N(c2ccccc2)c2ccc3c(c2)C2(c4ccccc4-3)c3ccccc3-c3c2c(N(c2ccccc2)c2cccc4sc5ccccc5c24)cc2ccccc32)cc1]

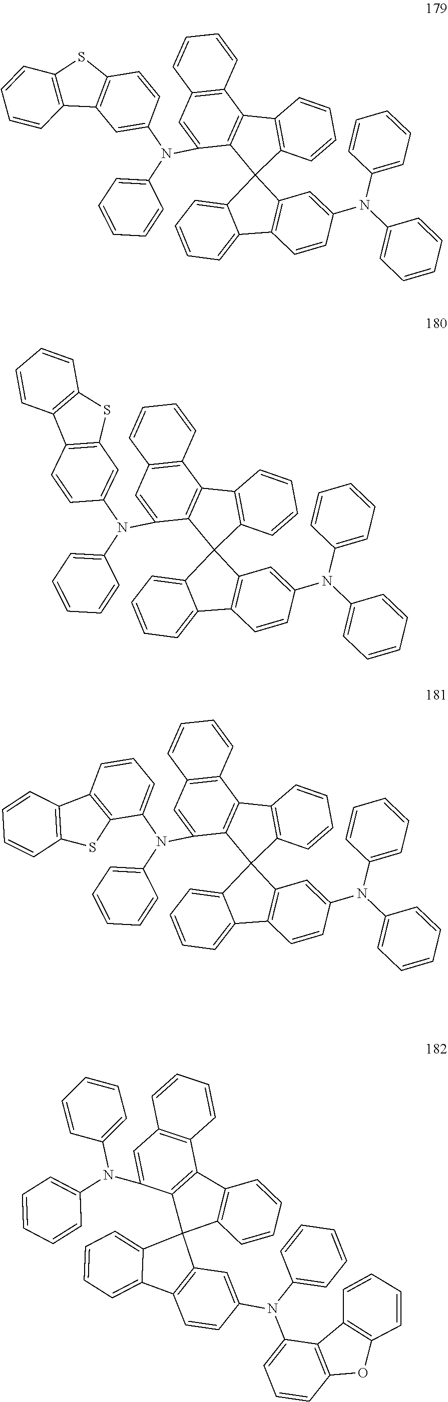 [SMILES: c1ccc(N(c2ccccc2)c2ccc3c(c2)C2(c4ccccc4-3)c3ccccc3-c3c2c(N(c2ccccc2)c2ccc4sc5ccccc5c4c2)cc2ccccc32)cc1 ; c1ccc(N(c2ccccc2)c2ccc3c(c2)C2(c4ccccc4-3)c3ccccc3-c3c2c(N(c2ccccc2)c2ccc4c(c2)sc2ccccc24)cc2ccccc32)cc1 ; c1ccc(N(c2ccccc2)c2ccc3c(c2)C2(c4ccccc4-3)c3ccccc3-c3c2c(N(c2ccccc2)c2cccc4c2sc2ccccc24)cc2ccccc32)cc1 ; c1ccc(N(c2ccccc2)c2cc3ccccc3c3c2C2(c4ccccc4-c4ccc(N(c5ccccc5)c5cccc6oc7ccccc7c56)cc42)c2ccccc2-3)cc1]

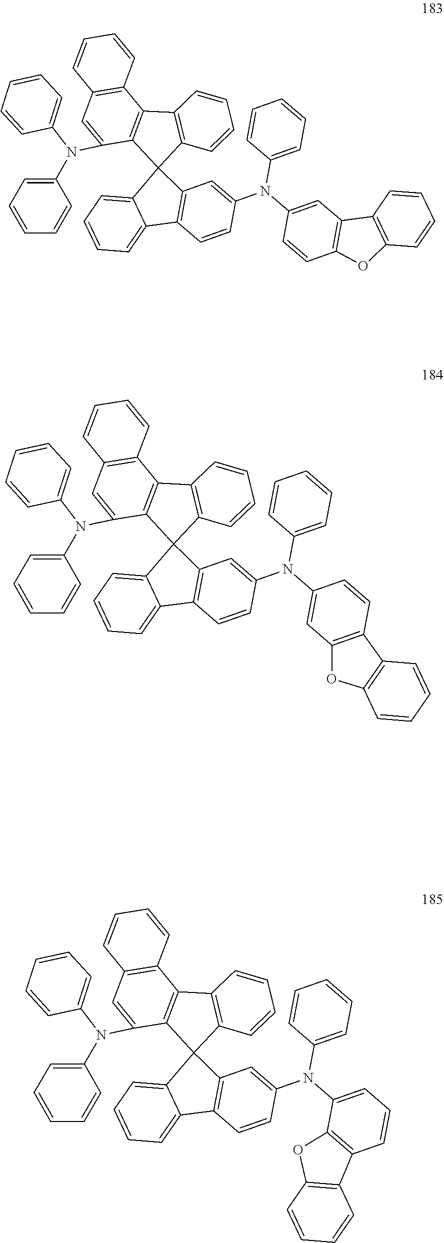 [SMILES: c1ccc(N(c2ccc3c(c2)C2(c4ccccc4-3)c3ccccc3-c3c2c(N(c2ccccc2)c2ccccc2)cc2ccccc32)c2ccc3oc4ccccc4c3c2)cc1 ; c1ccc(N(c2ccc3c(c2)C2(c4ccccc4-3)c3ccccc3-c3c2c(N(c2ccccc2)c2ccccc2)cc2ccccc32)c2ccc3c(c2)oc2ccccc23)cc1 ; c1ccc(N(c2ccccc2)c2cc3ccccc3c3c2C2(c4ccccc4-c4ccc(N(c5ccccc5)c5cccc6c5oc5ccccc56)cc42)c2ccccc2-3)cc1]

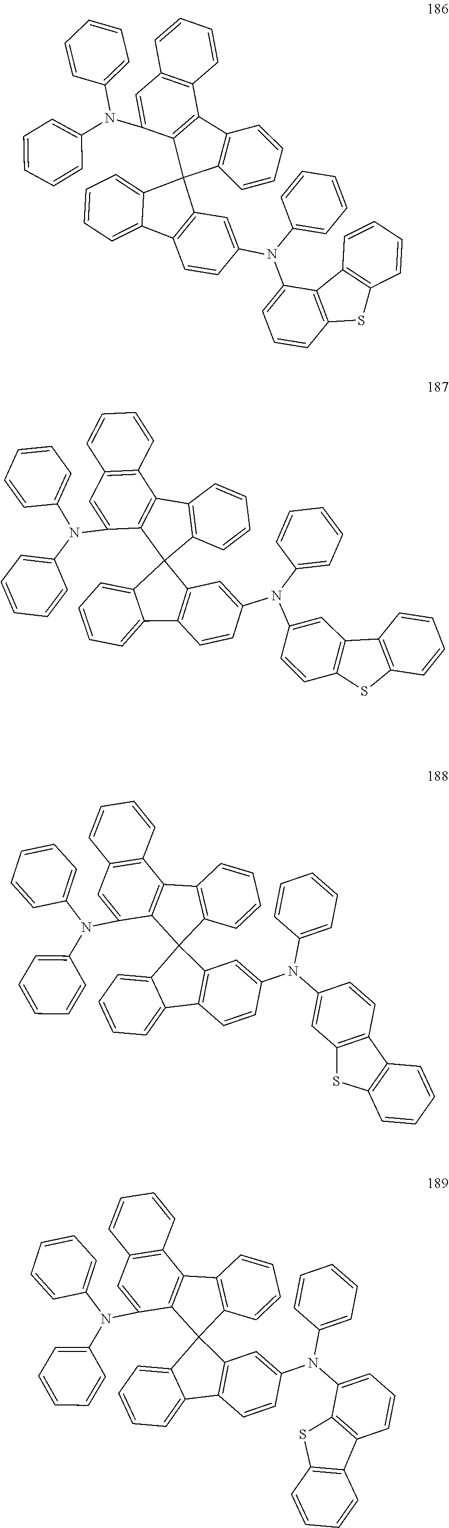 [SMILES: c1ccc(N(c2ccccc2)c2cc3ccccc3c3c2C2(c4ccccc4-c4ccc(N(c5ccccc5)c5cccc6sc7ccccc7c56)cc42)c2ccccc2-3)cc1 ; c1ccc(N(c2ccc3c(c2)C2(c4ccccc4-3)c3ccccc3-c3c2c(N(c2ccccc2)c2ccccc2)cc2ccccc32)c2ccc3sc4ccccc4c3c2)cc1 ; c1ccc(N(c2ccc3c(c2)C2(c4ccccc4-3)c3ccccc3-c3c2c(N(c2ccccc2)c2ccccc2)cc2ccccc32)c2ccc3c(c2)sc2ccccc23)cc1 ; c1ccc(N(c2ccccc2)c2cc3ccccc3c3c2C2(c4ccccc4-c4ccc(N(c5ccccc5)c5cccc6c5sc5ccccc56)cc42)c2ccccc2-3)cc1]

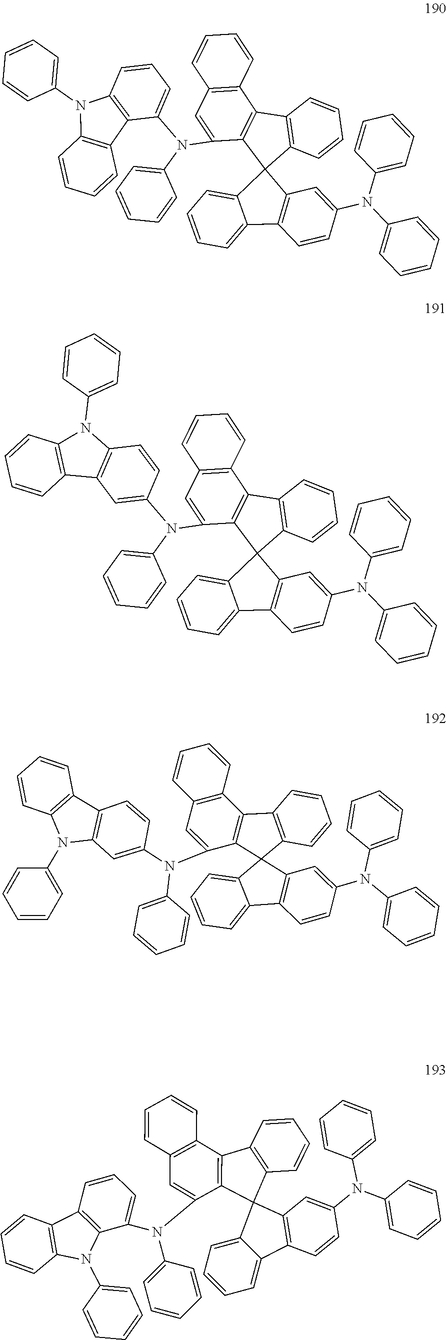 [SMILES: c1ccc(N(c2ccccc2)c2ccc3c(c2)C2(c4ccccc4-3)c3ccccc3-c3c2c(N(c2ccccc2)c2cccc4c2c2ccccc2n4-c2ccccc2)cc2ccccc32)cc1 ; c1ccc(N(c2ccccc2)c2ccc3c(c2)C2(c4ccccc4-3)c3ccccc3-c3c2c(N(c2ccccc2)c2ccc4c(c2)c2ccccc2n4-c2ccccc2)cc2ccccc32)cc1 ; c1ccc(N(c2ccccc2)c2ccc3c(c2)C2(c4ccccc4-3)c3ccccc3-c3c2c(N(c2ccccc2)c2ccc4c5ccccc5n(-c5ccccc5)c4c2)cc2ccccc32)cc1 ; c1ccc(N(c2ccccc2)c2ccc3c(c2)C2(c4ccccc4-3)c3ccccc3-c3c2c(N(c2ccccc2)c2cccc4c5ccccc5n(-c5ccccc5)c24)cc2ccccc32)cc1]

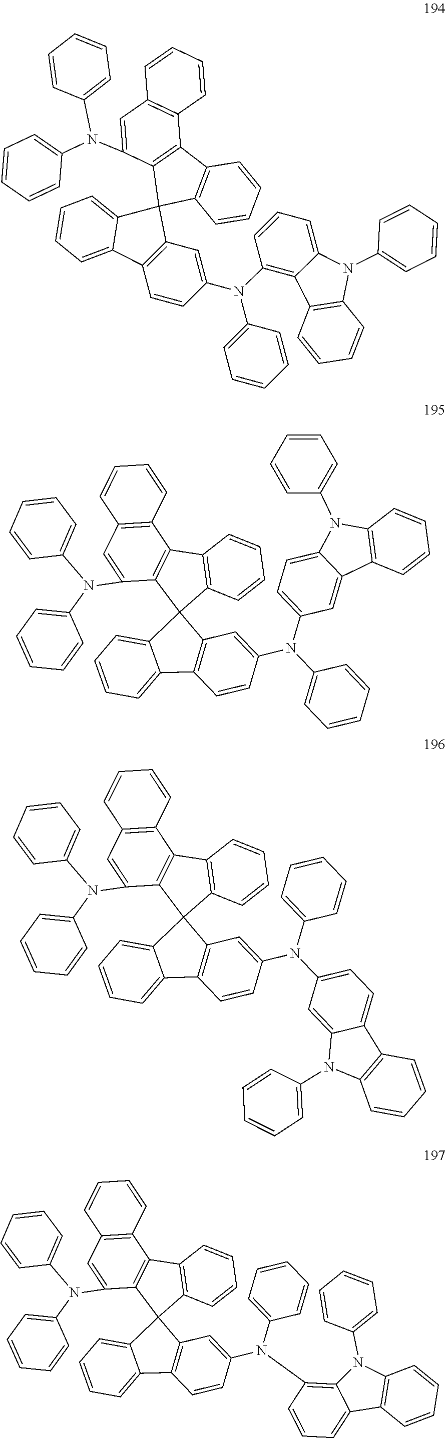 [SMILES: c1ccc(N(c2ccccc2)c2cc3ccccc3c3c2C2(c4ccccc4-c4ccc(N(c5ccccc5)c5cccc6c5c5ccccc5n6-c5ccccc5)cc42)c2ccccc2-3)cc1 ; c1ccc(N(c2ccc3c(c2)C2(c4ccccc4-3)c3ccccc3-c3c2c(N(c2ccccc2)c2ccccc2)cc2ccccc32)c2ccc3c(c2)c2ccccc2n3-c2ccccc2)cc1 ; c1ccc(N(c2ccc3c(c2)C2(c4ccccc4-3)c3ccccc3-c3c2c(N(c2ccccc2)c2ccccc2)cc2ccccc32)c2ccc3c4ccccc4n(-c4ccccc4)c3c2)cc1 ; c1ccc(N(c2ccccc2)c2cc3ccccc3c3c2C2(c4ccccc4-c4ccc(N(c5ccccc5)c5cccc6c7ccccc7n(-c7ccccc7)c56)cc42)c2ccccc2-3)cc1]

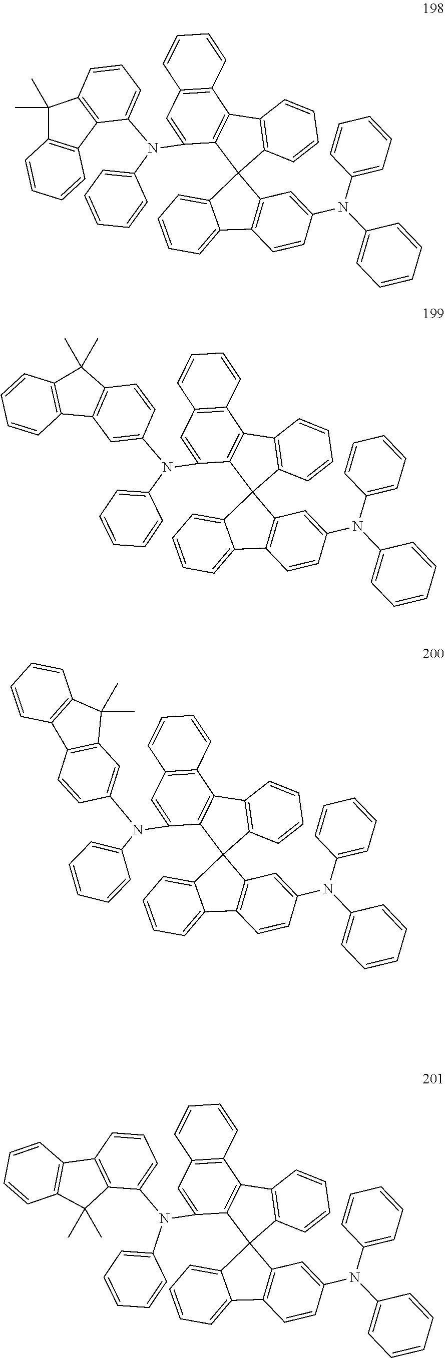 [SMILES: CC1(C)c2ccccc2-c2c(N(c3ccccc3)c3cc4ccccc4c4c3C3(c5ccccc5-c5ccc(N(c6ccccc6)c6ccccc6)cc53)c3ccccc3-4)cccc21 ; CC1(C)c2ccccc2-c2cc(N(c3ccccc3)c3cc4ccccc4c4c3C3(c5ccccc5-c5ccc(N(c6ccccc6)c6ccccc6)cc53)c3ccccc3-4)ccc21 ; CC1(C)c2ccccc2-c2ccc(N(c3ccccc3)c3cc4ccccc4c4c3C3(c5ccccc5-c5ccc(N(c6ccccc6)c6ccccc6)cc53)c3ccccc3-4)cc21 ; CC1(C)c2ccccc2-c2cccc(N(c3ccccc3)c3cc4ccccc4c4c3C3(c5ccccc5-c5ccc(N(c6ccccc6)c6ccccc6)cc53)c3ccccc3-4)c21]

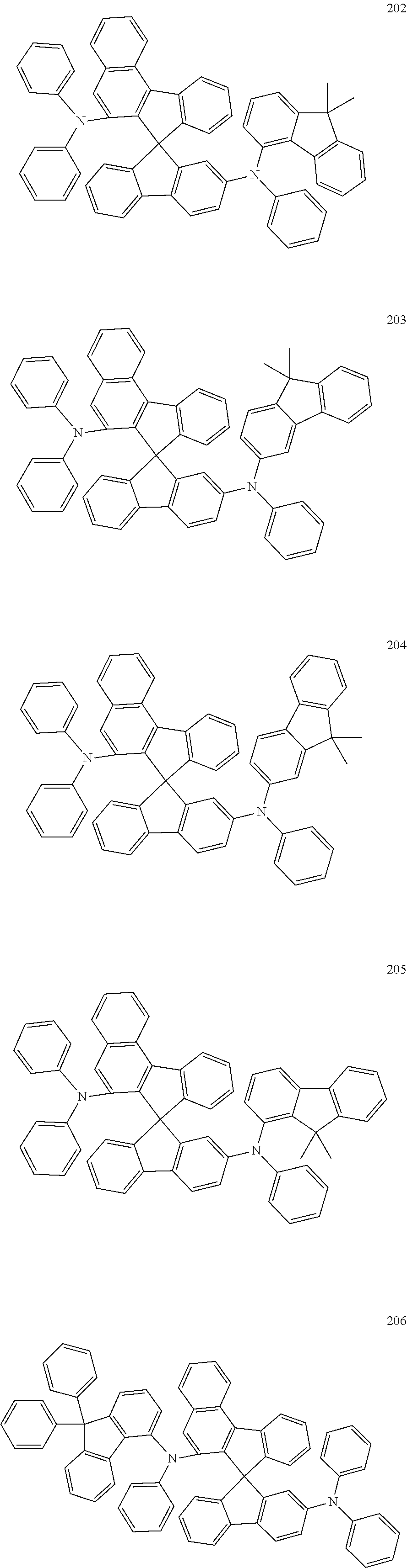

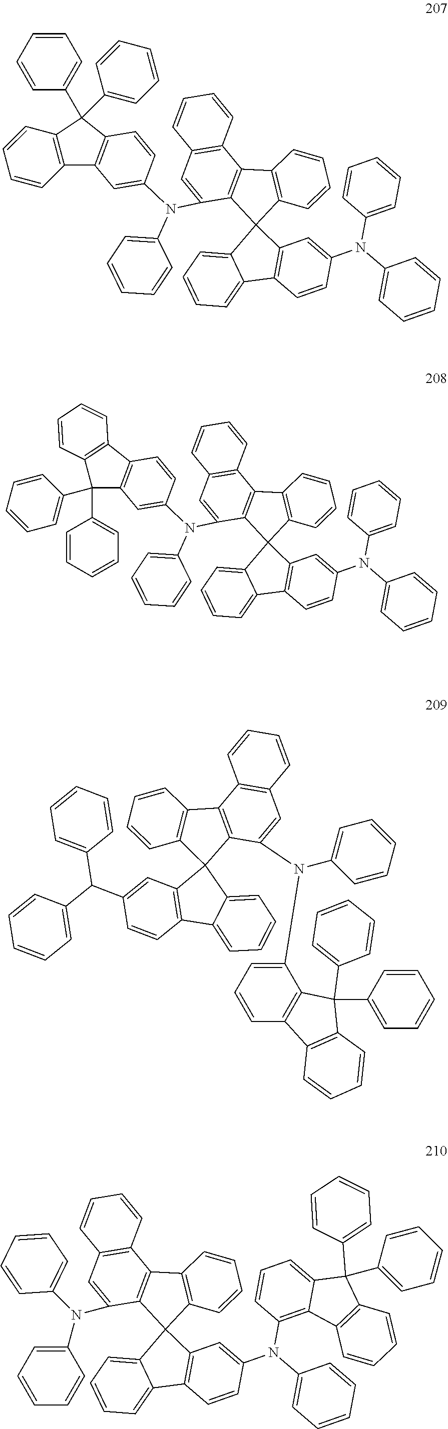

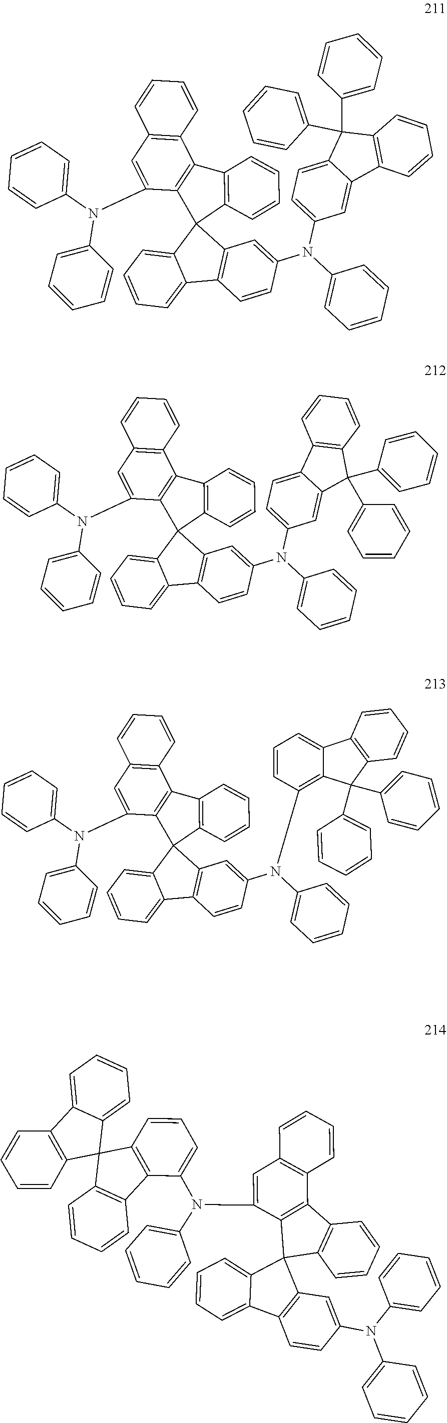

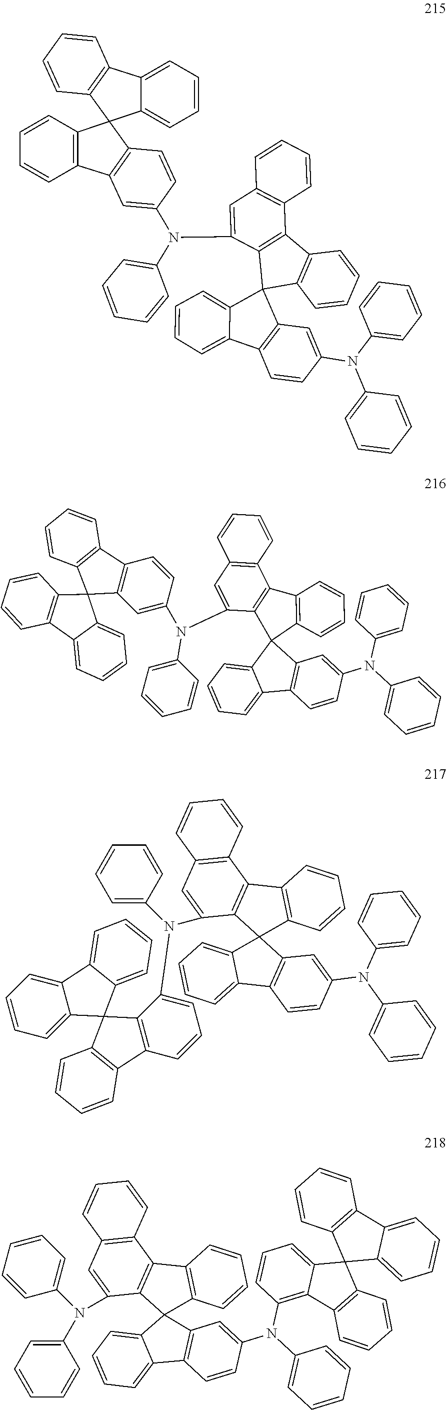

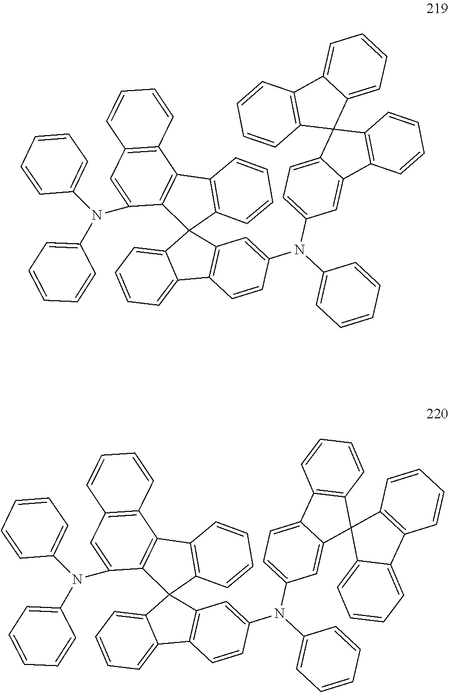 [SMILES: c1ccc(N(c2ccc3c(c2)-c2ccccc2C32c3ccccc3-c3ccccc32)c2ccc3c(c2)C2(c4ccccc4-3)c3ccccc3-c3c2c(N(c2ccccc2)c2ccccc2)cc2ccccc32)cc1 ; c1ccc(N(c2ccc3c(c2)C2(c4ccccc4-c4ccccc42)c2ccccc2-3)c2ccc3c(c2)C2(c4ccccc4-3)c3ccccc3-c3c2c(N(c2ccccc2)c2ccccc2)cc2ccccc32)cc1]

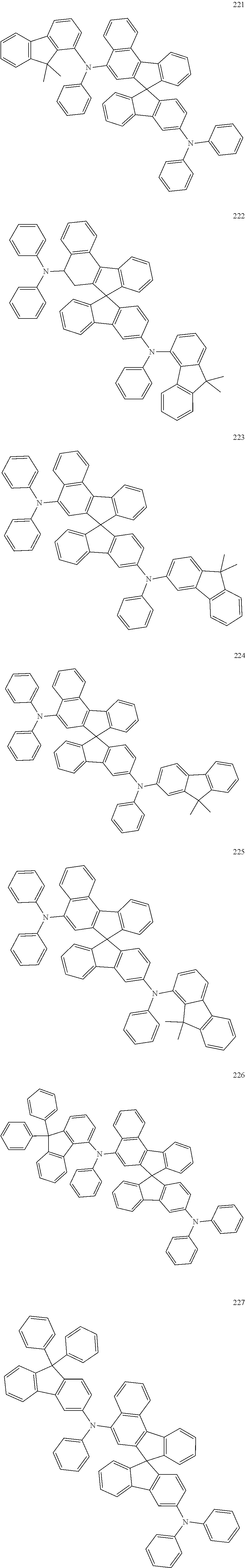

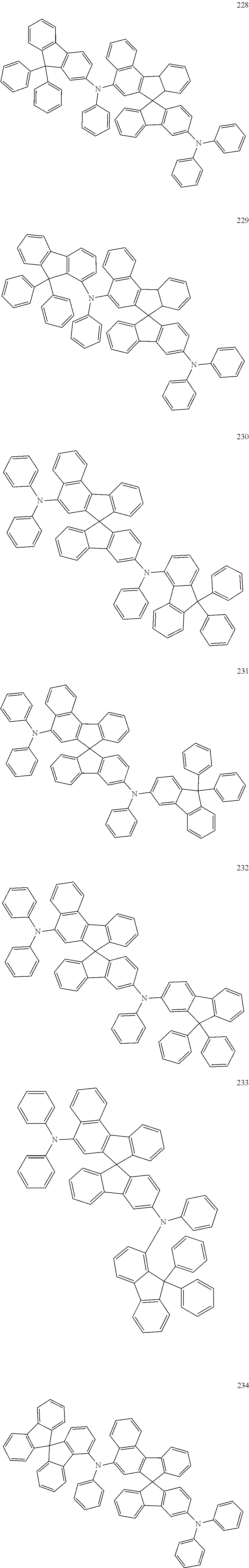

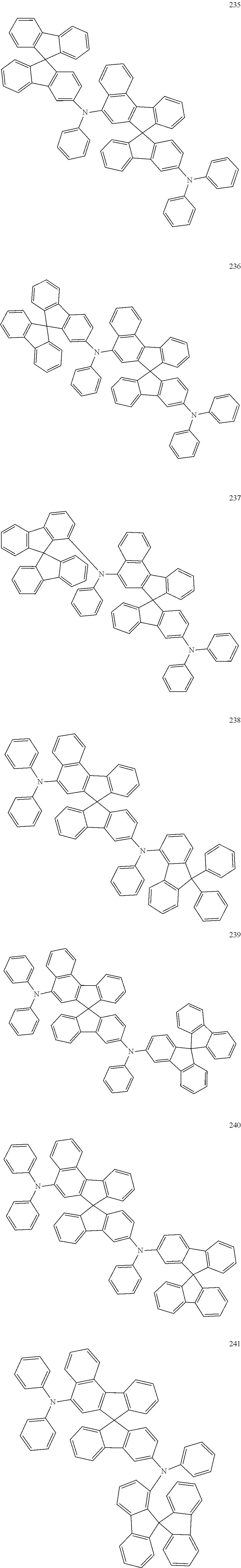

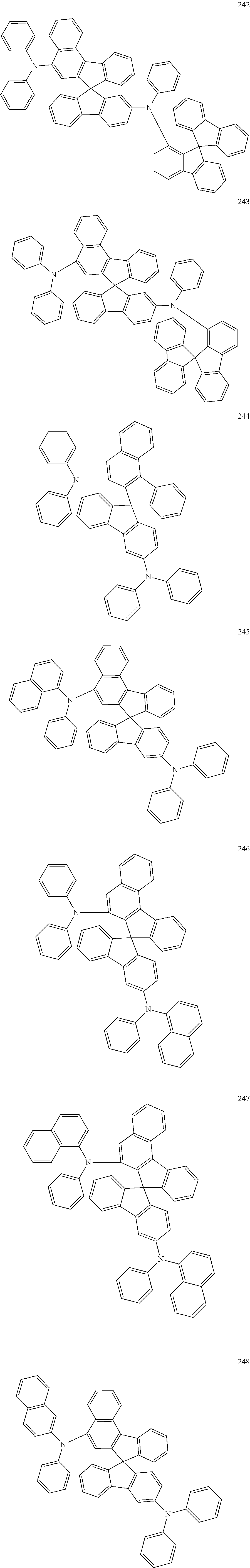

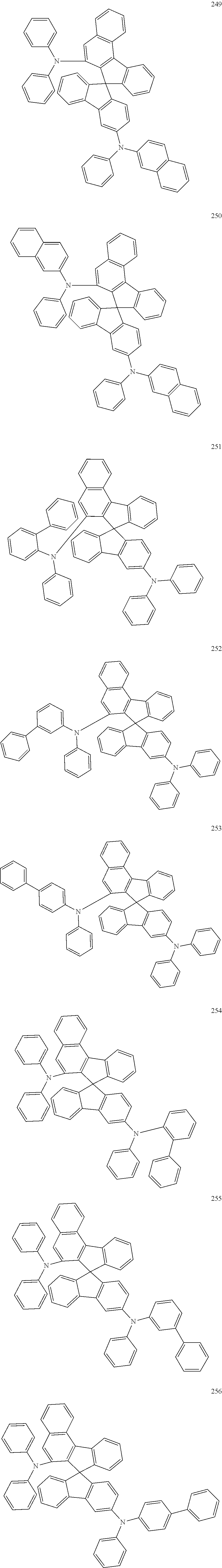

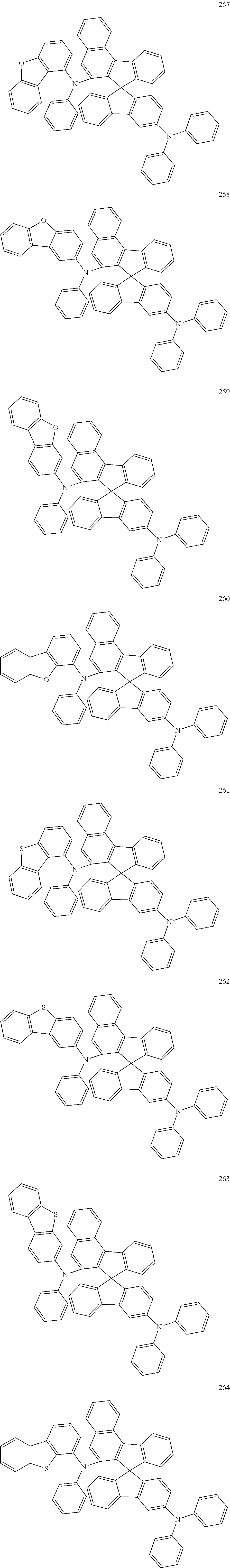

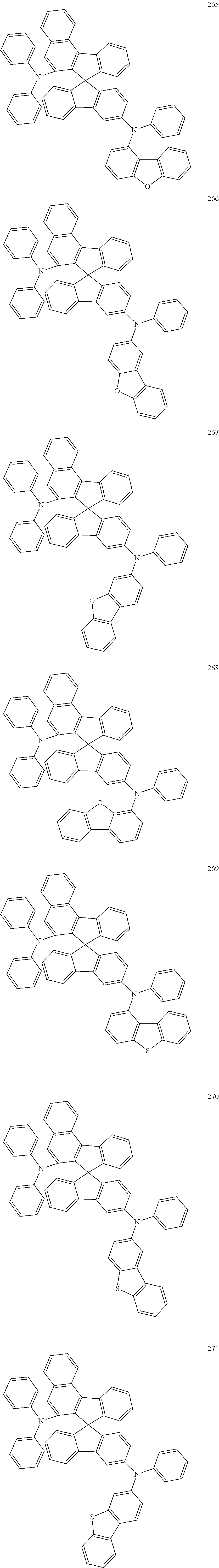

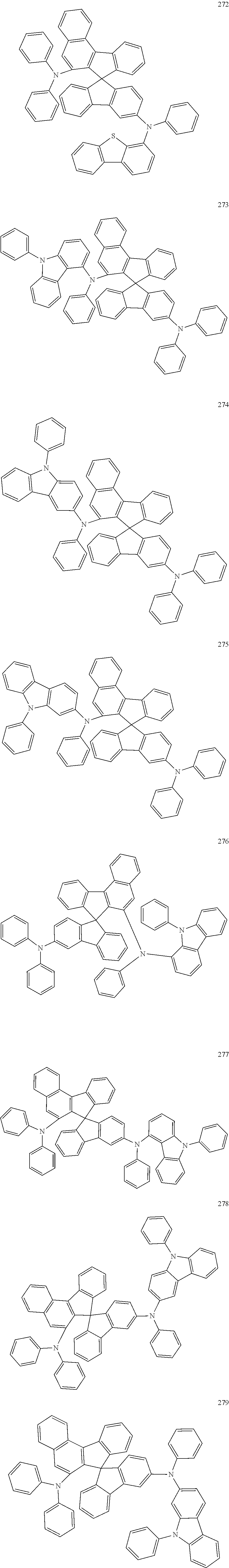

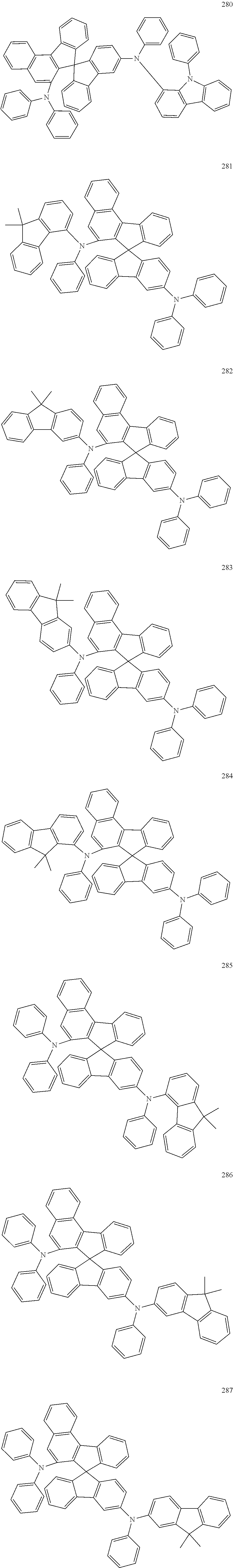

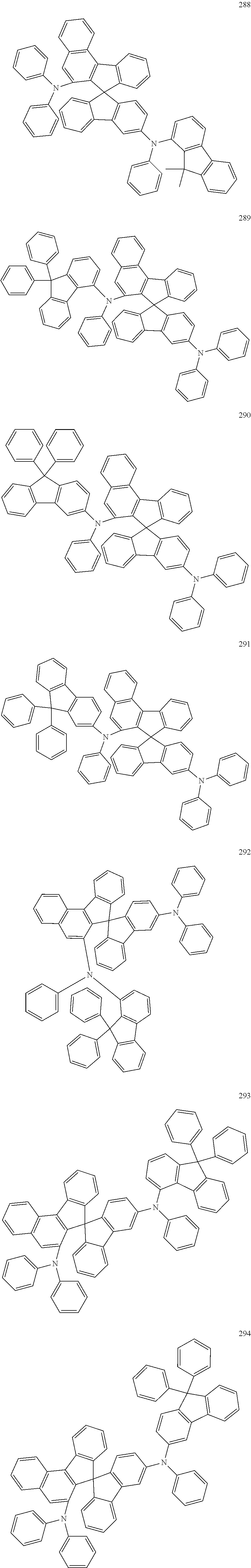

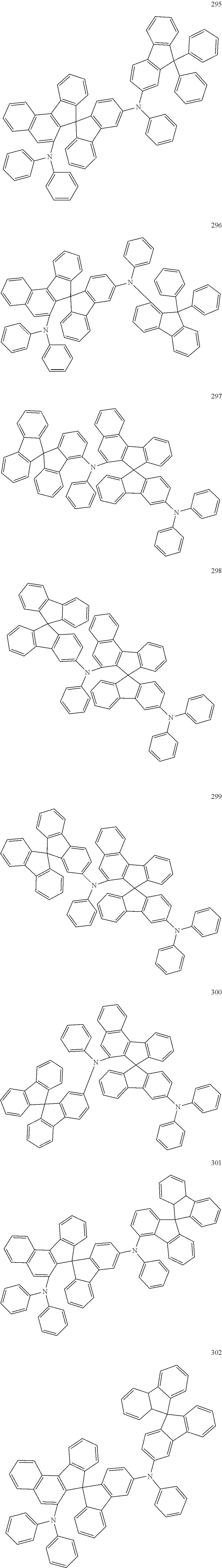

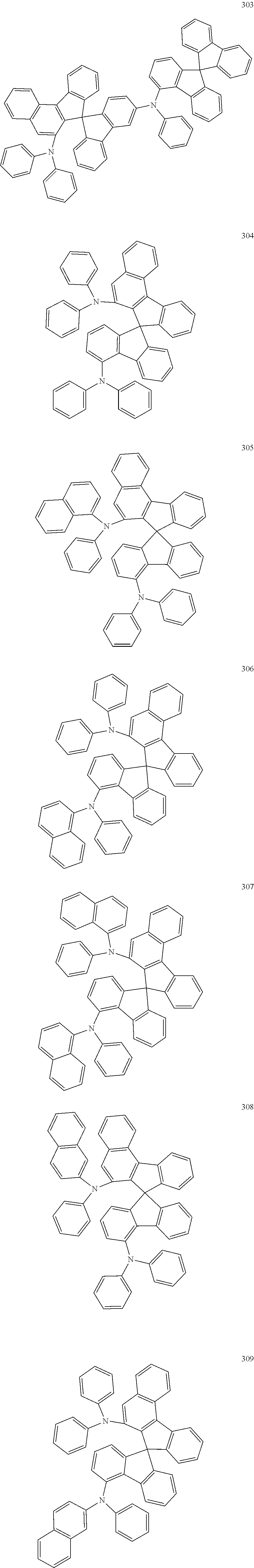

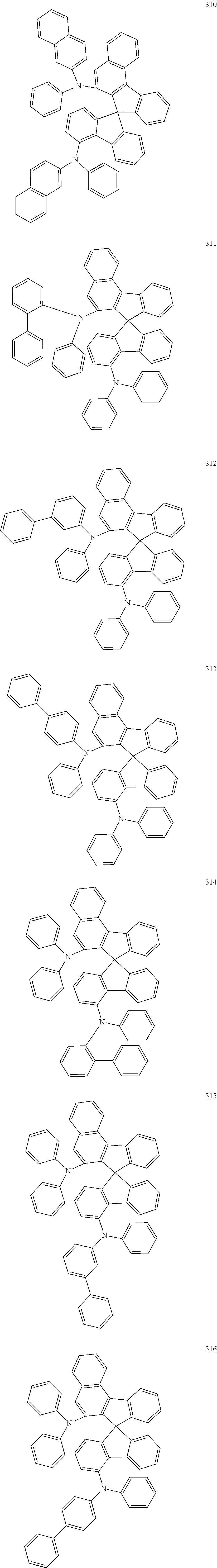

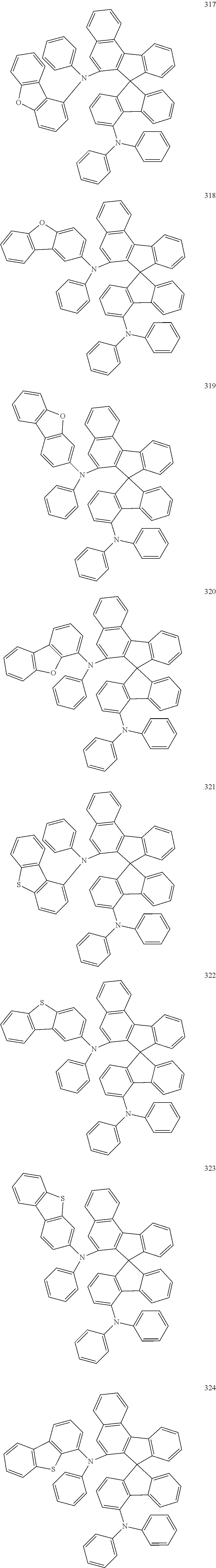

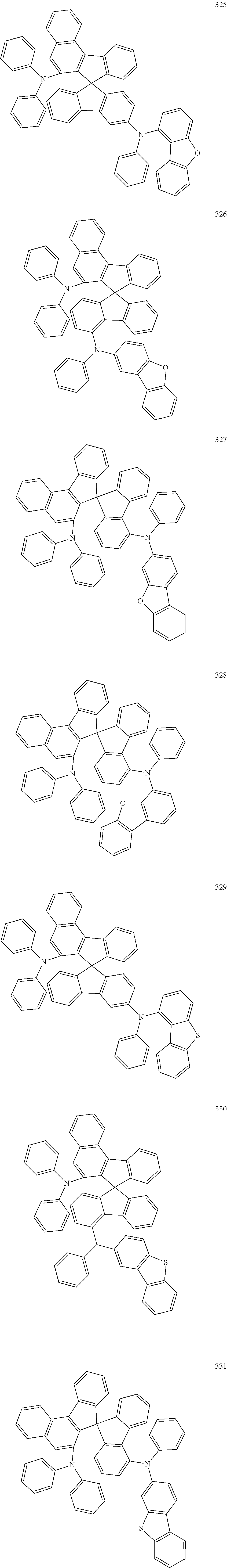

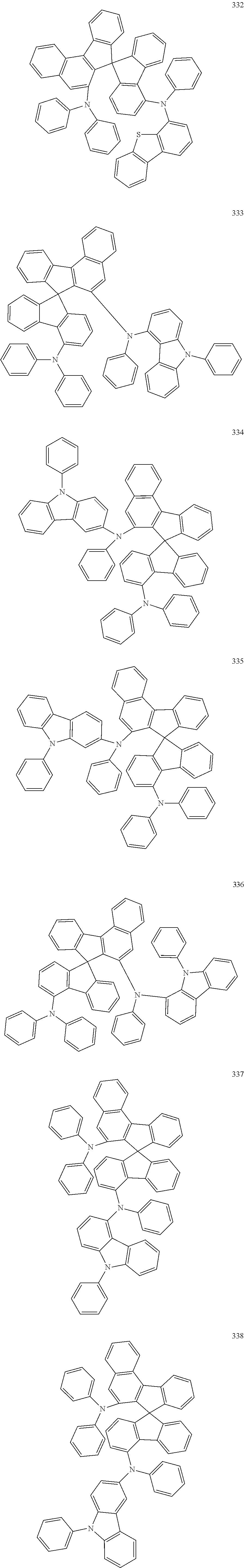

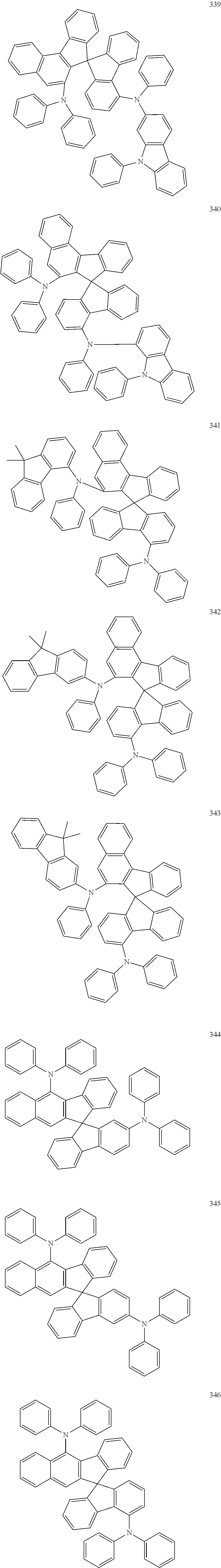

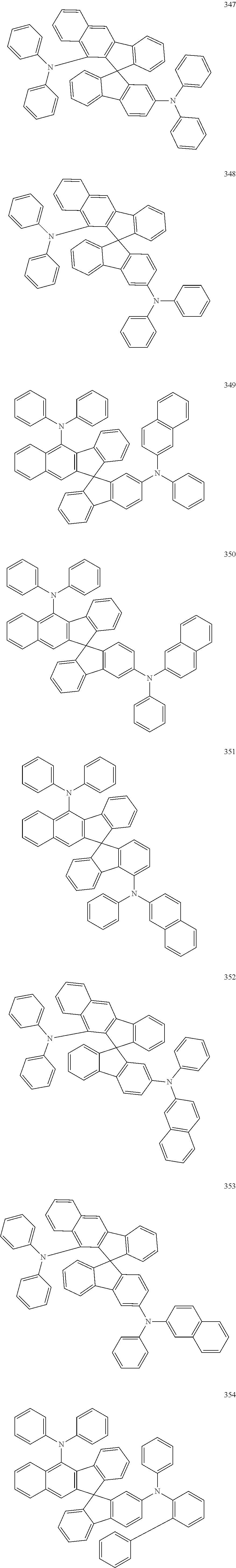

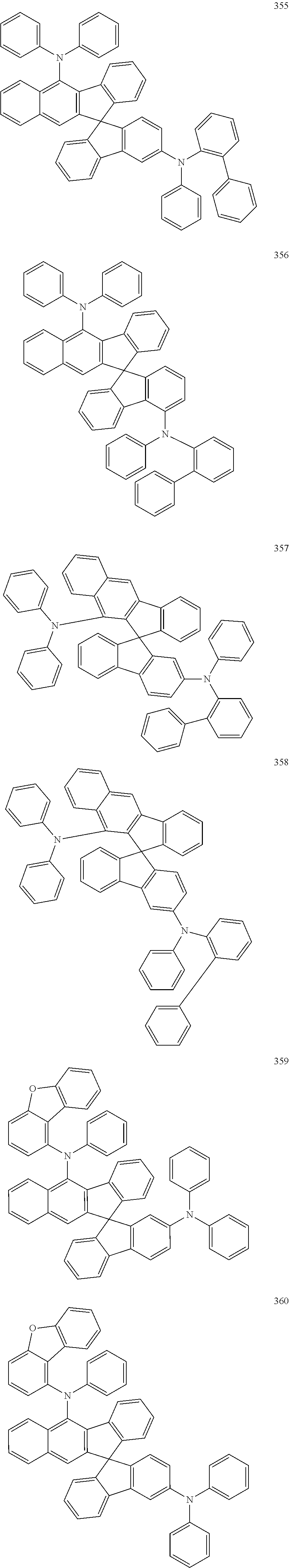

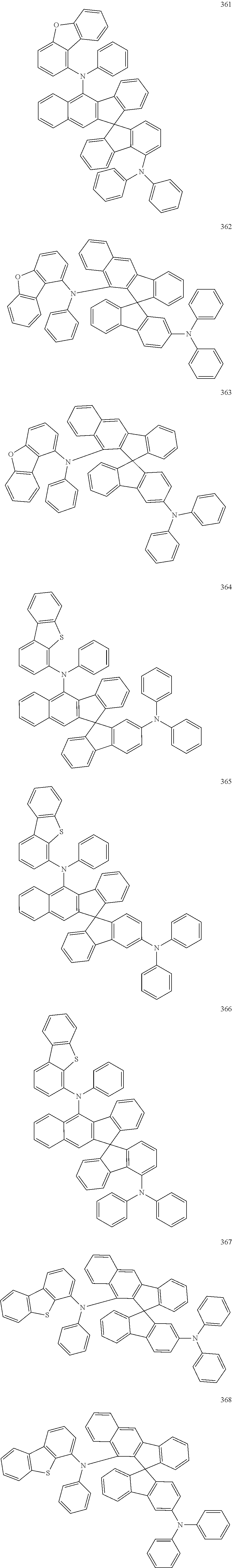

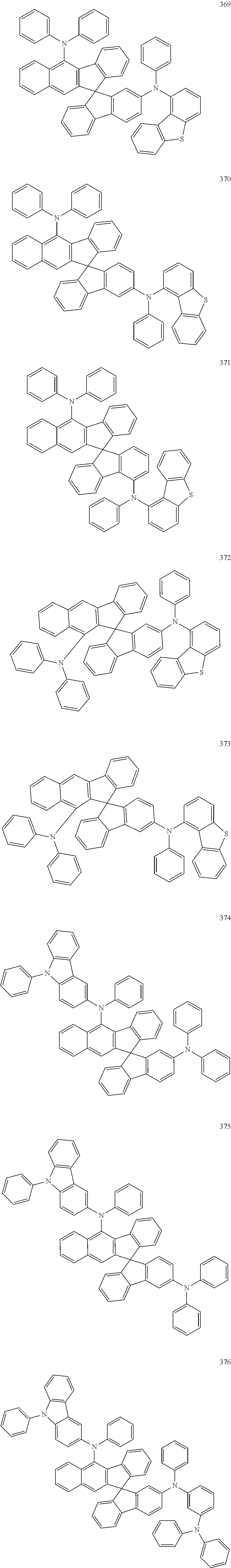

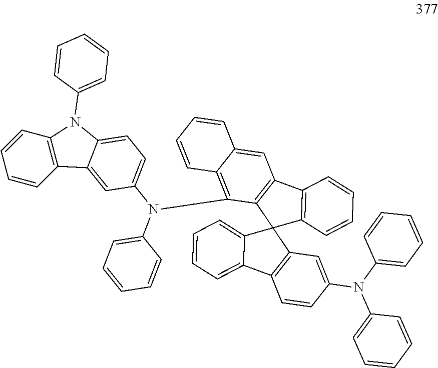 [SMILES: c1ccc(N(c2ccccc2)c2ccc3c(c2)C2(c4ccccc4-3)c3ccccc3-c3cc4ccccc4c(N(c4ccccc4)c4ccc5c(c4)c4ccccc4n5-c4ccccc4)c32)cc1]

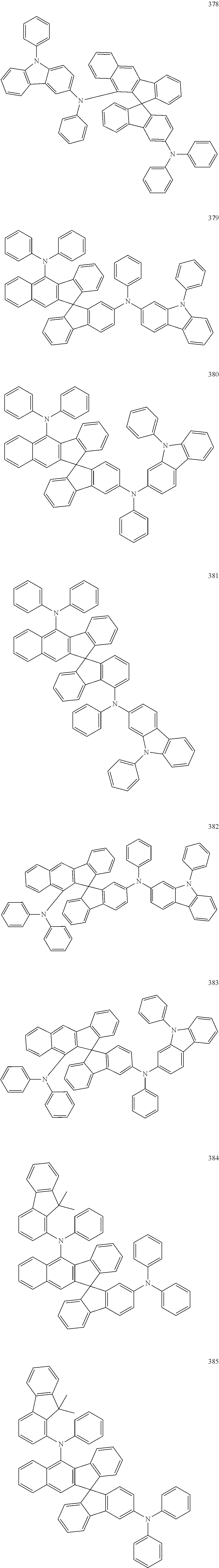

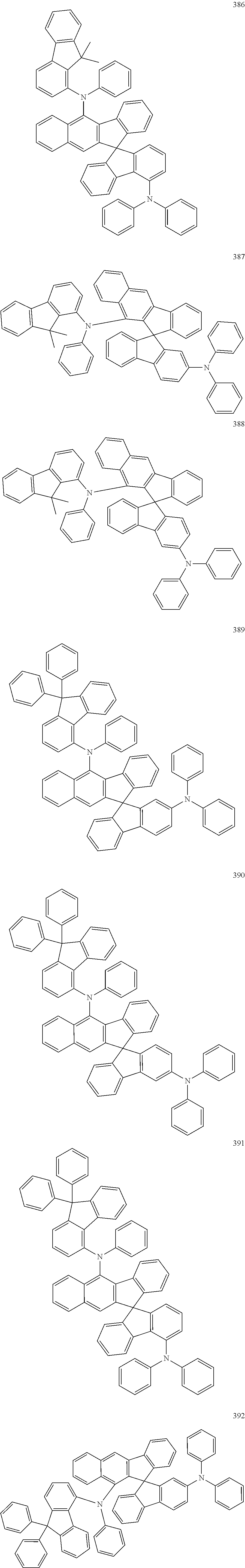

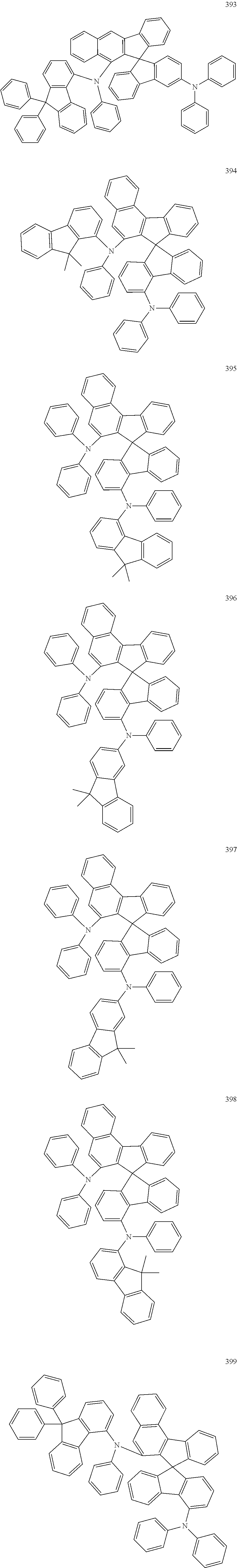

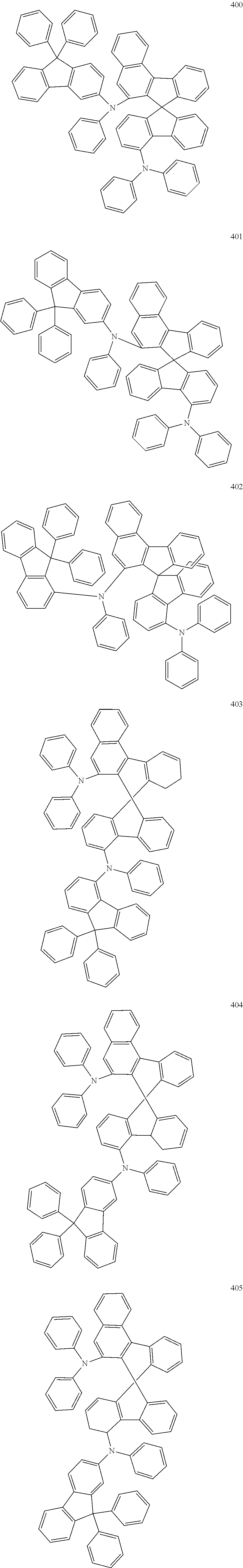

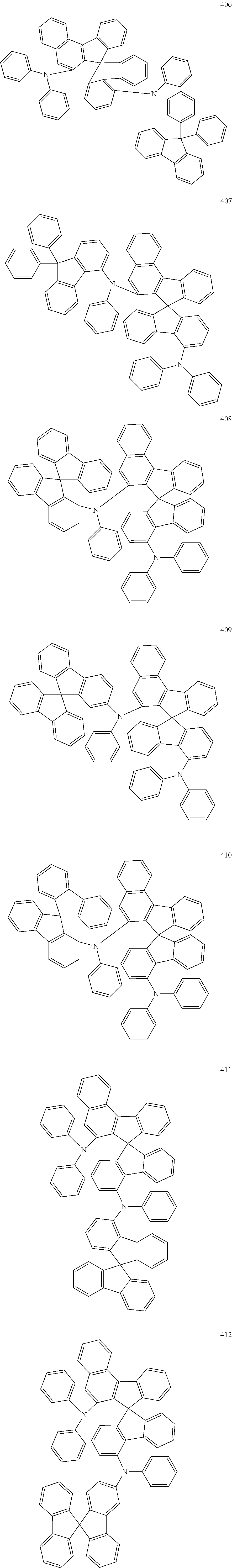

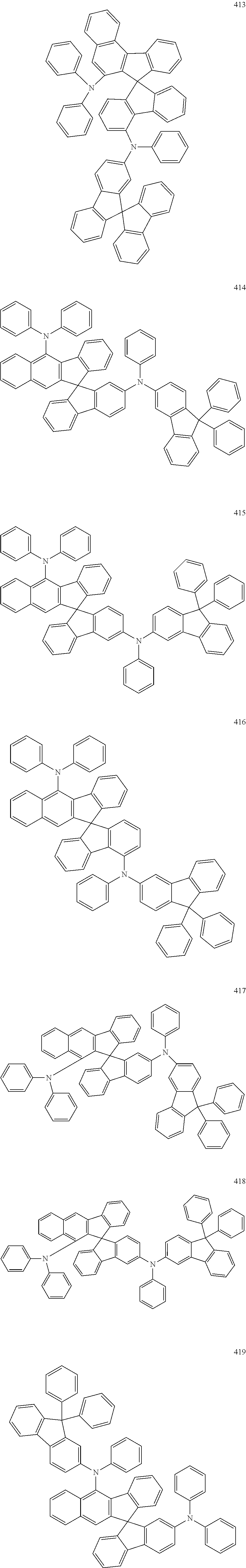

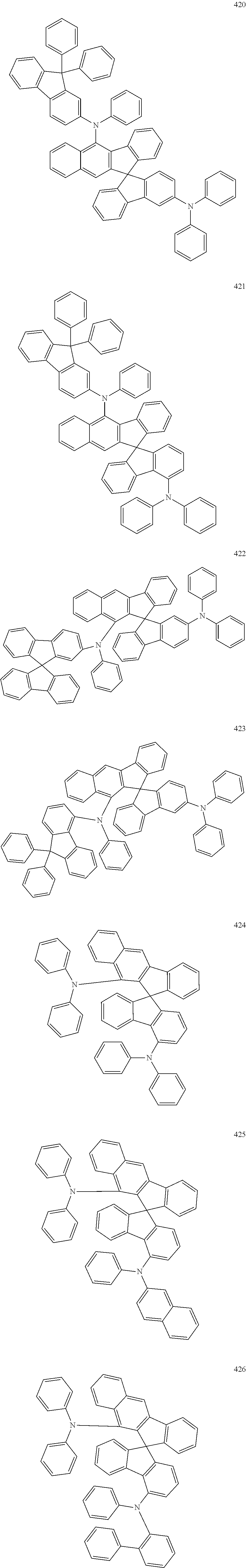

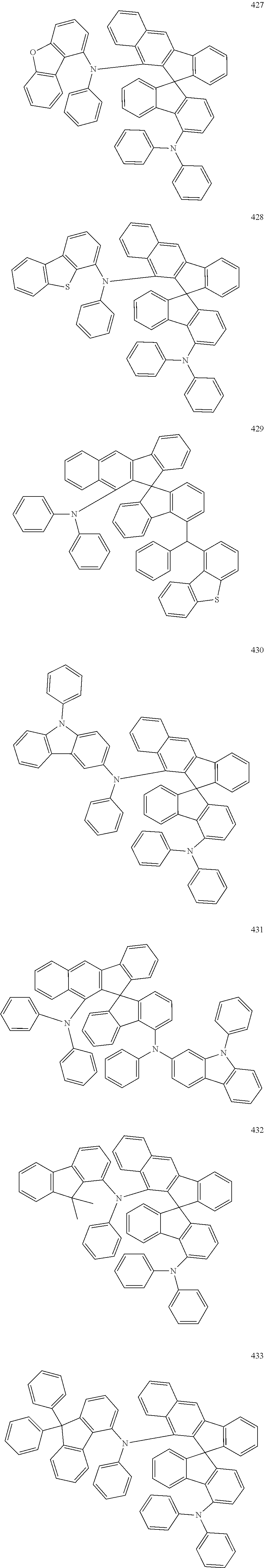

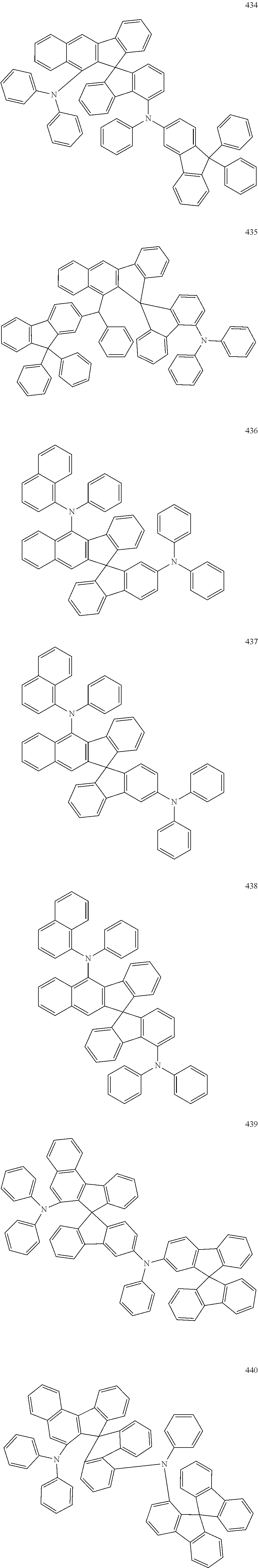

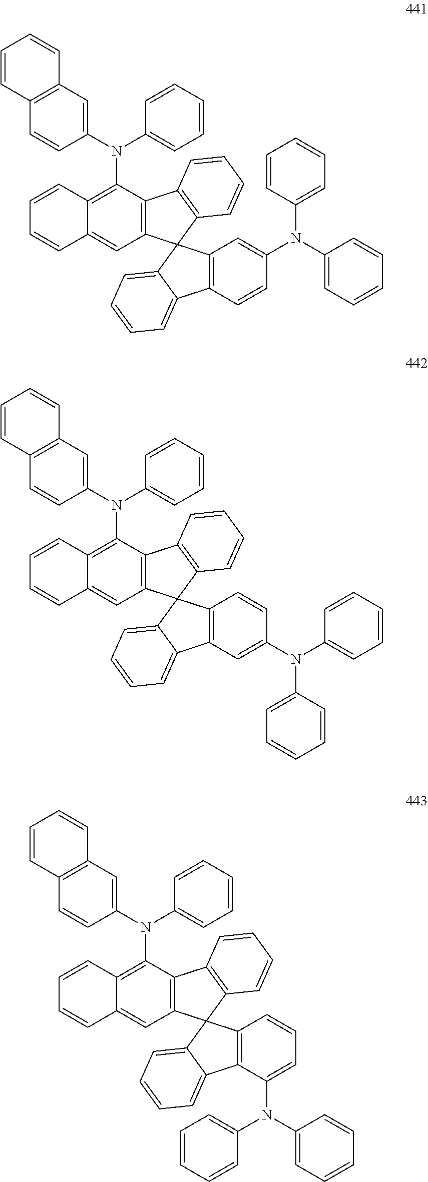 [SMILES: c1ccc(N(c2ccccc2)c2ccc3c(c2)C2(c4ccccc4-3)c3ccccc3-c3c2cc2ccccc2c3N(c2ccccc2)c2ccc3ccccc3c2)cc1 ; c1ccc(N(c2ccccc2)c2ccc3c(c2)-c2ccccc2C32c3ccccc3-c3c2cc2ccccc2c3N(c2ccccc2)c2ccc3ccccc3c2)cc1 ; c1ccc(N(c2ccccc2)c2cccc3c2-c2ccccc2C32c3ccccc3-c3c2cc2ccccc2c3N(c2ccccc2)c2ccc3ccccc3c2)cc1]

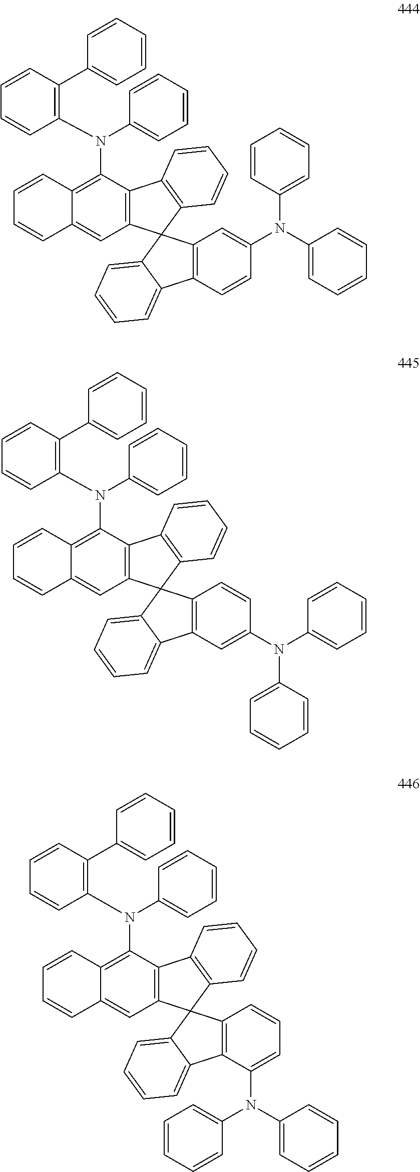 [SMILES: c1ccc(-c2ccccc2N(c2ccccc2)c2c3c(cc4ccccc24)C2(c4ccccc4-c4ccc(N(c5ccccc5)c5ccccc5)cc42)c2ccccc2-3)cc1 ; c1ccc(-c2ccccc2N(c2ccccc2)c2c3c(cc4ccccc24)C2(c4ccccc4-c4cc(N(c5ccccc5)c5ccccc5)ccc42)c2ccccc2-3)cc1 ; c1ccc(-c2ccccc2N(c2ccccc2)c2c3c(cc4ccccc24)C2(c4ccccc4-c4c(N(c5ccccc5)c5ccccc5)cccc42)c2ccccc2-3)cc1]

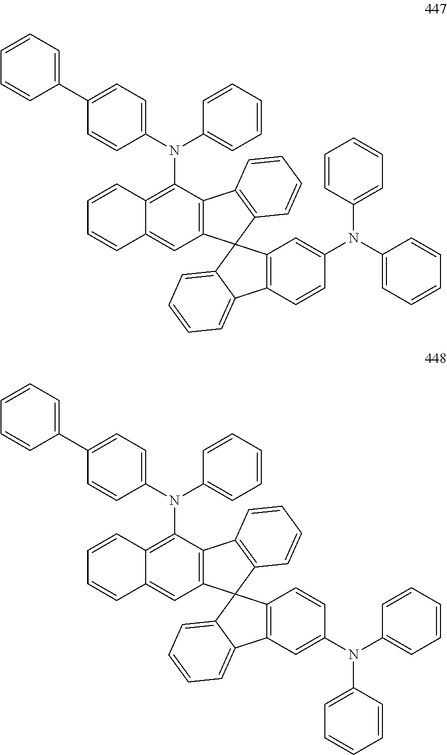 [SMILES: c1ccc(-c2ccc(N(c3ccccc3)c3c4c(cc5ccccc35)C3(c5ccccc5-c5ccc(N(c6ccccc6)c6ccccc6)cc53)c3ccccc3-4)cc2)cc1 ; c1ccc(-c2ccc(N(c3ccccc3)c3c4c(cc5ccccc35)C3(c5ccccc5-c5cc(N(c6ccccc6)c6ccccc6)ccc53)c3ccccc3-4)cc2)cc1]

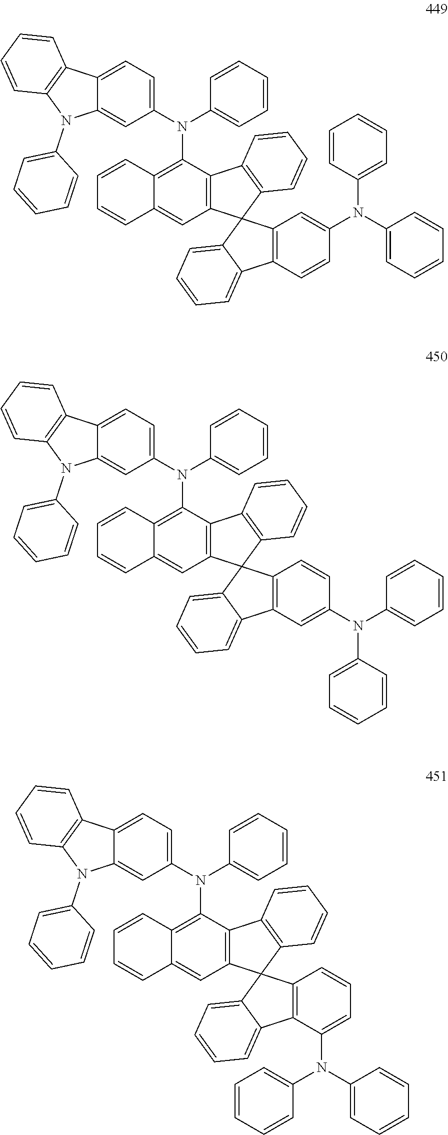 [SMILES: c1ccc(N(c2ccccc2)c2ccc3c(c2)C2(c4ccccc4-3)c3ccccc3-c3c2cc2ccccc2c3N(c2ccccc2)c2ccc3c4ccccc4n(-c4ccccc4)c3c2)cc1 ; c1ccc(N(c2ccccc2)c2ccc3c(c2)-c2ccccc2C32c3ccccc3-c3c2cc2ccccc2c3N(c2ccccc2)c2ccc3c4ccccc4n(-c4ccccc4)c3c2)cc1 ; c1ccc(N(c2ccccc2)c2cccc3c2-c2ccccc2C32c3ccccc3-c3c2cc2ccccc2c3N(c2ccccc2)c2ccc3c4ccccc4n(-c4ccccc4)c3c2)cc1]

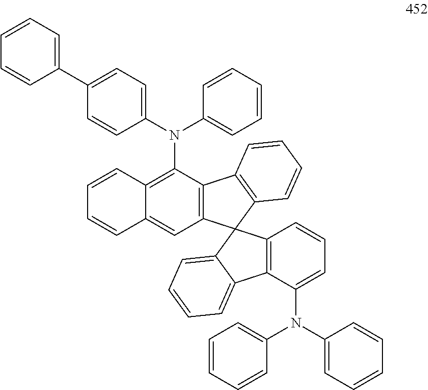 [SMILES: c1ccc(-c2ccc(N(c3ccccc3)c3c4c(cc5ccccc35)C3(c5ccccc5-c5c(N(c6ccccc6)c6ccccc6)cccc53)c3ccccc3-4)cc2)cc1]

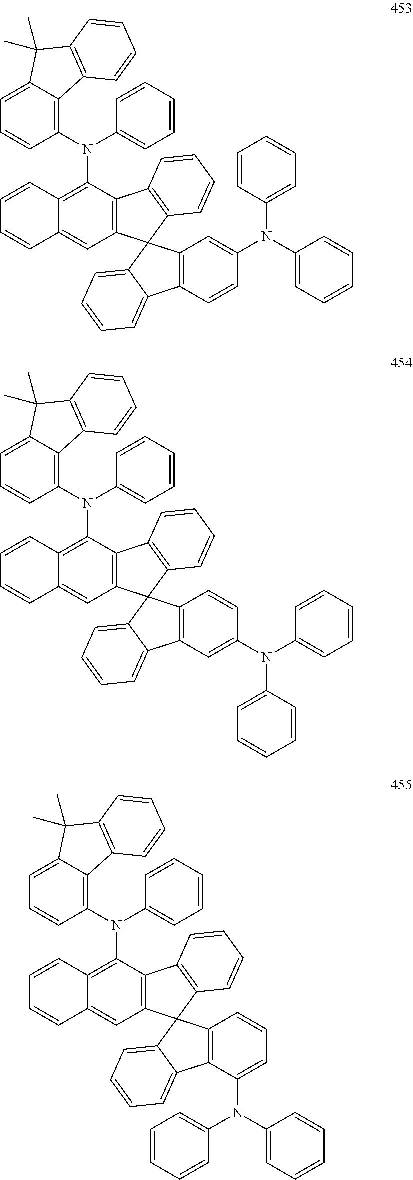

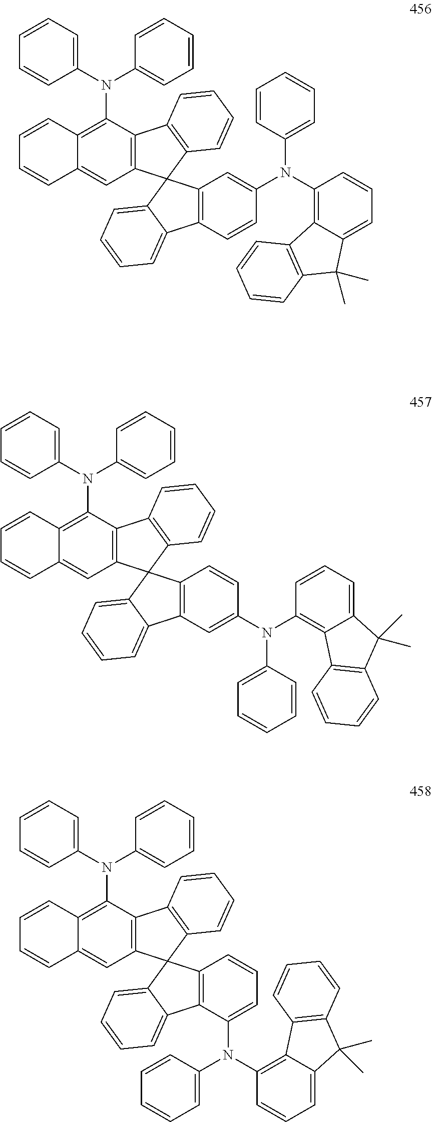 [SMILES: CC1(C)c2ccccc2-c2c(N(c3ccccc3)c3ccc4c(c3)C3(c5ccccc5-4)c4ccccc4-c4c3cc3ccccc3c4N(c3ccccc3)c3ccccc3)cccc21 ; CC1(C)c2ccccc2-c2c(N(c3ccccc3)c3ccc4c(c3)-c3ccccc3C43c4ccccc4-c4c3cc3ccccc3c4N(c3ccccc3)c3ccccc3)cccc21 ; CC1(C)c2ccccc2-c2c(N(c3ccccc3)c3cccc4c3-c3ccccc3C43c4ccccc4-c4c3cc3ccccc3c4N(c3ccccc3)c3ccccc3)cccc21]

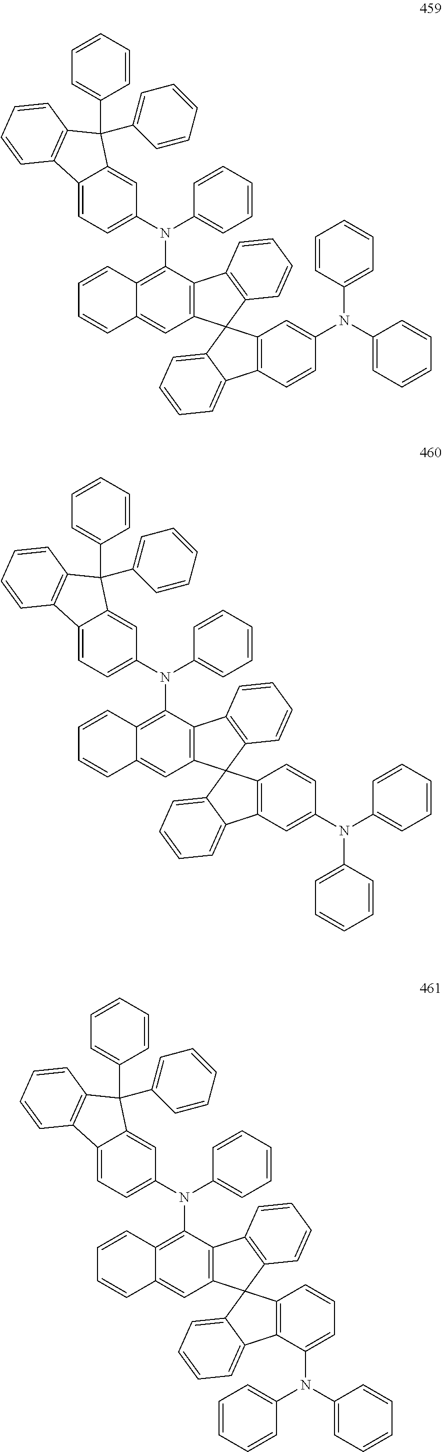 [SMILES: c1ccc(N(c2ccccc2)c2ccc3c(c2)C2(c4ccccc4-3)c3ccccc3-c3c2cc2ccccc2c3N(c2ccccc2)c2ccc3c(c2)C(c2ccccc2)(c2ccccc2)c2ccccc2-3)cc1 ; c1ccc(N(c2ccccc2)c2ccc3c(c2)-c2ccccc2C32c3ccccc3-c3c2cc2ccccc2c3N(c2ccccc2)c2ccc3c(c2)C(c2ccccc2)(c2ccccc2)c2ccccc2-3)cc1 ; c1ccc(N(c2ccccc2)c2cccc3c2-c2ccccc2C32c3ccccc3-c3c2cc2ccccc2c3N(c2ccccc2)c2ccc3c(c2)C(c2ccccc2)(c2ccccc2)c2ccccc2-3)cc1]

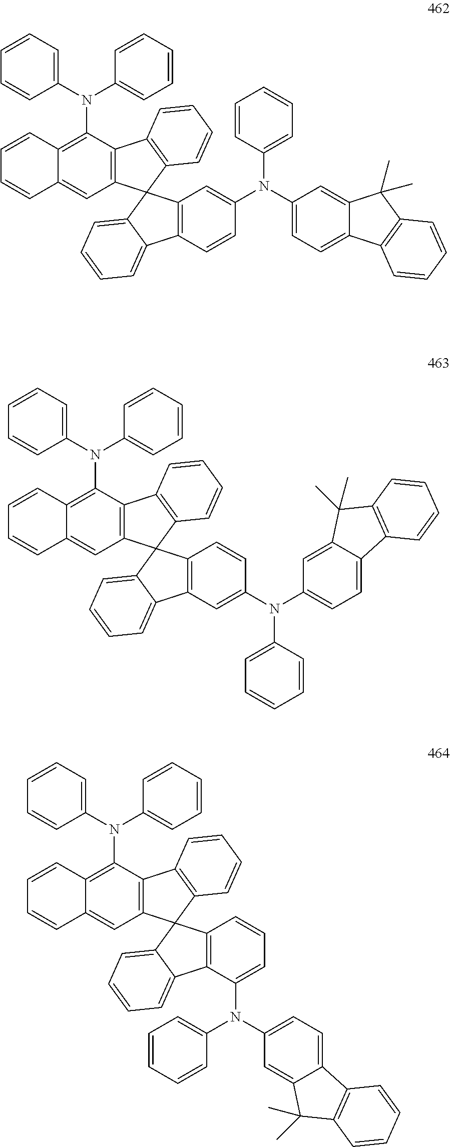 [SMILES: CC1(C)c2ccccc2-c2ccc(N(c3ccccc3)c3ccc4c(c3)C3(c5ccccc5-4)c4ccccc4-c4c3cc3ccccc3c4N(c3ccccc3)c3ccccc3)cc21 ; CC1(C)c2ccccc2-c2ccc(N(c3ccccc3)c3ccc4c(c3)-c3ccccc3C43c4ccccc4-c4c3cc3ccccc3c4N(c3ccccc3)c3ccccc3)cc21 ; CC1(C)c2ccccc2-c2ccc(N(c3ccccc3)c3cccc4c3-c3ccccc3C43c4ccccc4-c4c3cc3ccccc3c4N(c3ccccc3)c3ccccc3)cc21]

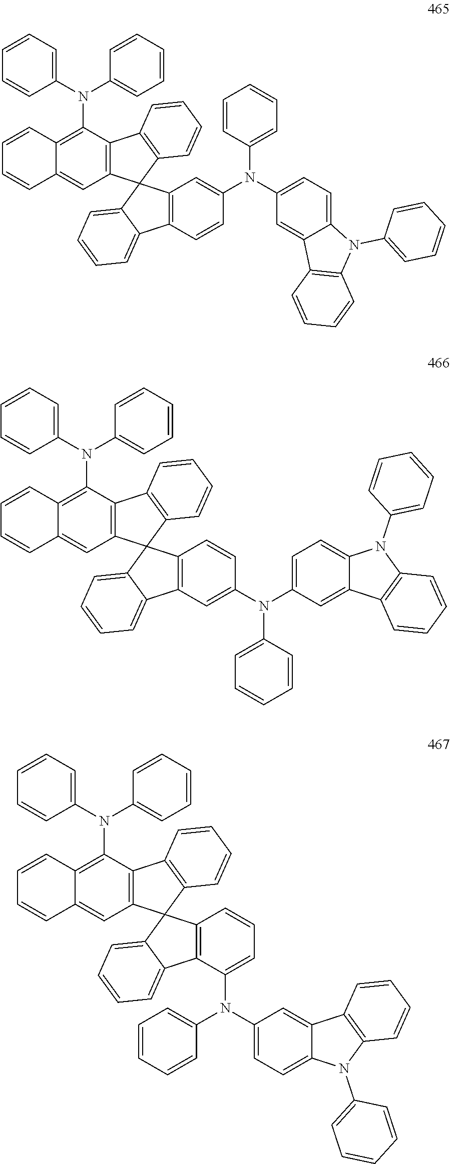 [SMILES: c1ccc(N(c2ccc3c(c2)C2(c4ccccc4-3)c3ccccc3-c3c2cc2ccccc2c3N(c2ccccc2)c2ccccc2)c2ccc3c(c2)c2ccccc2n3-c2ccccc2)cc1 ; c1ccc(N(c2ccc3c(c2)-c2ccccc2C32c3ccccc3-c3c2cc2ccccc2c3N(c2ccccc2)c2ccccc2)c2ccc3c(c2)c2ccccc2n3-c2ccccc2)cc1 ; c1ccc(N(c2ccc3c(c2)c2ccccc2n3-c2ccccc2)c2cccc3c2-c2ccccc2C32c3ccccc3-c3c2cc2ccccc2c3N(c2ccccc2)c2ccccc2)cc1]

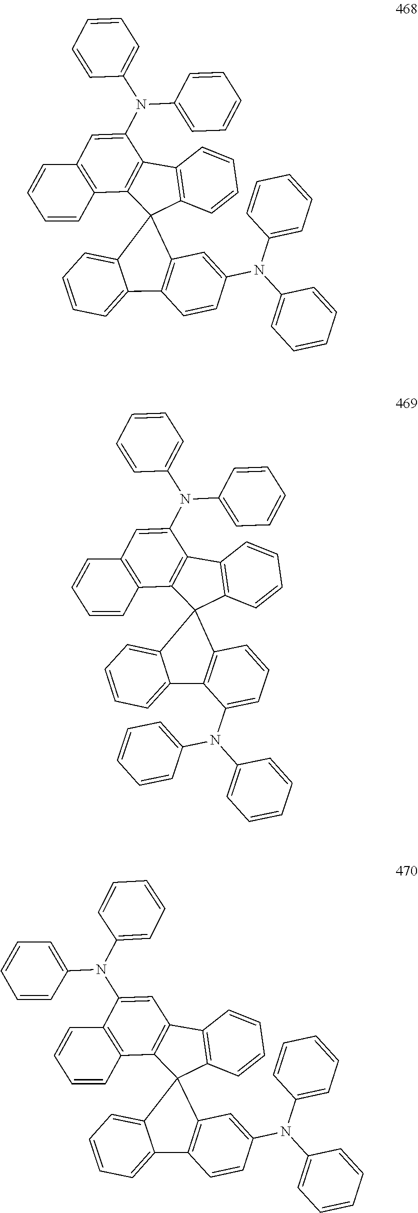 [SMILES: c1ccc(N(c2ccccc2)c2ccc3c(c2)C2(c4ccccc4-3)c3ccccc3-c3c(N(c4ccccc4)c4ccccc4)cc4ccccc4c32)cc1 ; c1ccc(N(c2ccccc2)c2cccc3c2-c2ccccc2C32c3ccccc3-c3c(N(c4ccccc4)c4ccccc4)cc4ccccc4c32)cc1 ; c1ccc(N(c2ccccc2)c2ccc3c(c2)C2(c4ccccc4-3)c3ccccc3-c3cc(N(c4ccccc4)c4ccccc4)c4ccccc4c32)cc1]

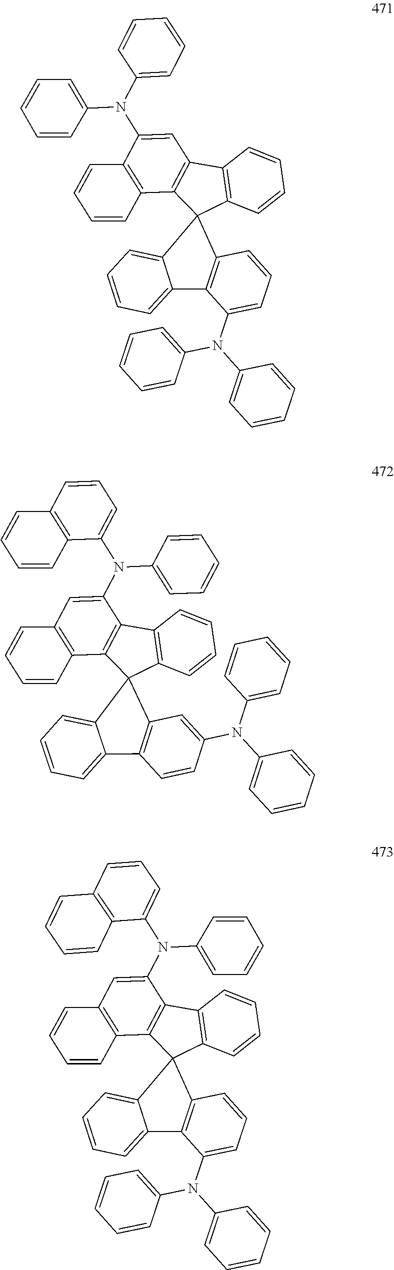 [SMILES: c1ccc(N(c2ccccc2)c2cccc3c2-c2ccccc2C32c3ccccc3-c3cc(N(c4ccccc4)c4ccccc4)c4ccccc4c32)cc1 ; c1ccc(N(c2ccccc2)c2ccc3c(c2)C2(c4ccccc4-3)c3ccccc3-c3c(N(c4ccccc4)c4cccc5ccccc45)cc4ccccc4c32)cc1 ; c1ccc(N(c2ccccc2)c2cccc3c2-c2ccccc2C32c3ccccc3-c3c(N(c4ccccc4)c4cccc5ccccc45)cc4ccccc4c32)cc1]

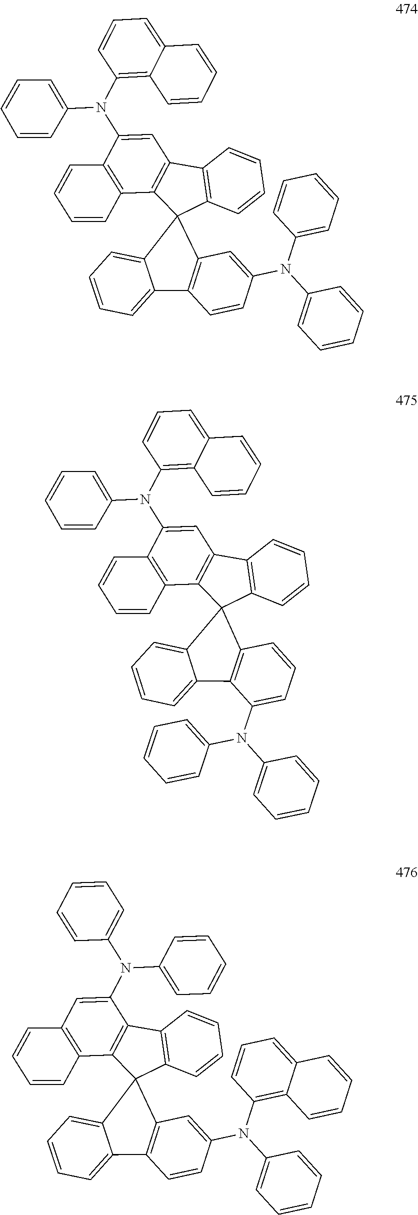 [SMILES: c1ccc(N(c2ccccc2)c2ccc3c(c2)C2(c4ccccc4-3)c3ccccc3-c3cc(N(c4ccccc4)c4cccc5ccccc45)c4ccccc4c32)cc1 ; c1ccc(N(c2ccccc2)c2cccc3c2-c2ccccc2C32c3ccccc3-c3cc(N(c4ccccc4)c4cccc5ccccc45)c4ccccc4c32)cc1 ; c1ccc(N(c2ccccc2)c2cc3ccccc3c3c2-c2ccccc2C32c3ccccc3-c3ccc(N(c4ccccc4)c4cccc5ccccc45)cc32)cc1]

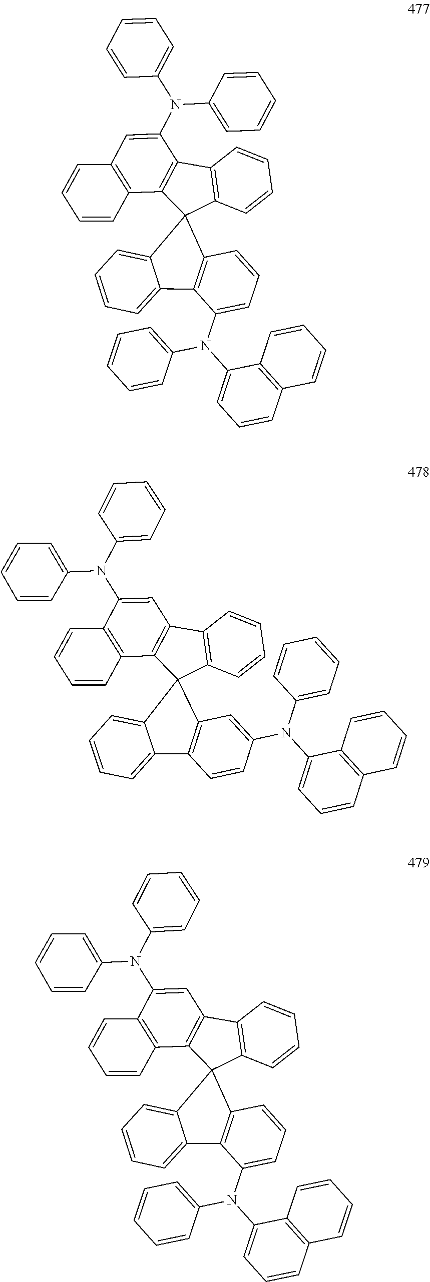 [SMILES: c1ccc(N(c2ccccc2)c2cc3ccccc3c3c2-c2ccccc2C32c3ccccc3-c3c(N(c4ccccc4)c4cccc5ccccc45)cccc32)cc1 ; c1ccc(N(c2ccc3c(c2)C2(c4ccccc4-3)c3ccccc3-c3cc(N(c4ccccc4)c4ccccc4)c4ccccc4c32)c2cccc3ccccc23)cc1 ; c1ccc(N(c2cccc3c2-c2ccccc2C32c3ccccc3-c3cc(N(c4ccccc4)c4ccccc4)c4ccccc4c32)c2cccc3ccccc23)cc1]

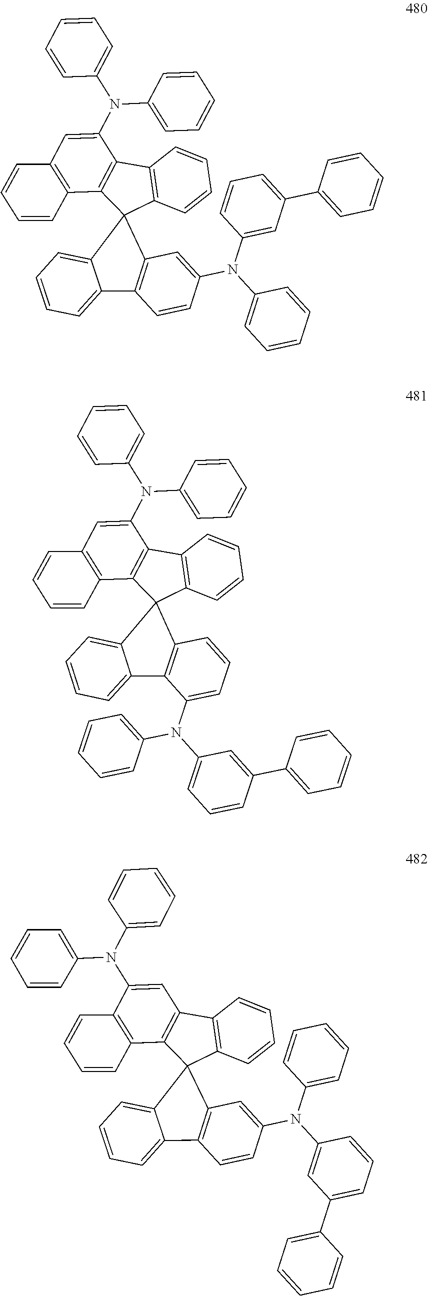 [SMILES: c1ccc(-c2cccc(N(c3ccccc3)c3ccc4c(c3)C3(c5ccccc5-4)c4ccccc4-c4c(N(c5ccccc5)c5ccccc5)cc5ccccc5c43)c2)cc1 ; c1ccc(-c2cccc(N(c3ccccc3)c3cccc4c3-c3ccccc3C43c4ccccc4-c4c(N(c5ccccc5)c5ccccc5)cc5ccccc5c43)c2)cc1 ; c1ccc(-c2cccc(N(c3ccccc3)c3ccc4c(c3)C3(c5ccccc5-4)c4ccccc4-c4cc(N(c5ccccc5)c5ccccc5)c5ccccc5c43)c2)cc1]

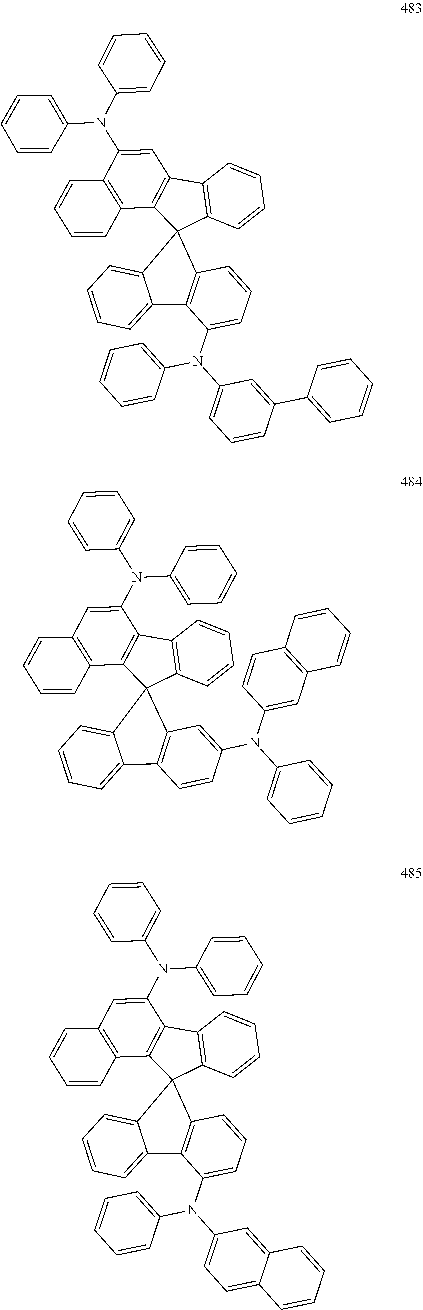 [SMILES: c1ccc(-c2cccc(N(c3ccccc3)c3cccc4c3-c3ccccc3C43c4ccccc4-c4cc(N(c5ccccc5)c5ccccc5)c5ccccc5c43)c2)cc1 ; c1ccc(N(c2ccc3c(c2)C2(c4ccccc4-3)c3ccccc3-c3c(N(c4ccccc4)c4ccccc4)cc4ccccc4c32)c2ccc3ccccc3c2)cc1 ; c1ccc(N(c2ccc3ccccc3c2)c2cccc3c2-c2ccccc2C32c3ccccc3-c3c(N(c4ccccc4)c4ccccc4)cc4ccccc4c32)cc1]

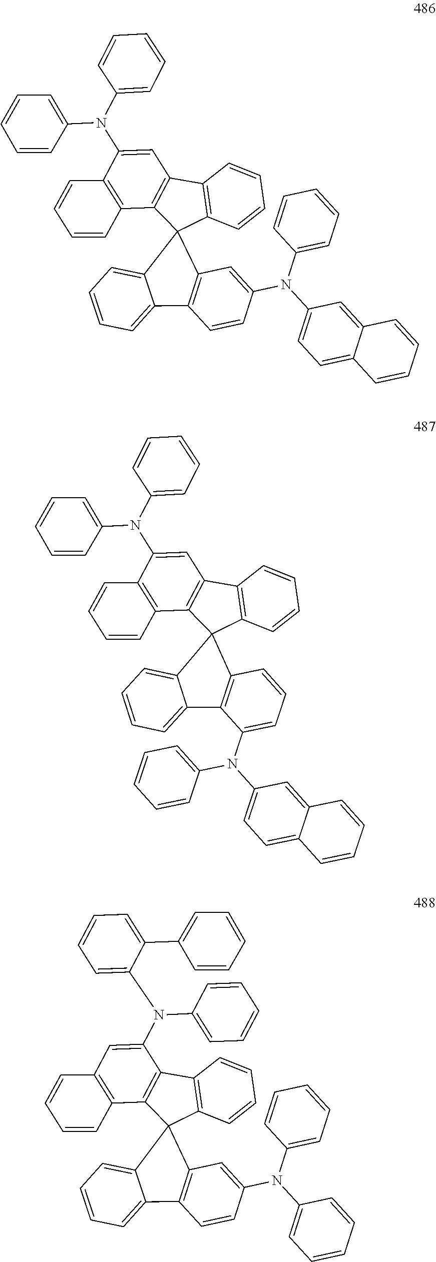 [SMILES: c1ccc(N(c2ccc3c(c2)C2(c4ccccc4-3)c3ccccc3-c3cc(N(c4ccccc4)c4ccccc4)c4ccccc4c32)c2ccc3ccccc3c2)cc1 ; c1ccc(N(c2ccc3ccccc3c2)c2cccc3c2-c2ccccc2C32c3ccccc3-c3cc(N(c4ccccc4)c4ccccc4)c4ccccc4c32)cc1 ; c1ccc(-c2ccccc2N(c2ccccc2)c2cc3ccccc3c3c2-c2ccccc2C32c3ccccc3-c3ccc(N(c4ccccc4)c4ccccc4)cc32)cc1]

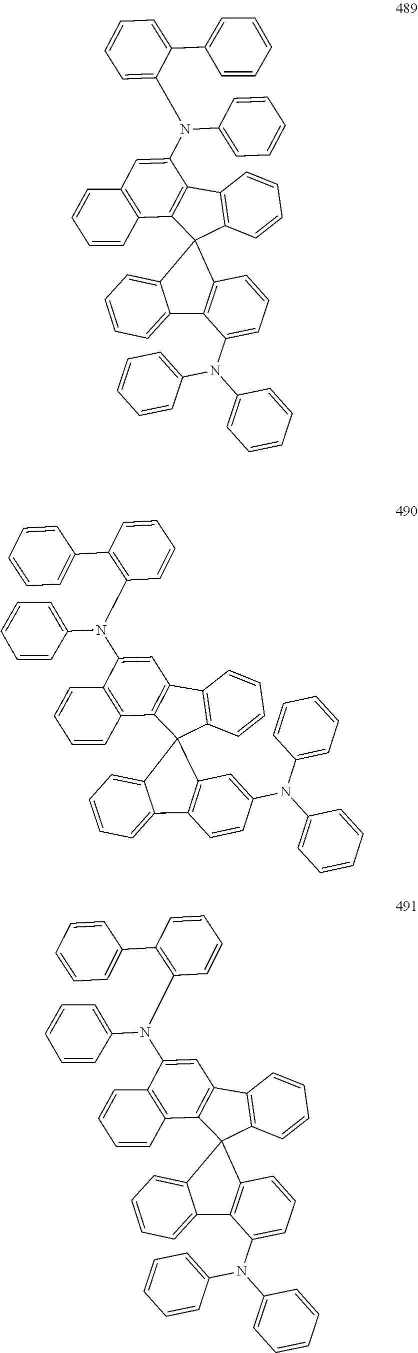 [SMILES: c1ccc(-c2ccccc2N(c2ccccc2)c2cc3ccccc3c3c2-c2ccccc2C32c3ccccc3-c3c(N(c4ccccc4)c4ccccc4)cccc32)cc1 ; c1ccc(-c2ccccc2N(c2ccccc2)c2cc3c(c4ccccc24)C2(c4ccccc4-c4ccc(N(c5ccccc5)c5ccccc5)cc42)c2ccccc2-3)cc1 ; c1ccc(-c2ccccc2N(c2ccccc2)c2cc3c(c4ccccc24)C2(c4ccccc4-3)c3ccccc3-c3c(N(c4ccccc4)c4ccccc4)cccc32)cc1]

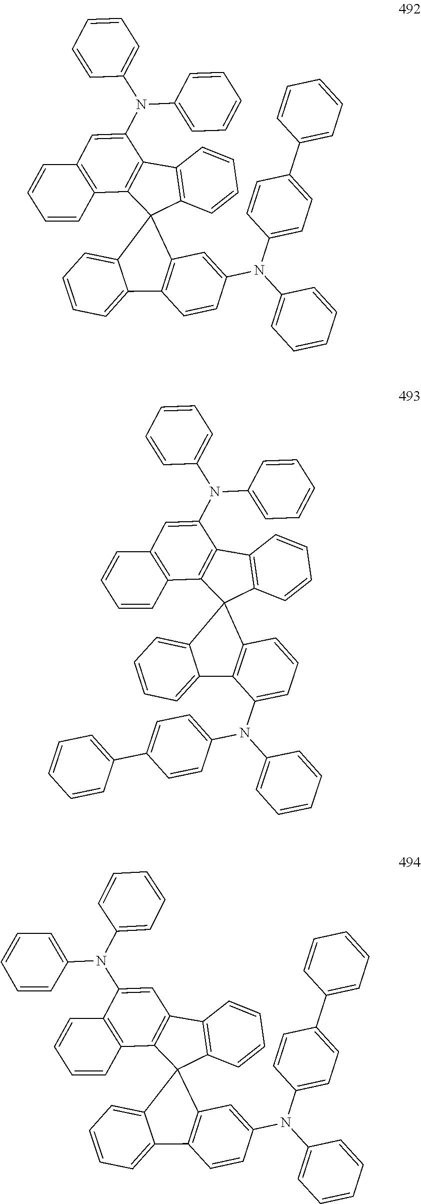 [SMILES: c1ccc(-c2ccc(N(c3ccccc3)c3ccc4c(c3)C3(c5ccccc5-4)c4ccccc4-c4c(N(c5ccccc5)c5ccccc5)cc5ccccc5c43)cc2)cc1 ; c1ccc(-c2ccc(N(c3ccccc3)c3cccc4c3-c3ccccc3C43c4ccccc4-c4c(N(c5ccccc5)c5ccccc5)cc5ccccc5c43)cc2)cc1 ; c1ccc(-c2ccc(N(c3ccccc3)c3ccc4c(c3)C3(c5ccccc5-4)c4ccccc4-c4cc(N(c5ccccc5)c5ccccc5)c5ccccc5c43)cc2)cc1]

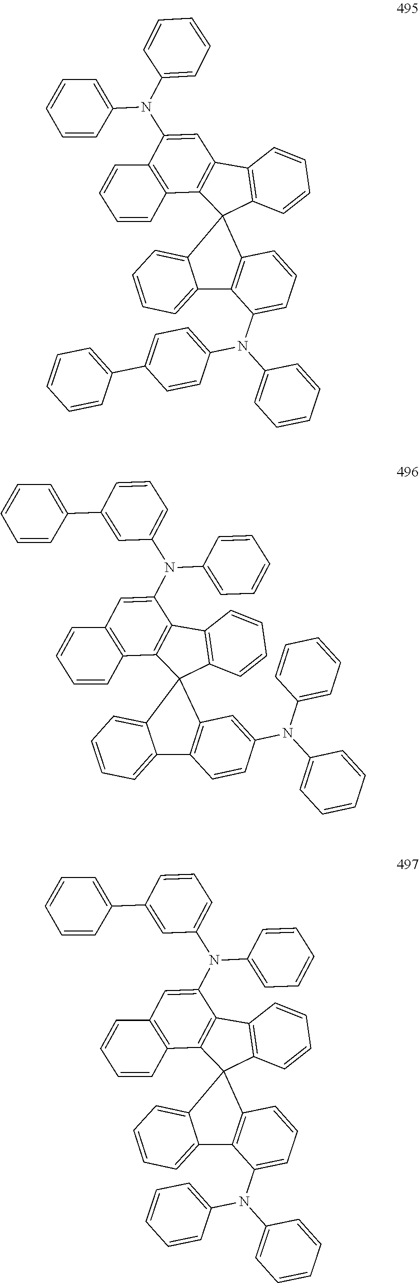 [SMILES: c1ccc(-c2ccc(N(c3ccccc3)c3cccc4c3-c3ccccc3C43c4ccccc4-c4cc(N(c5ccccc5)c5ccccc5)c5ccccc5c43)cc2)cc1 ; c1ccc(-c2cccc(N(c3ccccc3)c3cc4ccccc4c4c3-c3ccccc3C43c4ccccc4-c4ccc(N(c5ccccc5)c5ccccc5)cc43)c2)cc1 ; c1ccc(-c2cccc(N(c3ccccc3)c3cc4ccccc4c4c3-c3ccccc3C43c4ccccc4-c4c(N(c5ccccc5)c5ccccc5)cccc43)c2)cc1]

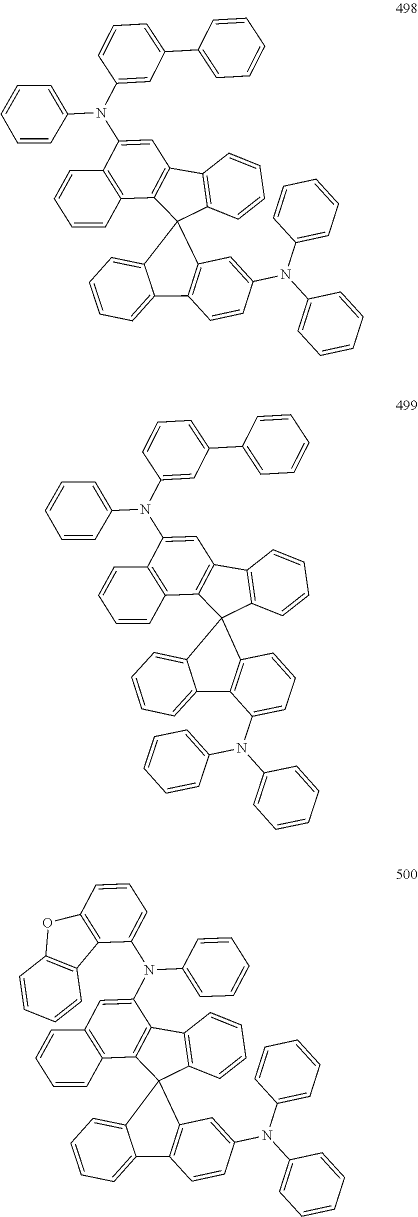 [SMILES: c1ccc(-c2cccc(N(c3ccccc3)c3cc4c(c5ccccc35)C3(c5ccccc5-c5ccc(N(c6ccccc6)c6ccccc6)cc53)c3ccccc3-4)c2)cc1 ; c1ccc(-c2cccc(N(c3ccccc3)c3cc4c(c5ccccc35)C3(c5ccccc5-4)c4ccccc4-c4c(N(c5ccccc5)c5ccccc5)cccc43)c2)cc1 ; c1ccc(N(c2ccccc2)c2ccc3c(c2)C2(c4ccccc4-3)c3ccccc3-c3c(N(c4ccccc4)c4cccc5oc6ccccc6c45)cc4ccccc4c32)cc1]

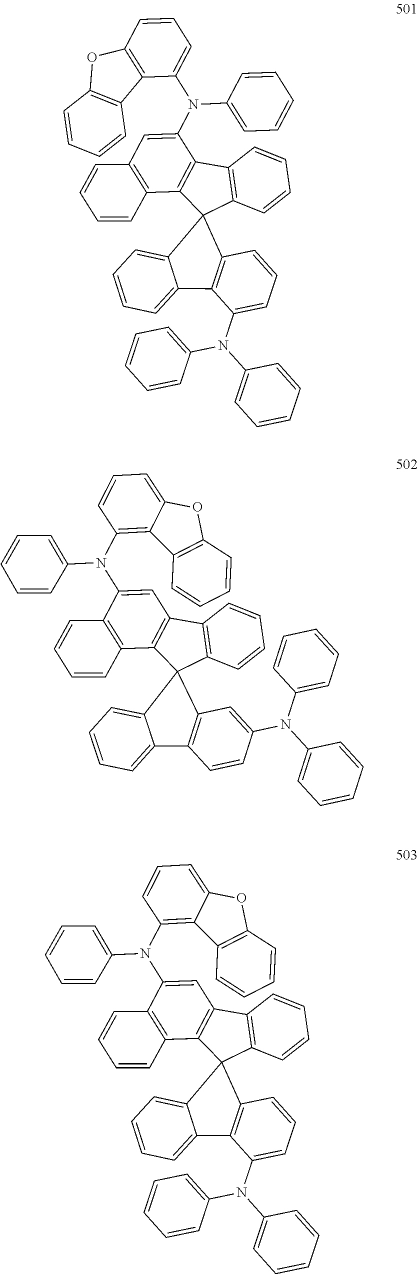 [SMILES: c1ccc(N(c2ccccc2)c2cccc3c2-c2ccccc2C32c3ccccc3-c3c(N(c4ccccc4)c4cccc5oc6ccccc6c45)cc4ccccc4c32)cc1 ; c1ccc(N(c2ccccc2)c2ccc3c(c2)C2(c4ccccc4-3)c3ccccc3-c3cc(N(c4ccccc4)c4cccc5oc6ccccc6c45)c4ccccc4c32)cc1 ; c1ccc(N(c2ccccc2)c2cccc3c2-c2ccccc2C32c3ccccc3-c3cc(N(c4ccccc4)c4cccc5oc6ccccc6c45)c4ccccc4c32)cc1]

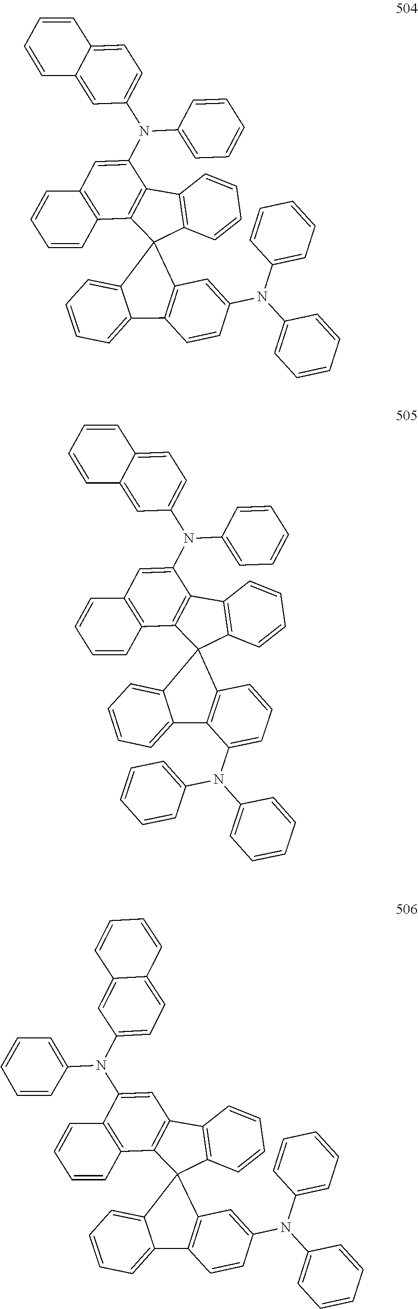 [SMILES: c1ccc(N(c2ccccc2)c2ccc3c(c2)C2(c4ccccc4-3)c3ccccc3-c3c(N(c4ccccc4)c4ccc5ccccc5c4)cc4ccccc4c32)cc1 ; c1ccc(N(c2ccccc2)c2cccc3c2-c2ccccc2C32c3ccccc3-c3c(N(c4ccccc4)c4ccc5ccccc5c4)cc4ccccc4c32)cc1 ; c1ccc(N(c2ccccc2)c2ccc3c(c2)C2(c4ccccc4-3)c3ccccc3-c3cc(N(c4ccccc4)c4ccc5ccccc5c4)c4ccccc4c32)cc1]

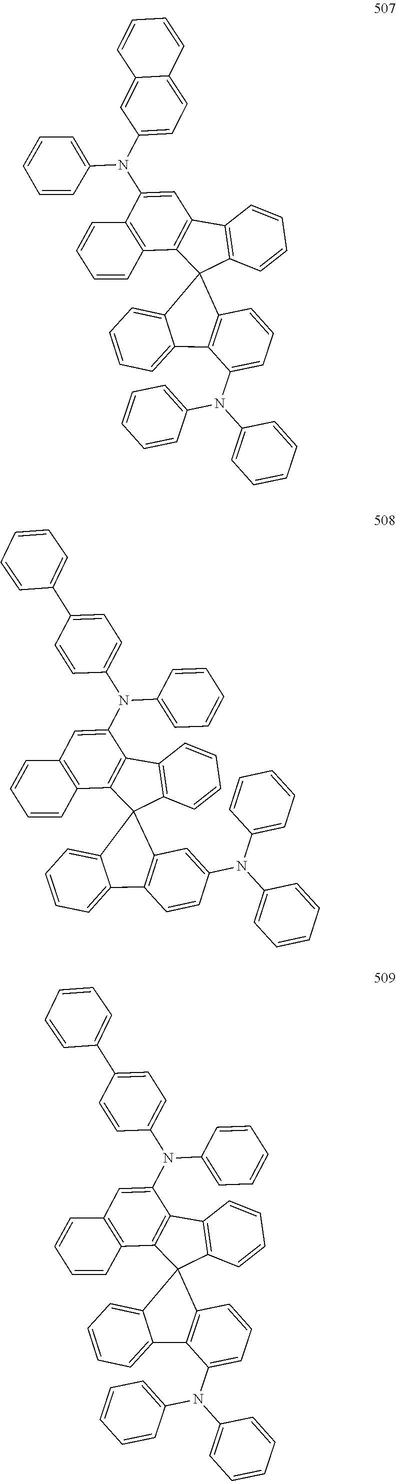 [SMILES: c1ccc(N(c2ccccc2)c2cccc3c2-c2ccccc2C32c3ccccc3-c3cc(N(c4ccccc4)c4ccc5ccccc5c4)c4ccccc4c32)cc1 ; c1ccc(-c2ccc(N(c3ccccc3)c3cc4ccccc4c4c3-c3ccccc3C43c4ccccc4-c4ccc(N(c5ccccc5)c5ccccc5)cc43)cc2)cc1 ; c1ccc(-c2ccc(N(c3ccccc3)c3cc4ccccc4c4c3-c3ccccc3C43c4ccccc4-c4c(N(c5ccccc5)c5ccccc5)cccc43)cc2)cc1]

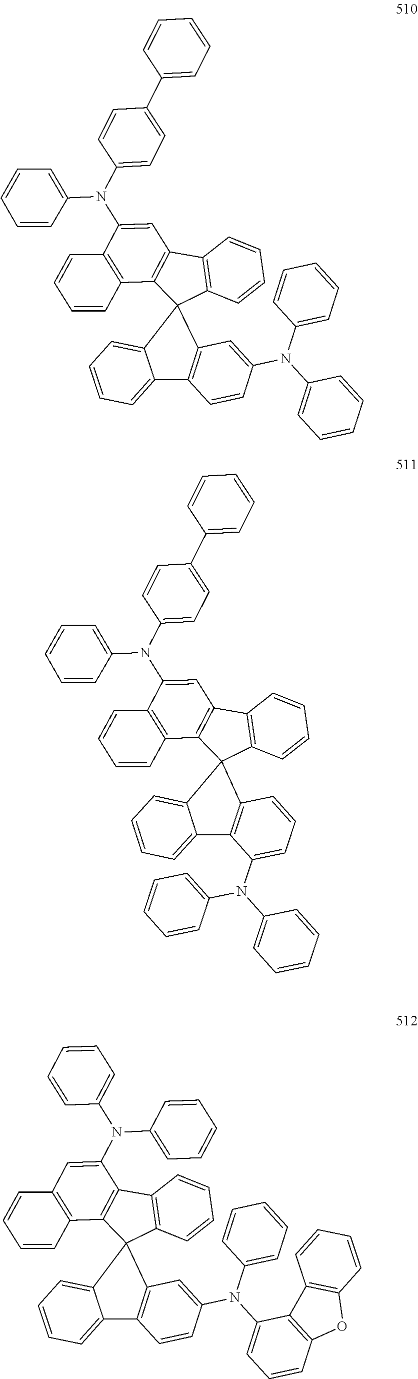 [SMILES: c1ccc(-c2ccc(N(c3ccccc3)c3cc4c(c5ccccc35)C3(c5ccccc5-c5ccc(N(c6ccccc6)c6ccccc6)cc53)c3ccccc3-4)cc2)cc1 ; c1ccc(-c2ccc(N(c3ccccc3)c3cc4c(c5ccccc35)C3(c5ccccc5-4)c4ccccc4-c4c(N(c5ccccc5)c5ccccc5)cccc43)cc2)cc1 ; c1ccc(N(c2ccccc2)c2cc3ccccc3c3c2-c2ccccc2C32c3ccccc3-c3ccc(N(c4ccccc4)c4cccc5oc6ccccc6c45)cc32)cc1]

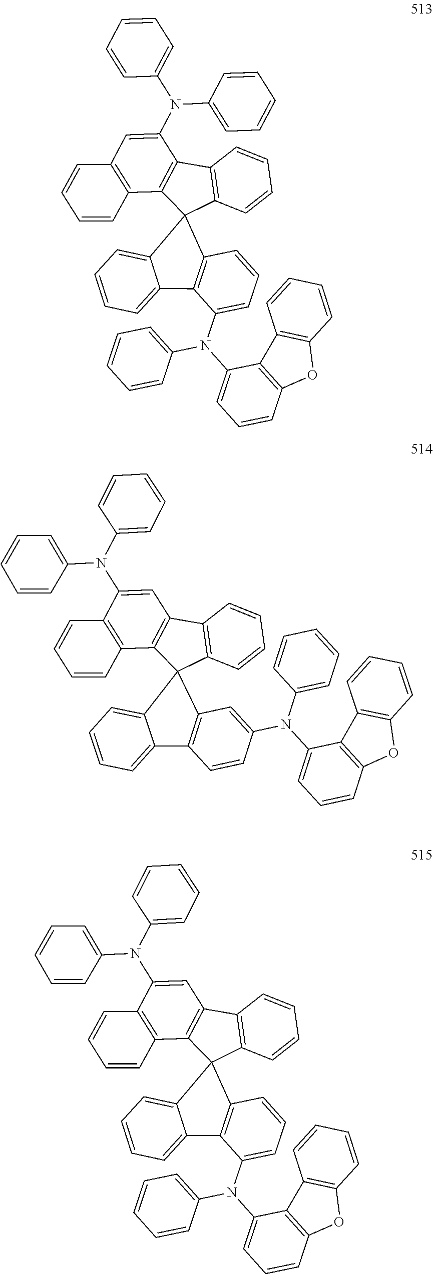 [SMILES: c1ccc(N(c2ccccc2)c2cc3ccccc3c3c2-c2ccccc2C32c3ccccc3-c3c(N(c4ccccc4)c4cccc5oc6ccccc6c45)cccc32)cc1 ; c1ccc(N(c2ccccc2)c2cc3c(c4ccccc24)C2(c4ccccc4-c4ccc(N(c5ccccc5)c5cccc6oc7ccccc7c56)cc42)c2ccccc2-3)cc1 ; c1ccc(N(c2ccccc2)c2cc3c(c4ccccc24)C2(c4ccccc4-3)c3ccccc3-c3c(N(c4ccccc4)c4cccc5oc6ccccc6c45)cccc32)cc1]

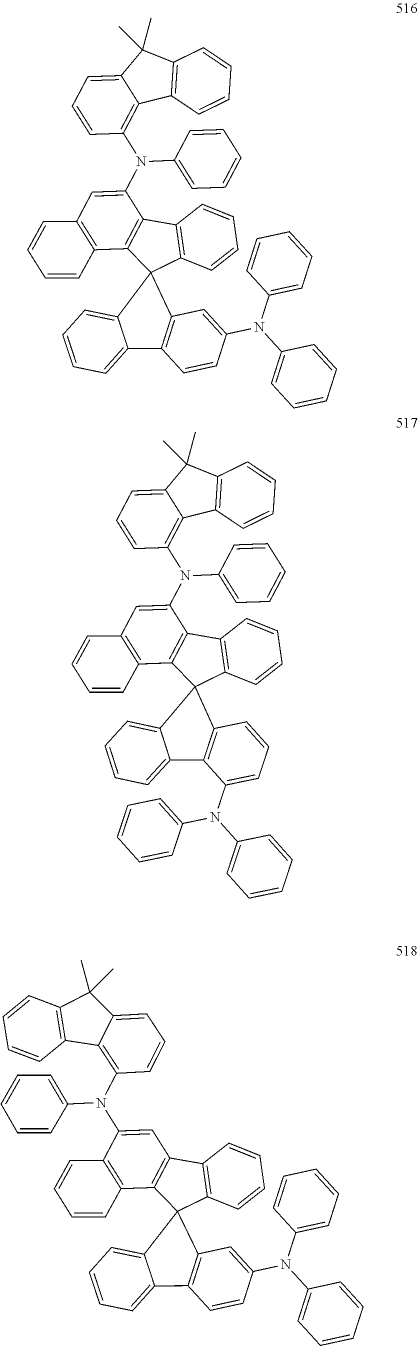 [SMILES: CC1(C)c2ccccc2-c2c(N(c3ccccc3)c3cc4ccccc4c4c3-c3ccccc3C43c4ccccc4-c4ccc(N(c5ccccc5)c5ccccc5)cc43)cccc21 ; CC1(C)c2ccccc2-c2c(N(c3ccccc3)c3cc4ccccc4c4c3-c3ccccc3C43c4ccccc4-c4c(N(c5ccccc5)c5ccccc5)cccc43)cccc21 ; CC1(C)c2ccccc2-c2c(N(c3ccccc3)c3cc4c(c5ccccc35)C3(c5ccccc5-c5ccc(N(c6ccccc6)c6ccccc6)cc53)c3ccccc3-4)cccc21]

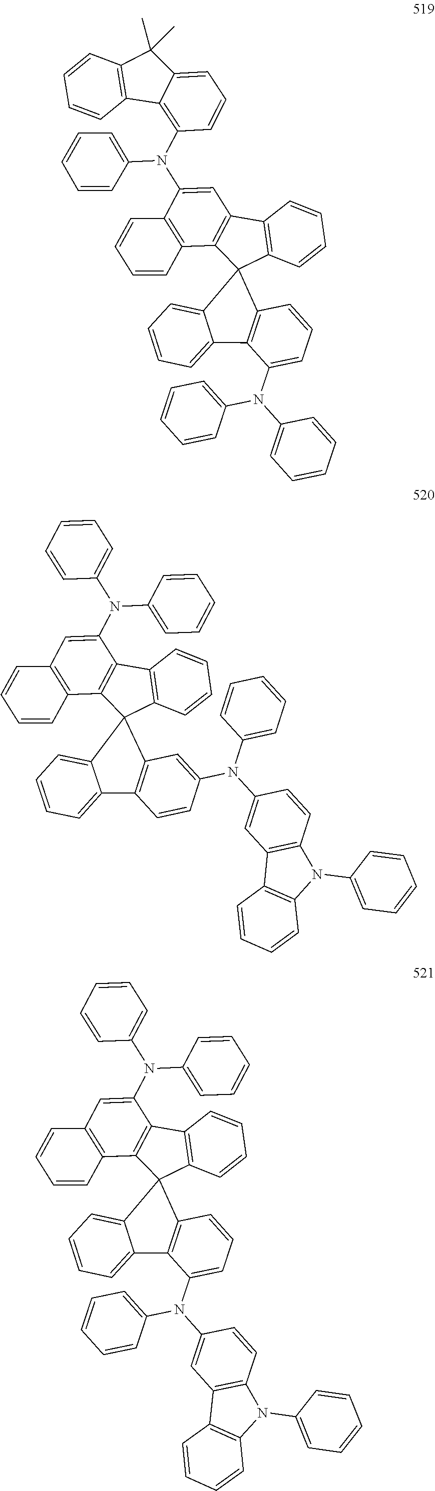 [SMILES: CC1(C)c2ccccc2-c2c(N(c3ccccc3)c3cc4c(c5ccccc35)C3(c5ccccc5-4)c4ccccc4-c4c(N(c5ccccc5)c5ccccc5)cccc43)cccc21 ; c1ccc(N(c2ccc3c(c2)C2(c4ccccc4-3)c3ccccc3-c3c(N(c4ccccc4)c4ccccc4)cc4ccccc4c32)c2ccc3c(c2)c2ccccc2n3-c2ccccc2)cc1 ; c1ccc(N(c2ccc3c(c2)c2ccccc2n3-c2ccccc2)c2cccc3c2-c2ccccc2C32c3ccccc3-c3c(N(c4ccccc4)c4ccccc4)cc4ccccc4c32)cc1]

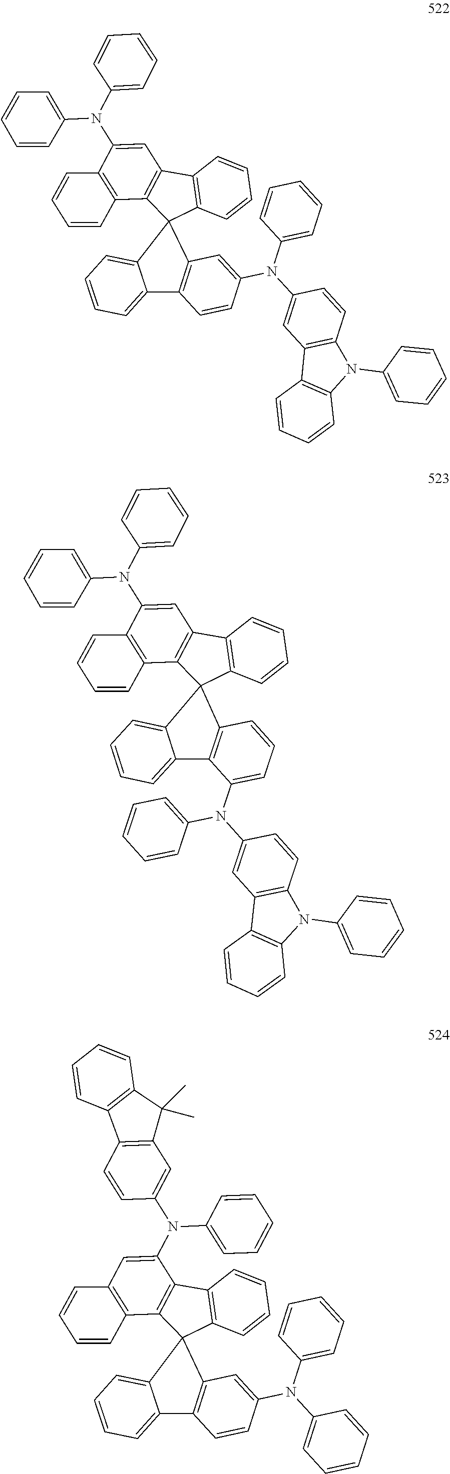 [SMILES: c1ccc(N(c2ccc3c(c2)C2(c4ccccc4-3)c3ccccc3-c3cc(N(c4ccccc4)c4ccccc4)c4ccccc4c32)c2ccc3c(c2)c2ccccc2n3-c2ccccc2)cc1 ; c1ccc(N(c2ccc3c(c2)c2ccccc2n3-c2ccccc2)c2cccc3c2-c2ccccc2C32c3ccccc3-c3cc(N(c4ccccc4)c4ccccc4)c4ccccc4c32)cc1 ; CC1(C)c2ccccc2-c2ccc(N(c3ccccc3)c3cc4ccccc4c4c3-c3ccccc3C43c4ccccc4-c4ccc(N(c5ccccc5)c5ccccc5)cc43)cc21]

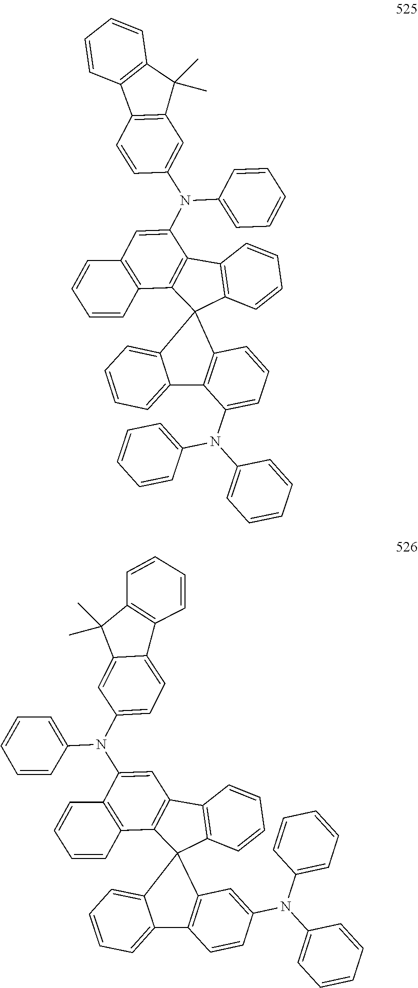 [SMILES: CC1(C)c2ccccc2-c2ccc(N(c3ccccc3)c3cc4ccccc4c4c3-c3ccccc3C43c4ccccc4-c4c(N(c5ccccc5)c5ccccc5)cccc43)cc21 ; CC1(C)c2ccccc2-c2ccc(N(c3ccccc3)c3cc4c(c5ccccc35)C3(c5ccccc5-c5ccc(N(c6ccccc6)c6ccccc6)cc53)c3ccccc3-4)cc21]

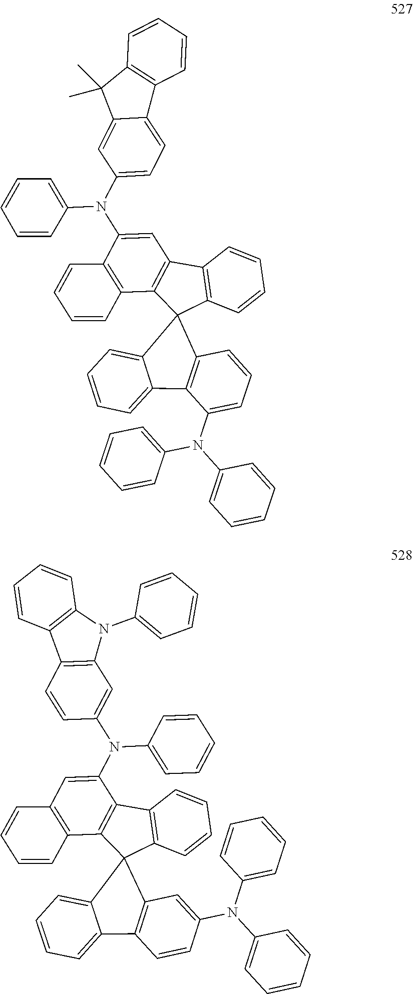 [SMILES: CC1(C)c2ccccc2-c2ccc(N(c3ccccc3)c3cc4c(c5ccccc35)C3(c5ccccc5-4)c4ccccc4-c4c(N(c5ccccc5)c5ccccc5)cccc43)cc21 ; c1ccc(N(c2ccccc2)c2ccc3c(c2)C2(c4ccccc4-3)c3ccccc3-c3c(N(c4ccccc4)c4ccc5c6ccccc6n(-c6ccccc6)c5c4)cc4ccccc4c32)cc1]

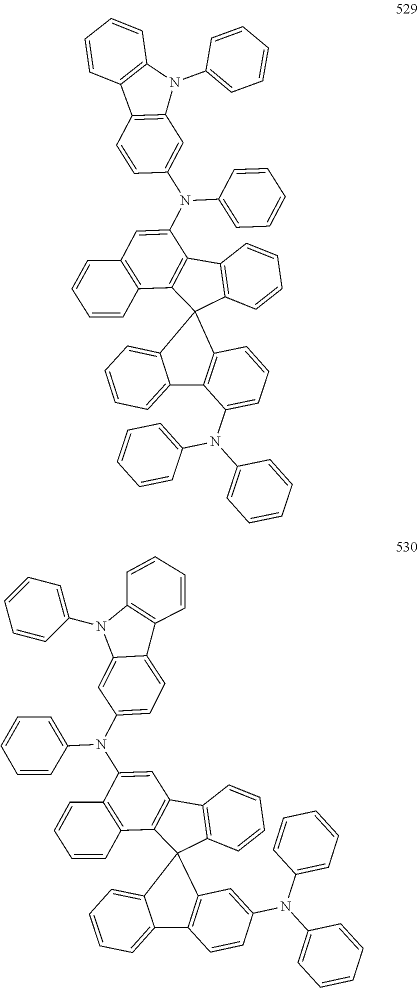 [SMILES: c1ccc(N(c2ccccc2)c2cccc3c2-c2ccccc2C32c3ccccc3-c3c(N(c4ccccc4)c4ccc5c6ccccc6n(-c6ccccc6)c5c4)cc4ccccc4c32)cc1 ; c1ccc(N(c2ccccc2)c2ccc3c(c2)C2(c4ccccc4-3)c3ccccc3-c3cc(N(c4ccccc4)c4ccc5c6ccccc6n(-c6ccccc6)c5c4)c4ccccc4c32)cc1]

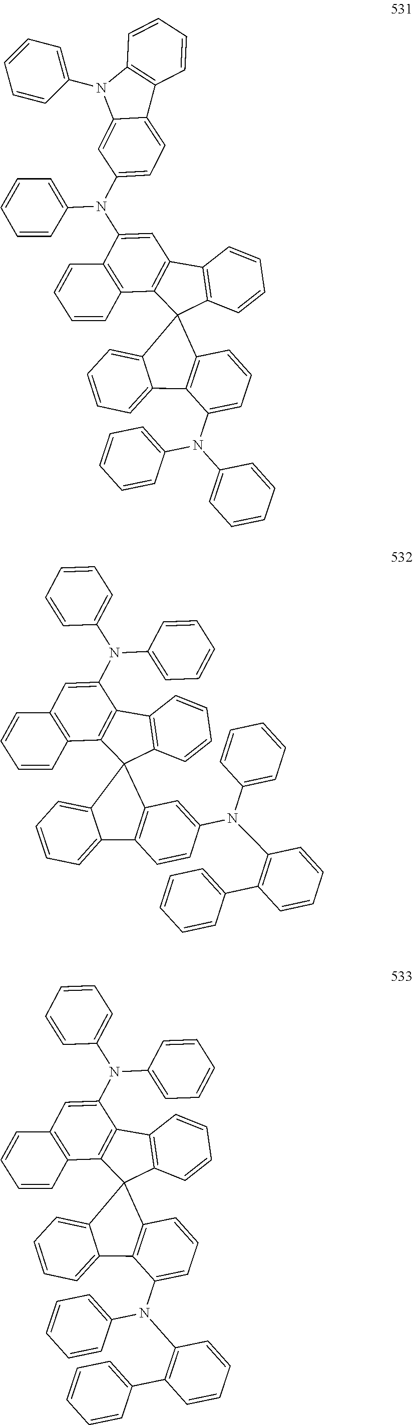 [SMILES: c1ccc(N(c2ccccc2)c2cccc3c2-c2ccccc2C32c3ccccc3-c3cc(N(c4ccccc4)c4ccc5c6ccccc6n(-c6ccccc6)c5c4)c4ccccc4c32)cc1 ; c1ccc(-c2ccccc2N(c2ccccc2)c2ccc3c(c2)C2(c4ccccc4-3)c3ccccc3-c3c(N(c4ccccc4)c4ccccc4)cc4ccccc4c32)cc1 ; c1ccc(-c2ccccc2N(c2ccccc2)c2cccc3c2-c2ccccc2C32c3ccccc3-c3c(N(c4ccccc4)c4ccccc4)cc4ccccc4c32)cc1]

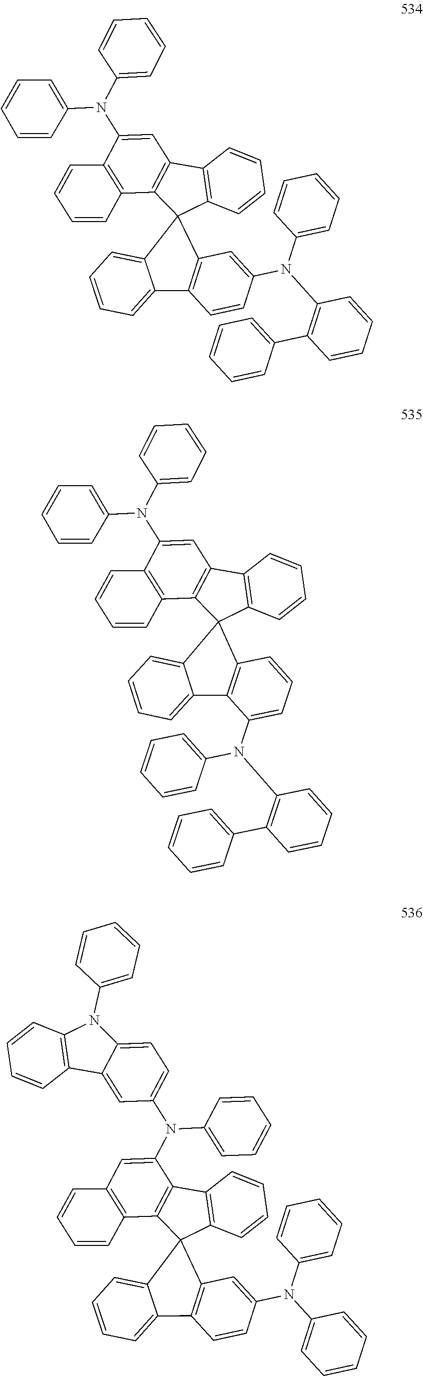 [SMILES: c1ccc(-c2ccccc2N(c2ccccc2)c2ccc3c(c2)C2(c4ccccc4-3)c3ccccc3-c3cc(N(c4ccccc4)c4ccccc4)c4ccccc4c32)cc1 ; c1ccc(-c2ccccc2N(c2ccccc2)c2cccc3c2-c2ccccc2C32c3ccccc3-c3cc(N(c4ccccc4)c4ccccc4)c4ccccc4c32)cc1 ; c1ccc(N(c2ccccc2)c2ccc3c(c2)C2(c4ccccc4-3)c3ccccc3-c3c(N(c4ccccc4)c4ccc5c(c4)c4ccccc4n5-c4ccccc4)cc4ccccc4c32)cc1]

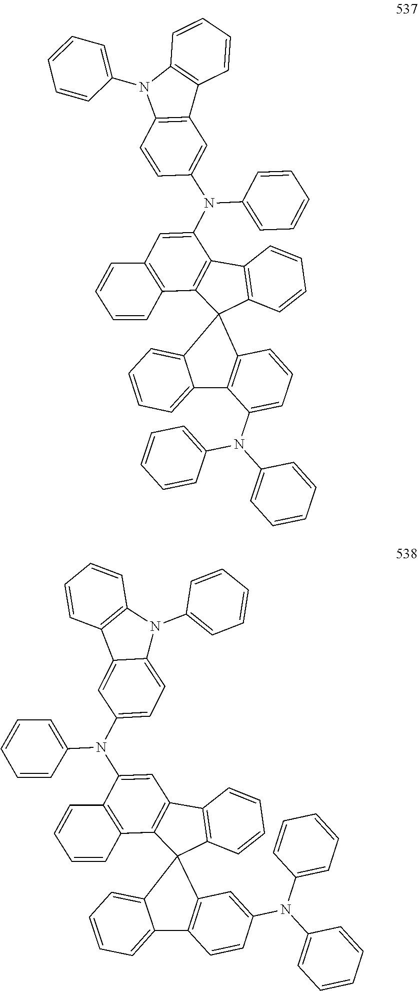 [SMILES: c1ccc(N(c2ccccc2)c2cccc3c2-c2ccccc2C32c3ccccc3-c3c(N(c4ccccc4)c4ccc5c(c4)c4ccccc4n5-c4ccccc4)cc4ccccc4c32)cc1 ; c1ccc(N(c2ccccc2)c2ccc3c(c2)C2(c4ccccc4-3)c3ccccc3-c3cc(N(c4ccccc4)c4ccc5c(c4)c4ccccc4n5-c4ccccc4)c4ccccc4c32)cc1]

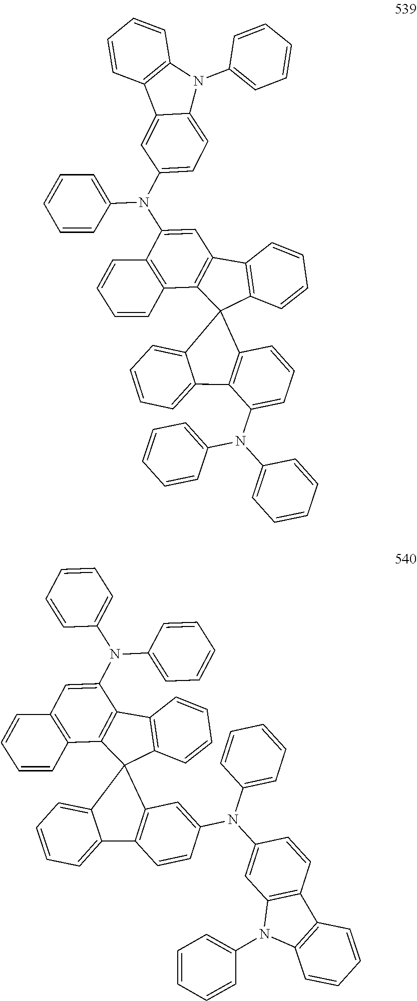 [SMILES: c1ccc(N(c2ccccc2)c2cccc3c2-c2ccccc2C32c3ccccc3-c3cc(N(c4ccccc4)c4ccc5c(c4)c4ccccc4n5-c4ccccc4)c4ccccc4c32)cc1 ; c1ccc(N(c2ccc3c(c2)C2(c4ccccc4-3)c3ccccc3-c3c(N(c4ccccc4)c4ccccc4)cc4ccccc4c32)c2ccc3c4ccccc4n(-c4ccccc4)c3c2)cc1]

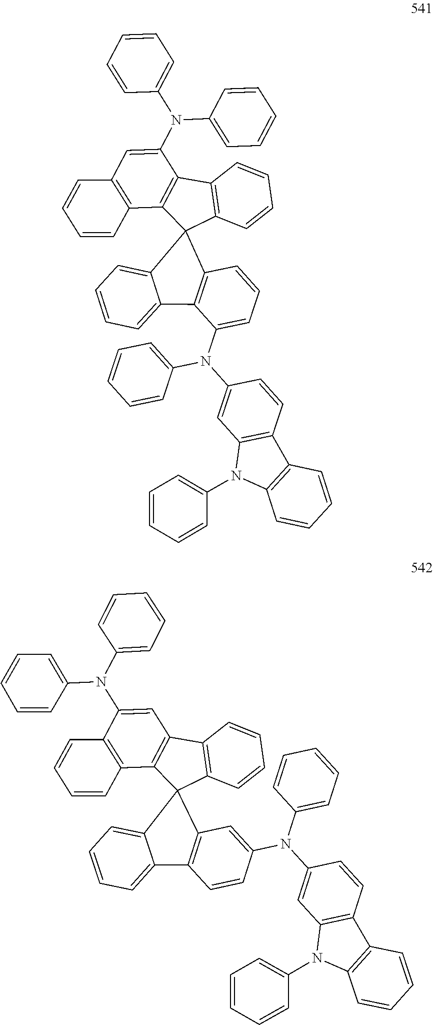 [SMILES: c1ccc(N(c2ccc3c4ccccc4n(-c4ccccc4)c3c2)c2cccc3c2-c2ccccc2C32c3ccccc3-c3c(N(c4ccccc4)c4ccccc4)cc4ccccc4c32)cc1 ; c1ccc(N(c2ccc3c(c2)C2(c4ccccc4-3)c3ccccc3-c3cc(N(c4ccccc4)c4ccccc4)c4ccccc4c32)c2ccc3c4ccccc4n(-c4ccccc4)c3c2)cc1]

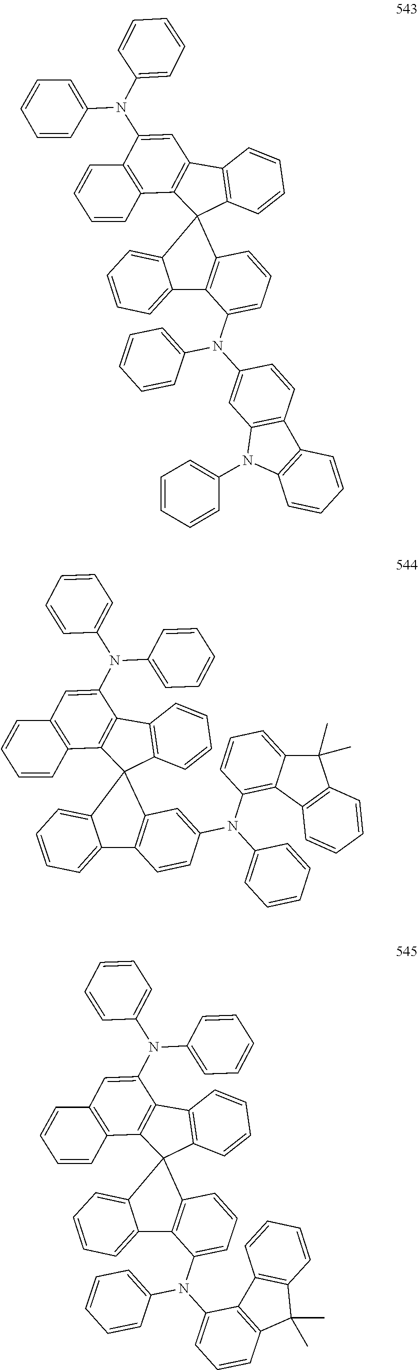 [SMILES: c1ccc(N(c2ccc3c4ccccc4n(-c4ccccc4)c3c2)c2cccc3c2-c2ccccc2C32c3ccccc3-c3cc(N(c4ccccc4)c4ccccc4)c4ccccc4c32)cc1 ; CC1(C)c2ccccc2-c2c(N(c3ccccc3)c3ccc4c(c3)C3(c5ccccc5-4)c4ccccc4-c4c(N(c5ccccc5)c5ccccc5)cc5ccccc5c43)cccc21 ; CC1(C)c2ccccc2-c2c(N(c3ccccc3)c3cccc4c3-c3ccccc3C43c4ccccc4-c4c(N(c5ccccc5)c5ccccc5)cc5ccccc5c43)cccc21]

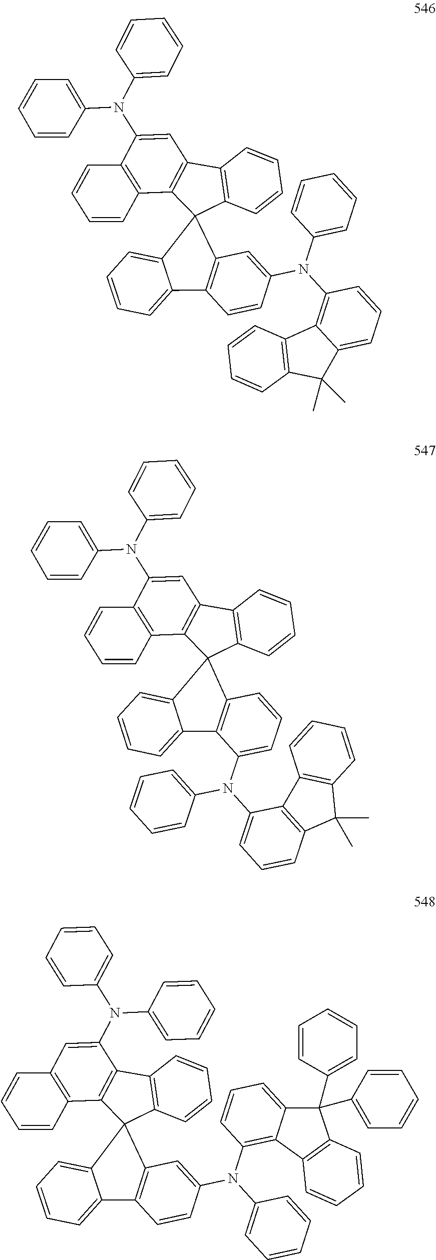 [SMILES: CC1(C)c2ccccc2-c2c(N(c3ccccc3)c3ccc4c(c3)C3(c5ccccc5-4)c4ccccc4-c4cc(N(c5ccccc5)c5ccccc5)c5ccccc5c43)cccc21 ; CC1(C)c2ccccc2-c2c(N(c3ccccc3)c3cccc4c3-c3ccccc3C43c4ccccc4-c4cc(N(c5ccccc5)c5ccccc5)c5ccccc5c43)cccc21 ; c1ccc(N(c2ccc3c(c2)C2(c4ccccc4-3)c3ccccc3-c3c(N(c4ccccc4)c4ccccc4)cc4ccccc4c32)c2cccc3c2-c2ccccc2C3(c2ccccc2)c2ccccc2)cc1]

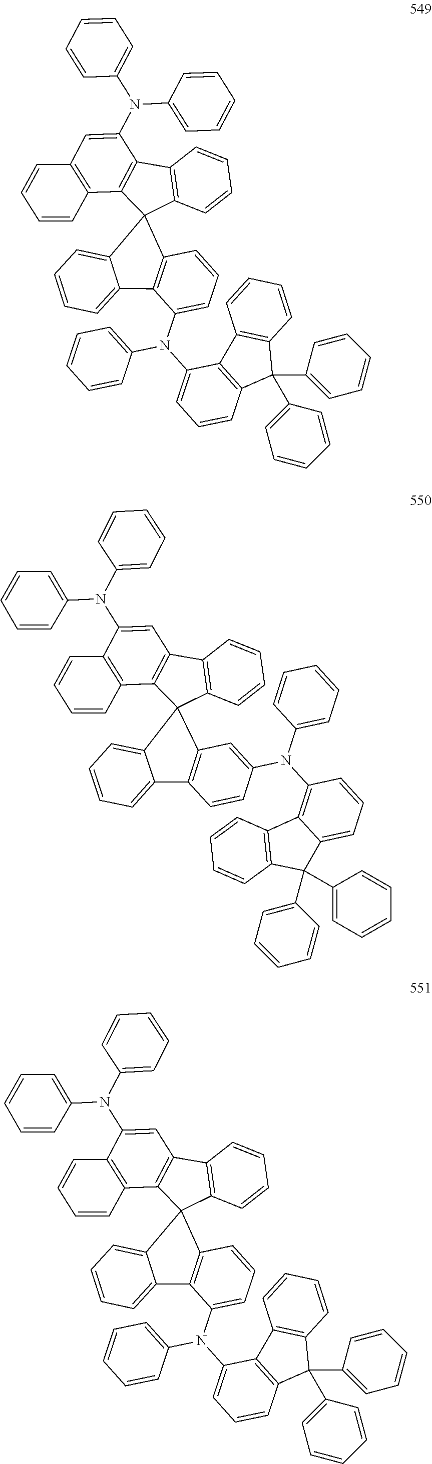 [SMILES: c1ccc(N(c2ccccc2)c2cc3ccccc3c3c2-c2ccccc2C32c3ccccc3-c3c(N(c4ccccc4)c4cccc5c4-c4ccccc4C5(c4ccccc4)c4ccccc4)cccc32)cc1 ; c1ccc(N(c2ccc3c(c2)C2(c4ccccc4-3)c3ccccc3-c3cc(N(c4ccccc4)c4ccccc4)c4ccccc4c32)c2cccc3c2-c2ccccc2C3(c2ccccc2)c2ccccc2)cc1 ; c1ccc(N(c2cccc3c2-c2ccccc2C3(c2ccccc2)c2ccccc2)c2cccc3c2-c2ccccc2C32c3ccccc3-c3cc(N(c4ccccc4)c4ccccc4)c4ccccc4c32)cc1]

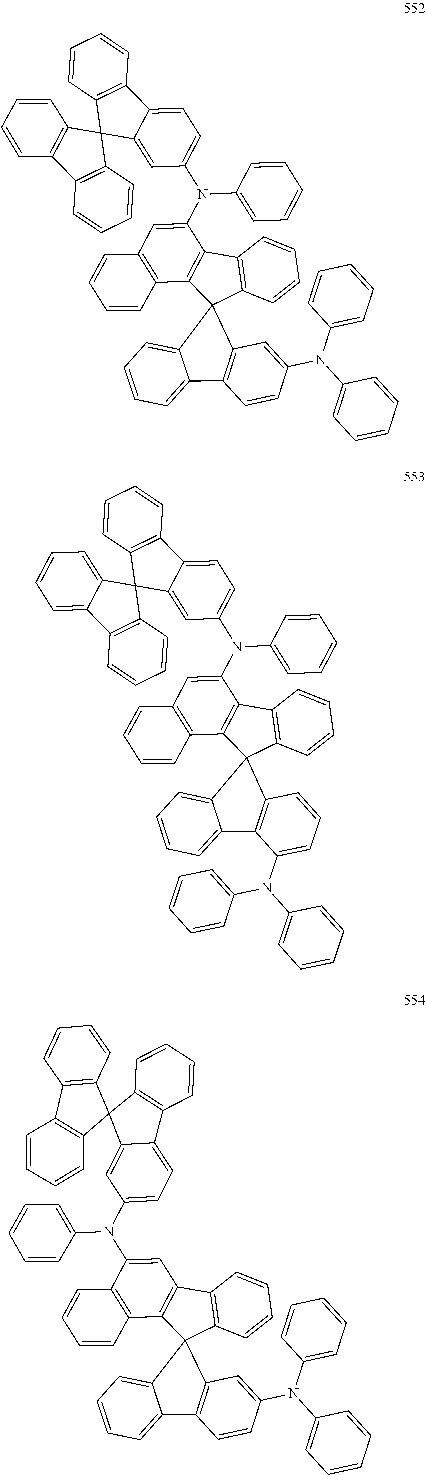 [SMILES: c1ccc(N(c2ccccc2)c2ccc3c(c2)C2(c4ccccc4-3)c3ccccc3-c3c(N(c4ccccc4)c4ccc5c(c4)C4(c6ccccc6-c6ccccc64)c4ccccc4-5)cc4ccccc4c32)cc1 ; c1ccc(N(c2ccccc2)c2cccc3c2-c2ccccc2C32c3ccccc3-c3c(N(c4ccccc4)c4ccc5c(c4)C4(c6ccccc6-c6ccccc64)c4ccccc4-5)cc4ccccc4c32)cc1 ; c1ccc(N(c2ccccc2)c2ccc3c(c2)C2(c4ccccc4-3)c3ccccc3-c3cc(N(c4ccccc4)c4ccc5c(c4)C4(c6ccccc6-c6ccccc64)c4ccccc4-5)c4ccccc4c32)cc1]

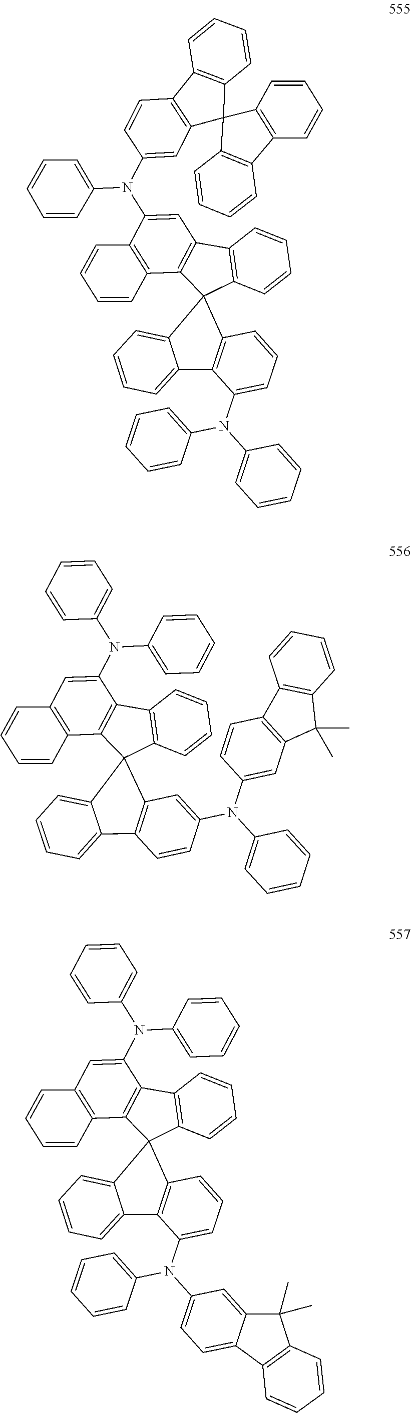 [SMILES: c1ccc(N(c2ccccc2)c2cccc3c2-c2ccccc2C32c3ccccc3-c3cc(N(c4ccccc4)c4ccc5c(c4)C4(c6ccccc6-c6ccccc64)c4ccccc4-5)c4ccccc4c32)cc1 ; CC1(C)c2ccccc2-c2ccc(N(c3ccccc3)c3ccc4c(c3)C3(c5ccccc5-4)c4ccccc4-c4c(N(c5ccccc5)c5ccccc5)cc5ccccc5c43)cc21 ; CC1(C)c2ccccc2-c2ccc(N(c3ccccc3)c3cccc4c3-c3ccccc3C43c4ccccc4-c4c(N(c5ccccc5)c5ccccc5)cc5ccccc5c43)cc21]

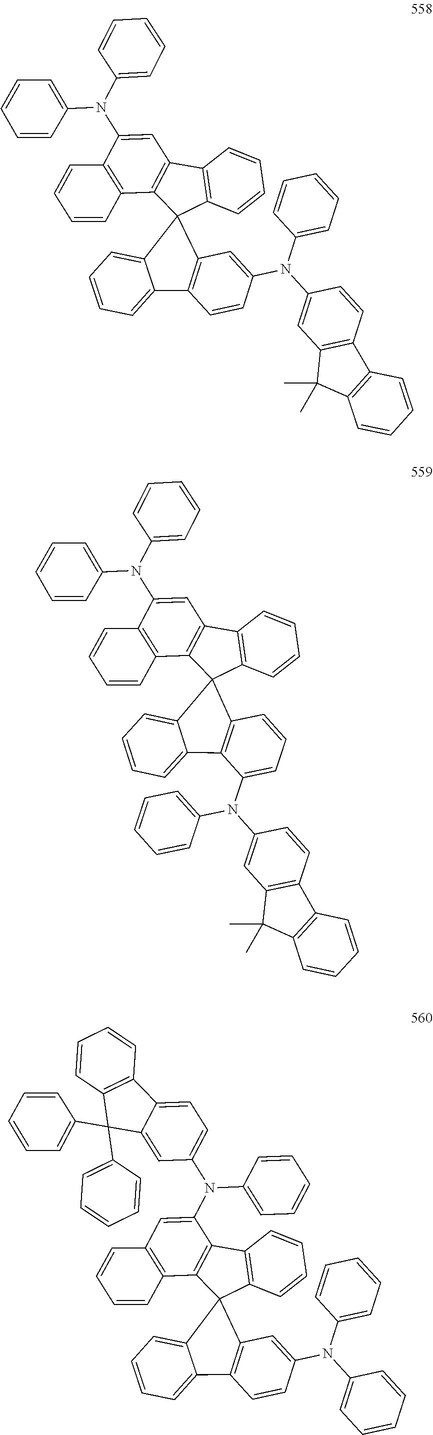 [SMILES: CC1(C)c2ccccc2-c2ccc(N(c3ccccc3)c3ccc4c(c3)C3(c5ccccc5-4)c4ccccc4-c4cc(N(c5ccccc5)c5ccccc5)c5ccccc5c43)cc21 ; CC1(C)c2ccccc2-c2ccc(N(c3ccccc3)c3cccc4c3-c3ccccc3C43c4ccccc4-c4cc(N(c5ccccc5)c5ccccc5)c5ccccc5c43)cc21 ; c1ccc(N(c2ccccc2)c2ccc3c(c2)C2(c4ccccc4-3)c3ccccc3-c3c(N(c4ccccc4)c4ccc5c(c4)C(c4ccccc4)(c4ccccc4)c4ccccc4-5)cc4ccccc4c32)cc1]

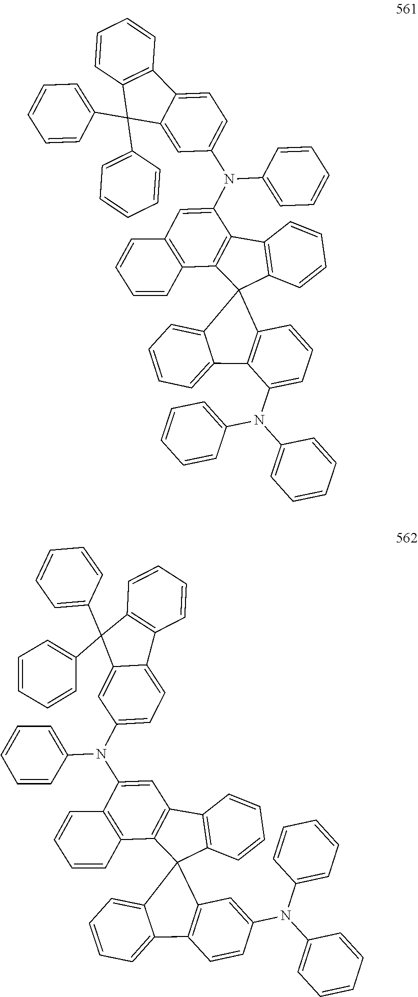 [SMILES: c1ccc(N(c2ccccc2)c2cccc3c2-c2ccccc2C32c3ccccc3-c3c(N(c4ccccc4)c4ccc5c(c4)C(c4ccccc4)(c4ccccc4)c4ccccc4-5)cc4ccccc4c32)cc1 ; c1ccc(N(c2ccccc2)c2ccc3c(c2)C2(c4ccccc4-3)c3ccccc3-c3cc(N(c4ccccc4)c4ccc5c(c4)C(c4ccccc4)(c4ccccc4)c4ccccc4-5)c4ccccc4c32)cc1]

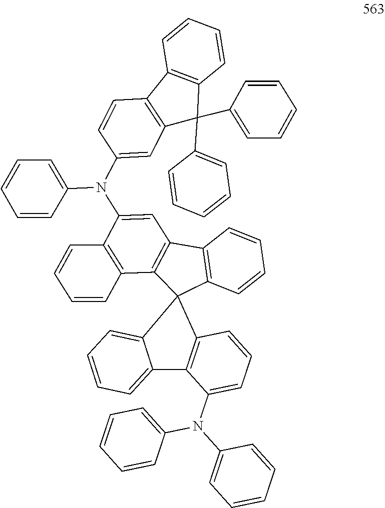 [SMILES: c1ccc(N(c2ccccc2)c2cccc3c2-c2ccccc2C32c3ccccc3-c3cc(N(c4ccccc4)c4ccc5c(c4)C(c4ccccc4)(c4ccccc4)c4ccccc4-5)c4ccccc4c32)cc1]

The amine-based compound represented by Formula 1 includes a core moiety that includes ring A₁ condensed with ring A₂, and includes an amine group bonded to ring A₁ and an amine group bonded to ring A₃ or ring A₄.

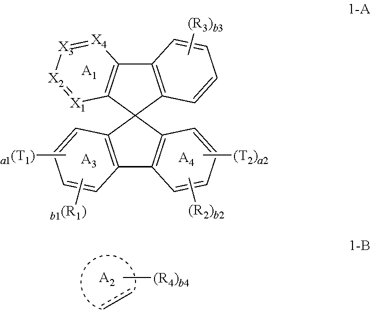

Formulae 1-A and 1-B are respectively the same as described in connection with Formulae 1 and 1A, and Formula 2 is the same as described in the specification.

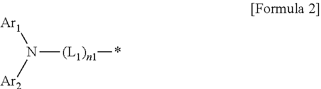

In the amine-based compound represented by Formula 1, a conjugated structure of the core moiety is not continuous, and a highest occupied molecular orbital (HOMO) energy level of the compound may be variously changed by introducing two amine groups and by variously changing substituents (e.g., Ar₁ and Ar₂ in Formula 2) of the amine groups. Accordingly, a hole injection layer and a hole transport layer may be variously changed, and an appropriate energy level may be maintained between the hole transport region and the emission layer to increase exciton generation efficiency inside the emission layer.

Therefore, by including the amine-based compound represented by Formula 1, a light-emitting device (for example, an organic light-emitting device) with low driving voltage, high luminance, high efficiency, and long lifespan may be manufactured.

Methods of synthesizing the amine-based compound represented by Formula 1 may be readily understood to those of ordinary skill in the art by referring to Synthesis Examples and/or Examples described herein.

At least one amine-based compound represented by Formula 1 may be used in a light-emitting device (for example, an organic light-emitting device). Therefore, provided is a light-emitting device which may include a first electrode, a second electrode, an interlayer between the first electrode and the second electrode and including an emission layer, and an amine-based compound represented by Formula 1 as described in the specification.

In an embodiment, the first electrode may be an anode, the second electrode may be a cathode, the interlayer may further include a hole transport region between the first electrode and the emission layer and an electron transport region between the emission layer and the second electrode layer, and the hole transport region may include the amine-based compound.

In an embodiment, the hole transport region may include a hole injection layer, a hole transport layer, an emission auxiliary layer, an electron blocking layer, or any combination thereof, and the electron transport region may include a buffer layer, a hole blocking layer, an electron control layer, an electron transport layer, an electron injection layer, or any combination thereof.

In an embodiment, the light-emitting device may further include a capping layer located outside the first electrode or located outside the second electrode.

In an embodiment, the light-emitting device may further include at least one of a first capping layer located outside the first electrode and a second capping layer located outside the second electrode, and the at least one of the first capping layer and the second capping layer may include the amine-based compound represented by Formula 1. More details for the first capping layer and/or second capping layer are respectively the same as described in the specification.

In an embodiment, the light-emitting device may further include: a first capping layer located outside the first electrode and including the amine-based compound represented by Formula 1; a second capping layer located outside the second electrode and including the amine-based compound represented by Formula 1; or the first capping layer and the second capping layer.

The wording “(interlayer and/or capping layer) includes an amine-based compound” as used herein may be understood as “(interlayer and/or capping layer) may include one kind of amine-based compound represented by Formula 1 or at least two different kinds of amine-based compounds, each represented by Formula 1”.

In an embodiment, the interlayer and/or the capping layer may include, as the amine-based compound, Compound 1 only. In this regard, Compound 1 may be included in the hole transport region in the light-emitting device. In an embodiment, the interlayer may include, as the amine-based compound, Compound 1 and Compound 2. In this regard, Compound 1 and Compound 2 may be present in a same layer (for example, both Compound 1 and Compound 2 may be present in the hole transport region), or may be present in different layers (for example, Compound 1 may be present in the hole injection layer, and Compound 2 may be present in the hole transport layer).

The term “interlayer” as used herein refers to a single layer and/or multiple layers located between the first electrode and the second electrode of the light-emitting device.

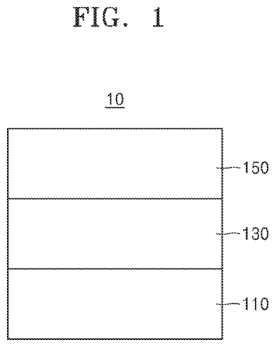

According to embodiments, provided is an electronic apparatus which may include the light-emitting device. The electronic apparatus may further include a thin-film transistor. In an embodiment, the electronic apparatus may further include a thin-film transistor including a source electrode and a drain electrode, wherein the first electrode of the light-emitting device may be electrically connected to the source electrode or the drain electrode. In an embodiment, the electronic apparatus may further include a color filter, a color conversion layer, a touch screen layer, a polarizing layer, or any combination thereof. More details for the electronic apparatus are as described in the specification.

[Description of FIG. 1]

FIG. 1 is a schematic cross-sectional view of a light-emitting device 10 according to an embodiment. The light-emitting device 10 includes a first electrode 110, an interlayer 130, and a second electrode 150.

Hereinafter, a structure of the light-emitting device 10 according to an embodiment and a method of manufacturing the light-emitting device 10 will be described in connection with FIG. 1.

[First Electrode 110]

In FIG. 1, a substrate may be further included under the first electrode 110 or above the second electrode 150. The substrate may be a glass substrate or a plastic substrate, but is not limited thereto. In an embodiment, the substrate may be a flexible substrate, and may include plastics with excellent heat resistance and durability, such as polyimide, polyethylene terephthalate (PET), polycarbonate, polyethylene napthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combinations thereof. In embodiments, when the first electrode 110 is a semi-transmissive electrode or a reflective electrode, magnesium (Mg), silver (Ag), aluminum (Al), aluminum-lithium (Al—Li), calcium (Ca), magnesium-indium (Mg—In), magnesium-silver (Mg—Ag), or any combinations thereof may be used as a material for forming a first electrode.

The first electrode 110 may have a structure consisting of a single layer or a structure including multiple layers. In an embodiment, the first electrode 110 may have a three-layered structure of ITO/Ag/ITO.

[Interlayer 130]

The interlayer 130 may be located on the first electrode 110. The interlayer 130 may include an emission layer.

The interlayer 130 may further include a hole transport region between the first electrode 110 and the emission layer and an electron transport region between the emission layer and the second electrode 150.

The interlayer 130 may further include metal-containing compounds such as organometallic compounds, inorganic materials such as quantum dots, and the like, in addition to various organic materials.

In embodiments, the interlayer 130 may include two or more emitting units stacked between the first electrode 110 and the second electrode 150, and at least one charge generation layer located between the two or more emitting units. When the interlayer 130 includes the two or more emitting units and the at least one charge generation layer as described above, the light-emitting device 10 may be a tandem light-emitting device.

[Hole Transport Region in Interlayer 130]

The hole transport region may have a structure consisting of a layer consisting of a single material, a structure consisting of a layer consisting of different materials, or a structure including multiple layers including different materials.

The hole transport region may include a hole injection layer, a hole transport layer, an emission auxiliary layer, an electron blocking layer, or any combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein the layers of each structure may be stacked from the first electrode 110 in its respective stated order, but the structure of the hole transport region is not limited thereto.

The hole transport region may include a compound represented by Formula 201, a compound represented by Formula 202, or any combination thereof:

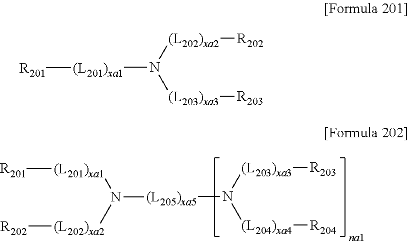

In Formulae 201 and 202,

L₂₀₁ to L₂₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,

L₂₀₅ may be *—O—*, *—S—*, *—N(Q₂₀₁)—*′, a C₁-C₂₀ alkylene group that is unsubstituted or substituted with at least one R₁₀ₐ, a C₂-C₂₀ alkenylene group that is unsubstituted or substituted with at least one R₁₀ₐ, a C₃-C₆₀ carbocyclic group that is unsubstituted or substituted with at least one R₁₀ₐ, or a C₁-C₆₀ heterocyclic group that is unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₃-C₆₀ carbocyclic group that is unsubstituted or substituted with at least one R₁₀ₐ, or a C₁-C₆₀ heterocyclic group that is unsubstituted or substituted with at least one R₁₀ₐ,

R₂₀₁ and R₂₀₂ may optionally be linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ, to form a C₈-C₆₀ polycyclic group (for example, a carbazole group or the like) unsubstituted or substituted with at least one R₁₀ₐ (for example, Compound HT16),

R₂₀₃ and R₂₀₄ may optionally be linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ, to form a C₈-C₆₀ polycyclic group that is unsubstituted or substituted with at least one R₁₀ₐ, and

na1 may be an integer from 1 to 4.

In an embodiment, each of Formulae 201 and 202 may include at least one of groups represented by Formulae CY201 to CY217:

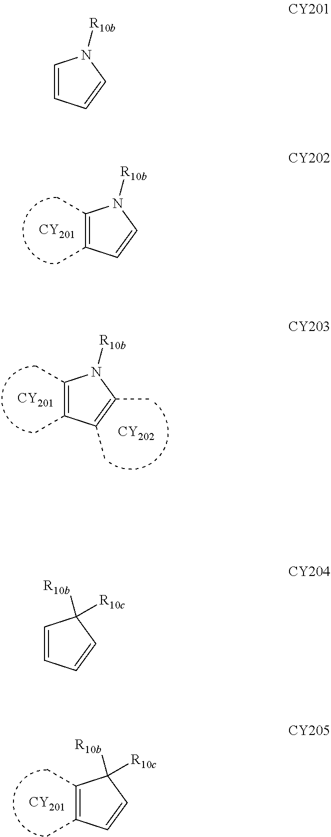

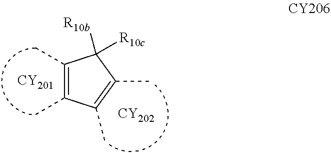

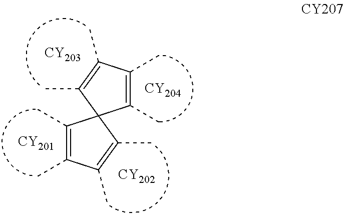

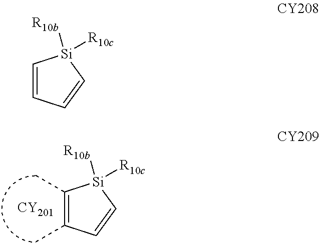

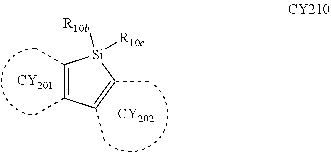

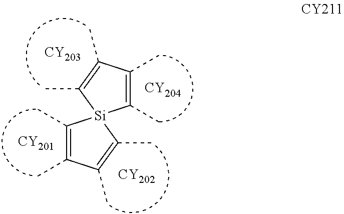

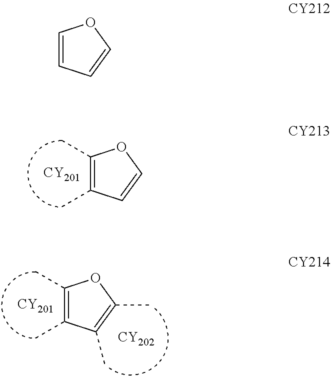

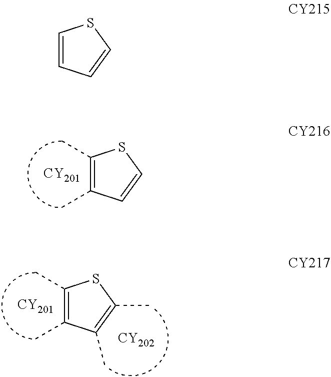

In Formulae CY201 to CY217, R_{10b} and R_{10c} may each independently be the same as described with respect to R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described herein.

In an embodiment, ring CY₂₀₁ to ring CY₂₀₄ in Formulae CY201 to CY217 may each independently be a benzene group, a naphthalene group, a phenanthrene group, or an anthracene group.

In an embodiment, each of Formulae 201 and 202 may include at least one of groups represented by Formulae CY201 to CY203.

In an embodiment, Formula 201 may include at least one of groups represented by Formulae CY201 to CY203 and at least one of groups represented by Formulae CY204 to CY217.

In an embodiment, in Formula 201, xa1 may be 1, R₂₀₁ may be a group represented by one of Formulae CY201 to CY203, xa2 may be 0, and R₂₀₂ may be a group represented by one of Formulae CY204 to CY207.

In an embodiment, each of Formulae 201 and 202 may not include groups represented by Formulae CY201 to CY203.

In an embodiment, each of Formulae 201 and 202 may not include groups represented by Formulae CY201 to CY203, and may include at least one of groups represented by Formulae CY204 to CY217.

In an embodiment, each of Formulae 201 and 202 may not include groups represented by Formulae CY201 to CY217.

In an embodiment, the hole transport region may include one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

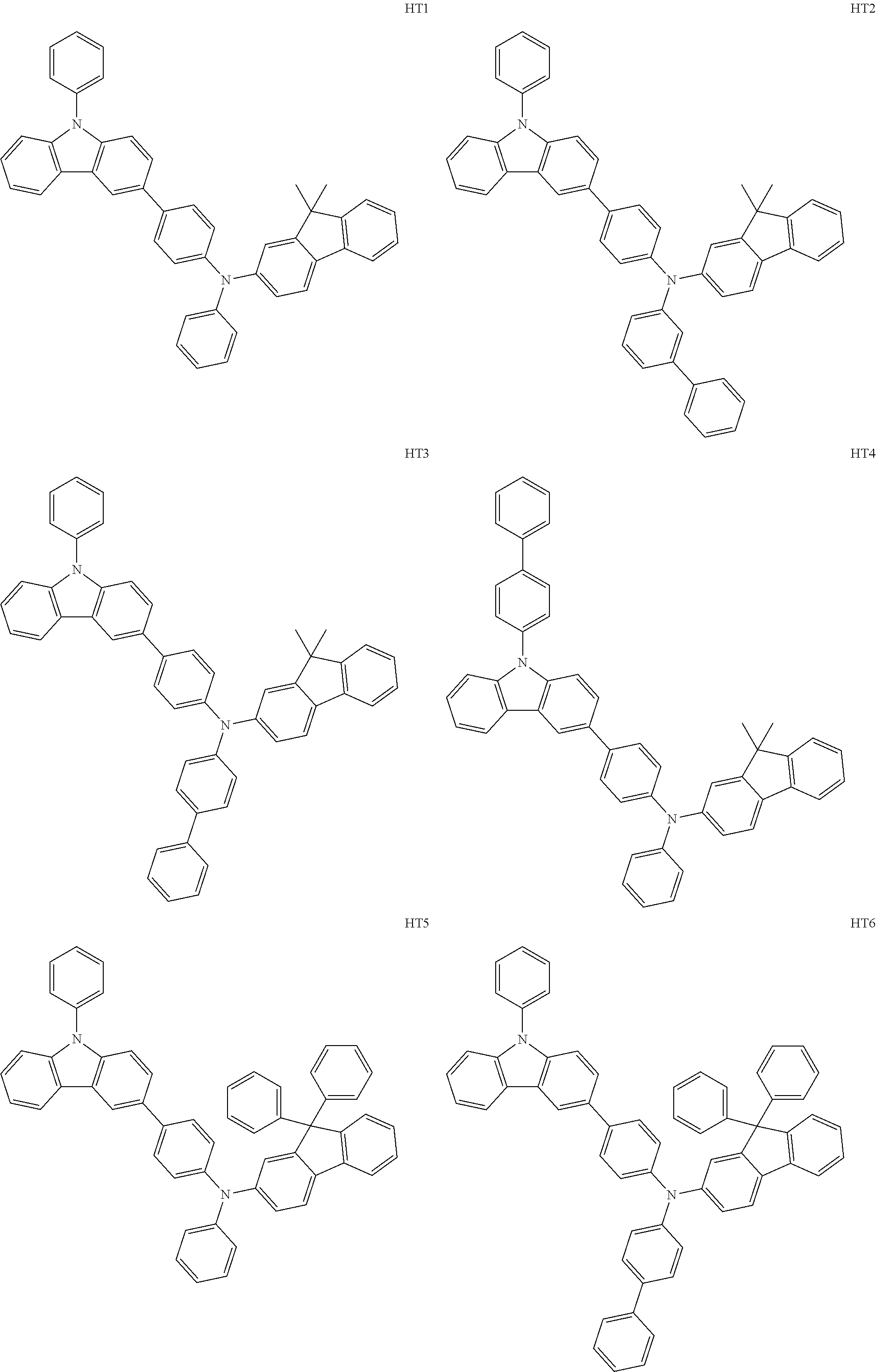

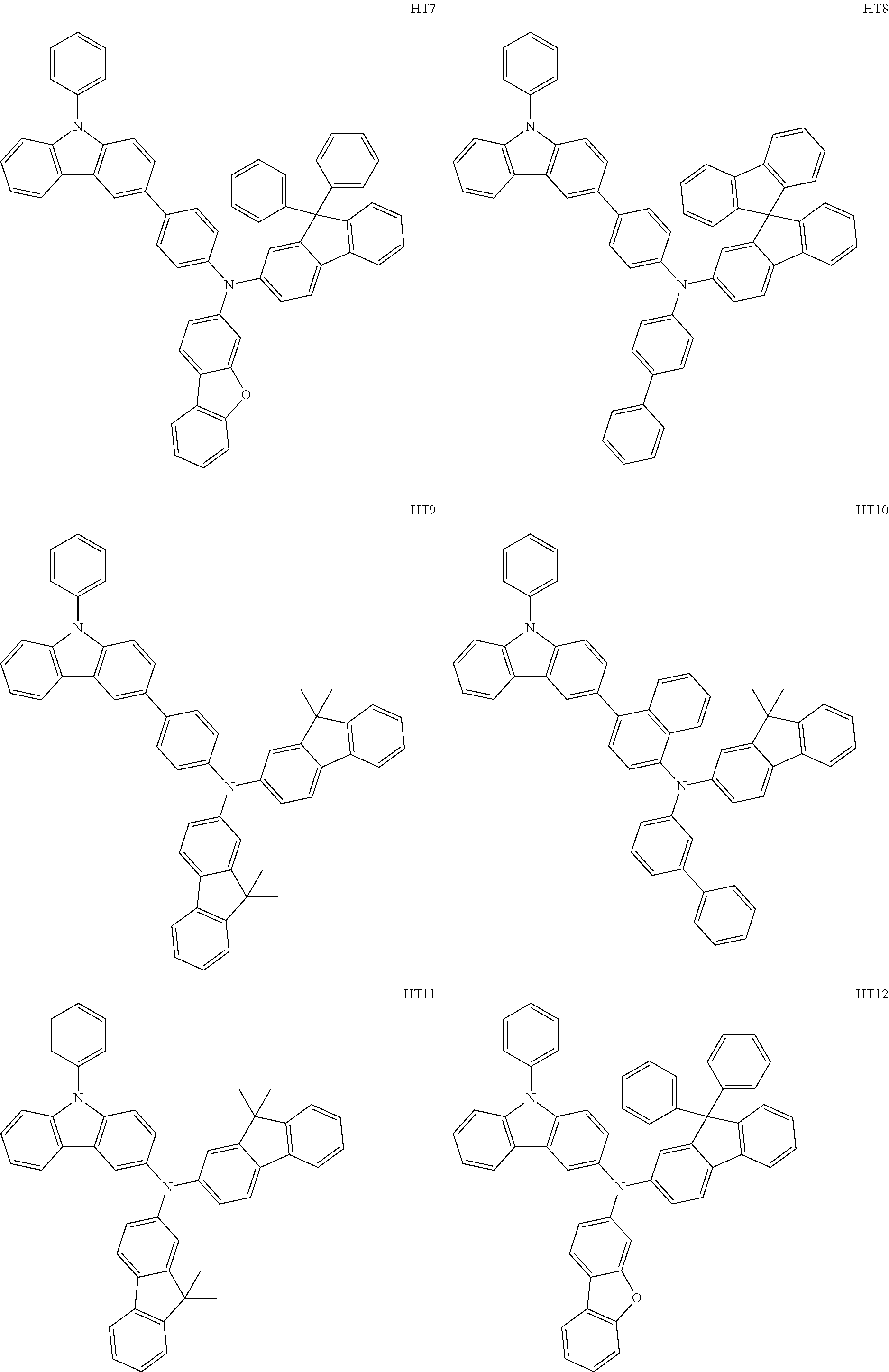

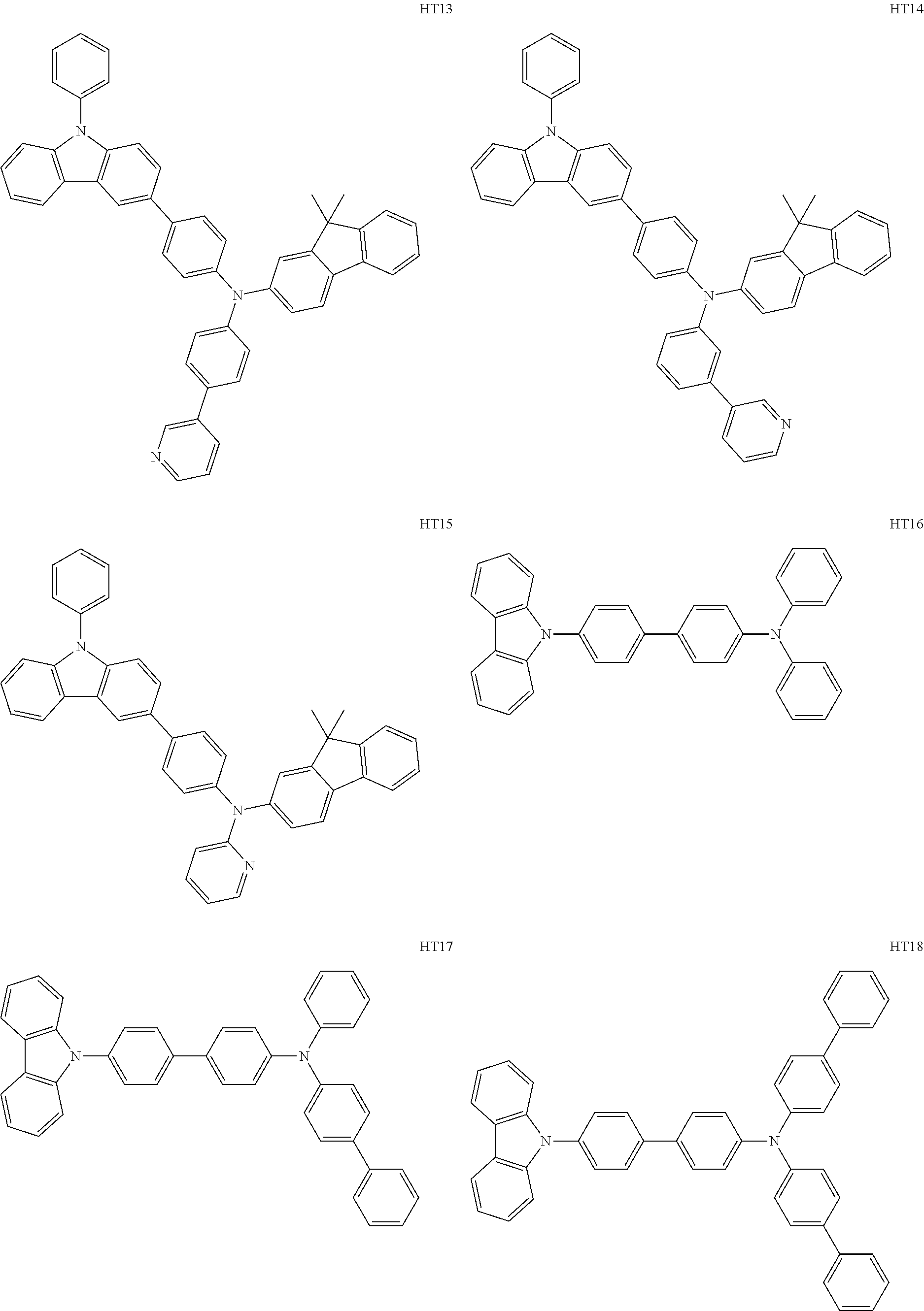 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

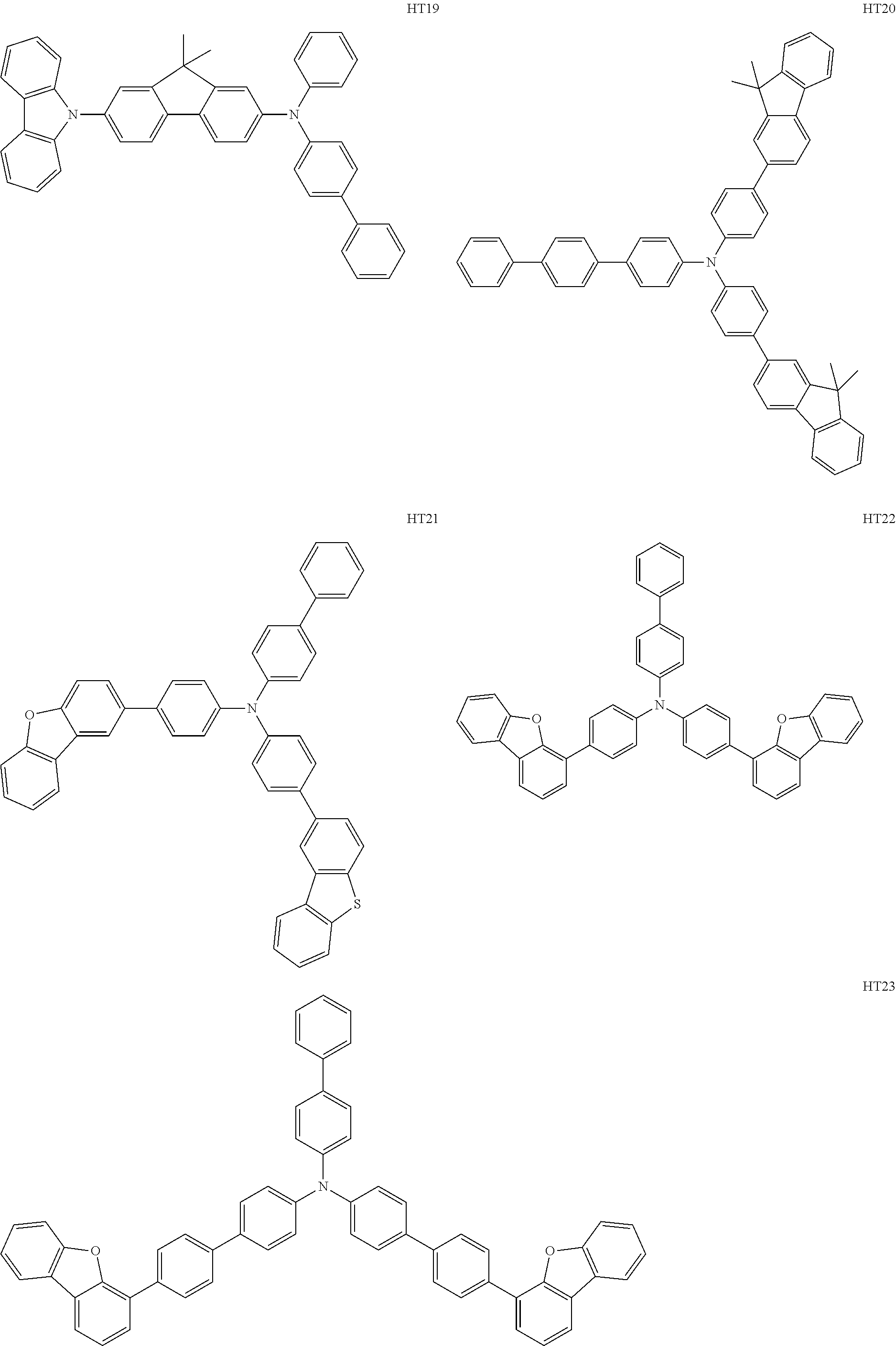

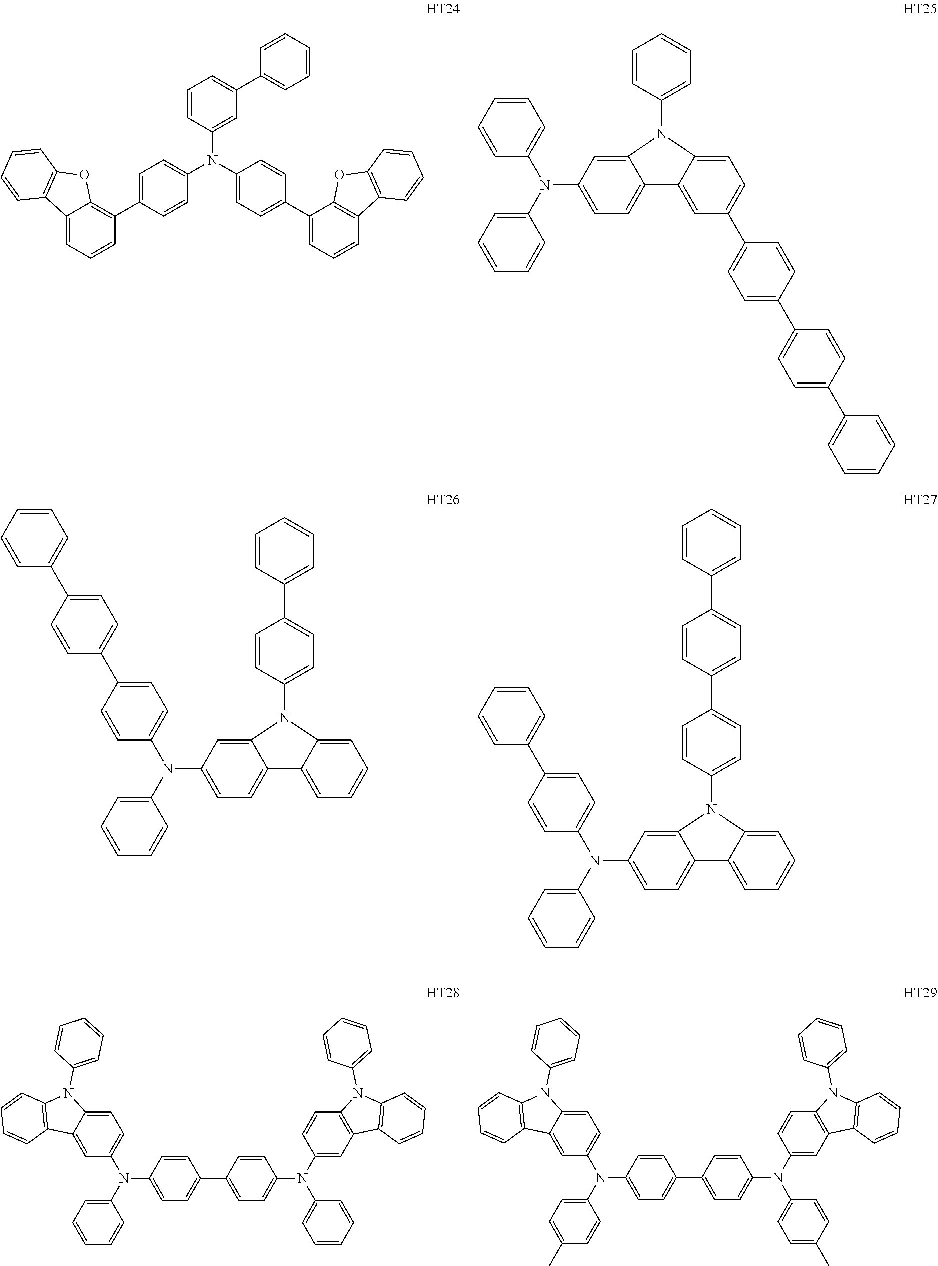

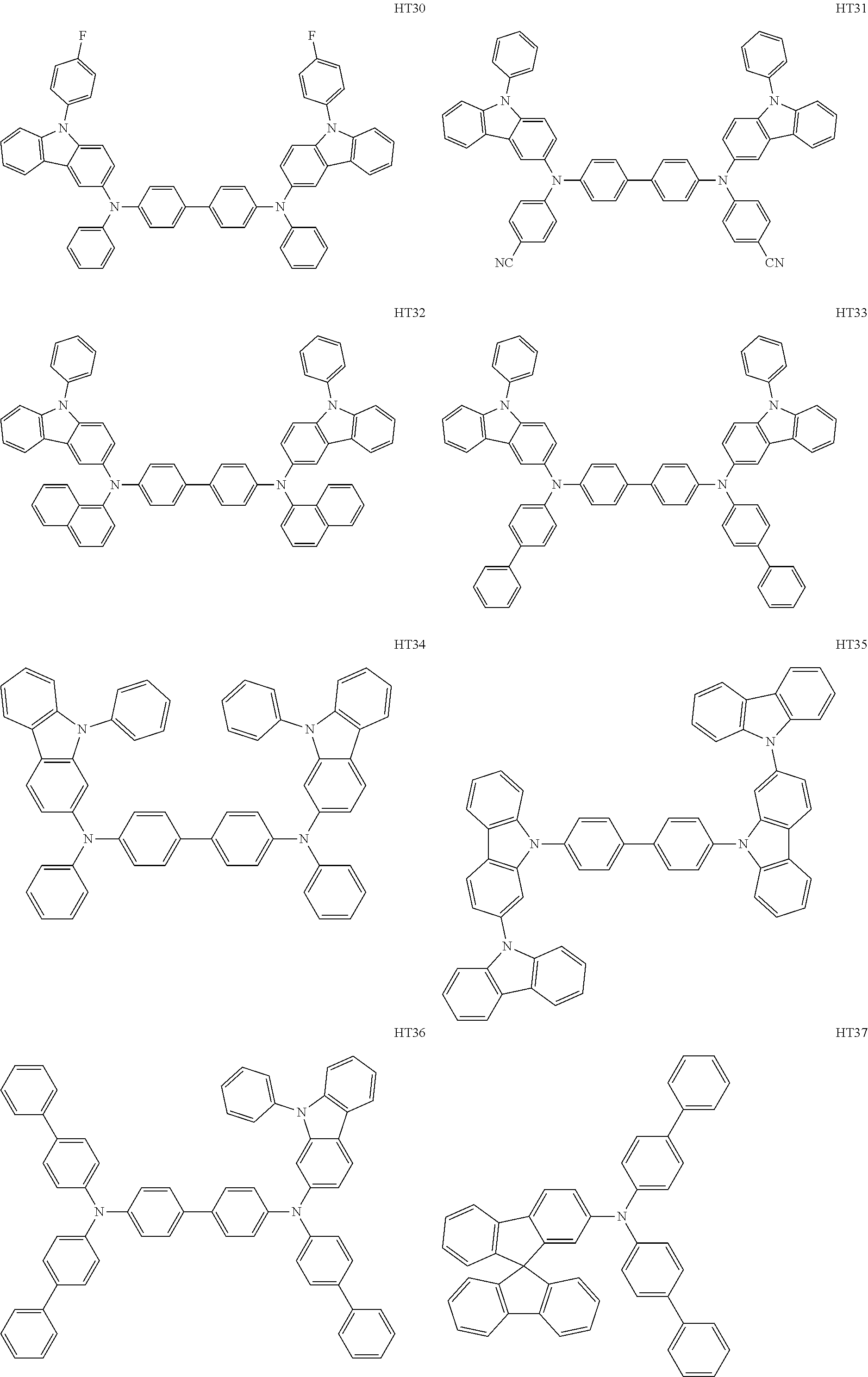

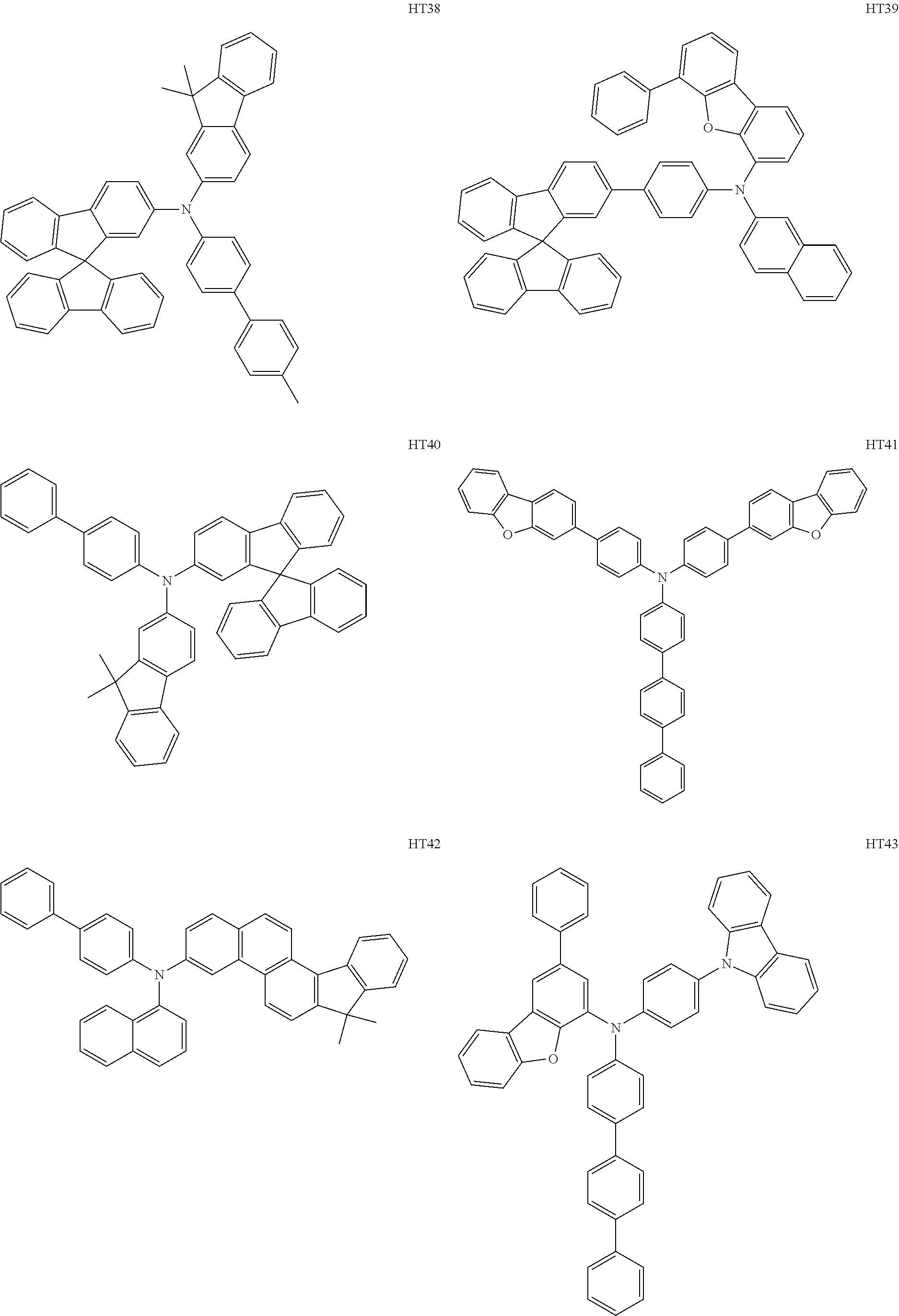

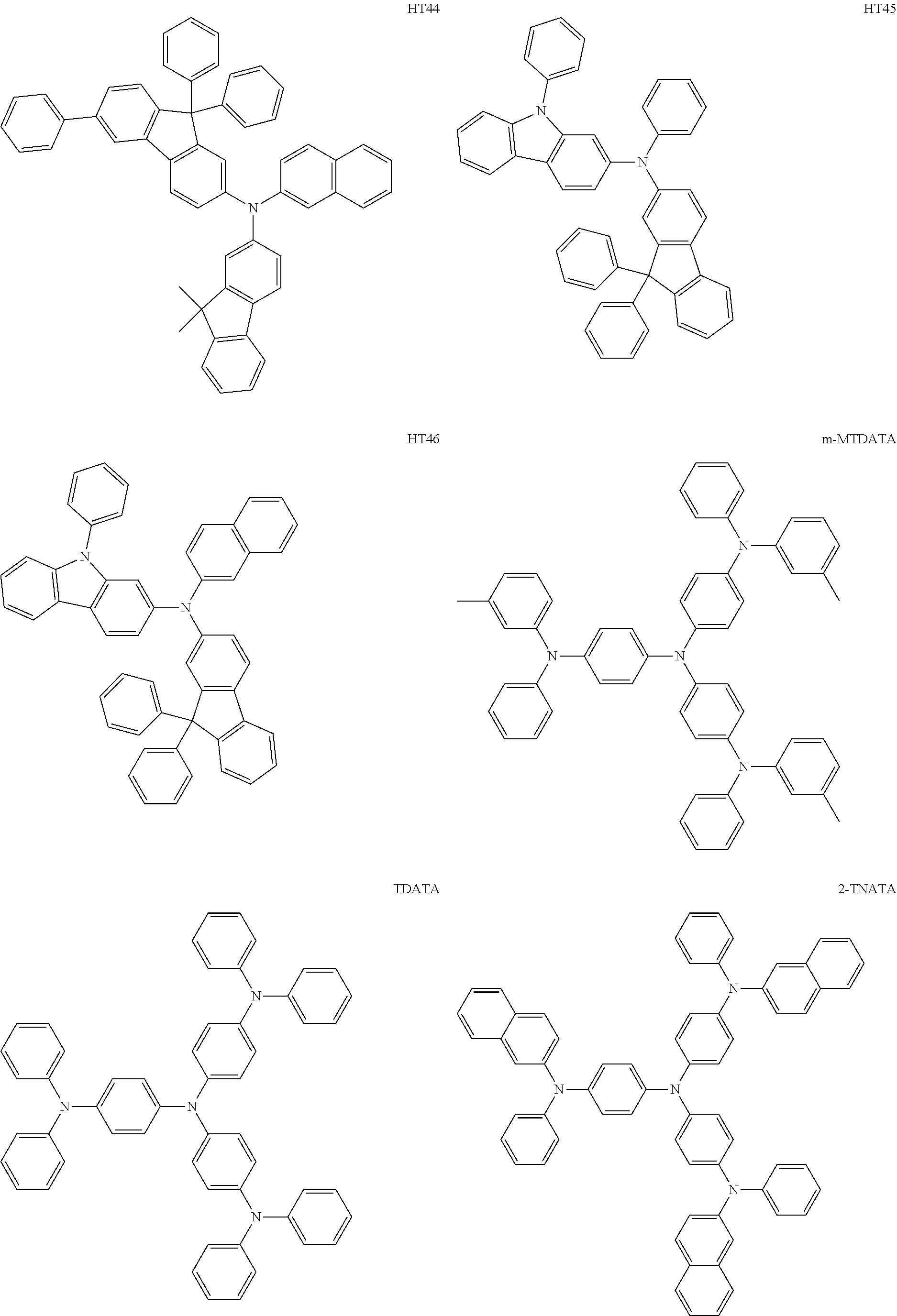

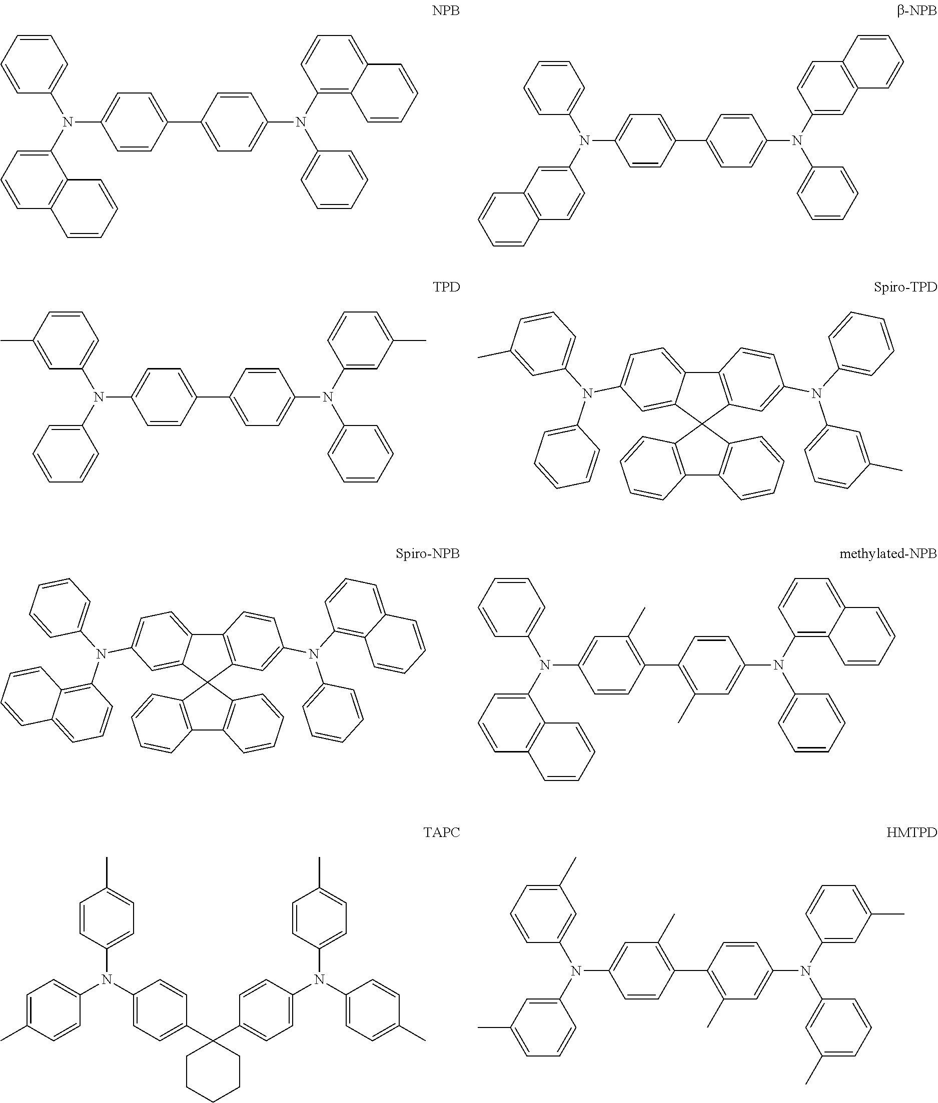

A thickness of the hole transport region may be in a range of about 50 Å to about 10,000 Å. For example, the thickness of the hole transport region may be in a range of about 100 Å to about 4,000 Å. When the hole transport region includes a hole injection layer, a hole transport layer, or any combination thereof, a thickness of the hole injection layer may be in a range of about 100 Å to about 9,000 Å, and a thickness of the hole transport layer may be in a range of about 50 Å to about 2,000 Å. For example, the thickness of the hole injection layer may be in a range of about 100 Å to about 1,000 Å. For example, the thickness of the hole transport layer may be in a range of about 100 Å to about 1,500 Å. When the thicknesses of the hole transport region, the hole injection layer, and the hole transport layer are within these ranges, satisfactory hole-transporting characteristics may be obtained without a substantial increase in driving voltage.

The emission auxiliary layer may increase light-emission efficiency by compensating for an optical resonance distance according to a wavelength of light emitted by an emission layer, and the electron blocking layer may block the leakage of electrons from an emission layer to a hole transport region. Materials that may be included in the hole transport region may be included in the emission auxiliary layer and the electron blocking layer.

[p-Dopant]

The hole transport region may further include, in addition to these materials, a charge-generation material for the improvement of conductive properties. The charge-generation material may be uniformly or non-uniformly dispersed in the hole transport region (for example, in the form of a single layer consisting of a charge-generation material).

The charge-generation material may be, for example, a p-dopant.

In an embodiment, a LUMO energy level of the p-dopant may be equal to or less than about −3.5 eV.

In an embodiment, the p-dopant may include a quinone derivative, a cyano group-containing compound, a compound containing element EL1 and element EL2, or any combination thereof.

Examples of the quinone derivative may include TCNQ, F4-TCNQ, and the like.

Examples of the cyano group-containing compound may include HAT-CN, a compound represented by Formula 221, and the like.

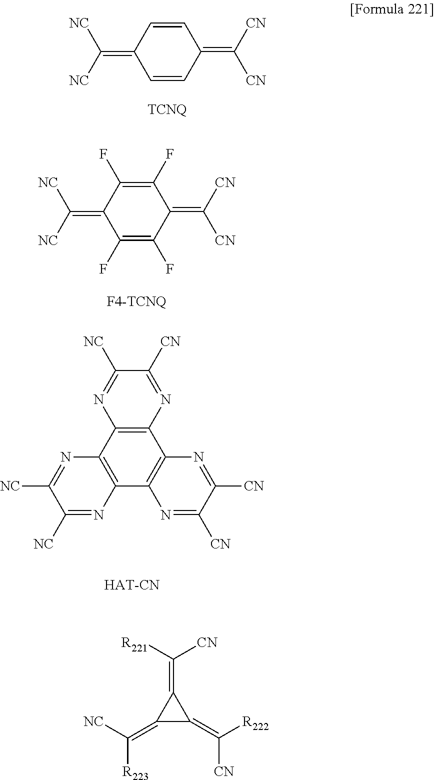

In Formula 221,

R₂₁ to R₂₂₃ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ, and

at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be a metal, a metalloid, or a combination thereof, and element EL2 may be a non-metal, a metalloid, or a combination thereof.

Examples of the metal may include: an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Examples of the metalloid may include silicon (Si), antimony (Sb), and tellurium (Te).

Examples of the non-metal may include oxygen (O) and a halogen (for example, F, Cl, Br, I, etc.).

In an embodiment, examples of the compound containing element EL1 and element EL2 may include a metal oxide, a metal halide (for example, a metal fluoride, a metal chloride, a metal bromide, or a metal iodide), a metalloid halide (for example, a metalloid fluoride, a metalloid chloride, a metalloid bromide, or a metalloid iodide), a metal telluride, or any combination thereof.

Examples of the metal oxide may include tungsten oxide (for example, WO, W₂O₃, WO₂, WO, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), and rhenium oxide (for example, ReO₃, etc.).

Examples of the metal halide may include an alkali metal halide, an alkaline earth metal halide, a transition metal halide, a post-transition metal halide, and a lanthanide metal halide.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, and CsI.

Examples of the alkaline earth metal halide may include BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, and BaI₂.

Examples of the transition metal halide may include a titanium halide (for example, TiF₄, TiCl₄, TiBr₄, TiI₄, etc.), a zirconium halide (for example, ZrF₄, ZrCl₄, ZrBr₄, ZrI₄, etc.), a hafnium halide (for example, HfF₄, HfCl₄, HfBr₄, Hfl₄, etc.), a vanadium halide (for example, VF₃, VCl₃, VBr₃, VI₃, etc.), a niobium halide (for example, NbF₃, NbCl₃, NbBr₃, NbI₃, etc.), a tantalum halide (for example, TaF₃, TaCl₃, TaBr₃, TaI₃, etc.), a chromium halide (for example, CrF₃, CrCl₃, CrBr₃, CrI₃, etc.), a molybdenum halide (for example, MoF₃, MoCl₃, MoBr₃, MoI₃, etc.), a tungsten halide (for example, WF₃, WCl₃, WBr₃, WI₃, etc.), a manganese halide (for example, MnF₂, MnCl₂, MnBr₂, MnI₂, etc.), a technetium halide (for example, TcF₂, TcCl₂, TcBr₂, TcI₂, etc.), a rhenium halide (for example, ReF₂, ReCl₂, ReBr₂, ReI₂, etc.), an iron halide (for example, FeF₂, FeCl₂, FeBr₂, FeI₂, etc.), a ruthenium halide (for example, RuF₂, RuCl₂, RuBr₂, RuI₂, etc.), an osmium halide (for example, OsF₂, OsCl₂, OsBr₂, OsI₂, etc.), a cobalt halide (for example, CoF₂, CoCl₂, CoBr₂, CoI₂, etc.), a rhodium halide (for example, RhF₂, RhCl₂, RhBr₂, RhI₂, etc.), an iridium halide (for example, IrF₂, IrCl₂, IrBr₂, IrI₂, etc.), a nickel halide (for example, NiF₂, NiCl₂, NiBr₂, NiI₂, etc.), a palladium halide (for example, PdF₂, PdCl₂, PdBr₂, PdI₂, etc.), a platinum halide (for example, PtF₂, PtCl₂, PtBr₂, PtI₂, etc.), a copper halide (for example, CuF, CuCl, CuBr, CuI, etc.), a silver halide (for example, AgF, AgCl, AgBr, AgI, etc.), and a gold halide (for example, AuF, AuCl, AuBr, AuI, etc.).

Examples of the post-transition metal halide may include a zinc halide (for example, ZnF₂, ZnCl₂, ZnBr₂, ZnI₂, etc.), an indium halide (for example, InI₃, etc.), and a tin halide (for example, SnI₂, etc.).

Examples of the lanthanide metal halide may include YbF, YbF₂, YbF₃, SmF₃, YbCl, YbCl₂, YbCl₃ SmCl₃, YbBr, YbBr₂, YbBr₃, SmBr₃, YbI, YbI₂, YbI₃, and SmI₃.

Examples of the metalloid halide may include an antimony halide (for example, SbCl₅, etc.).

Examples of the metal telluride may include an alkali metal telluride (for example, Li₂Te, Na₂Te, K₂Te, Rb₂Te, Cs₂Te, etc.), an alkaline earth metal telluride (for example, BeTe, MgTe, CaTe, SrTe, BaTe, etc.), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (for example, ZnTe, etc.), and a lanthanide metal telluride (for example, LaTe, CeTe, PrTe, NdTe, PmTe, EuTe, GdTe, TbTe, DyTe, HoTe, ErTe, TmTe, YbTe, LuTe, etc.).

[Emission Layer in Interlayer 130]

When the light-emitting device 10 is a full-color light-emitting device, the emission layer may be patterned into a red emission layer, a green emission layer, and/or a blue emission layer, according to a subpixel. In an embodiment, the emission layer may have a stacked structure of two or more layers of a red emission layer, a green emission layer, and a blue emission layer, in which the two or more layers may contact each other or may be separated from each other. In embodiments, the emission layer may include two or more materials of a red light-emitting material, a green light-emitting material, and a blue light-emitting material, in which the two or more materials are mixed with each other in a single layer to emit white light.

The emission layer may include a host and a dopant. The dopant may include a phosphorescent dopant, a fluorescent dopant, or any combination thereof.

An amount of the dopant in the emission layer may be in a range of about 0.01 parts by weight to about 15 parts by weight, based on 100 parts by weight of the host.

In an embodiment, the emission layer may include a quantum dot.

In an embodiment, the emission layer may include a delayed fluorescence material. The delayed fluorescence material may serve as a host or as a dopant in the emission layer.

A thickness of the emission layer may be in a range of about 100 Å to about 1,000 Å. For example, the thickness of the emission layer may be in a range of about 200 Å to about 600 Å. When the thickness of the emission layer is within these range, excellent light-emission characteristics may be obtained without a substantial increase in driving voltage.

[Host]

The host may include a compound represented by Formula 301:

[Ar₃₀₁]_{xb11}-[(La₃₀₁)_{xb1}-R₃₀₁]_{xb21} [Formula 301]

In Formula 301,

Ar₃₀₁ and La₃₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,

xb11 may be 1, 2, or 3,

xb1 may be an integer from 0 to 5,

Ra₃₀₁ may be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer from 1 to 5, and

Q₃₀₁ to Q₃₀₃ are each independently the same as described in connection with Q₁.

In an embodiment, in Formula 301, when xb11 is 2 or more, two or more of Ar₃₀₁(s) may be linked to each other via a single bond.

In an embodiment, the host may include a compound represented by Formula 301-1, a compound represented by Formula 301-2, or any combination thereof:

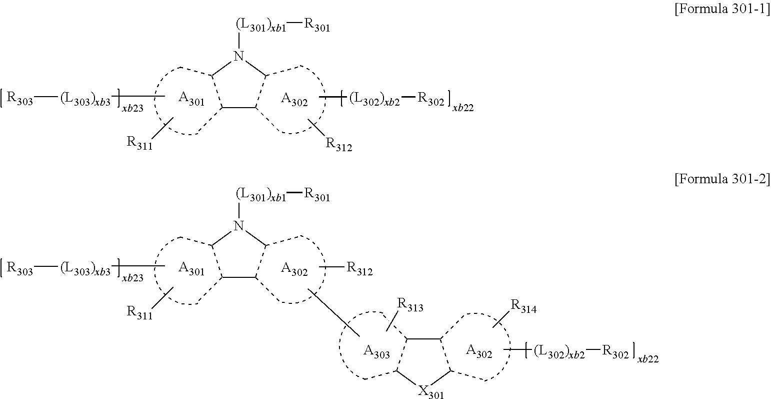

In Formulae 301-1 and 301-2,

ring A₃₀₁ to ring A₃₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,

X₃₀₁ may be O, S, N-[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ are each the same as described in the specification,

L₃₀₂ to L₃₀₄ are each independently the same as described in connection with L₃₀₁,

xb2 to xb4 are each independently the same as described in connection with xb1, and

R₃₀₀ to R₃₀₅ and R₃₁₁ to R₃₁₄ are each independently the same as described in connection with R₃₀₁.

In an embodiment, the host may include an alkali earth metal complex, a post-transition metal complex, or a combination thereof. In an embodiment, the host may include a Be complex (for example, Compound H55), an Mg complex, a Zn complex, or a combination thereof.

In an embodiment, the host may include one of Compounds H1 to H124, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

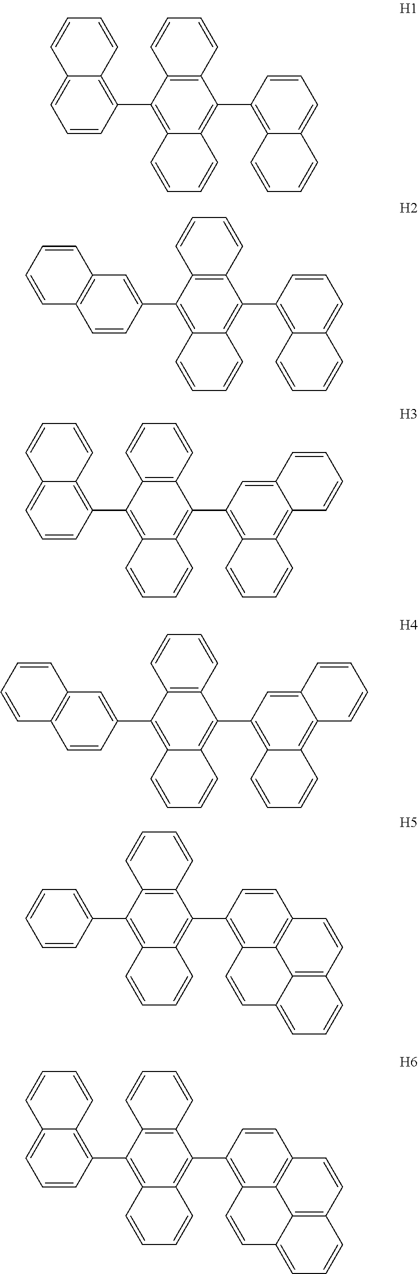

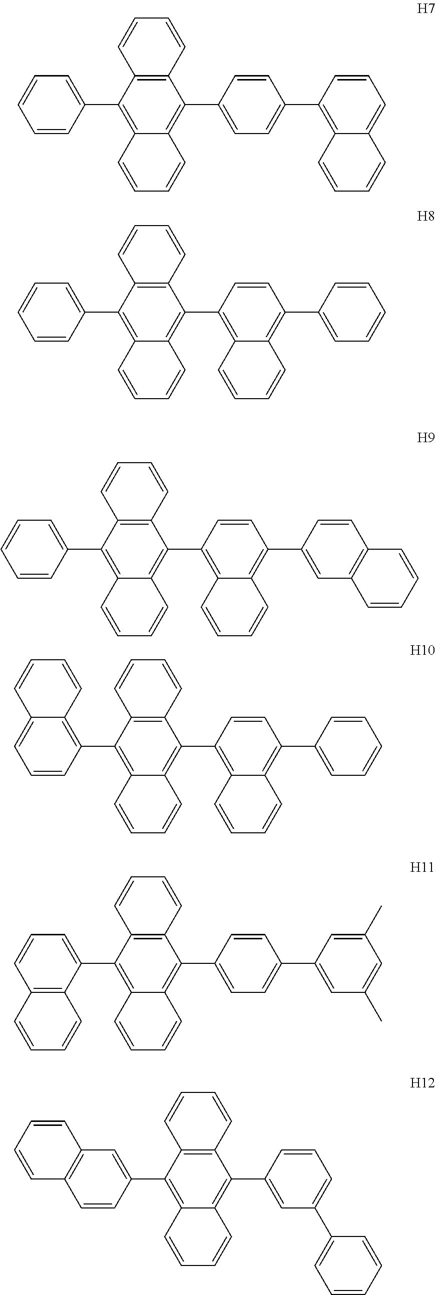

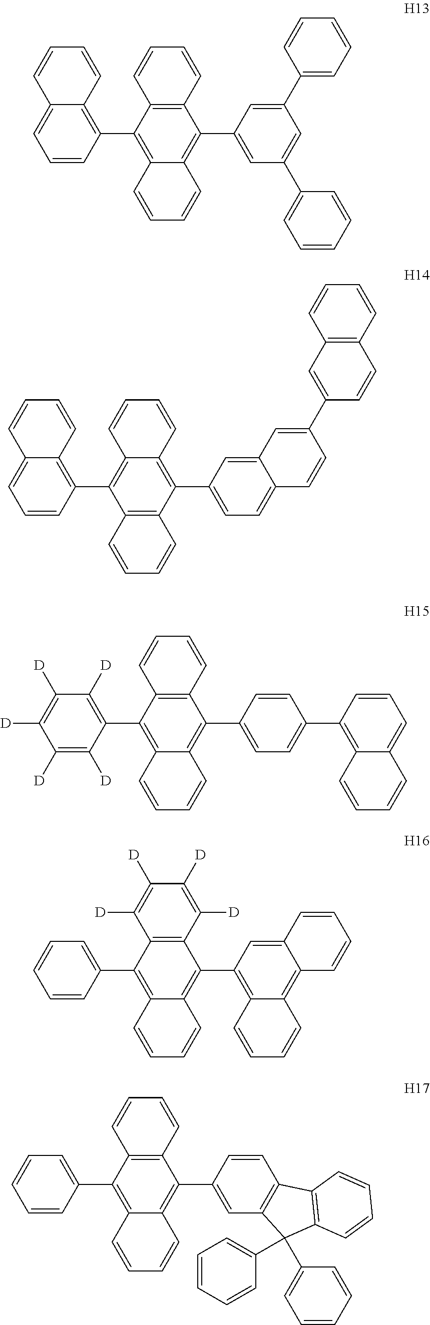

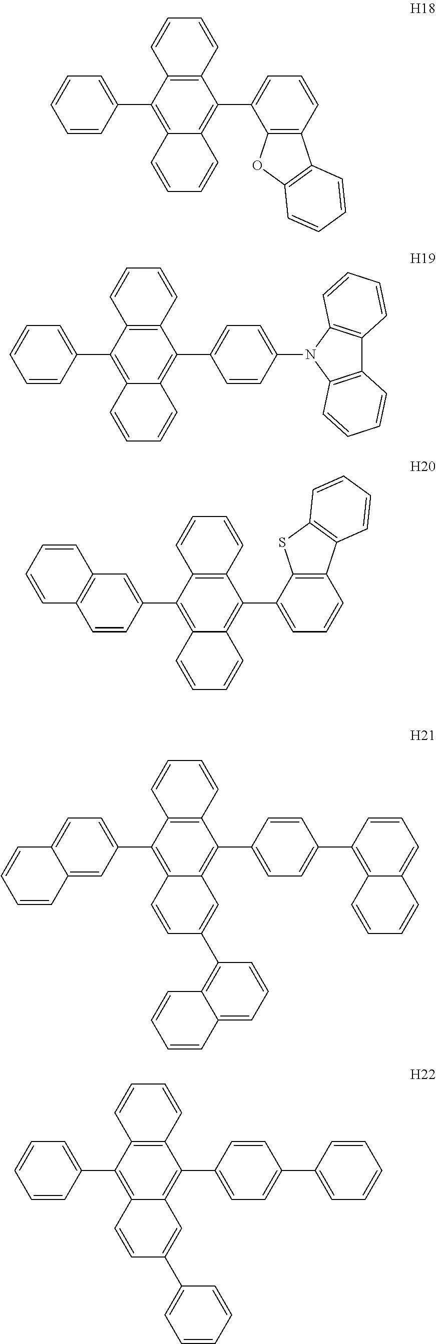

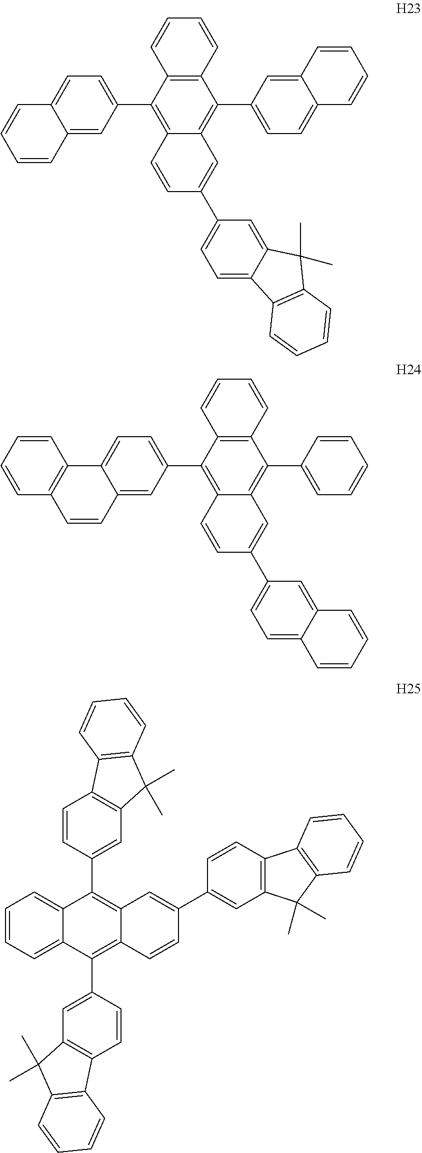

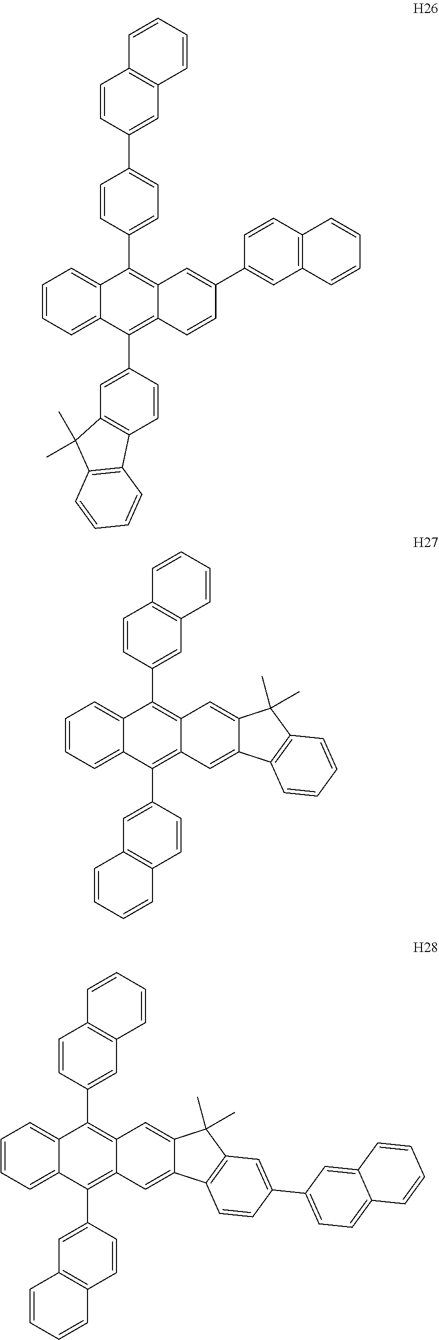

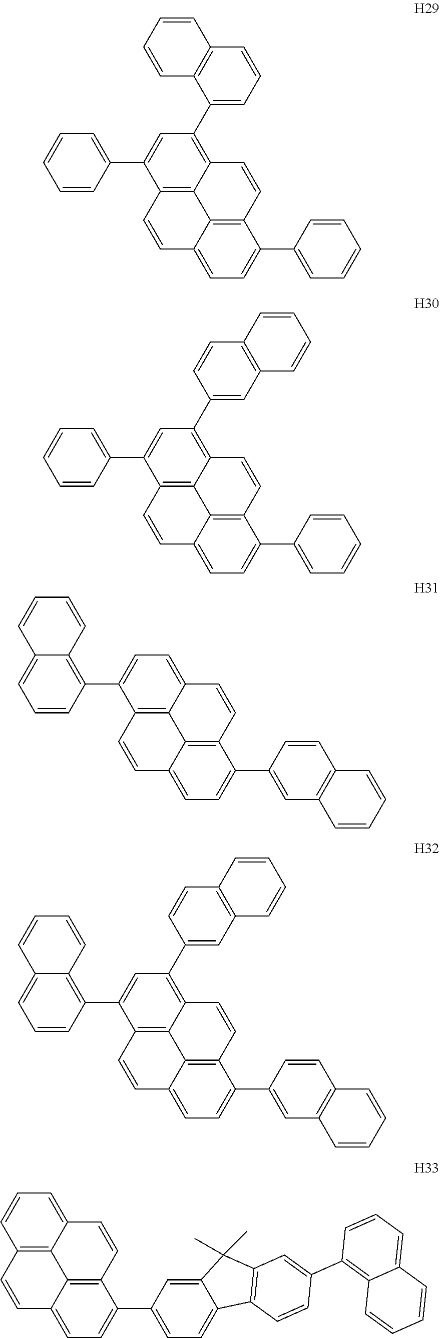

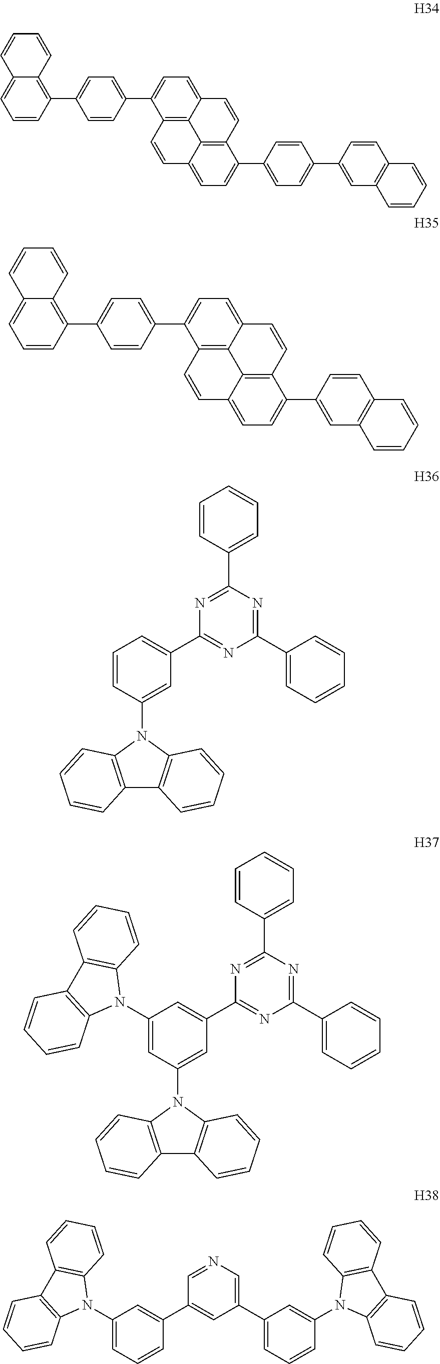

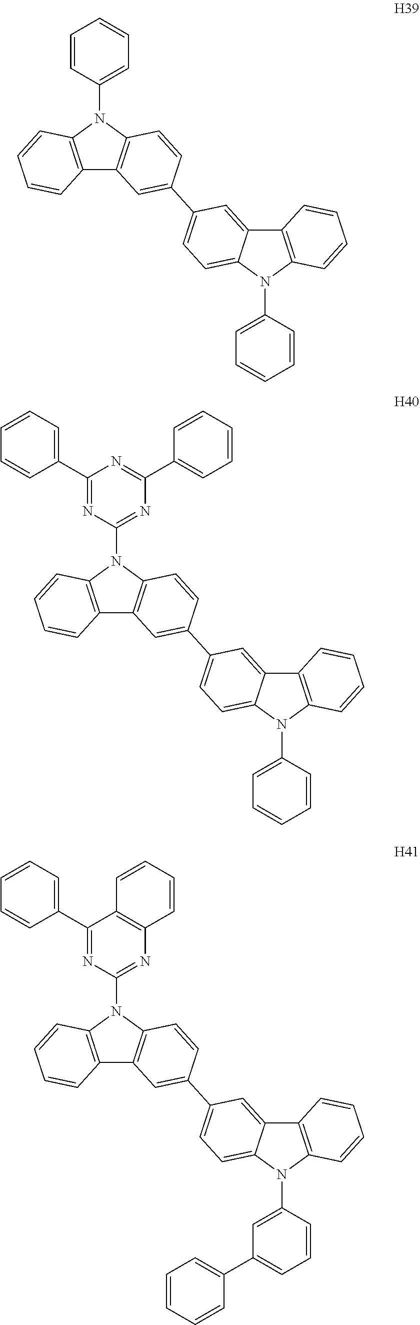 [SMILES: c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1]

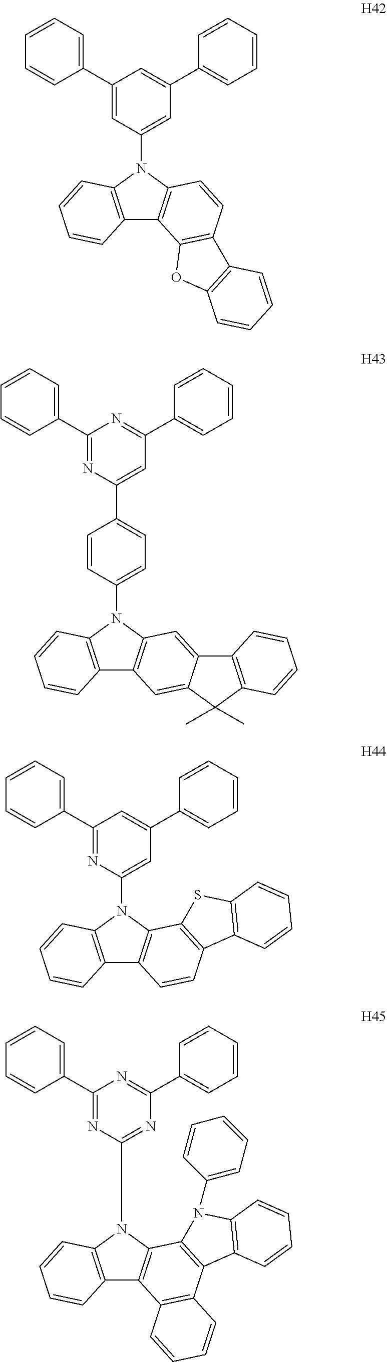

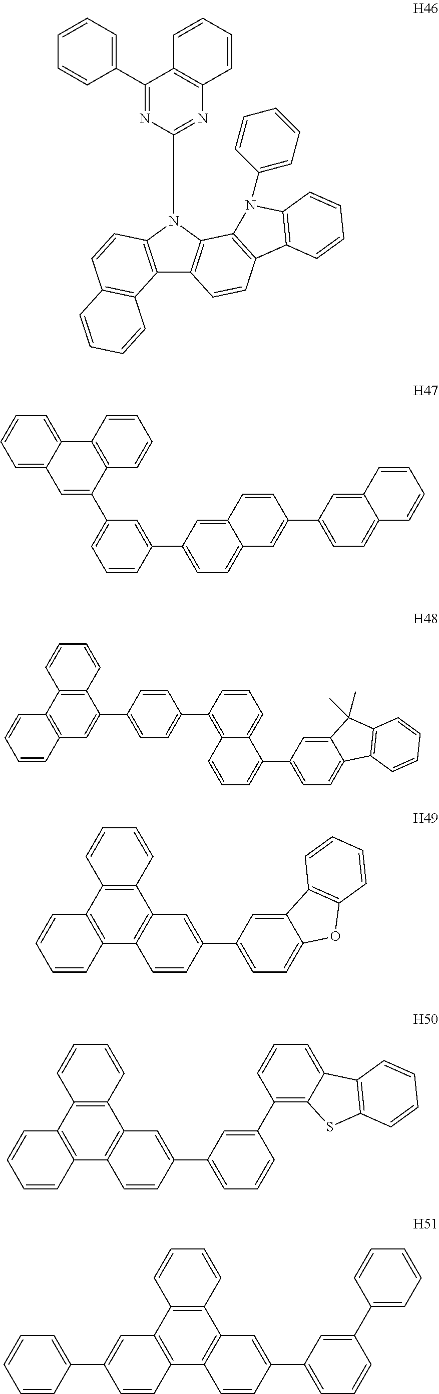

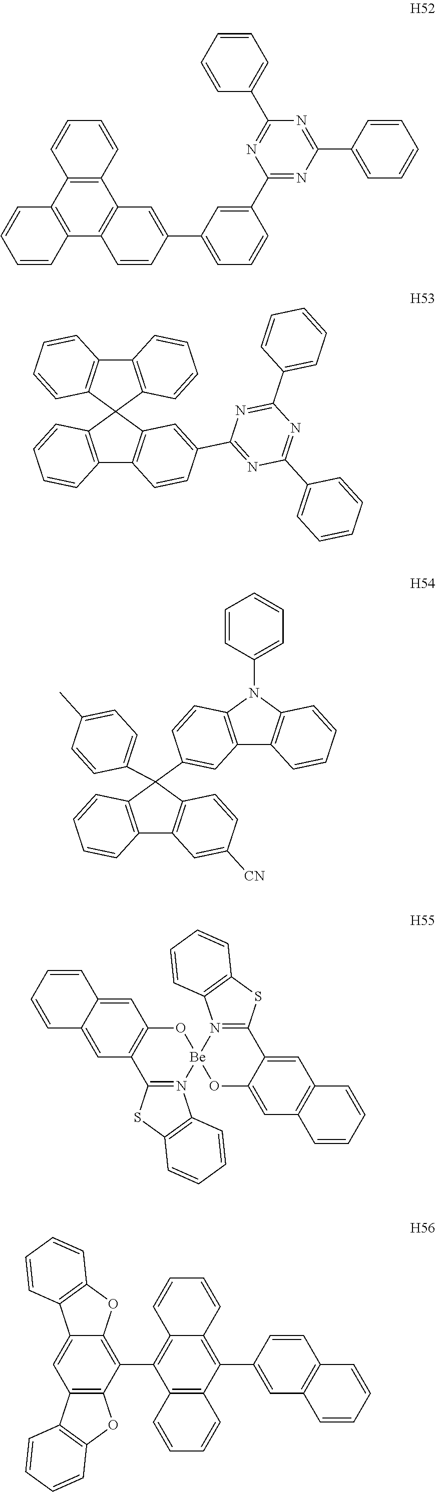

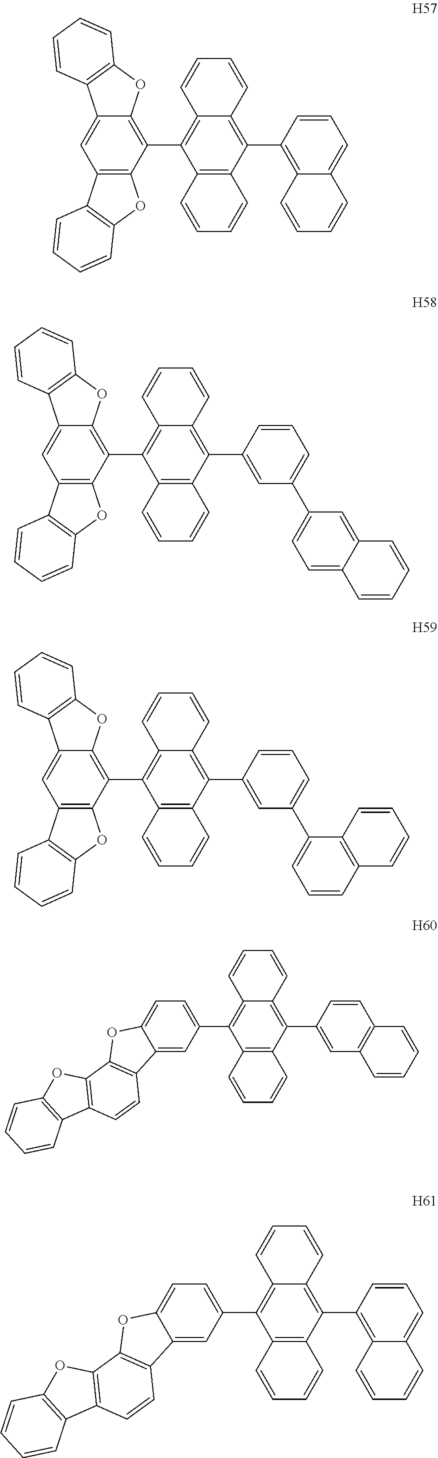 [SMILES: c1ccc2c(-c3c4ccccc4c(-c4c5oc6ccccc6c5cc5c4oc4ccccc45)c4ccccc34)cccc2c1 ; c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1]

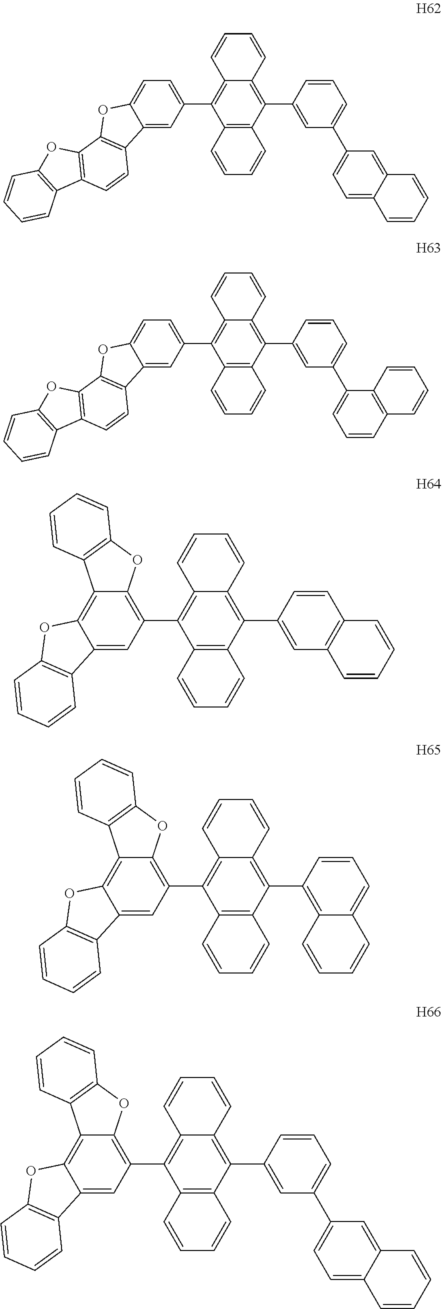

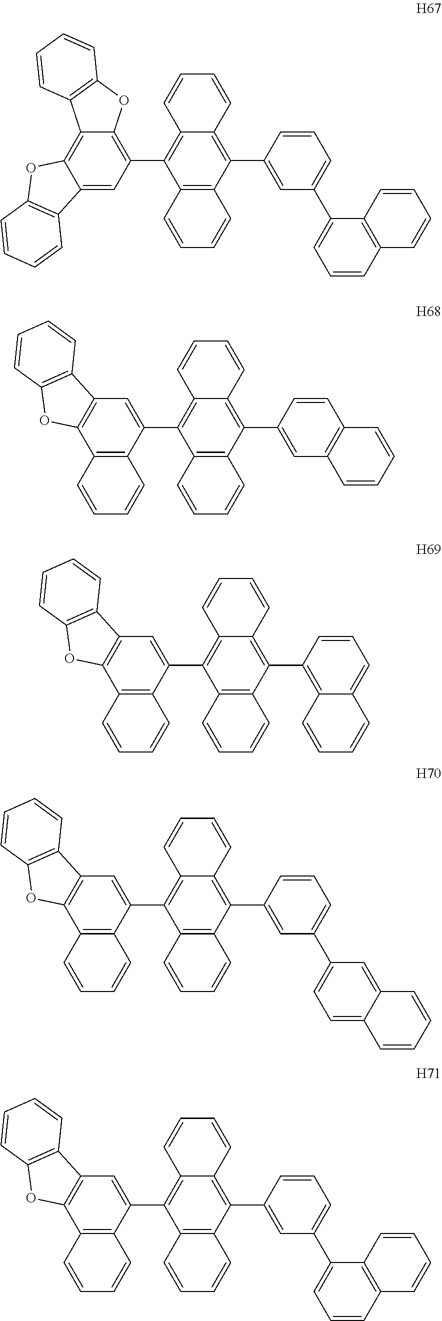 [SMILES: c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1]

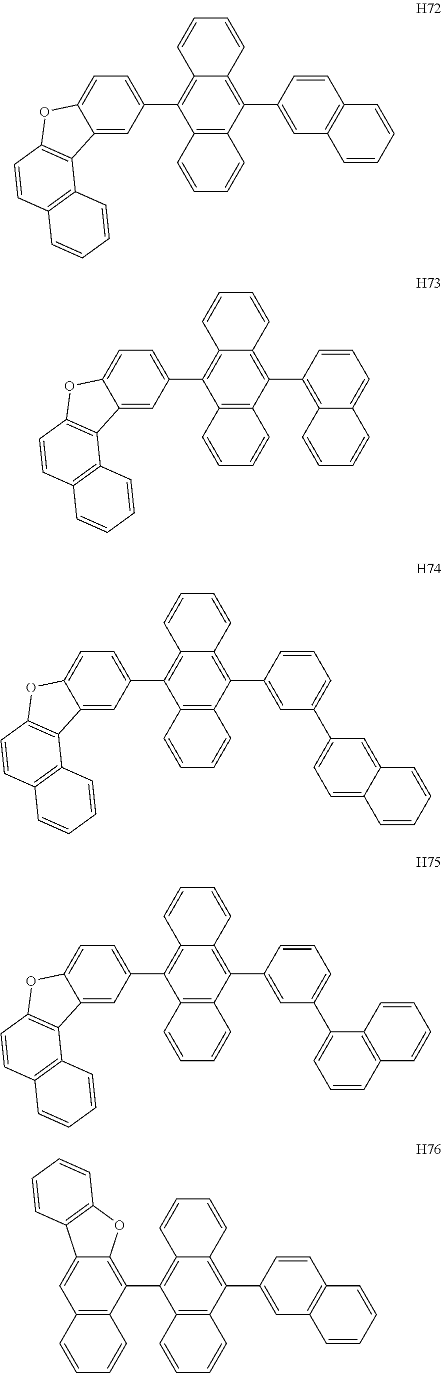

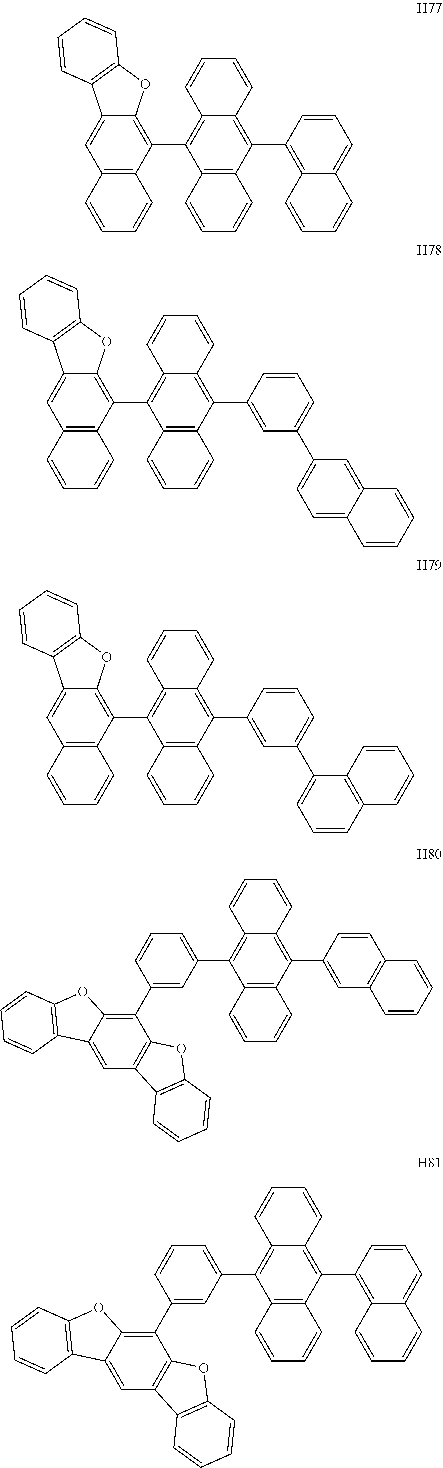 [SMILES: c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1]

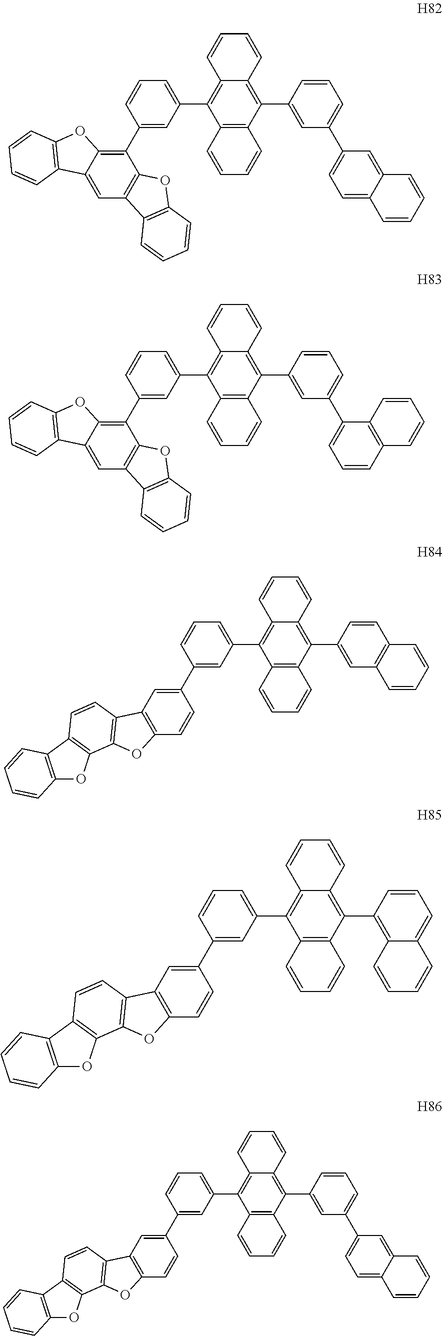 [SMILES: c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1]

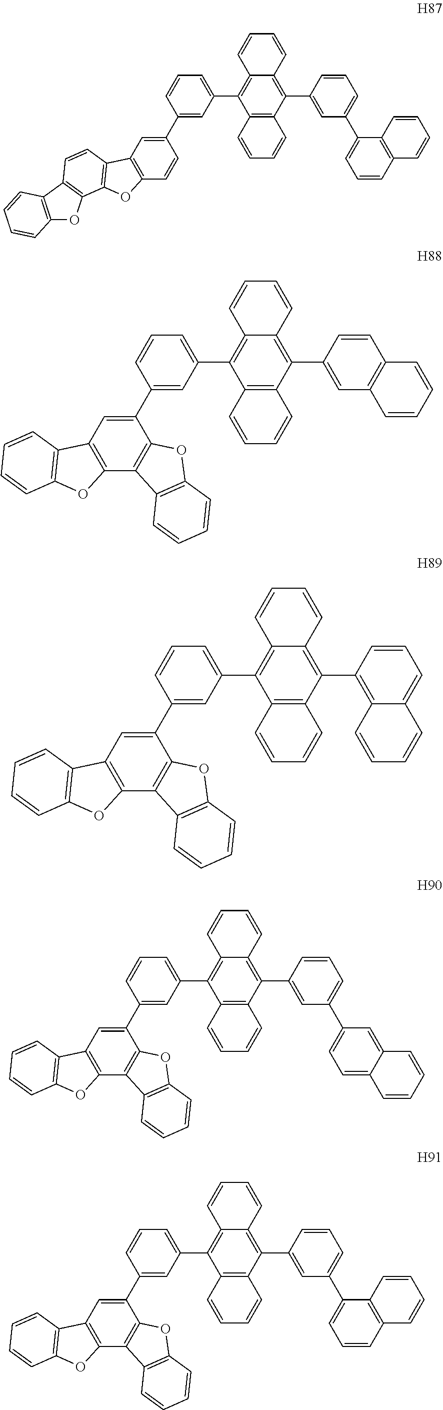 [SMILES: c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1]

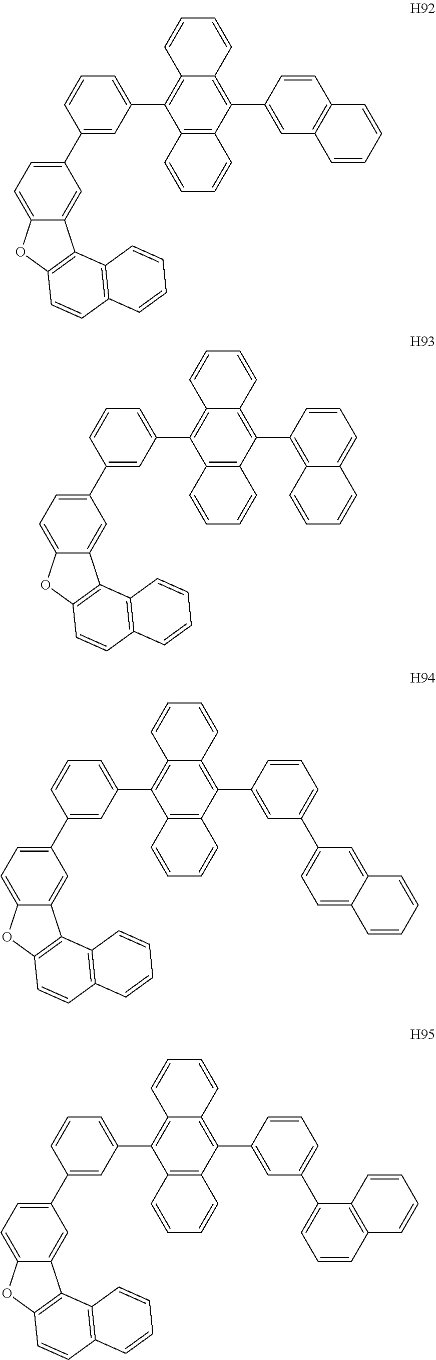 [SMILES: c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1]

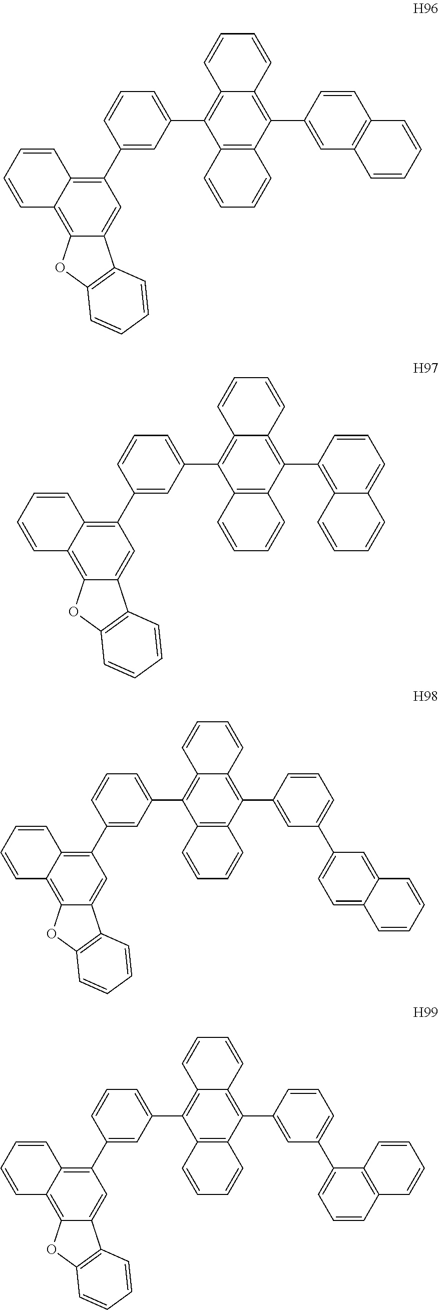 [SMILES: c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1]

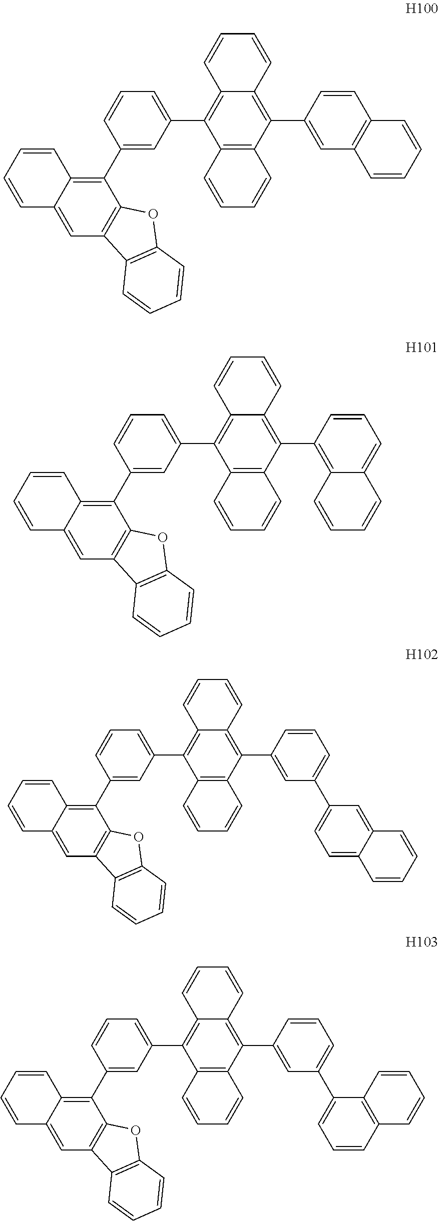 [SMILES: c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1]

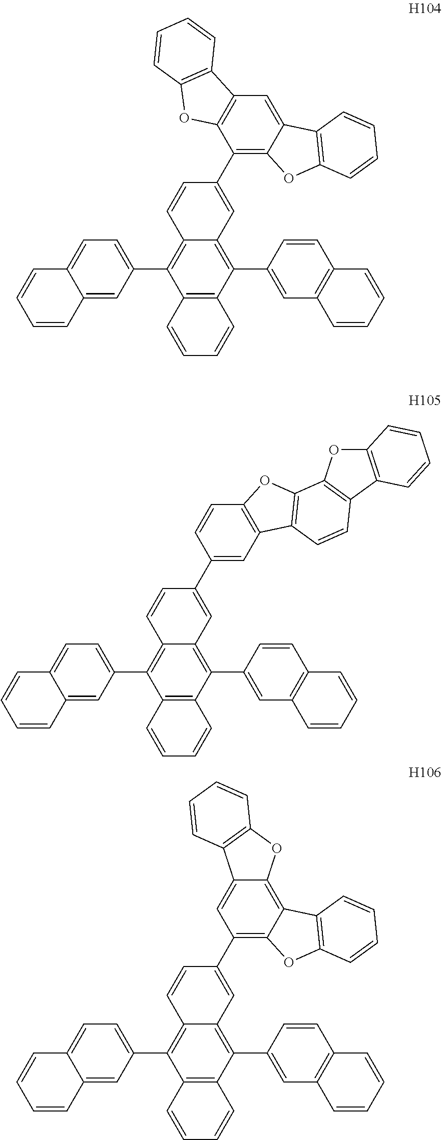

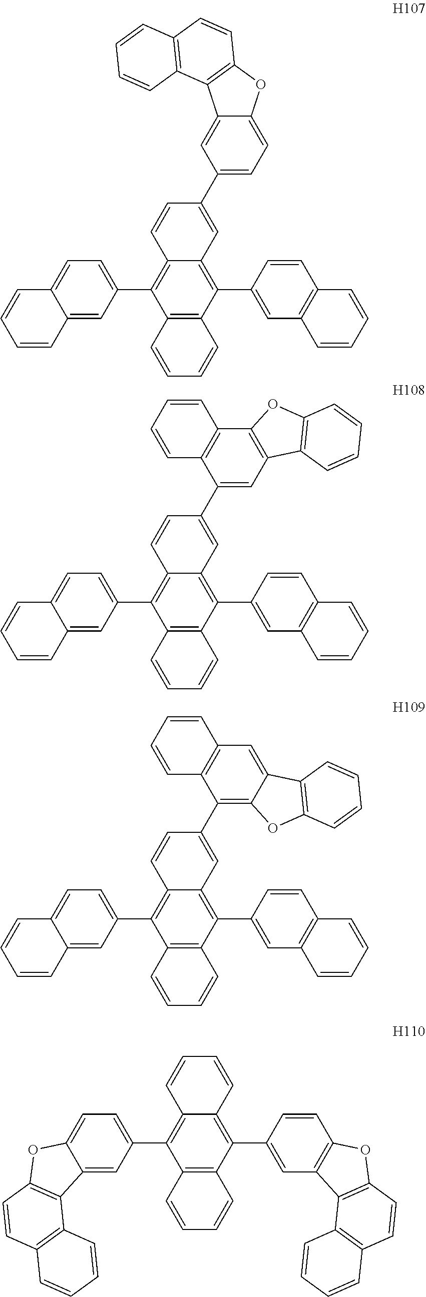

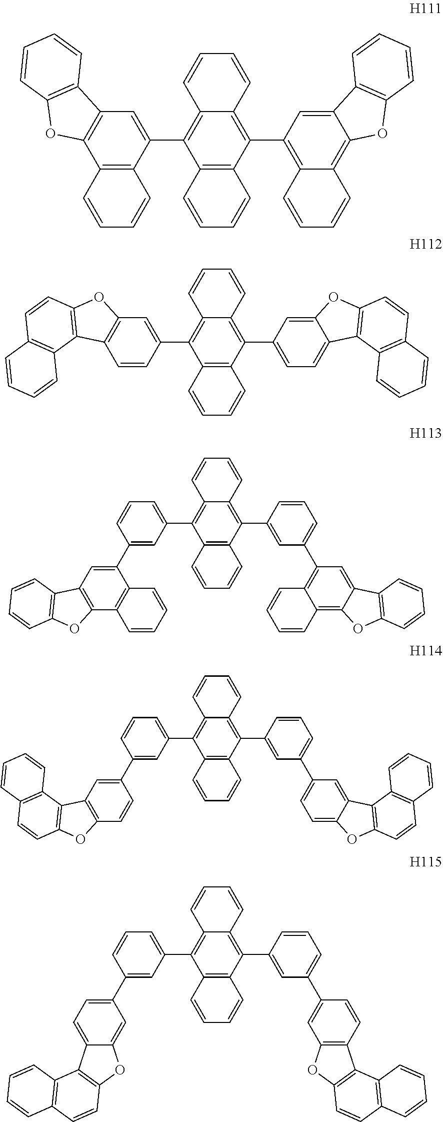 [SMILES: c1ccc2c(c1)oc1c3ccccc3c(-c3c4ccccc4c(-c4cc5c6ccccc6oc5c5ccccc45)c4ccccc34)cc21 ; c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1]

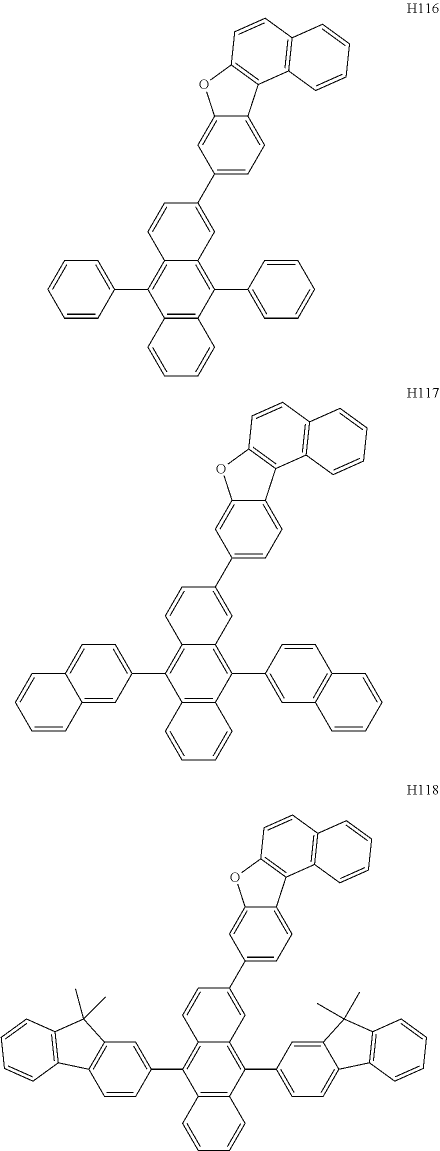 [SMILES: c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

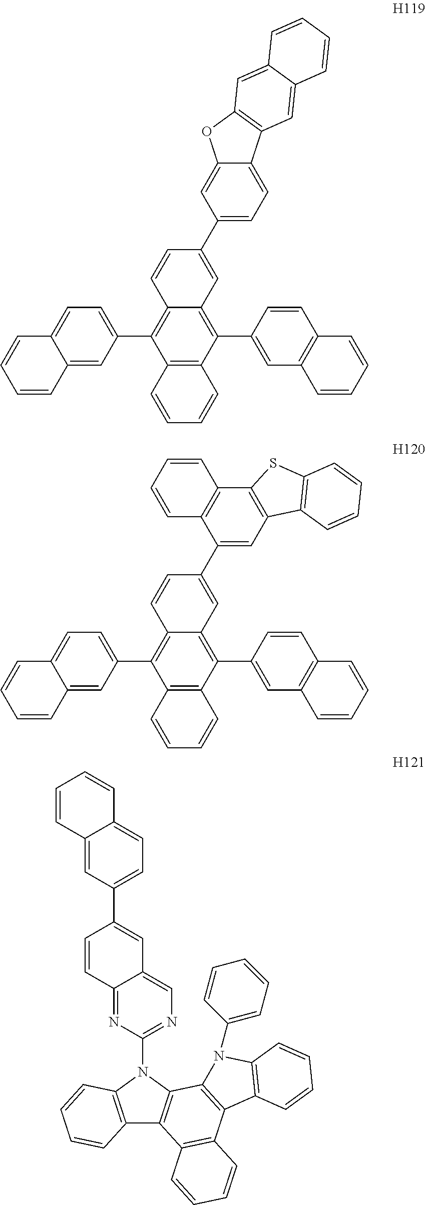

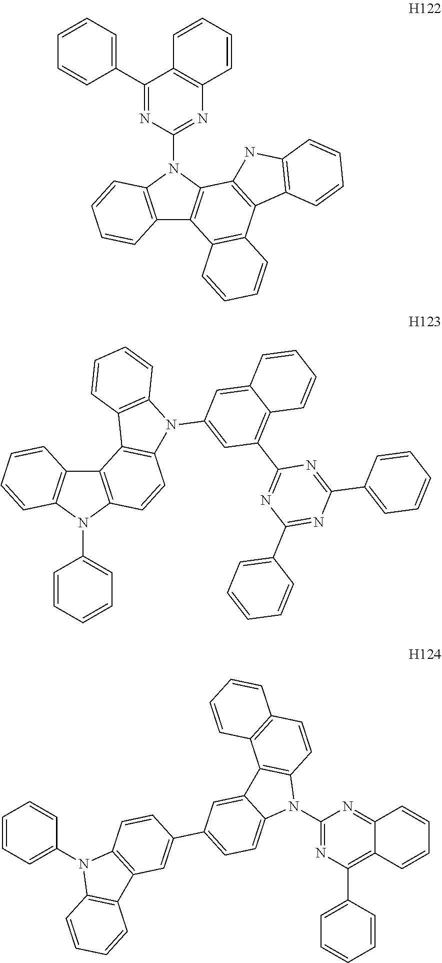 [SMILES: c1ccc(-c2nc(-n3c4ccccc4c4c5ccccc5c5c6ccccc6[nH]c5c43)nc3ccccc23)cc1 ; c1ccc(-c2nc(-c3ccccc3)nc(-c3cc(-n4c5ccccc5c5c6c7ccccc7n(-c7ccccc7)c6ccc54)cc4ccccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

[Phosphorescent Dopant]

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

In an embodiment, the phosphorescent dopant may include an organometallic compound represented by Formula 401:

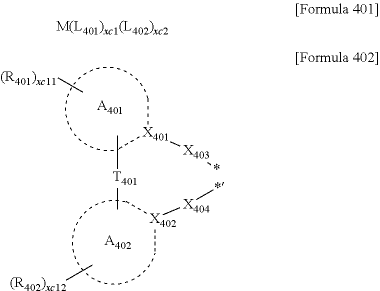

In Formulae 401 and 402,

M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),

L₄₀₁ may be a ligand represented by Formula 402, and xc1 may be 1, 2, or 3, wherein, when xc1 is two or more, two or more of L₄₀₁(s) may be identical to or different from each other,

L₄₀₂ may be an organic ligand, and xc2 may be 0, 1, 2, 3, or 4, wherein, when xc2 is 2 or more, two or more of L₄₀₂(s) may be identical to or different from each other,

X₄₀₁ and X₄₀₂ may each independently be nitrogen or carbon,

ring A₄₀₁ and ring A₄₀₂ may each independently be a C₃-C₆₀ carbocyclic group or a C₁-C₆₀ heterocyclic group,

T₄₀₁ may be a single bond, *—O—*, *—S—*, *—C(═O)—*′, *—N(Q₄₁₁)—*′, *—C(Q₄₁₁)(Q₄₁₂)—*′, *—C(Q₄₁₁)═C(Q₄₁₂)—*′, *—C(Q₄₁₁)═*′, or *═C═*,

X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ are each independently the same as described in connection with Q₁,

R₄₀₁ and R₄₀₂ may each independently be hydrogen, deuterium, —F, —Cl, —Br, —I, a hydroxyl group, a cyano group, a nitro group, a C₁-C₂₀ alkyl group that is unsubstituted or substituted with at least one R₁₀ₐ, a C₁-C₂₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ are each independently the same as described in connection with Q₁,

xc11 and xc12 may each independently be an integer from 0 to 10, and

* and *′ in Formula 402 each indicate a binding site to M in Formula 401.

In an embodiment, in Formula 402, X₄₀₁ may be nitrogen and X₄₀₂ may be carbon, or each of X₄₀₁ and X₄₀₂ may be nitrogen.

In an embodiment, in Formula 401, when xc1 is 2 or more, two ring A₄₀₁ in two or more of L₄₀₁(s) may be optionally linked to each other via T₄₀₂, which is a linking group, and two ring A₄₀₂ may optionally be linked to each other via T₄₀₃, which is a linking group (see Compounds PD1 to PD4 and PD7). T₄₀₂ and T₄₀₃ are each independently the same as described in connection with T₄₀₁.

In Formula 401, L₄₀₂ may be an organic ligand. In an embodiment, L₄₀₂ may include a halogen group, a diketone group (for example, an acetylacetonate group), a carboxylic acid group (for example, a picolinate group), —C(═O), an isonitrile group, —CN group, a phosphorus group (for example, a phosphine group, a phosphite group, etc.), or any combination thereof.

The phosphorescent dopant may include, for example, one of compounds PD1 to PD39, or any combination thereof:

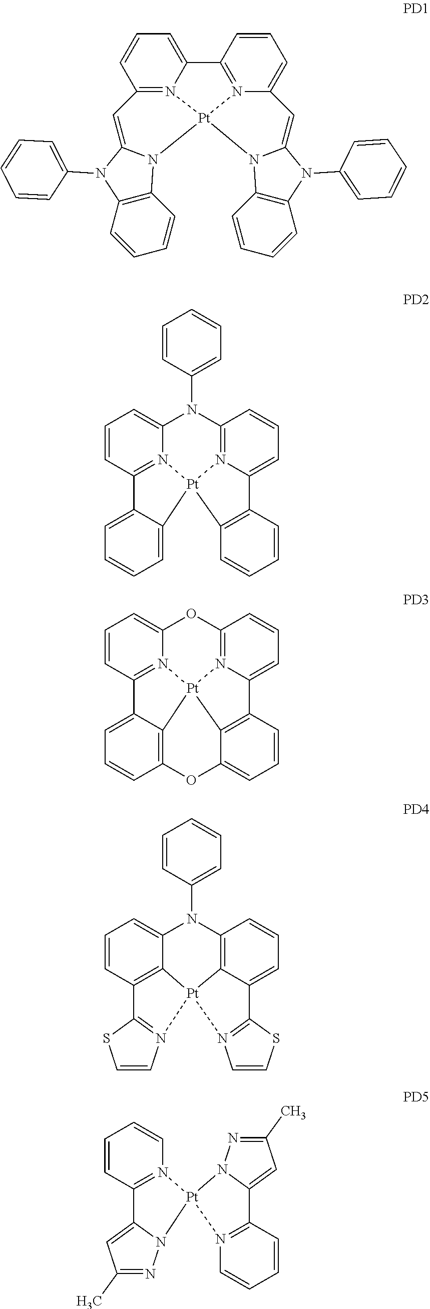

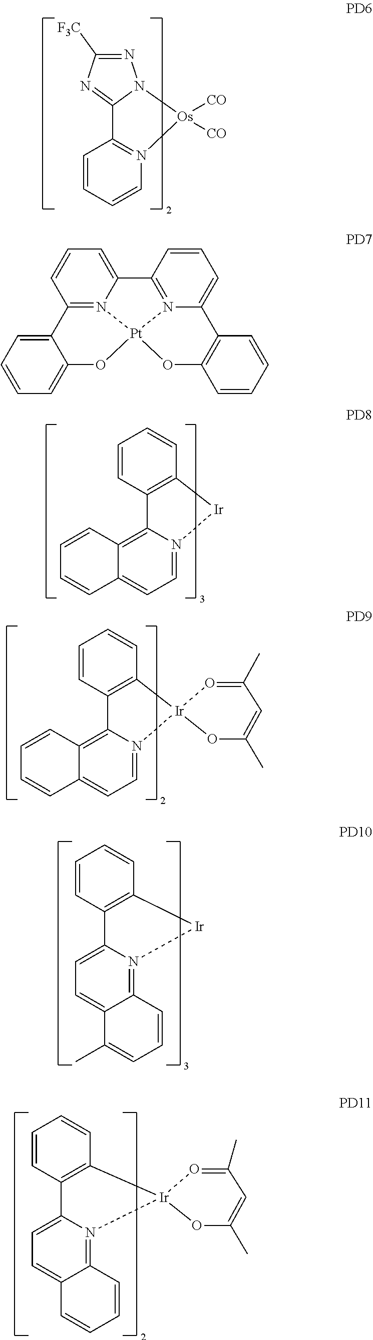

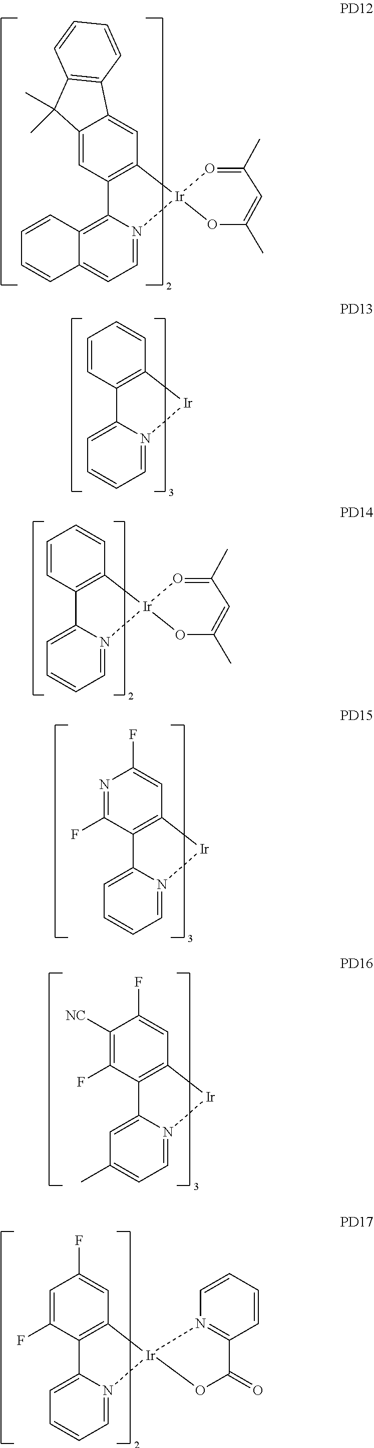

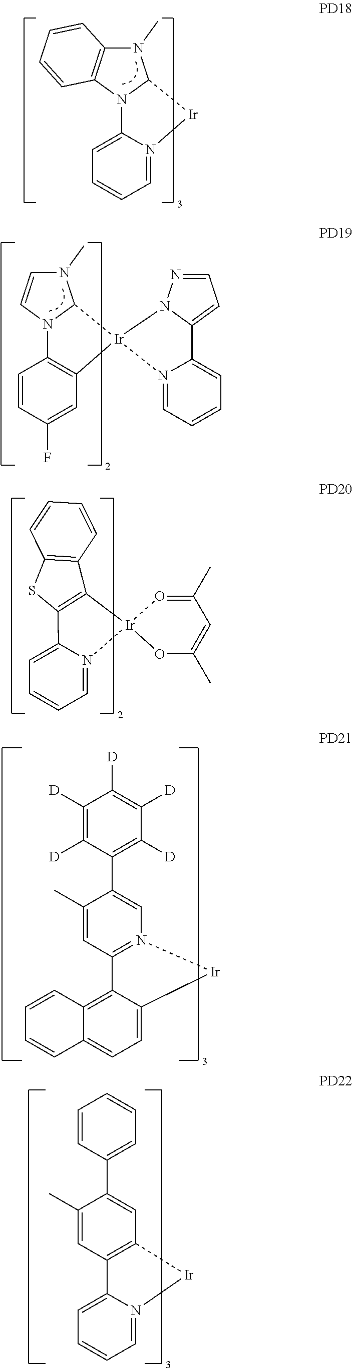

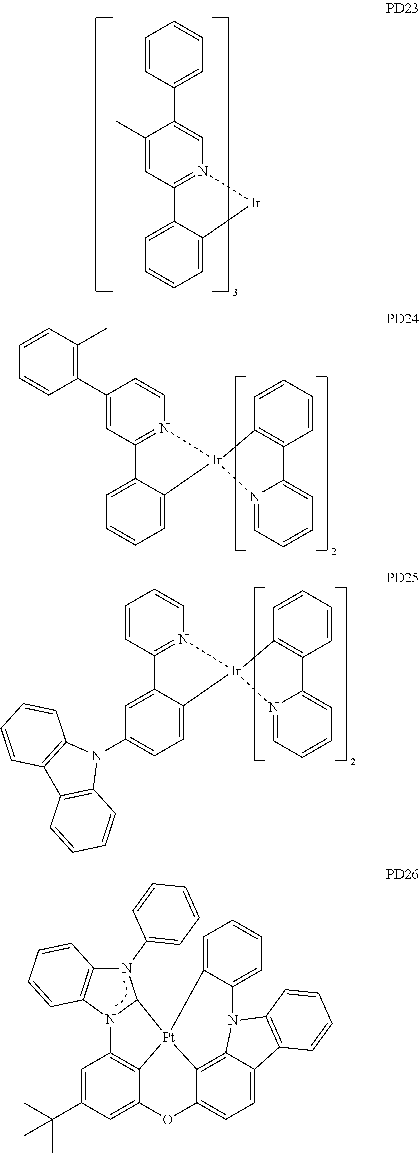

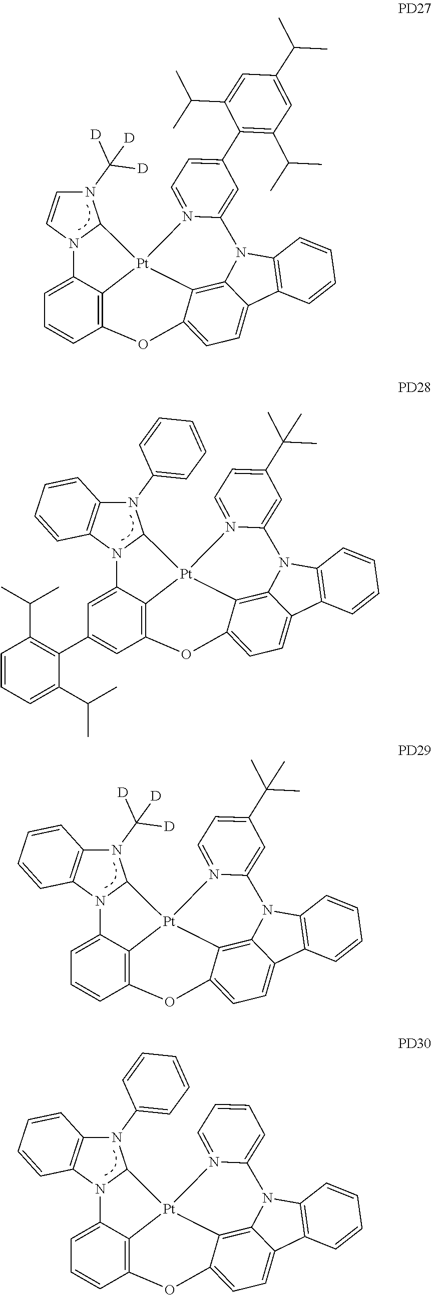

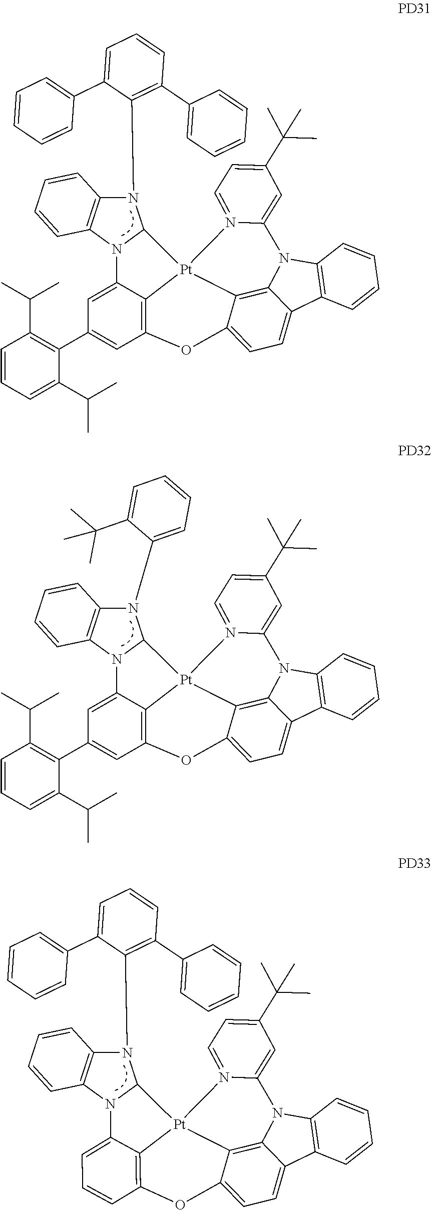

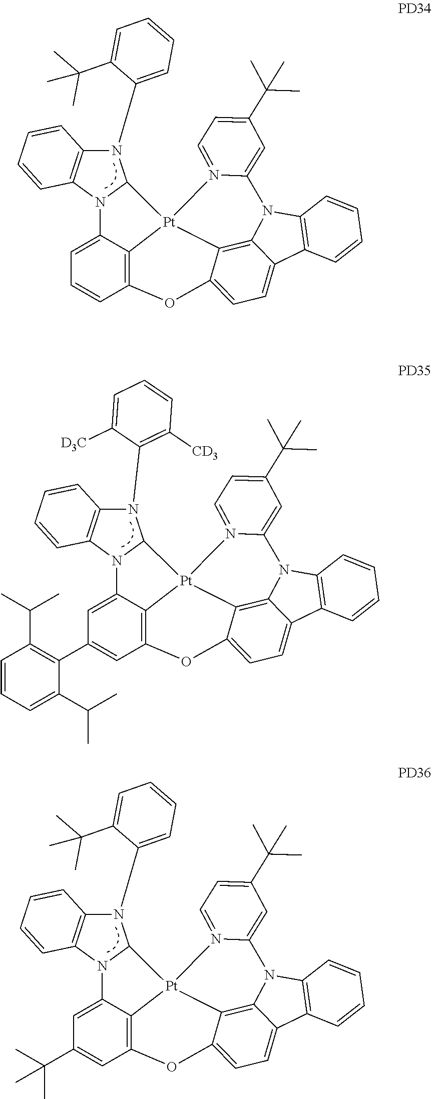

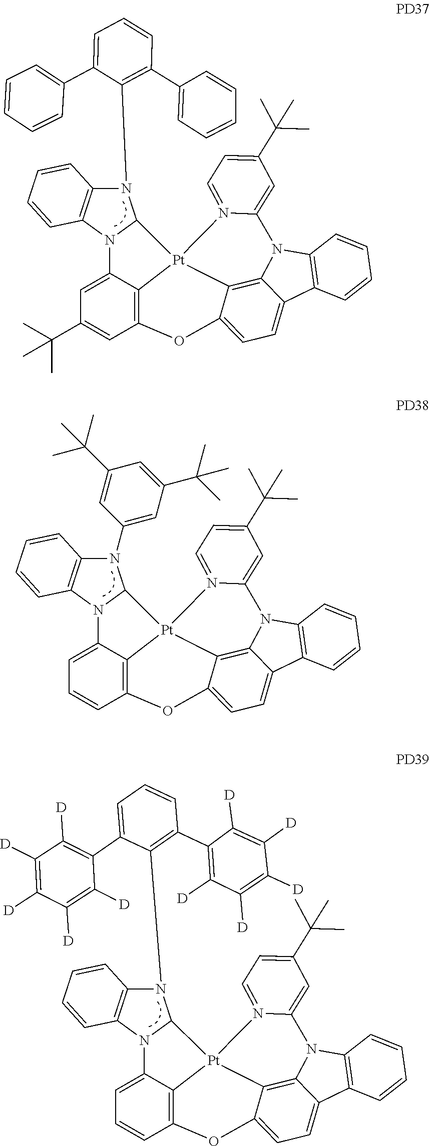

[Fluorescent Dopant]

The fluorescent dopant may include an amine group-containing compound, a styryl group-containing compound, or any combination thereof.

In an embodiment, the fluorescent dopant may include a compound represented by Formula 501:

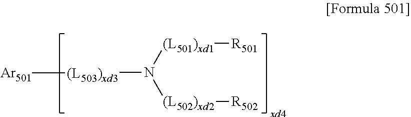

In Formula 501,

Ar₅₀₁, L₅₀₁ to L₅₀₃, R₅₀₁, and R₅₀₂ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,

xd1 to xd3 may each independently be 0, 1, 2, or 3, and

xd4 may be 1, 2, 3, 4, 5, or 6.

In an embodiment, in Formula 501, Ar₅₀₁ may be a condensed cyclic group (for example, an anthracene group, a chrysene group, or a pyrene group) in which three or more monocyclic groups are condensed together.

In an embodiment, in Formula 501, xd4 may be 2.

In an embodiment, the fluorescent dopant may include one of Compounds FD1 to FD36, DPVBi, DPAVBi, or any combination thereof:

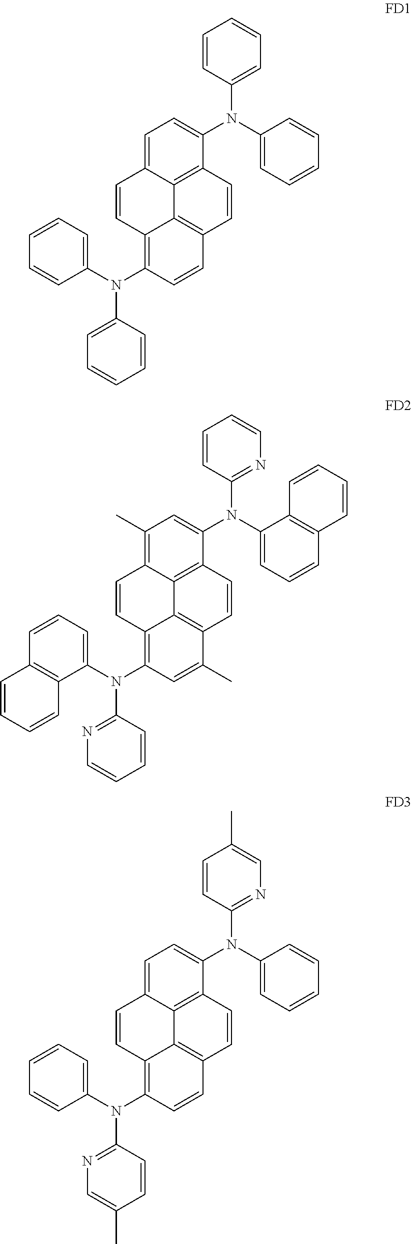 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1]

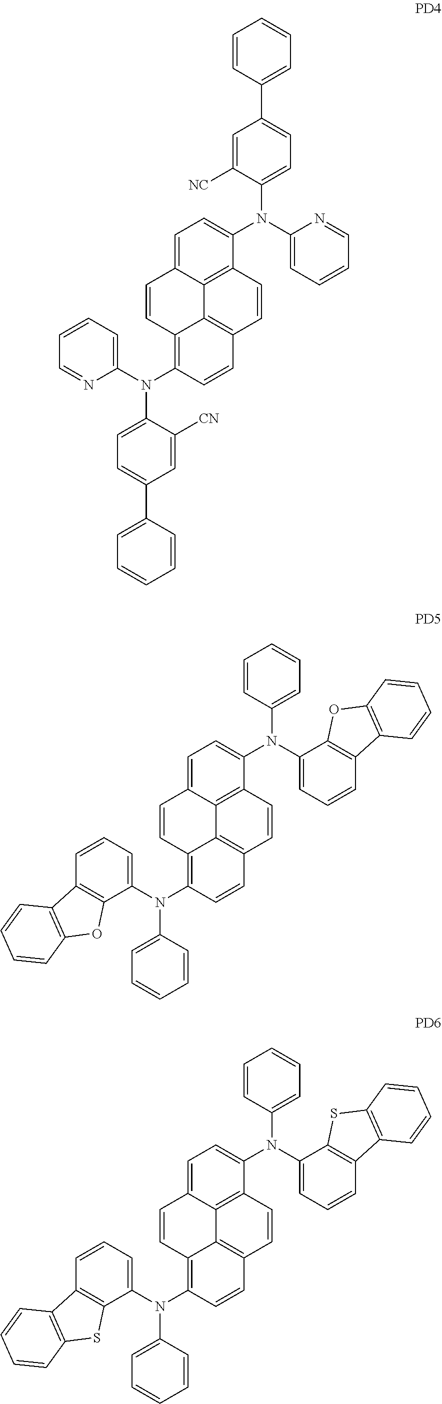 [SMILES: N#Cc1cc(-c2ccccc2)ccc1N(c1ccccn1)c1ccc2ccc3c(N(c4ccccn4)c4ccc(-c5ccccc5)cc4C#N)ccc4ccc1c2c43 ; c1ccc(N(c2ccc3ccc4c(N(c5ccccc5)c5cccc6c5oc5ccccc56)ccc5ccc2c3c54)c2cccc3c2oc2ccccc23)cc1 ; c1ccc(N(c2ccc3ccc4c(N(c5ccccc5)c5cccc6c5sc5ccccc56)ccc5ccc2c3c54)c2cccc3c2sc2ccccc23)cc1]

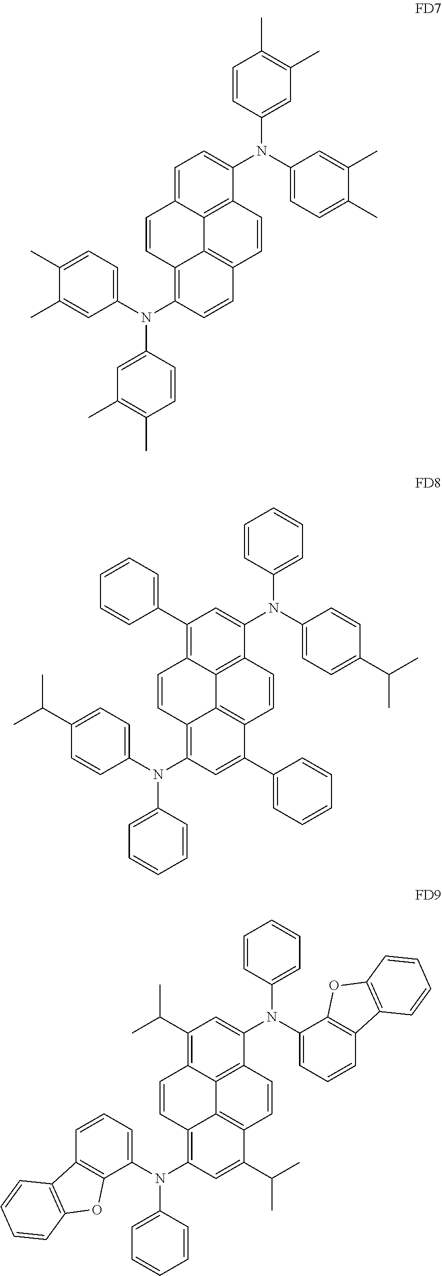

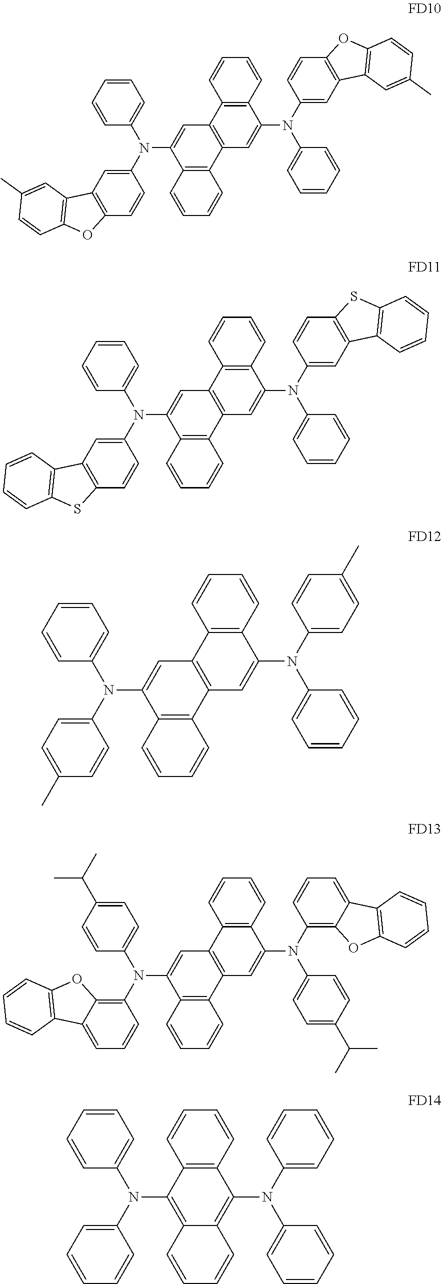

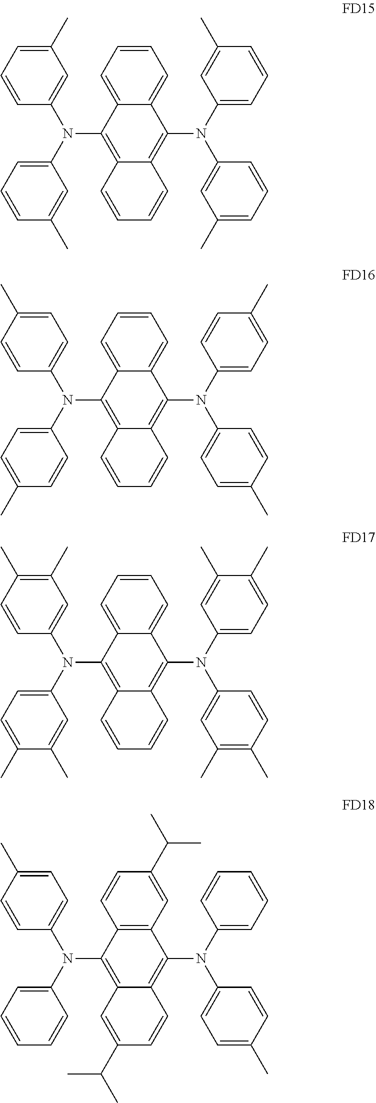

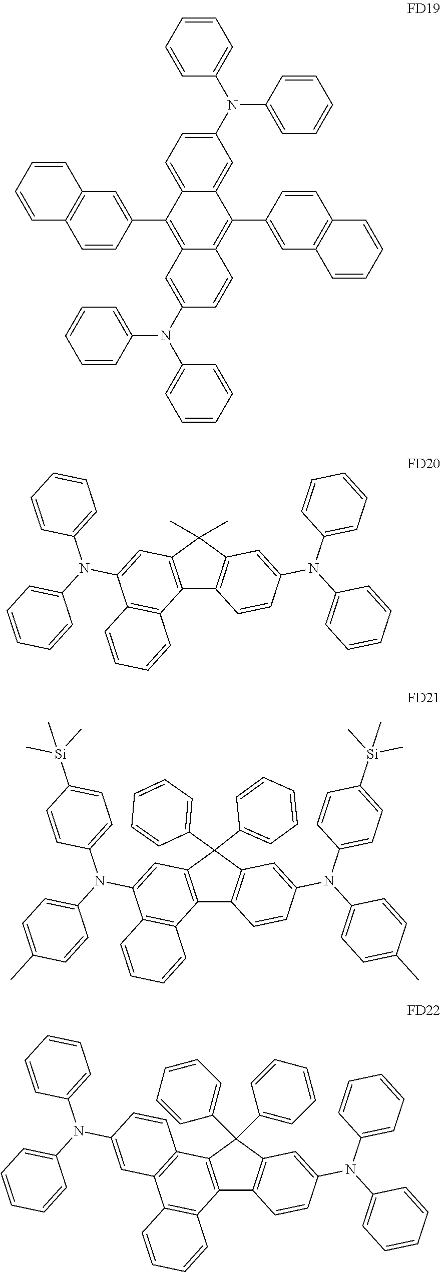

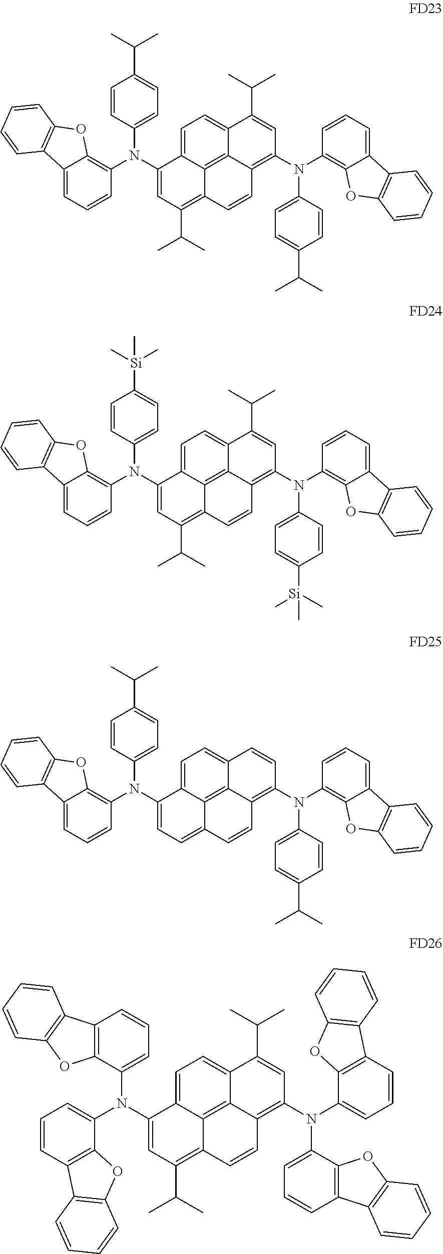 [SMILES: CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; CC(C)c1cc(N(c2ccc([Si](C)(C)C)cc2)c2cccc3c2oc2ccccc23)c2ccc3c(C(C)C)cc(N(c4ccc([Si](C)(C)C)cc4)c4cccc5c4oc4ccccc45)c4ccc1c2c34 ; CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1 ; CC(C)c1cc(N(c2cccc3c2oc2ccccc23)c2cccc3c2oc2ccccc23)c2ccc3c(C(C)C)cc(N(c4cccc5c4oc4ccccc45)c4cccc5c4oc4ccccc45)c4ccc1c2c34]

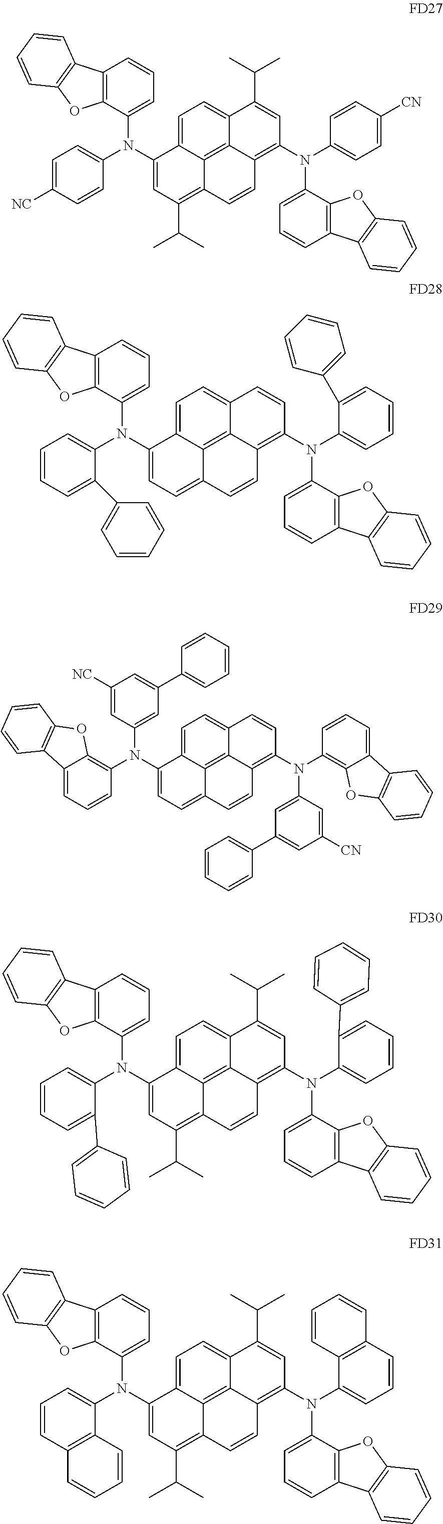

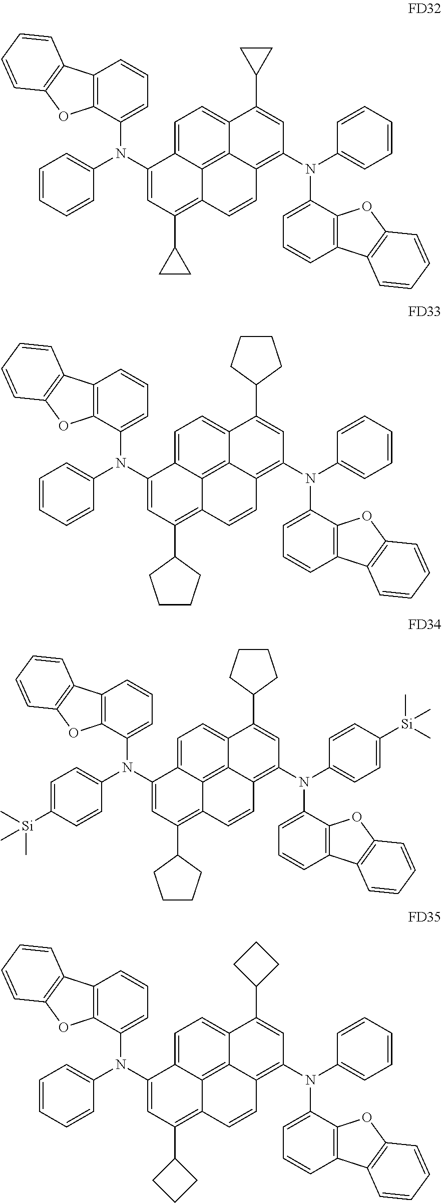

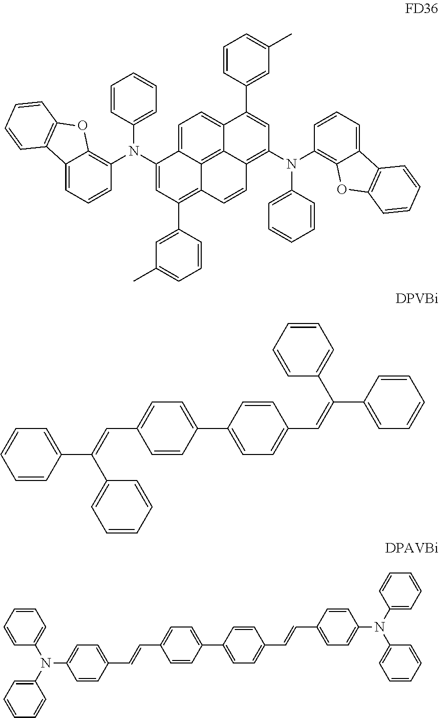

[Delayed Fluorescence Material]

The emission layer may include a delayed fluorescence material.

In the specification, the delayed fluorescence material may be selected from compounds capable of emitting delayed fluorescence based on a delayed fluorescence emission mechanism.

The delayed fluorescence material included in the emission layer may serve as a host or as a dopant, depending on the type of other materials included in the emission layer.

In an embodiment, a difference between a triplet energy level (eV) of the delayed fluorescence material and a singlet energy level (eV) of the delayed fluorescence material may be greater than or equal to about 0 eV and less than or equal to about 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively occur, and thus, the luminescence efficiency of the light-emitting device 10 may be improved.

In an embodiment, the delayed fluorescence material may include a material including at least one electron donor (for example, a T electron-rich C₃-C₆₀ cyclic group, such as a carbazole group) and at least one electron acceptor (for example, a sulfoxide group, a cyano group, or a π electron-deficient nitrogen-containing C₁-C₆₀ cyclic group); or a material including a C₈-C₆₀ polycyclic group in which two or more cyclic groups are condensed while sharing boron (B).

Examples of the delayed fluorescence material may include at least one of Compounds DF1 to DF9:

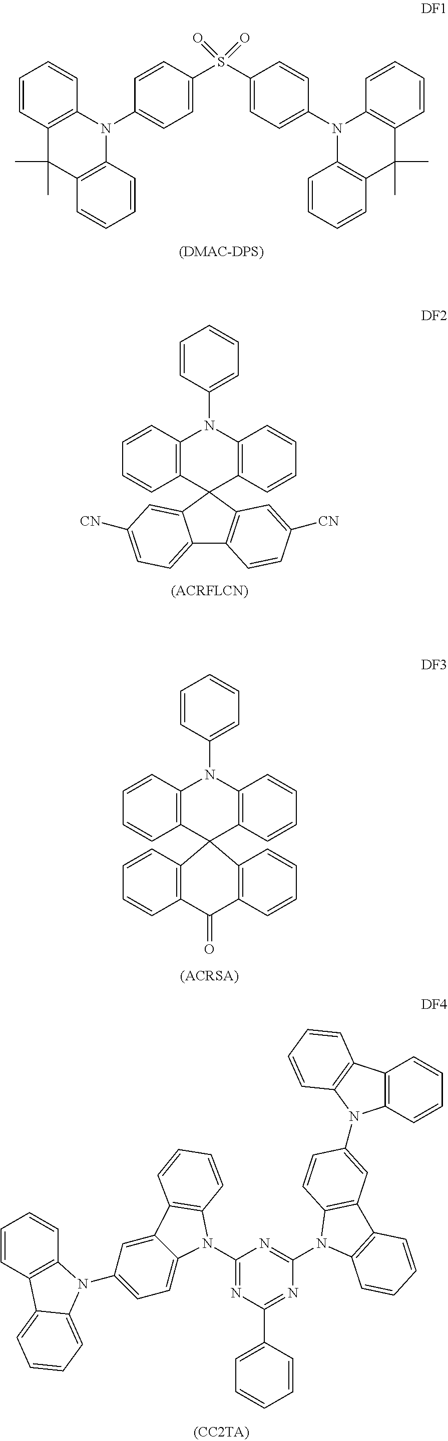

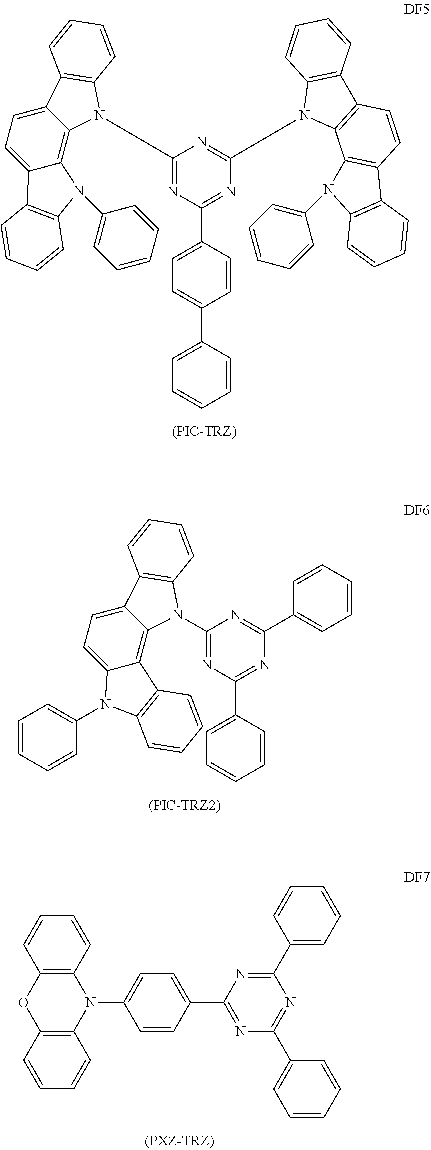 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1]

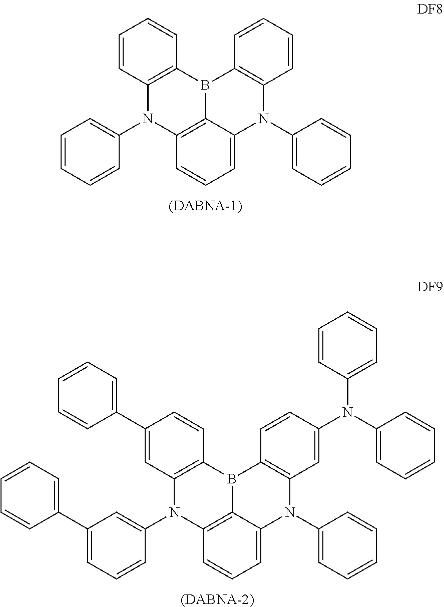

[Quantum Dot]

The emission layer may include a quantum dot.

In the specification, a quantum dot may be a crystal of a semiconductor compound, and may include any material capable of emitting light of various emission wavelengths according to a size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, or any process similar thereto.

According to the wet chemical process, a precursor material is mixed with an organic solvent to grow a quantum dot particle crystal. When the crystal grows, the organic solvent naturally acts as a dispersant coordinated on the surface of the quantum dot crystal and controls the growth of the crystal so that the growth of quantum dot particles may be controlled through a process which is more readily performed than vapor deposition methods, such as metal organic chemical vapor deposition (MOCVD) or molecular beam epitaxy (MBE), and which requires low costs.

The quantum dot may include a Group II-VI semiconductor compound, a Group III-V semiconductor compound, a Group III-VI semiconductor compound, a Group I-III-VI semiconductor compound, a Group IV-VI semiconductor compound, a Group IV element or compound, or any combination thereof.

Examples of the Group III-VI semiconductor compound may include: a binary compound, such as CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any combination thereof.

Examples of the Group III-V semiconductor compound may include: a binary compound, such as GaN, GaP, GaAs, GaSb, AlN, AlP, AlAs, AlSb, InN, InP, InAs, or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, or InAlPSb; or any combination thereof. In an embodiment, the Group III-V semiconductor compound may further include a Group II element. Examples of the Group III-V semiconductor compound further including a Group II element may include InZnP, InGaZnP, InAlZnP, and the like.

Examples of the Group III-VI semiconductor compound may include: a binary compound, such as GaS, GaSe, Ga₂Se₃, GaTe, InS, InSe, In₂S₃, In₂Se₃, or InTe; a ternary compound, such as InGaS₃, or InGaSe₃; or any combination thereof.

Examples of the Group I-III-VI semiconductor compound may include: a ternary compound, such as AgInS, AgInS₂, CuInS, CuInS₂, CuGaO₂, AgGaO₂, or AgAlO₂; or any combination thereof.

Examples of the Group IV-VI semiconductor compound may include: a binary compound, such as SnS, SnSe, SnTe, PbS, PbSe, PbTe, or the like; a ternary compound, such as SnSeS, SnSeTe, SnSTe, PbSeS, PbSeTe, PbSTe, SnPbS, SnPbSe, SnPbTe, or the like; a quaternary compound, such as SnPbSSe, SnPbSeTe, SnPbSTe, or the like; or any combination thereof.

Examples of the Group IV element or compound may include: a single element material, such as Si or Ge; a binary compound, such as SiC or SiGe; or any combination thereof.

Each element included in a multi-element compound such as a binary compound, a ternary compound, and a quaternary compound, may be present in a particle at a uniform concentration or at a non-uniform concentration.

In an embodiment, the quantum dot may have a single structure or a core-shell structure. In case that the quantum dot has a single structure, a concentration of each element included in the corresponding quantum dot may be uniform. In case that the quantum dot has a core-shell structure, a material contained in the core and a material contained in the shell may be different from each other.

The shell of the quantum dot may serve as a protective layer that prevents chemical degeneration of the core to maintain semiconductor characteristics and/or may serve as a charging layer that imparts electrophoretic characteristics to the quantum dot. The shell may be a single layer or a multi-layer. An interface between the core and the shell of the quantum dot may have a concentration gradient in which the concentration of an element that is present in the shell decreases toward the center of the quantum dot.

Examples of the shell of the quantum dot may include a metal oxide, a metalloid oxide, a non-metal oxide, a semiconductor compound, and any combination thereof. Examples of the metal oxide, the metalloid oxide, or the non-metal oxide may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; or any combination thereof. Examples of the semiconductor compound may include, as described herein, a Group II-VI semiconductor compound, a Group III-V semiconductor compound, a Group III-VI semiconductor compound, a Group I-III-VI semiconductor compound, a Group IV-VI semiconductor compound, or any combination thereof. Examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of an emission wavelength spectrum of the quantum dot may be equal to or less than about 45 nm. For example, a FWHM of an emission wavelength spectrum of the quantum dot may be equal to or less than about 40 nm. For example, a FWHM of an emission wavelength spectrum of the quantum dot may be equal to or less than about 30 nm. Within these ranges, color purity or color reproducibility may be increased. Light emitted through the quantum dot may be emitted in all directions, so that a wide viewing angle can be improved.

The quantum dot may be a spherical particle, a pyramidal particle, a multi-arm particle, a cubic nanoparticle, a nanotube particle, a nanowire particle, a nanofiber particle, or a nanoplate particle.

Since the energy band gap can be adjusted by controlling the size of the quantum dot, light having various wavelength bands can be obtained from the quantum dot emission layer. Therefore, by using quantum dots of different sizes, a light-emitting device that emits light of various wavelengths may be implemented. In an embodiment, the size of the quantum dot may be selected to emit red, green, and/or blue light. The size of the quantum dot may be configured to emit white light by combining light of various colors.

[Electron Transport Region in Interlayer 130]

The electron transport region may have a structure consisting of a layer consisting of a single material, a structure consisting of a layer consisting of different materials, or a structure including multiple layers including different materials.

The electron transport region may include a buffer layer, a hole blocking layer, an electron control layer, an electron transport layer, an electron injection layer, or any combination thereof.

For example, the electron transport region may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, wherein the layers of each structure may be stacked from an emission layer in its respective stated order, but the structure of the electron transport region is not limited thereto.

In an embodiment, the electron transport region (for example, the buffer layer, the hole blocking layer, the electron control layer, or the electron transport layer in the electron transport region) may include a metal-free compound including at least one Tr electron-deficient nitrogen-containing C₁-C₆₀ cyclic group.

In an embodiment, the electron transport region may include a compound represented by Formula 601:

[Ar₆₀₁]ₓₑ₁₁-[(L₆₀₁)ₓₑ₁—R₆₀₁]ₓₑ₂₁ [Formula 601]

In Formula 601,

Ar₆₀₁ and L₆₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ are each independently the same as described in connection with Q₁,

xe21 may be 1, 2, 3, 4, or 5, and

at least one of Ar₆₀₁, L₆₀₁, and R₆₀₁ may each independently be a π electron-deficient nitrogen-containing C₁-C₆₀ cyclic group that is unsubstituted or substituted with at least one R₁₀ₐ.

In an embodiment, in Formula 601, when xe11 is 2 or more, two or more of Ar₆₀₁(s) may be linked via a single bond.

In an embodiment, in Formula 601, Ar₆₀₁ may be a substituted or unsubstituted anthracene group.

In an embodiment, the electron transport region may include a compound represented by Formula 601-1:

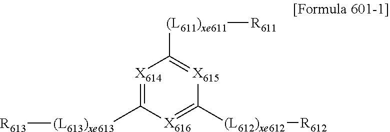

In Formula 601-1,

X₆₁₄ may be N or C(R₆₁₄), X₆₁₅ may be N or C(R₆₁₅), X₆₁₆ may be N or C(R₆₁₆), and at least one of X₆₁₄ to X₆₁₈ may be N,

L₆₁₁ to L₆₁₃ are each independently the same as described in connection with L₆₀₁,

xe611 to xe613 are each independently the same as described in connection with xe1,

R₆₁₁ to R₆₁₃ are each independently the same as described in connection with R₆₀₁, and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group that is unsubstituted or substituted with at least one R₁₀ₐ, or a C₁-C₆₀ heterocyclic group that is unsubstituted or substituted with at least one R₁₀ₐ.

In an embodiment, in Formulae 601 and 601-1, xe1 and xe611 to xe613 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq3, BAlq, TAZ, NTAZ, or any combination thereof:

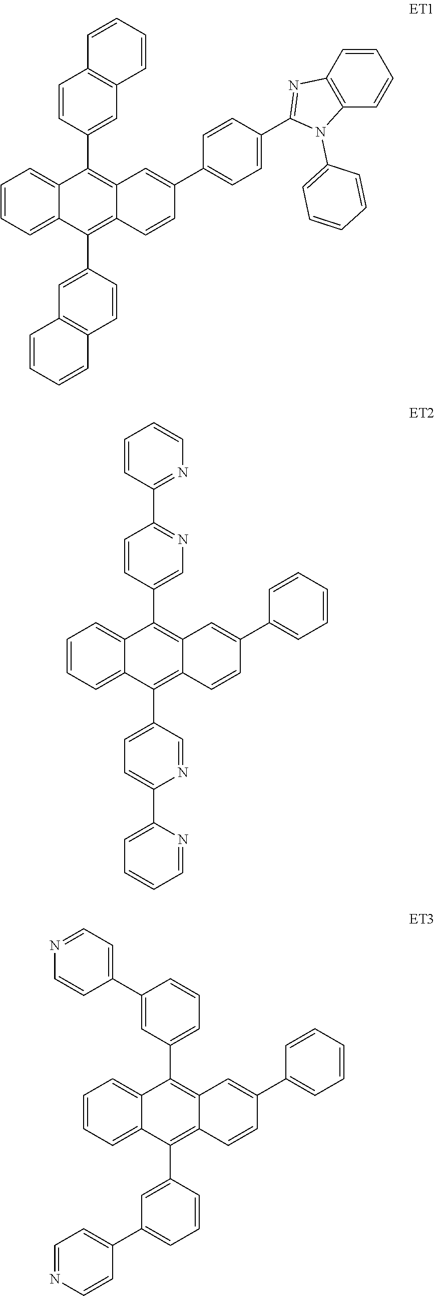 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

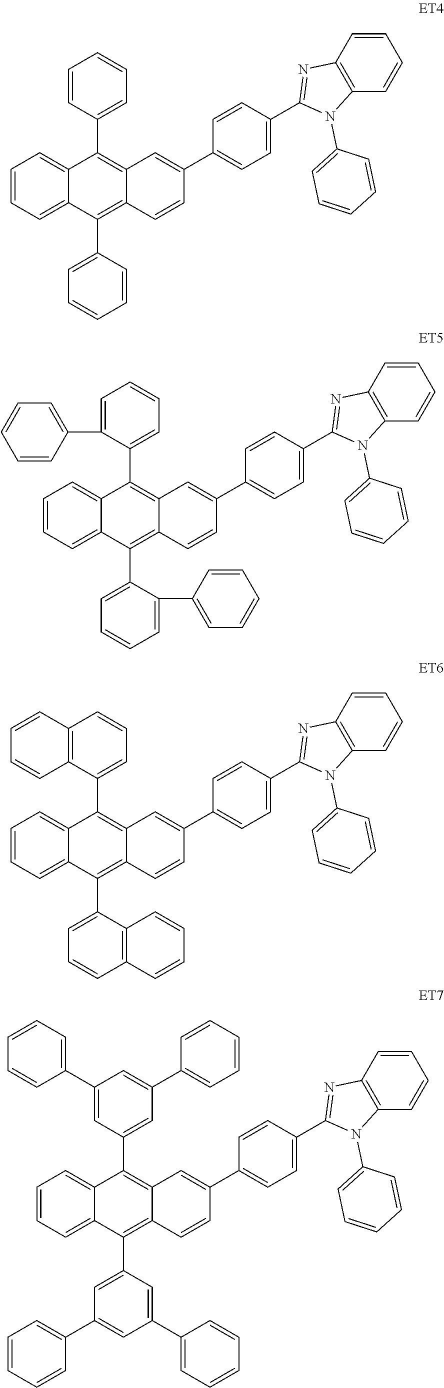 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

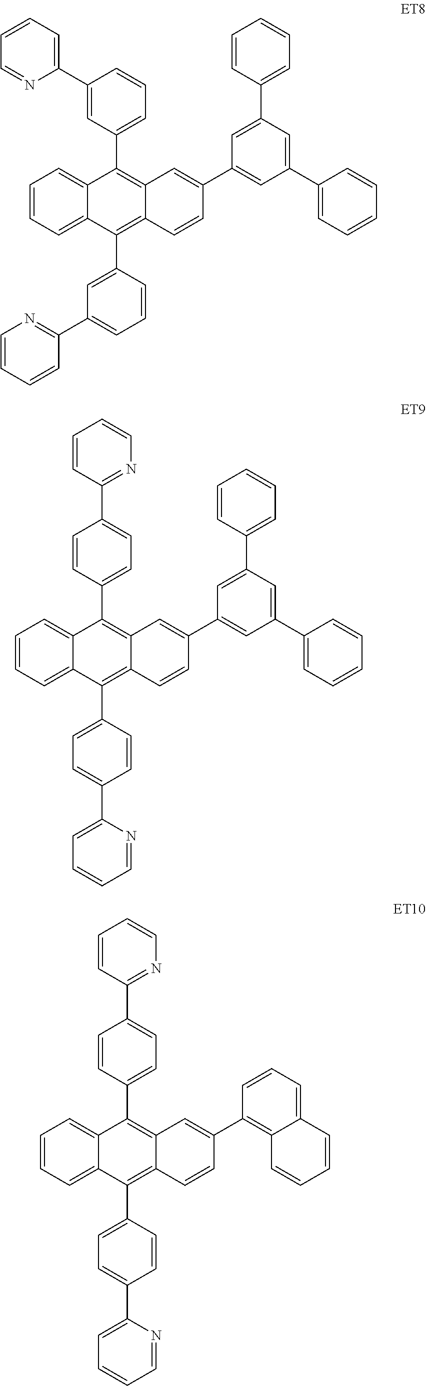

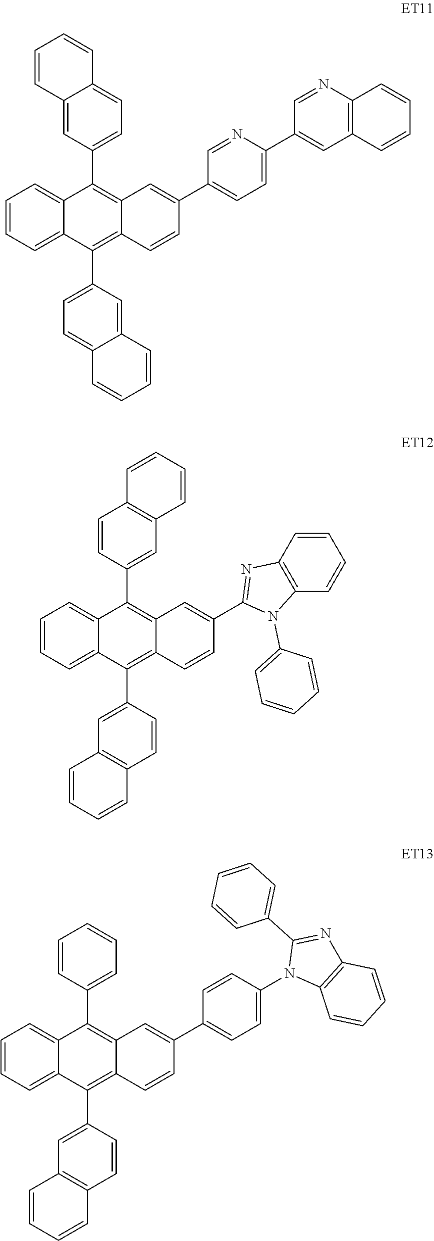 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

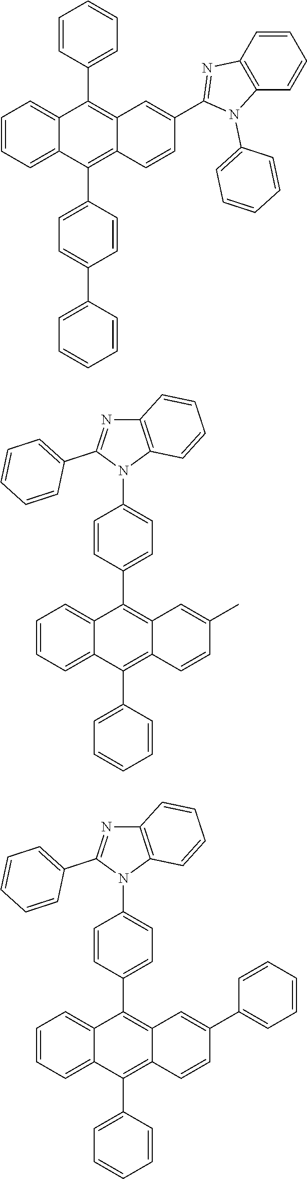 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

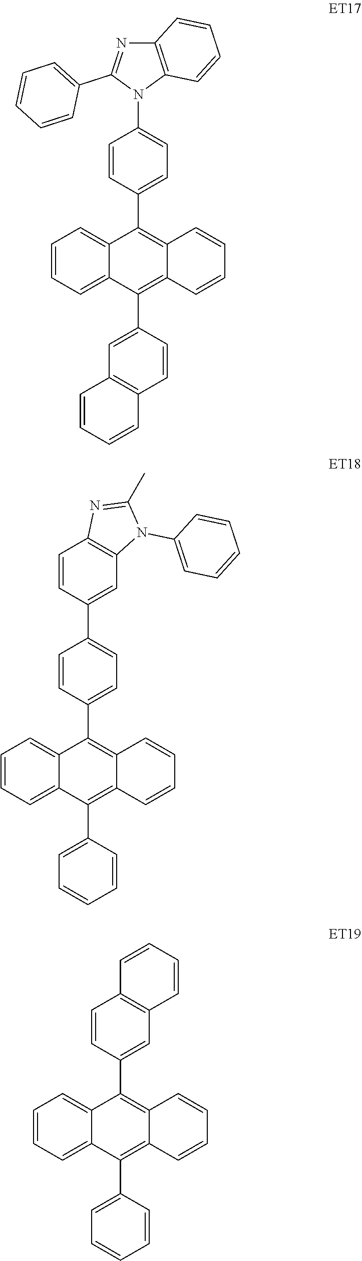

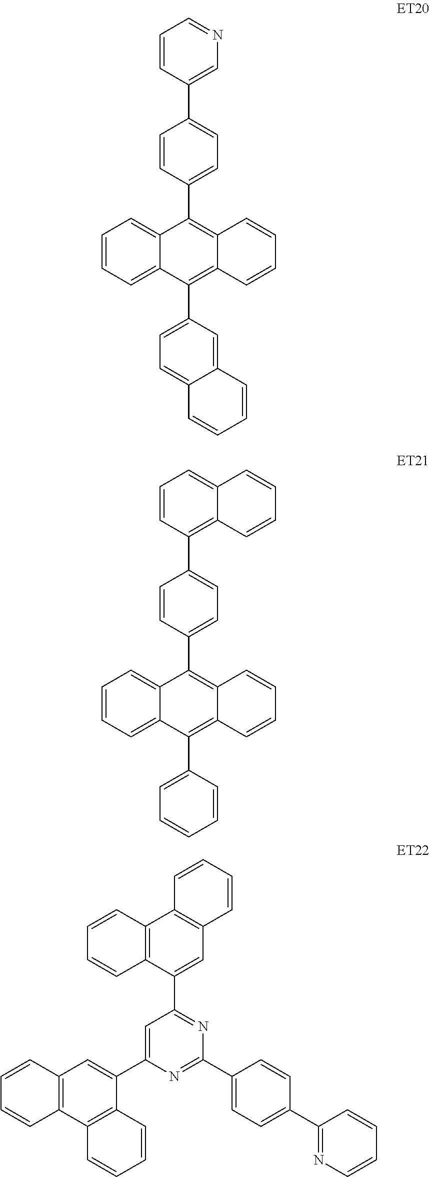

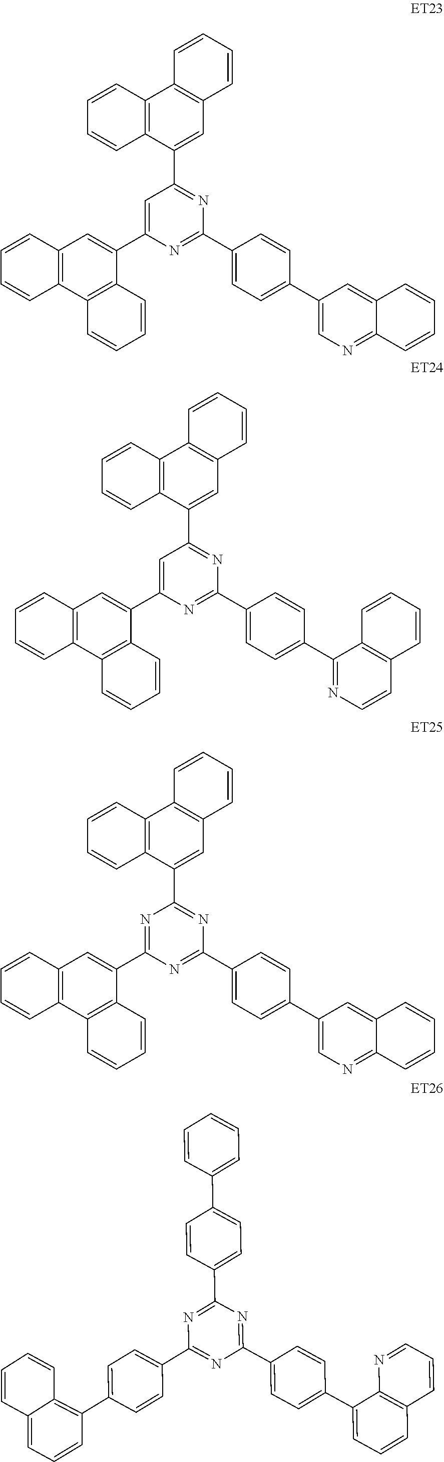 [SMILES: c1ccc2ncc(-c3ccc(-c4nc(-c5cc6ccccc6c6ccccc56)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6ccccc56)cc4)nc(-c4ccc(-c5cccc6cccnc56)cc4)n3)cc2)cc1]

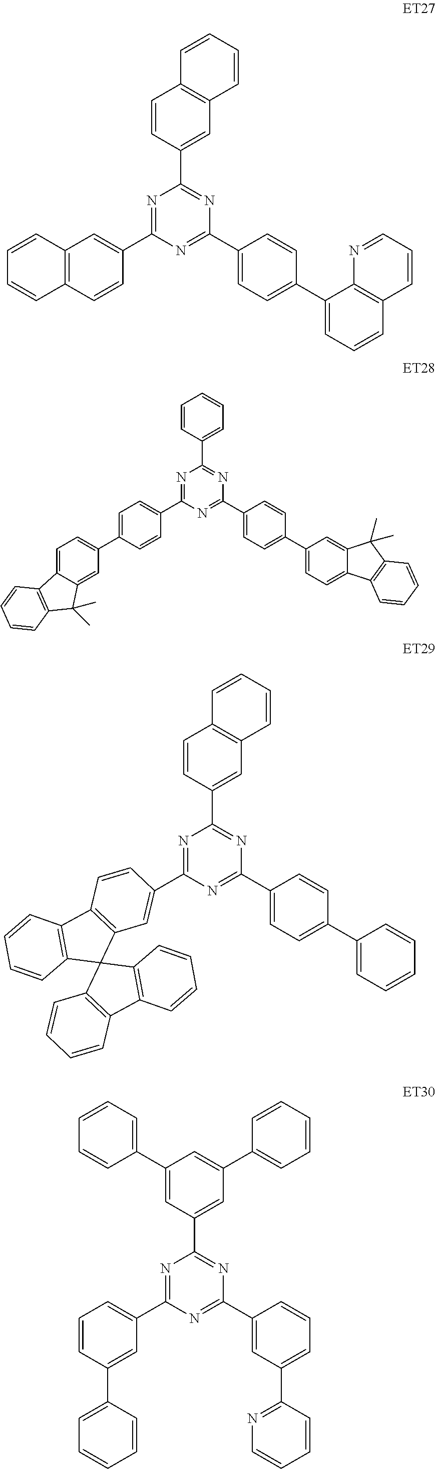 [SMILES: c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3nc(-c4ccc5c(c4)C4(c6ccccc6-c6ccccc64)c4ccccc4-5)nc(-c4ccc5ccccc5c4)n3)cc2)cc1 ; c1ccc(-c2cccc(-c3nc(-c4cc(-c5ccccc5)cc(-c5ccccc5)c4)nc(-c4cccc(-c5ccccn5)c4)n3)c2)cc1]

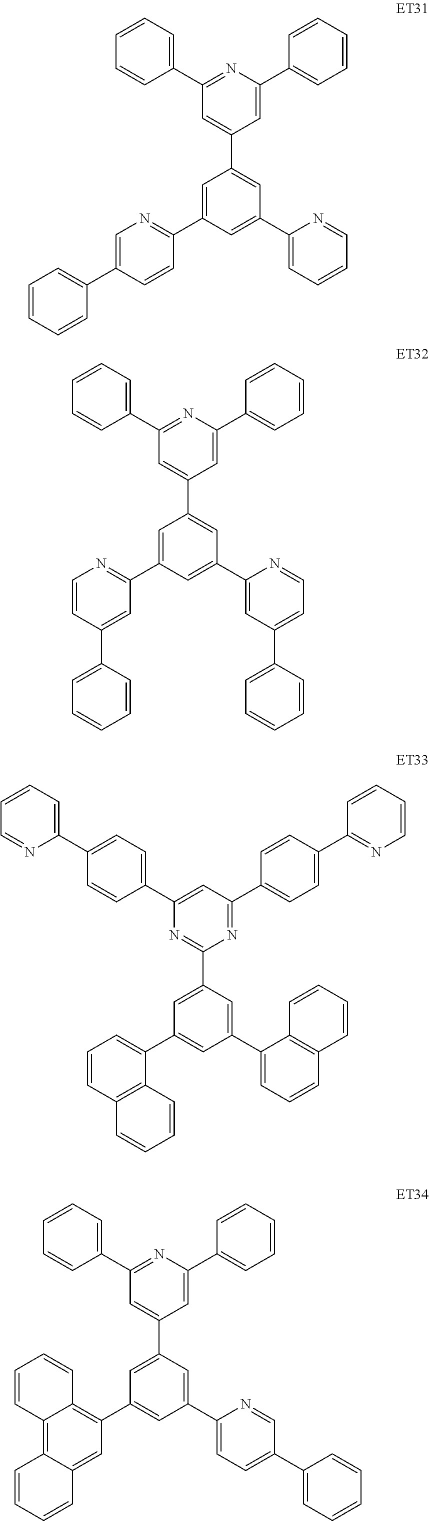 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

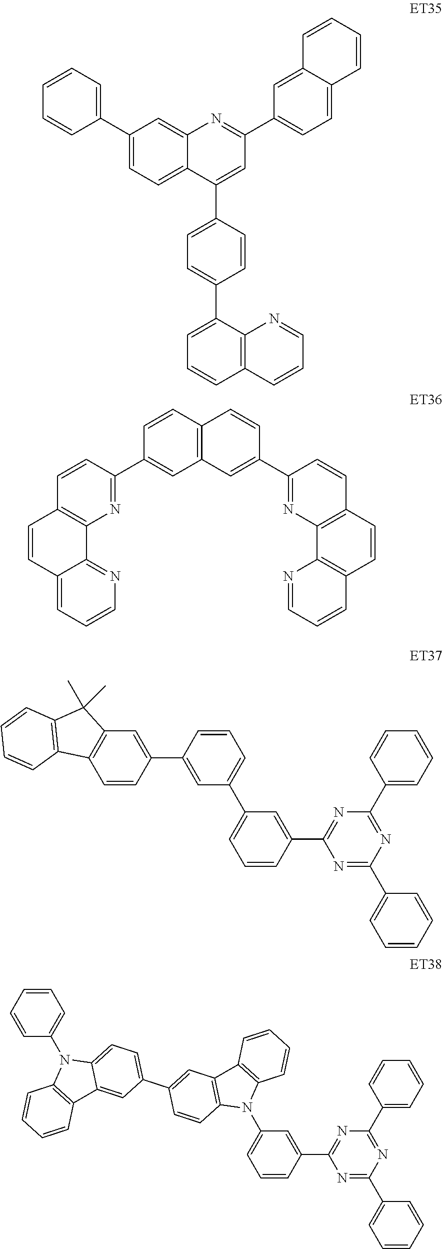

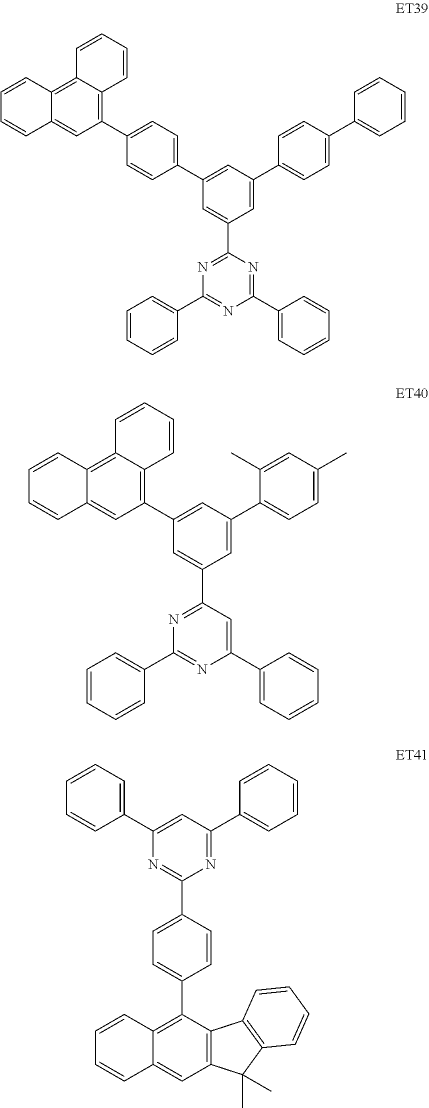 [SMILES: c1ccc(-c2ccc(-c3cc(-c4ccc(-c5cc6ccccc6c6ccccc56)cc4)cc(-c4nc(-c5ccccc5)nc(-c5ccccc5)n4)c3)cc2)cc1 ; Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1]

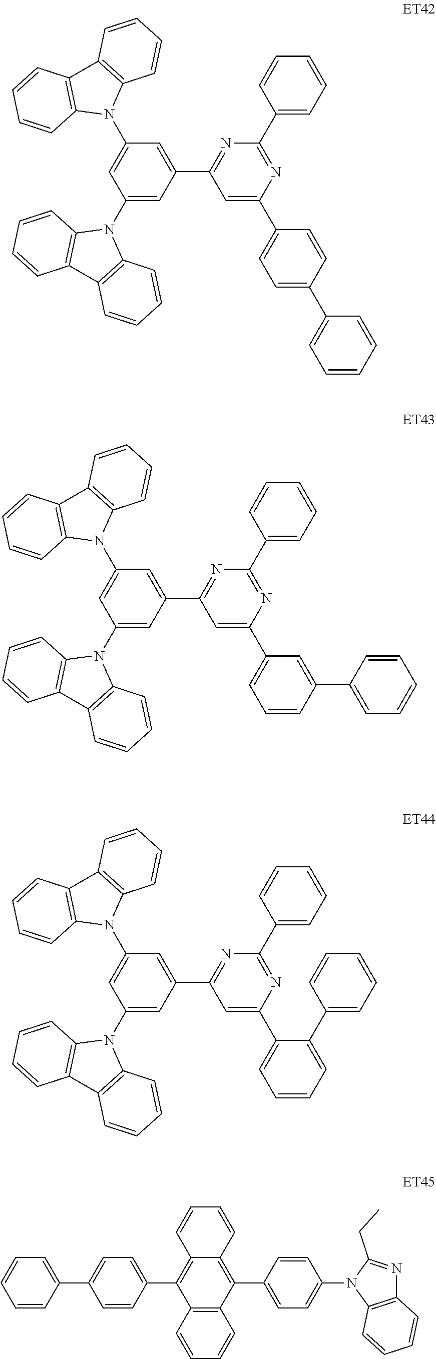 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

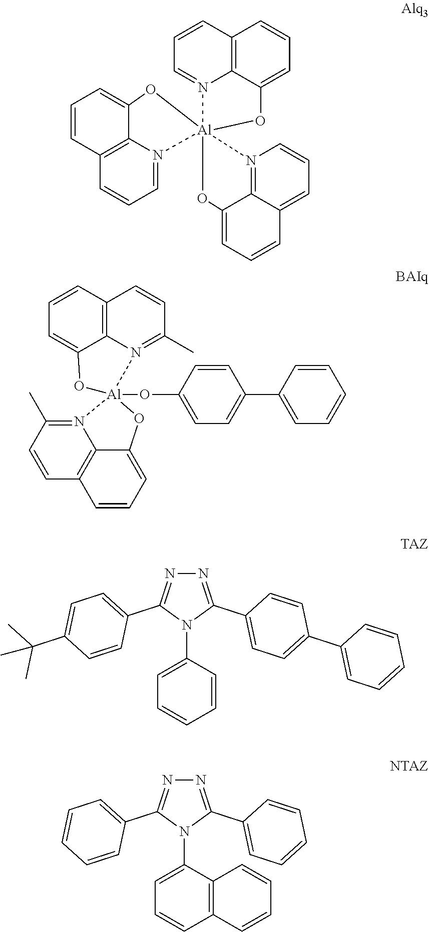

A thickness of the electron transport region may be in a range of about 100 Å to about 5,000 Å. For example, the thickness of the electron transport region may be in a range of about 160 Å to about 4,000 Å. When the electron transport region includes a buffer layer, a hole blocking layer, an electron control layer, an electron transport layer, or any combination thereof, a thickness of the buffer layer, the hole blocking layer, or the electron control layer may each independently be in a range of about 20 Å to about 1,000 Å, and a thickness of the electron transport layer may be in a range of about 100 Å to about 1,000 Å. For example, the thickness of the buffer layer, the hole blocking layer, or the electron control layer may each independently be in a range of about 30 Å to about 300 Å. For example, the thickness of the electron transport layer may be in a range of about 150 Å to about 500 Å. When the thickness of the buffer layer, the hole blocking layer, the electron control layer, the electron transport layer, and/or the electron transport region are within these ranges, satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the metal ion of the alkaline earth-metal complex may each independently include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

In an embodiment, the metal-containing material may include a Li complex. The Li complex may include, for example, Compound ET-D1 (LiQ) or Compound ET-D2:

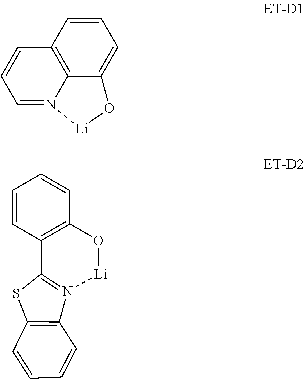

The electron transport region may include an electron injection layer that facilitates the injection of electrons from the second electrode 150. The electron injection layer may directly contact the second electrode 150.

The electron injection layer may have a structure consisting of a layer consisting of a single material, a structure consisting of a layer consisting of different materials, or a structure including multiple layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (for example, fluorides, chlorides, bromides, or iodides), or tellurides of the alkali metal, the alkaline earth metal, and the rare earth metal, or any combination thereof.

The alkali metal-containing compound may include: alkali metal oxides, such as Li₂O, Cs₂O, or K₂O; alkali metal halides, such as LiF, NaF, CsF, KF, LiI, NaI, CsI, or KI; or any combination thereof. The alkaline earth metal-containing compound may include an alkaline earth metal compound, such as BaO, SrO, CaO, BaₓSr₁₋ₓO (x is a real number satisfying the condition of 0<x<1), BaₓCa₁₋ₓO (x is a real number satisfying the condition of 0<x<1), or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In an embodiment, the rare earth metal-containing compound may include a lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include one of ions of the alkali metal, ions of the alkaline earth metal, and ions of the rare earth metal, and a ligand bonded to the metal ion (for example, a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenyl benzimidazole, a hydroxyphenylbenzothiazole, a bipyridine, a phenanthroline, a cyclopentadiene, or any combination thereof).

The electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In an embodiment, the electron injection layer may further include an organic material (for example, a compound represented by Formula 601).

In an embodiment, the electron injection layer may consist of an alkali metal-containing compound (for example, an alkali metal halide); or the electron injection layer may consist of an alkali metal-containing compound (for example, an alkali metal halide), and an alkali metal, an alkaline earth metal, a rare earth metal, or any combination thereof. For example, the electron injection layer may be a KI:Yb co-deposited layer, a RbI:Yb co-deposited layer, a LiF:Yb co-deposited, or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be homogeneously or non-homogeneously dispersed in a matrix including the organic material.

A thickness of the electron injection layer may be in a range of about 1 Å to about 100 Å. For example, the thickness of the electron injection layer may be in a range of about 3 Å to about 90 Å. When the thickness of the electron injection layer is within the ranges described above, satisfactory electron injection characteristics may be obtained without a substantial increase in driving voltage.

[Second Electrode 150]

The second electrode 150 may be located on the interlayer 130 having a structure as described above. The second electrode 150 may be a cathode, which is an electron injection electrode. The second electrode 150 may include a material having a low-work function, for example, a metal, an alloy, an electrically conductive compound, or any combination thereof.

In an embodiment, the second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or a combination thereof. The second electrode 150 may be a transmissive electrode, a semi-transmissive electrode, or a reflective electrode.

The second electrode 150 may have a single-layered structure or a multi-layered structure including two or more layers.

[Capping Layer]

The light-emitting device 10 may include a first capping layer located outside the first electrode 110, and/or a second capping layer located outside the second electrode 150. For example, the light-emitting device 10 may have a structure in which the first capping layer, the first electrode 110, the interlayer 130, and the second electrode 150 are stacked in this stated order, a structure in which the first electrode 110, the interlayer 130, the second electrode 150, and the second capping layer are stacked in this stated order, or a structure in which the first capping layer, the first electrode 110, the interlayer 130, the second electrode 150, and the second capping layer are stacked in this stated order.

Light generated in an emission layer of the interlayer 130 of the light-emitting device 10 may be extracted toward the outside through the first electrode 110, which may be a semi-transmissive electrode or a transmissive electrode, and through the first capping layer. Light generated in an emission layer of the interlayer 130 of the light-emitting device 10 may be extracted toward the outside through the second electrode 150, which may be a semi-transmissive electrode or a transmissive electrode, and through the second capping layer.

The first capping layer and the second capping layer may each increase external emission efficiency according to the principle of constructive interference. Accordingly, the light extraction efficiency of the light-emitting device 10 may be increased, so that the luminescence efficiency of the light-emitting device 10 may be improved.

The first capping layer and the second capping layer may each include a material having a refractive index equal to or greater than about 1.6 (with respect to a wavelength of about 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and the second capping layer may each independently include carbocyclic compounds, heterocyclic compounds, amine group-containing compounds, porphyrin derivatives, phthalocyanine derivatives, naphthalocyanine derivatives, alkali metal complexes, alkaline earth metal complexes, or any combination thereof. The carbocyclic compound, the heterocyclic compound, and the amine group-containing compound may be optionally substituted with a substituent containing O, N, S, Se, Si, F, Cl, Br, I, or any combination thereof.

In an embodiment, at least one of the first capping layer and the second capping layer may each independently include an amine group-containing compound.

In an embodiment, at least one of the first capping layer and the second capping layer may each independently include a compound represented by Formula 201, a compound represented by Formula 202, or any combination thereof.

In an embodiment, at least one of the first capping layer and the second capping layer may each independently include one of Compounds HT28 to HT33, one of Compounds CP1 to CP6, β-NPB, or any combination thereof:

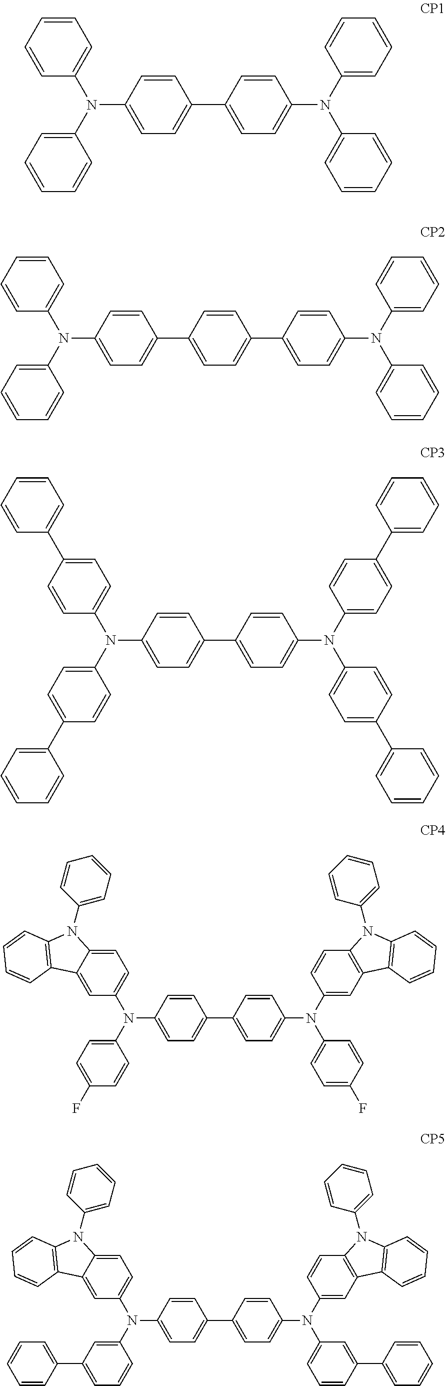 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

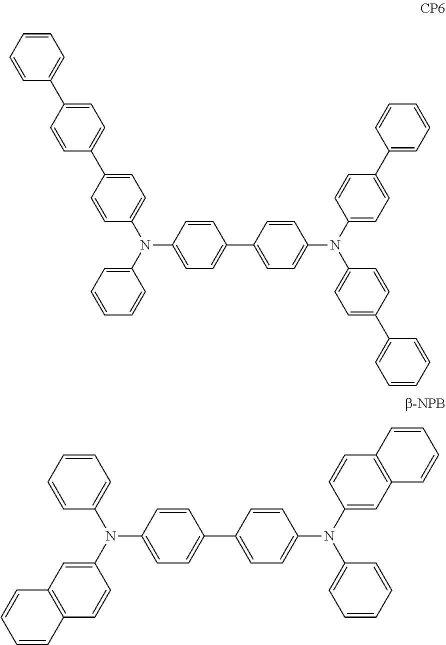 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

[Film]

The amine-based compound represented by Formula 1 may be included in various films. According to embodiments, a film including the amine-based compound represented by Formula 1 may be provided. The film may be, for example, an optical member (or a light control means) (for example, a color filter, a color conversion member, a capping layer, a light extraction efficiency enhancement layer, a selective light absorbing layer, a polarizing layer, a quantum dot-containing layer, or like), a light-blocking member (for example, a light reflective layer, a light absorbing layer, or the like), or a protective member (for example, an insulating layer, a dielectric layer, or the like).

[Electronic Apparatus]

The light-emitting device may be included in various electronic apparatuses. In an embodiment, the electronic apparatus including the light-emitting device may be a light-emitting apparatus, an authentication apparatus, or the like.

The electronic apparatus (for example, a light-emitting apparatus) may further include, in addition to the light-emitting device, a color filter, a color conversion layer, or a color filter and a color conversion layer. The color filter and/or the color conversion layer may be located in at least one traveling direction of light emitted from the light-emitting device. For example, the light emitted from the light-emitting device may be blue light or white light. The light-emitting device may be the same as described herein. In an embodiment, the color conversion layer may include quantum dots. The quantum dot may be, for example, a quantum dot as described herein.

The electronic apparatus may include a first substrate. The first substrate may include subpixels, the color filter may include color filter areas respectively corresponding to the subpixels, and the color conversion layer may include color conversion areas respectively corresponding to the subpixels.

A pixel-defining film may be located between the subpixels to define each subpixel.

The color filter may further include color filter areas and light-shielding patterns located between the color filter areas, and the color conversion layer may include color conversion areas and light-shielding patterns located between the color conversion areas.

The color filter areas (or the color conversion areas) may include a first area emitting first color light, a second area emitting second color light, and/or a third area emitting third color light, and the first color light, the second color light, and/or the third color light may have different maximum emission wavelengths from one another. In an embodiment, the first color light may be red light, the second color light may be green light, and the third color light may be blue light. In an embodiment, the color filter areas (or the color conversion areas) may include quantum dots. For example, the first area may include a red quantum dot, the second area may include a green quantum dot, and the third area may not include a quantum dot. The quantum dot may be the same as described in the specification. The first area, the second area, and/or the third area may each further include a scatterer.

In an embodiment, the light-emitting device may emit first light, the first area may absorb the first light to emit first first-color light, the second area may absorb the first light to emit second first-color light, and the third area may absorb the first light to emit third first-color light. The first first-color light, the second first-color light, and the third first-color light may have different maximum emission wavelengths from one another. For example, the first light may be blue light, the first first-color light may be red light, the second first-color light may be green light, and the third first-color light may be blue light.

The electronic apparatus may further include a thin-film transistor in addition to the light-emitting device as described herein. The thin-film transistor may include a source electrode, a drain electrode, and an active layer, wherein any one of the source electrode and the drain electrode may be electrically connected to any one of the first electrode and the second electrode of the light-emitting device.

The thin-film transistor may further include a gate electrode, a gate insulating film, etc.

The active layer may include crystalline silicon, amorphous silicon, organic semiconductor, oxide semiconductor, or the like.

The electronic apparatus may further include a sealing portion for sealing the light-emitting device. The sealing portion may be located between the color filter and/or the color conversion layer, and the light-emitting device. The sealing portion may allow light from the light-emitting device to be extracted to the outside, and may simultaneously prevent ambient air and moisture from penetrating into the light-emitting device. The sealing portion may be a sealing substrate including a transparent glass substrate or a plastic substrate. The sealing portion may be a thin-film encapsulation layer including an organic layer and/or an inorganic layer. When the sealing portion is a thin film encapsulation layer, the electronic apparatus may be flexible.

Various functional layers may be further included on the sealing portion, in addition to the color filter and/or the color conversion layer, according to the use of the electronic apparatus. The functional layers may include a touch screen layer, a polarizing layer, an authentication apparatus, and the like. The touch screen layer may be a pressure-sensitive touch screen layer, a capacitive touch screen layer, or an infrared touch screen layer.

The authentication apparatus may further include, in addition to the light-emitting device, a biometric information collector. The authentication apparatus may be, for example, a biometric authentication apparatus that authenticates an individual by using biometric information of a living body (for example, fingertips, pupils, etc.).

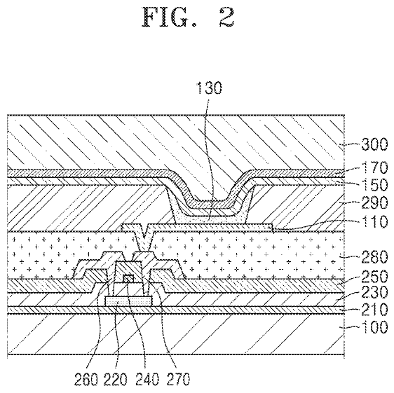

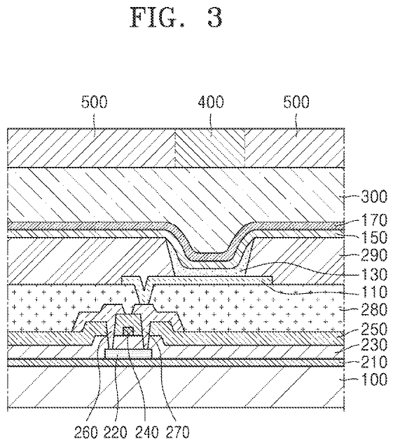

The electronic apparatus may be applied to various displays, light sources, lighting, personal computers (for example, a mobile personal computer), mobile phones, digital cameras, electronic diaries, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, various measuring instruments, meters (for example, meters for a vehicle, an aircraft, and a vessel), projectors, and the like.

[Description of FIGS. 2 and 3]

FIG. 2 is a schematic cross-sectional view of an electronic apparatus according to an embodiment.

The electronic apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be formed on the substrate 100. The buffer layer 210 may prevent penetration of impurities through the substrate 100 and may provide a flat surface on the substrate 100.

A TFT may be located on the buffer layer 210. The TFT may include an active layer 220, a gate electrode 240, a source electrode 260, and a drain electrode 270.

The active layer 220 may include an inorganic semiconductor such as silicon or polysilicon, an organic semiconductor, or an oxide semiconductor, and may include a source region, a drain region, and a channel region.

A gate insulating film 230 for insulating the active layer 220 from the gate electrode 240 may be located on the active layer 220, and the gate electrode 240 may be located on the gate insulating film 230.

An interlayer insulating film 250 is located on the gate electrode 240. The interlayer insulating film 250 may be placed between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be located on the interlayer insulating film 250. The interlayer insulating film 250 and the gate insulating film 230 may be formed to expose the source region and the drain region of the active layer 220, and the source electrode 260 and the drain electrode 270 may respectively contact the exposed portions of the source region and the drain region of the active layer 220.

The TFT is electrically connected to a light-emitting device to drive the light-emitting device, and is covered by a passivation layer 280. The passivation layer 280 may include an inorganic insulating film, an organic insulating film, or a combination thereof. A light-emitting device is provided on the passivation layer 280. The light-emitting device may include a first electrode 110, an interlayer 130, and a second electrode 150.

The first electrode 110 may be formed on the passivation layer 280. The passivation layer 280 may not completely cover the drain electrode 270 and may expose a portion of the drain electrode 270, and the first electrode 110 may be electrically connected to the exposed portion of the drain electrode 270.

A pixel-defining layer 290 containing an insulating material may be located on the first electrode 110. The pixel-defining layer 290 may expose a region of the first electrode 110, and an interlayer 130 may be formed in the exposed region of the first electrode 110. The pixel-defining layer 290 may be a polyimide or polyacrylic organic film. Although not shown in FIG. 2, at least some layers of the interlayer 130 may extend beyond the upper portion of the pixel-defining layer 290 to be provided in the form of a common layer.

The second electrode 150 may be located on the interlayer 130, and a capping layer 170 may be additionally formed on the second electrode 150. The capping layer 170 may be formed to cover the second electrode 150.

The encapsulation portion 300 may be located on the capping layer 170. The encapsulation portion 300 may be located on a light-emitting device to protect the light-emitting device from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, or the like), an epoxy-based resin (for example, aliphatic glycidyl ether (AGE), or the like), or a combination thereof; or a combination of the inorganic film and the organic film.

FIG. 3 is a schematic cross-sectional view of an electronic apparatus according to another embodiment.

The electronic apparatus of FIG. 3 may differ from the electronic apparatus of FIG. 2, at least in that a light-shielding pattern 500 and a functional region 400 are further included on the encapsulation portion 300. The functional region 400 may be a color filter area, a color conversion area, or a combination of the color filter area and the color conversion area. In an embodiment, the light-emitting device included in the electronic apparatus of FIG. 3 may be a tandem light-emitting device.

[Manufacturing Method]

Respective layers included in the hole transport region, the emission layer, and respective layers included in the electron transport region may be formed in a specific region by using one or more suitable methods selected from vacuum deposition, spin coating, casting, Langmuir-Blodgett (LB) deposition, ink-jet printing, laser-printing, and laser-induced thermal imaging.

When layers constituting the hole transport region, an emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

[Definitions of Terms]

The term “C₃-C₆₀ carbocyclic group” as used herein may be a cyclic group consisting of carbon as the only ring-forming atoms and having three to sixty carbon atoms, and the term “C₁-C₆₀ heterocyclic group” as used herein may be a cyclic group that has one to sixty carbon atoms and further has, in addition to carbon, at least one heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. In an embodiment, the C₁-C₆₀ heterocyclic group may have 3 to 61 ring-forming atoms.

The term “cyclic group” as used herein may include the C₃-C₆₀ carbocyclic group or the C₁-C₆₀ heterocyclic group.

The term “π electron-rich C₃-C₆₀ cyclic group” as used herein may be a cyclic group that has three to sixty carbon atoms and may not include *—N═*′ as a ring-forming moiety, and the term “π electron-deficient nitrogen-containing C₁-C₆₀ cyclic group” as used herein may be a heterocyclic group that has one to sixty carbon atoms and may include *—N═*′ as a ring-forming moiety.

In embodiments,

the C₃-C₆₀ carbocyclic group may be a T1 group or a cyclic group in which two or more T1 groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be a T2 group, a cyclic group in which two or more T2 groups are condensed with each other, or a cyclic group in which at least one T2 group and at least one T1 group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the π electron-rich C₃-C₆₀ cyclic group may be a T1 group, a cyclic group in which two or more T1 groups are condensed with each other, a T3 group, a cyclic group in which two or more T3 groups are condensed with each other, or a cyclic group in which at least one T3 group and at least one T1 group are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),

the π electron-deficient nitrogen-containing C₁-C₆₀ cyclic group may be a T4 group, a cyclic group in which two or more T4 groups are condensed with each other, a cyclic group in which at least one T4 group and at least one T1 group are condensed with each other, a cyclic group in which at least one T4 group and at least one T3 group are condensed with each other, or a cyclic group in which at least one T4 group, at least one T1 group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

wherein the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,

the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

the T3 group may be a furan group, a thiophene group, a 1H-pyrrole group, a silole group, or a borole group, and

the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group”, “C₃-C₆₀ carbocyclic group”, “C₁-C₆₀ heterocyclic group”, “π electron-rich C₃-C₆₀ cyclic group”, or “π electron-deficient nitrogen-containing C₁-C₆₀ cyclic group” as used herein may each be a group condensed to any cyclic group, a monovalent group, or a polyvalent group (for example, a divalent group, a trivalent group, a tetravalent group, etc.), depending on the structure of a formula for which the corresponding term is used. For example, a “benzene group” may be a benzo group, a phenyl group, a phenylene group, or the like, which may be readily understood by one of ordinary skill in the art according to the structure of a formula including the “benzene group.”

Examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of the divalent C₃-C₆₀ carbocyclic group and the divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as used herein may be a linear or branched aliphatic hydrocarbon monovalent group that has one to sixty carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein may be a divalent group having a same structure as the C₁-C₆₀ alkyl group.

The term “C₂-C₆₀ alkenyl group” as used herein may be a monovalent hydrocarbon group having at least one carbon-carbon double bond in the middle or at a terminus of the C₂-C₆₀ alkyl group, and examples thereof may include an ethenyl group, a propenyl group, and a butenyl group. The term “C₂-C₆₀ alkenylene group” as used herein may be a divalent group having a same structure as the C₂-C₆₀ alkenyl group.

The term “C₂-C₆₀ alkynyl group” as used herein may be a monovalent hydrocarbon group having at least one carbon-carbon triple bond in the middle or at a terminus of the C₂-C₆₀ alkyl group, and examples thereof may include an ethynyl group and a propynyl group. The term “C₂-C₆₀ alkynylene group” as used herein may be a divalent group having a same structure as the C₂-C₆₀ alkynyl group.

The term “C₁-C₆₀ alkoxy group” as used herein may be a monovalent group represented by —O(A₁₀₁) (wherein A₁₀₁ may be a C₁-C₆₀ alkyl group), and examples thereof may include a methoxy group, an ethoxy group, and an isopropyloxy group.

The term “C₃-C₁₀ cycloalkyl group” as used herein may be a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantanyl group, a norbomanyl group (or a bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as used herein may be a divalent group having a same structure as the C₃-C₁₀ cycloalkyl group.

The term “C₁-C₁₀ heterocycloalkyl group” as used herein may be a monovalent cyclic group that further includes, in addition to a carbon atom, at least one heteroatom as a ring-forming atom and has 1 to 10 carbon atoms, and examples thereof may include a 1,2,3,4-oxatriazolidinyl group, a tetrahydrofuranyl group, and a tetrahydrothiophenyl group. The term “C₁-C₁₀ heterocycloalkylene group” as used herein may be a divalent group having a same structure as the C₁-C₁₀ heterocycloalkyl group.

The term “C₃-C₁₀ cycloalkenyl group” as used herein may be a monovalent cyclic group that has three to ten carbon atoms and at least one carbon-carbon double bond in the ring thereof and no aromaticity, and examples thereof may include a cyclopentenyl group, a cyclohexenyl group, and a cycloheptenyl group. The term “C₃-C₁₀ cycloalkenylene group” as used herein may be a divalent group having a same structure as the C₃-C₁₀ cycloalkenyl group.

The term “C₁-C₁₀ heterocycloalkenyl group” as used herein may be a monovalent cyclic group that has, in addition to a carbon atom, at least one heteroatom as a ring-forming atom, 1 to 10 carbon atoms, and at least one carbon-carbon double bond in the cyclic structure thereof. Examples of the C₁-C₁₀ heterocycloalkenyl group may include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein may be a divalent group having a same structure as the C₁-C₁₀ heterocycloalkenyl group.

The term “C₆-C₆₀ aryl group” as used herein may be a monovalent group having a carbocyclic aromatic system having six to sixty carbon atoms, and the term “C₆-C₆₀ arylene group” as used herein may be a divalent group having a carbocyclic aromatic system having six to sixty carbon atoms. Examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the respective rings may be condensed with each other.

The term “C₁-C₆₀ heteroaryl group” as used herein may be a monovalent group having a heterocyclic aromatic system that has, in addition to a carbon atom, at least one heteroatom as a ring-forming atom, and 1 to 60 carbon atoms. The term “C₁-C₆₀ heteroarylene group” as used herein may be a divalent group having a heterocyclic aromatic system that has, in addition to a carbon atom, at least one heteroatom as a ring-forming atom, and 1 to 60 carbon atoms. Examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the respective rings may be condensed with each other.

The term “monovalent non-aromatic condensed polycyclic group” as used herein may be a monovalent group having two or more rings condensed to each other, only carbon atoms (for example, having 8 to 60 carbon atoms) as ring-forming atoms, and non-aromaticity in its molecular structure when considered as a whole. Examples of the monovalent non-aromatic condensed polycyclic group may include an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as used herein may be a divalent group having a same structure as a monovalent non-aromatic condensed polycyclic group.

The term “monovalent non-aromatic condensed heteropolycyclic group” as used herein may be a monovalent group having two or more rings condensed to each other, at least one heteroatom other than carbon atoms (for example, having 1 to 60 carbon atoms), as a ring-forming atom, and non-aromaticity in its molecular structure when considered as a whole. Examples of the monovalent non-aromatic condensed heteropolycyclic group may include a pyrrolyl group, a thiophenyl group, a furanyl group, an indolyl group, a benzoindolyl group, a naphtho indolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as used herein may be a divalent group having a same structure as a monovalent non-aromatic condensed heteropolycyclic group.

The term “C₆-C₆₀ aryloxy group” as used herein may be represented by —O(A₁₀₂) (wherein A₁₂ may be a C₆-C₆₀ aryl group), and the term “C₆-C₆₀ arylthio group” as used herein may be represented by —S(A₁₀₃) (wherein A₁₀₃ may be a C₆-C₆₀ aryl group).

The term “C₇-C₆₀ aryl alkyl group” as used herein may be represented by —(A₁₀₄)(A₁₀₅) (wherein A₁₀₄ may be a C₁-C₅₄ alkylene group, and A₁₀₅ may be a C₆-C₅₉ aryl group), and the term “C₂-C₆₀ heteroaryl alkyl group” as used herein may be represented by —(A₁₀₆)(A₁₀₇) (wherein A₁₀₆ may be a C₁-C₅₉ alkylene group, and A₁₀₇ may be a C₁-C₅₉ heteroaryl group).

The term R₁₀ₐ as used herein may be:

deuterium (—D), —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁—CoO heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

The groups Q₁ to Q₃, Q₁₁ to Q₁₃, Q₂₁ to Q₂₃, and Q₃₁ to Q₃₃ as used herein may each independently be: hydrogen; deuterium; —F; —Cl; —Br; —I; a hydroxyl group; a cyano group; a nitro group; a C₁-C₆₀ alkyl group, a C₂-C₆₀ alkenyl group, a C₂-C₆₀ alkynyl group, a C₁-C₆₀ alkoxy group, a C₃-C₆₀ carbocyclic group or a C₁—CoO heterocyclic group, each unsubstituted or substituted with deuterium, —F, a cyano group, a C₁-C₆₀ alkyl group, a C₁-C₆₀ alkoxy group, a phenyl group, a biphenyl group, a pyridinyl group, a pyrimidinyl group, a pyridazinyl group, a pyrazinyl group, a triazinyl group or any combination thereof; a C₇-C₆₀ aryl alkyl group; or a C₂-C₆₀ heteroaryl alkyl group.

The term “heteroatom” as used herein may be any atom other than a carbon atom or a hydrogen atom. Examples of the heteroatom may include O, S, N, P, Si, B, Ge, Se, or any combination thereof.

The term “Ph” as used herein refers to a phenyl group, the term “Me” as used herein refers to a methyl group, the term “Et” as used herein refers to an ethyl group, the terms “tert-Bu” or “But” as used herein each refer to a tert-butyl group, and the term “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein may be a phenyl group substituted with a biphenyl group. The term “terphenyl group” as used herein may be a substituted phenyl group having, as a substituent, a C₆-C₆₀ aryl group substituted with a C₆-C₆₀ aryl group.

The symbols * and *′ as used herein, unless defined otherwise, each refer to a binding site to a neighboring atom in a corresponding formula or moiety.

Hereinafter, compounds according to embodiments and light-emitting devices according to embodiments will be described in detail with reference to the Synthesis Examples and the Examples. The wording “B was used instead of A” used in describing Synthesis Examples means that an identical molar equivalent of B was used in place of A.

### EXAMPLES

### Synthesis Example 1: Synthesis of Compound 1

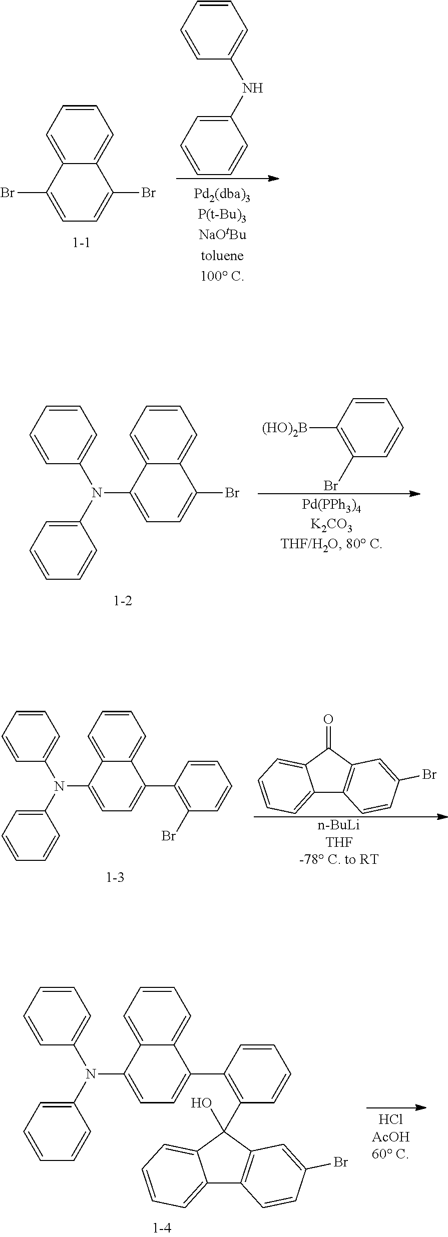

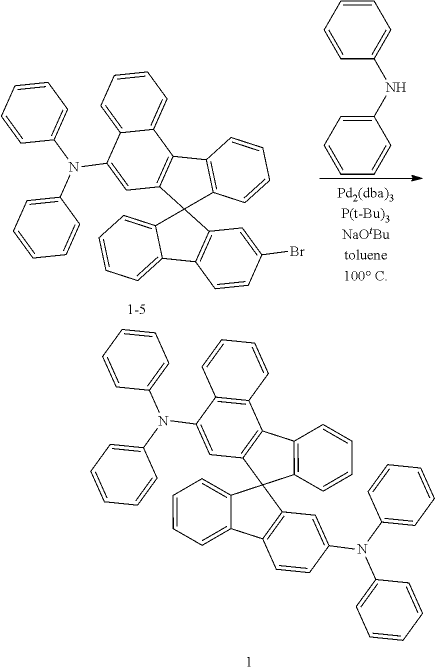 [SMILES: Brc1ccc2c(c1)C1(c3ccccc3-2)c2ccccc2-c2c1cc(N(c1ccccc1)c1ccccc1)c1ccccc21 ; c1ccc(Nc2ccccc2)cc1 ; c1ccc(N(c2ccccc2)c2ccc3c(c2)C2(c4ccccc4-3)c3ccccc3-c3c2cc(N(c2ccccc2)c2ccccc2)c2ccccc32)cc1]

Synthesis of Intermediate 1-2

Intermediate 1-1 (2.85 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 1-2 (3.06 g, yield: 82%).

Synthesis of Intermediate 1-3

Intermediate 1-2 (3.74 g), Pd(PPh₃)₄ (0.56 g), K₂CO₃ (3.45 g), and (2-bromophenyl)boronic acid (2.00 g) were dissolved in THF/H₂O (100 ml/25 ml) and stirred for 12 hours at 80° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 1-3 (2.70 g, yield: 60%).

Synthesis of Intermediate 1-4

A solution obtained by dissolving Intermediate 1-3 (4.50 g) in THF (50 ml) was stirred at −78° C. 4 ml of n-BuLi (2.5M in hexane) was slowly added dropwise to the reaction solution at −78° C. and stirred for 30 minutes. 2-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 1-4 (4.41 g, yield: 70%).

Synthesis of Intermediate 1-5

Intermediate 1-4 (6.30 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 1-5 (5.01 g, yield: 82%).

Synthesis of Compound 1

Intermediate 1-5 (6.12 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 1 (5.67 g, yield: 81%).

### Synthesis Example 2: Synthesis of Compound 32

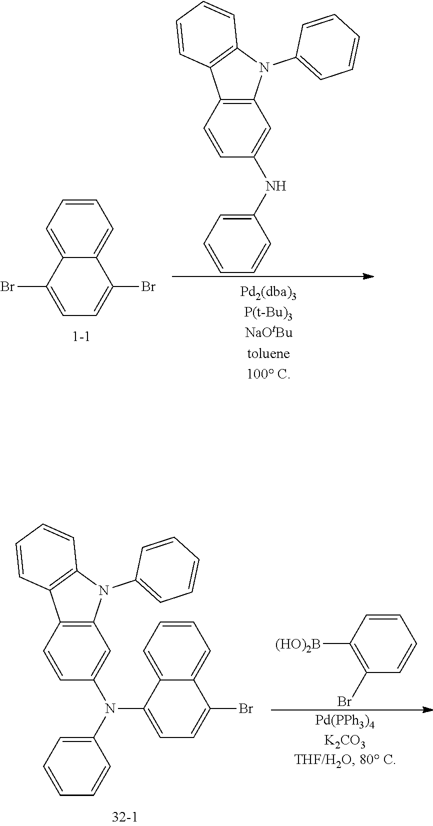 [SMILES: Brc1ccc(Br)c2ccccc12 ; c1ccc(Nc2ccc3c4ccccc4n(-c4ccccc4)c3c2)cc1 ; Brc1ccc(N(c2ccccc2)c2ccc3c4ccccc4n(-c4ccccc4)c3c2)c2ccccc12 ; OB(O)c1ccccc1Br]

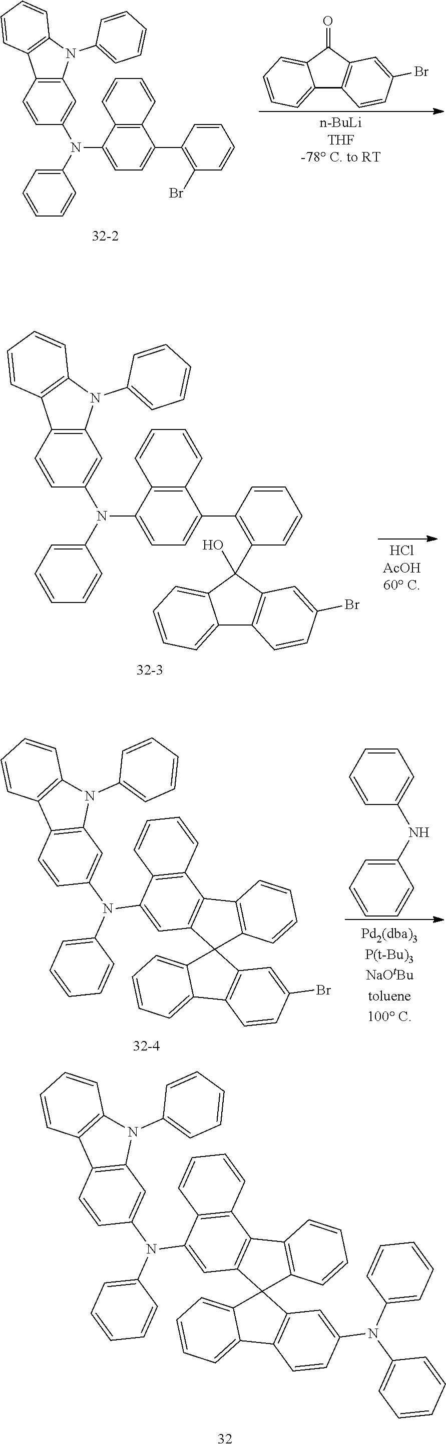 [SMILES: Brc1ccccc1-c1ccc(N(c2ccccc2)c2ccc3c4ccccc4n(-c4ccccc4)c3c2)c2ccccc12 ; O=C1c2ccccc2-c2ccc(Br)cc21 ; OC1(c2ccccc2-c2ccc(N(c3ccccc3)c3ccc4c5ccccc5n(-c5ccccc5)c4c3)c3ccccc23)c2ccccc2-c2ccc(Br)cc21 ; Brc1ccc2c(c1)C1(c3ccccc3-2)c2ccccc2-c2c1cc(N(c1ccccc1)c1ccc3c4ccccc4n(-c4ccccc4)c3c1)c1ccccc21 ; c1ccc(Nc2ccccc2)cc1 ; c1ccc(N(c2ccccc2)c2ccc3c(c2)C2(c4ccccc4-3)c3ccccc3-c3c2cc(N(c2ccccc2)c2ccc4c5ccccc5n(-c5ccccc5)c4c2)c2ccccc32)cc1]

Synthesis of Intermediate 32-1

Intermediate 1-1 (2.85 g), N,9-diphenyl-9H-carbazol-2-amine (3.34 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 32-1 (4.15 g, yield: 77%).

Synthesis of Intermediate 32-2

Intermediate 32-1 (5.39 g), Pd(PPh₃)₄ (0.56 g), K₂CO₃ (3.45 g), and (2-bromophenyl)boronic acid (2.00 g) were dissolved in THF/H₂O (100 ml/25 ml) and stirred for 12 hours at 80° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 32-2 (4.24 g, yield: 69%).

Synthesis of Intermediate 32-3

A solution obtained by dissolving Intermediate 32-2 (6.15 g) in THF (50 ml) was stirred at −78° C. 4 ml of n-BuLi (2.5M in hexane) was slowly added dropwise to the reaction solution at −78° C. and stirred for 30 minutes. 2-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 32-3 (5.80 g, yield: 73%).

Synthesis of Intermediate 32-4

Intermediate 32-3 (7.95 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 32-4 (6.44 g, yield: 83%).

Synthesis of Compound 32

Intermediate 32-4 (7.77 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 32 (7.01 g, yield: 81%).

### Synthesis Example 3: Synthesis of Compound 36

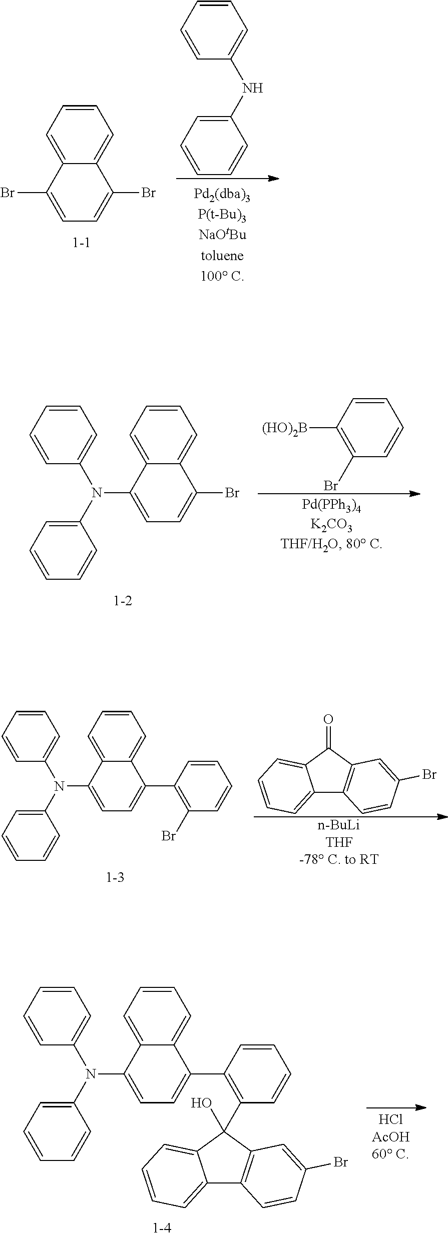

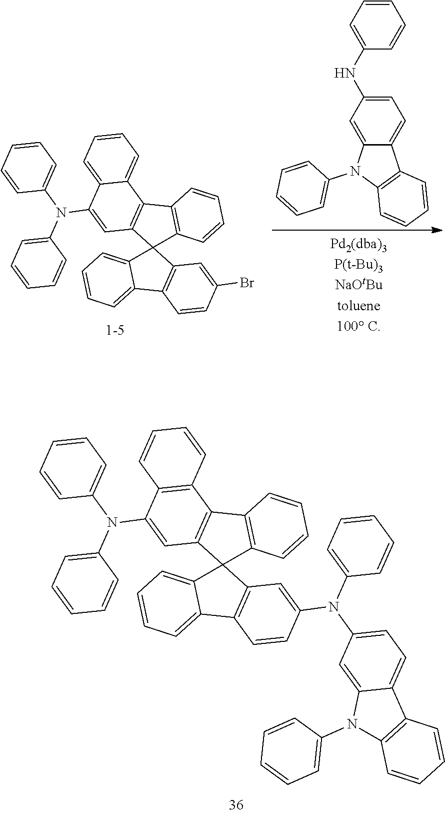 [SMILES: Brc1ccc2c(c1)C1(c3ccccc3-2)c2ccccc2-c2c1cc(N(c1ccccc1)c1ccccc1)c1ccccc21 ; c1ccc(Nc2ccc3c4ccccc4n(-c4ccccc4)c3c2)cc1 ; c1ccc(N(c2ccc3c(c2)C2(c4ccccc4-3)c3ccccc3-c3c2cc(N(c2ccccc2)c2ccccc2)c2ccccc32)c2ccc3c4ccccc4n(-c4ccccc4)c3c2)cc1]

Synthesis of Compound 36

Intermediate 1-5 (6.12 g), N,9-diphenyl-9H-carbazol-2-amine (1.69 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 36 (6.66 g, yield: 77%).

### Synthesis Example 4: Synthesis of Compound 40

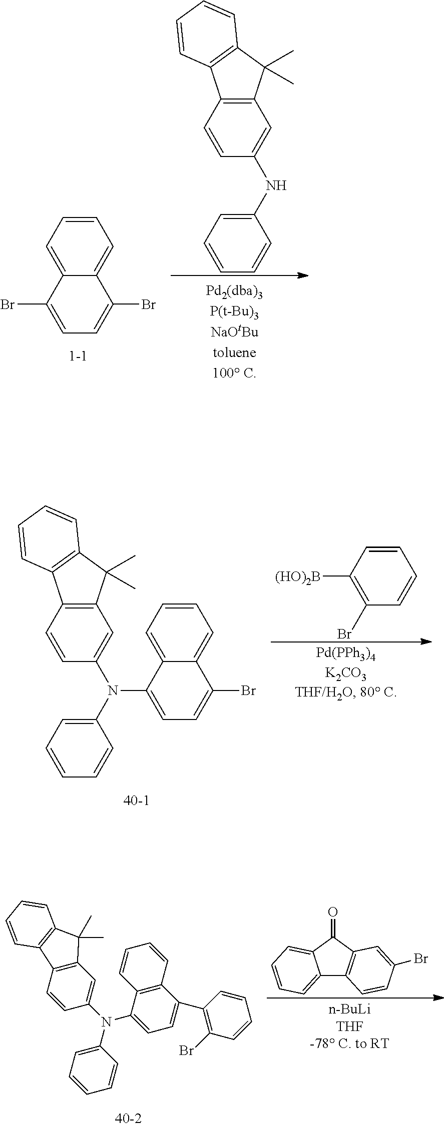

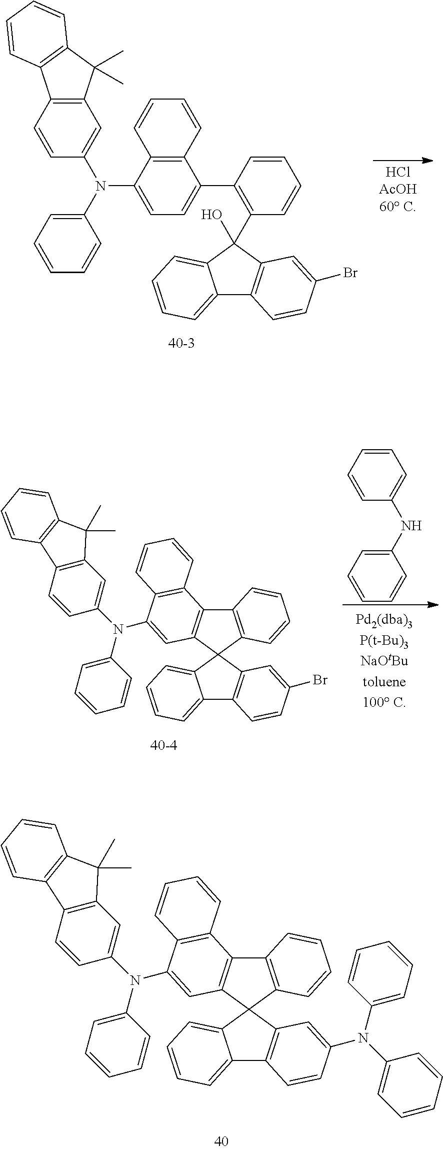 [SMILES: CC1(C)c2ccccc2-c2ccc(N(c3ccccc3)c3ccc(-c4ccccc4C4(O)c5ccccc5-c5ccc(Br)cc54)c4ccccc34)cc21 ; CC1(C)c2ccccc2-c2ccc(N(c3ccccc3)c3cc4c(c5ccccc35)-c3ccccc3C43c4ccccc4-c4ccc(Br)cc43)cc21 ; c1ccc(Nc2ccccc2)cc1 ; CC1(C)c2ccccc2-c2ccc(N(c3ccccc3)c3cc4c(c5ccccc35)-c3ccccc3C43c4ccccc4-c4ccc(N(c5ccccc5)c5ccccc5)cc43)cc21]

Synthesis of Intermediate 40-1

Intermediate 1-1 (2.85 g), 9,9-dimethyl-N-phenyl-9H-fluoren-2-amine (2.85 g), Pd₂(dba)₃ (0.46 g), P(t-Bu)ₐ(0.21 g), and NaOtBu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 40-1 (3.72 g, yield: 76%).

Synthesis of Intermediate 40-2

Intermediate 40-1 (4.90 g), Pd(PPh₃)₄ (0.56 g), K₂CO₃ (3.45 g), and (2-bromophenyl)boronic acid (2.00 g) were dissolved in THF/H₂O (100 ml/25 ml) and stirred for 12 hours at 80° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 40-2 (3.16 g, yield: 56%).

Synthesis of Intermediate 40-3

A solution obtained by dissolving Intermediate 40-2 (5.66 g) in THF (50 ml) was stirred at −78° C. 4 ml of n-BuLi (2.5M in hexane) was slowly added dropwise to the reaction solution at −78° C. and stirred for 30 minutes. 2-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 40-3 (3.95 g, yield: 53%).

Synthesis of Intermediate 40-4

Intermediate 40-3 (7.46 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 40-4 (6.11 g, yield: 84%).

Synthesis of Compound 40

Intermediate 40-4 (7.28 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 40 (6.37 g, yield: 78%).

### Synthesis Example 5: Synthesis of Compound 92

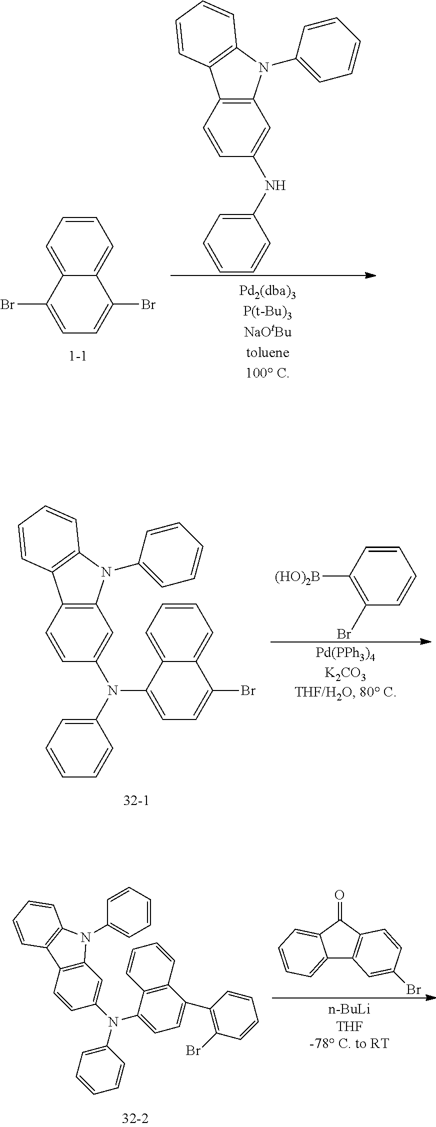

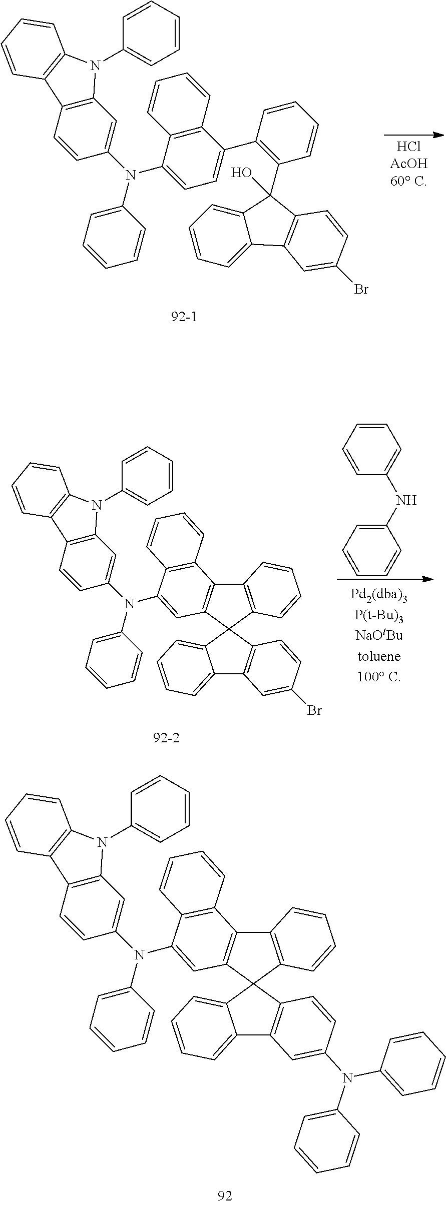 [SMILES: OC1(c2ccccc2-c2ccc(N(c3ccccc3)c3ccc4c5ccccc5n(-c5ccccc5)c4c3)c3ccccc23)c2ccccc2-c2cc(Br)ccc21 ; Brc1ccc2c(c1)-c1ccccc1C21c2ccccc2-c2c1cc(N(c1ccccc1)c1ccc3c4ccccc4n(-c4ccccc4)c3c1)c1ccccc21 ; c1ccc(Nc2ccccc2)cc1 ; c1ccc(N(c2ccccc2)c2ccc3c(c2)-c2ccccc2C32c3ccccc3-c3c2cc(N(c2ccccc2)c2ccc4c5ccccc5n(-c5ccccc5)c4c2)c2ccccc32)cc1]

Synthesis of Intermediate 92-1

A solution obtained by dissolving Intermediate 32-2 (6.15 g) in THF (50 ml) was stirred at −78° C. 4 ml of n-BuLi (2.5M in hexane) was slowly added dropwise to the reaction solution at −78° C. and stirred for 30 minutes. 3-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 92-1 (4.45 g, yield: 56%).

Synthesis of Intermediate 92-2

Intermediate 92-1 (7.95 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 92-2 (6.60 g, yield: 85%).

Synthesis of Compound 92

Intermediate 92-2 (7.77 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 92 (6.58 g, yield: 76%).

### Synthesis Example 6: Synthesis of Compound 95

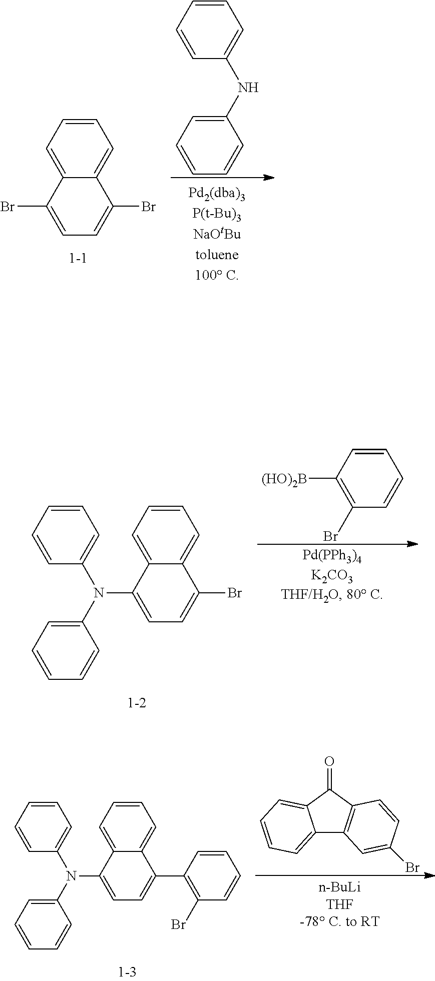

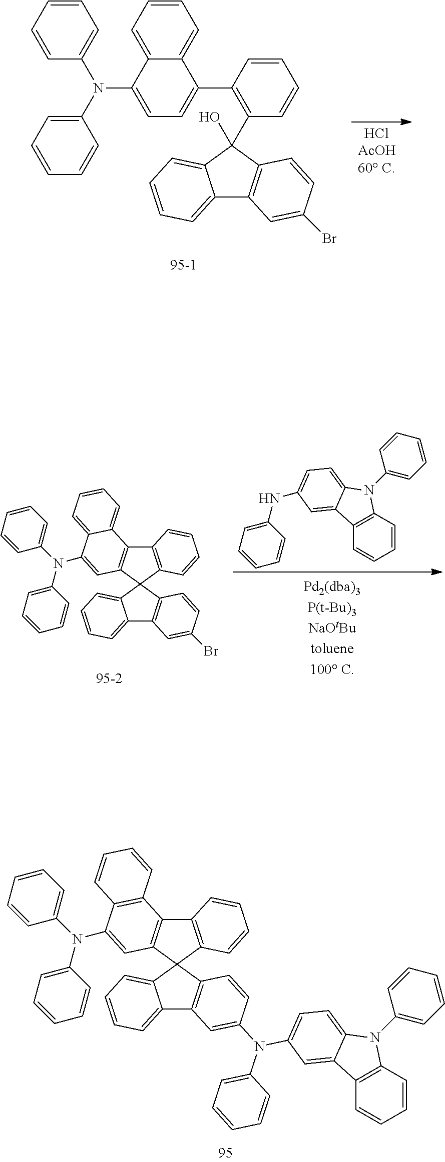

Synthesis of Intermediate 95-1

A solution obtained by dissolving Intermediate 1-3 (4.50 g) in THF (50 ml) was stirred at −78° C. 4 ml of n-BuLi (2.5M in hexane) was slowly added dropwise to the reaction solution at −78° C. and stirred for 30 minutes. 3-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 95-1 (2.89 g, yield: 46%).

Synthesis of Intermediate 95-2

Intermediate 95-1 (7.95 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 95-2 (5.38 g, yield: 88%).

Synthesis of Compound 95

Intermediate 95-2 (6.12 g), N,9-diphenyl-9H-carbazol-3-amine (3.34 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 95 (6.14 g, yield: 71%).

### Synthesis Example 7: Synthesis of Compound 124

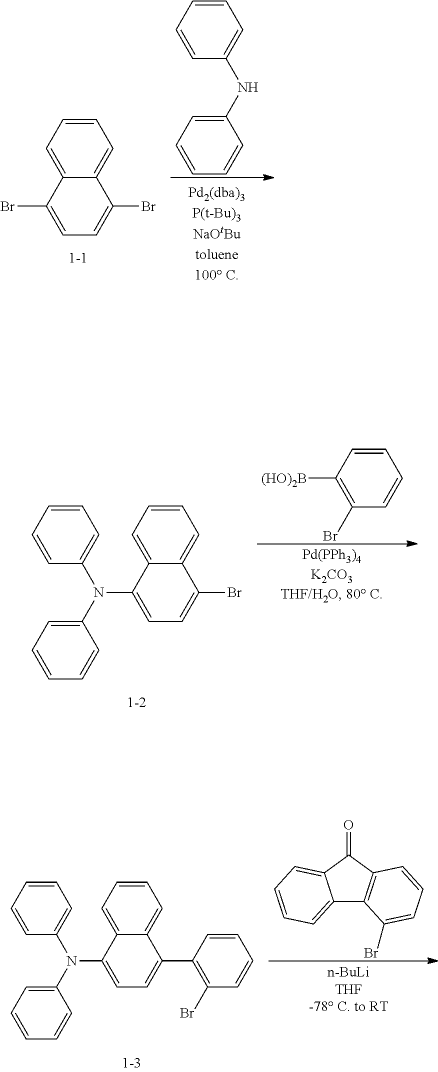 [SMILES: Brc1ccc(Br)c2ccccc12 ; c1ccc(Nc2ccccc2)cc1 ; Brc1ccc(N(c2ccccc2)c2ccccc2)c2ccccc12 ; OB(O)c1ccccc1Br ; Brc1ccccc1-c1ccc(N(c2ccccc2)c2ccccc2)c2ccccc12 ; O=C1c2ccccc2-c2c(Br)cccc21]

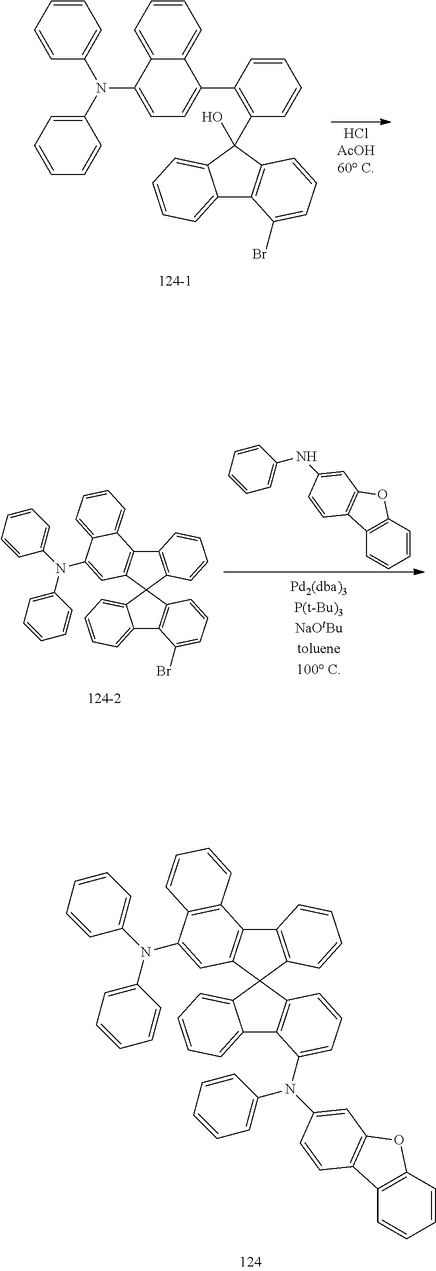 [SMILES: OC1(c2ccccc2-c2ccc(N(c3ccccc3)c3ccccc3)c3ccccc23)c2ccccc2-c2c(Br)cccc21 ; Brc1cccc2c1-c1ccccc1C21c2ccccc2-c2c1cc(N(c1ccccc1)c1ccccc1)c1ccccc21 ; c1ccc(Nc2ccc3c(c2)oc2ccccc23)cc1 ; c1ccc(N(c2ccc3c(c2)oc2ccccc23)c2cccc3c2-c2ccccc2C32c3ccccc3-c3c2cc(N(c2ccccc2)c2ccccc2)c2ccccc32)cc1]

Synthesis of Intermediate 124-1

A solution obtained by dissolving Intermediate 1-3 (4.50 g) in THF (50 ml) was stirred at −78° C. 4 ml of n-BuLi (2.5M in hexane) was slowly added dropwise to the reaction solution at −78° C. and stirred for 30 minutes. 4-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 124-1 (3.84 g, yield: 61%).

Synthesis of Intermediate 124-2

Intermediate 124-1 (6.30 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 124-2 (5.14 g, yield: 84%).

Synthesis of Compound 124

Intermediate 124-2 (6.12 g), N-phenyldibenzo[b,d]furan-3-amine (2.59 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 124 (5.68 g, yield: 72%).

### Synthesis Example 8: Synthesis of Compound 139

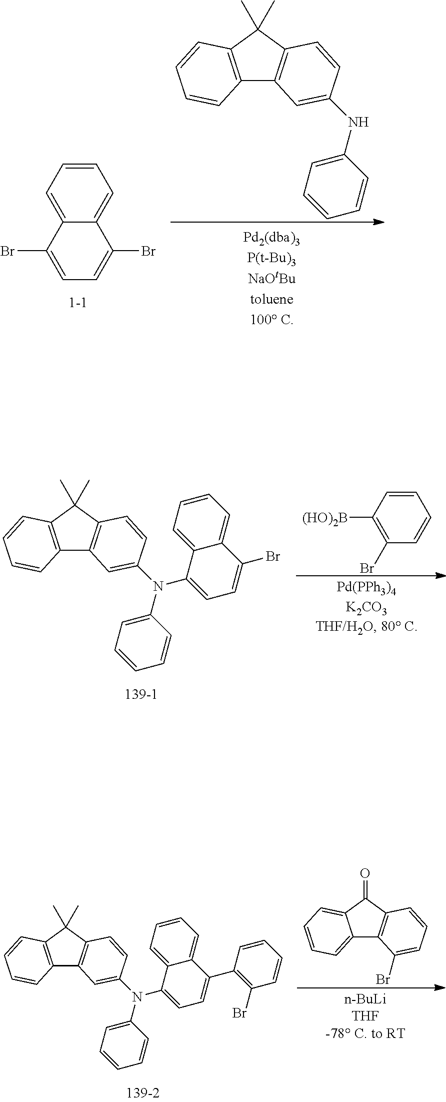

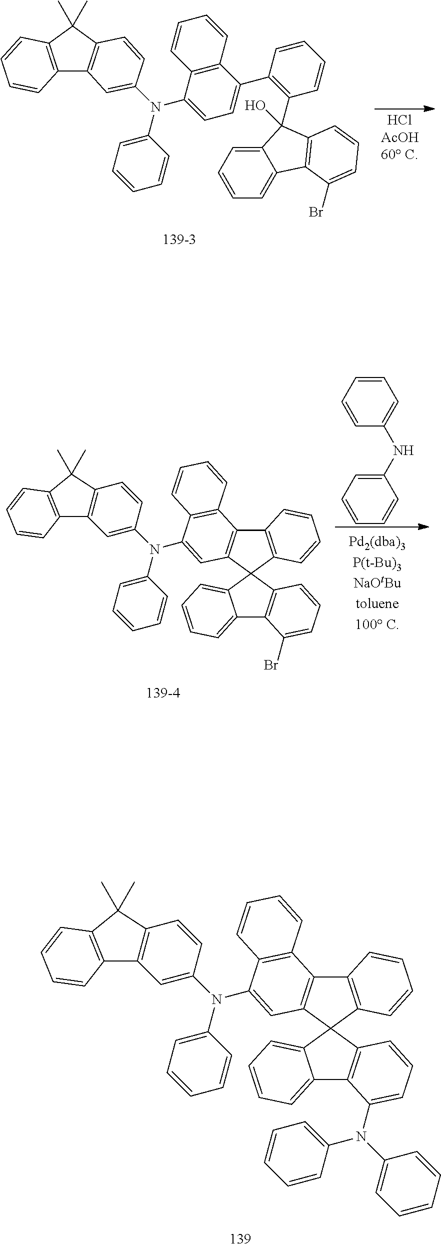

Synthesis of Intermediate 139-1

Intermediate 1-1 (2.85 g), 9,9-dimethyl-N-phenyl-9H-fluoren-3-amine (2.85 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was coaled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 139-1 (3.57 g, yield: 73%).

Synthesis of Intermediate 139-2

Intermediate 139-1 (4.90 g), Pd(PPh₃)₄ (0.56 g), K₂CO₃ (3.45 g), and (2-bromophenyl)boronic acid (2.00 g) were dissolved in THF/H₂O (100 ml/25 ml) and stirred for hour at 0° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 139-2 (3.45 g, yield: 61%).

Synthesis of Intermediate 139-3

A solution obtained by dissolving Intermediate 139-2 (5.66 g) in THF (50 ml) was stirred at −78° C. 4 ml of n-BuLi (2.5M in hexane) was slowly added dropwise to the reaction solution at −78° C. and stirred for 30 minutes. 4-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 139-3 (4.17 g, yield: 56%).

Synthesis of Intermediate 139-4

Intermediate 139-3 (7.46 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 139-4 (6.40 g, yield: 88%).

Synthesis of Compound 139

Intermediate 139-4 (7.28 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 139 (6.04 g, yield: 74%).

### Synthesis Example 9: Synthesis of Compound 147

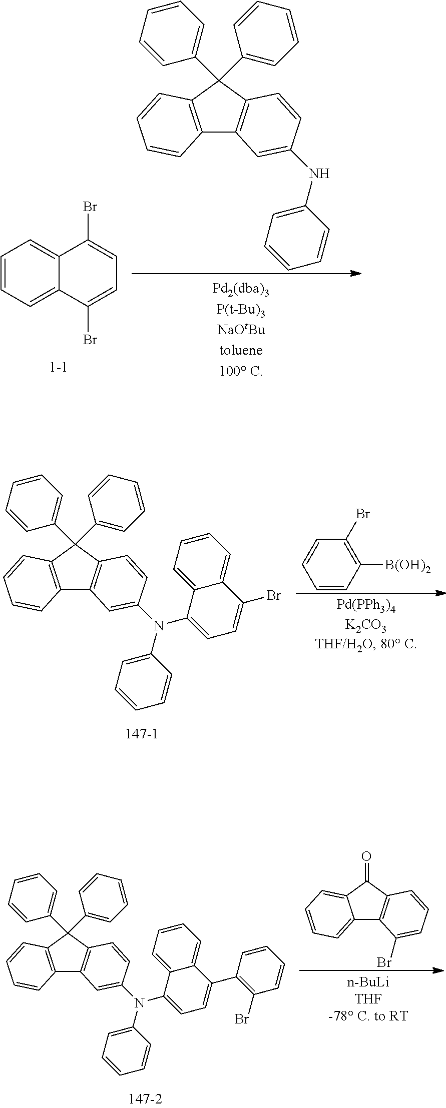

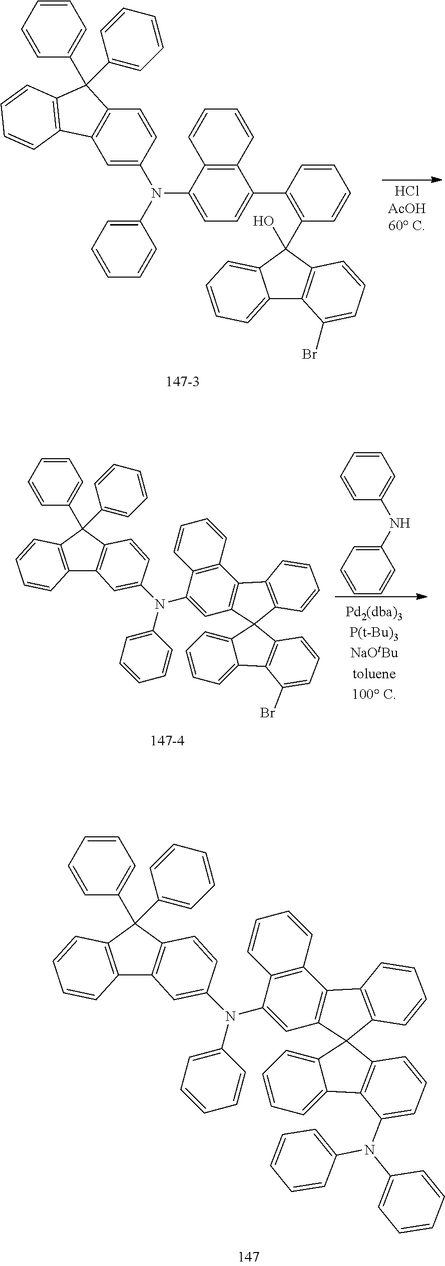

Synthesis of Intermediate 147-1

Intermediate 1-1 (2.85 g) N,9,9-triphenyl-9H-fluoren-3-amine (4.09 g) Pd₂(dba)₃ (0.46 g), P(t-Bu)₃ (0.21 g), and NaOtBu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 147-1 (4.29 g, yield: 70%).

Synthesis of Intermediate 147-2

Intermediate 147-1 (6.14 g), Pd(PPh₃)₄ (0.56 g), K₂CO₃ (3.45 g), and (2-bromophenyl)boronic acid (2.00 g) were dissolved in THF/H₂O (100 ml/25 ml) and stirred for 12 hours at 80° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 147-2 (4.62 g, yield: 67%).

Synthesis of Intermediate 147-3

A solution obtained by dissolving Intermediate 147-2 (6.90 g) in THF (50 ml) was stirred at −78° C. 4 ml of n-BuLi (2.5M in hexane) was slowly added dropwise to the reaction solution at −78° C. and stirred for 30 minutes. 4-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 147-3 (5.22 g, yield: 60%).

Synthesis of Intermediate 147-4

Intermediate 147-3 (8.70 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 147-4 (6.73 g, yield: 79%).

Synthesis of Compound 147

Intermediate 147-4 (8.52 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 147 (6.68 g, yield: 71%).

### Synthesis Example 10: Synthesis of Compound 191

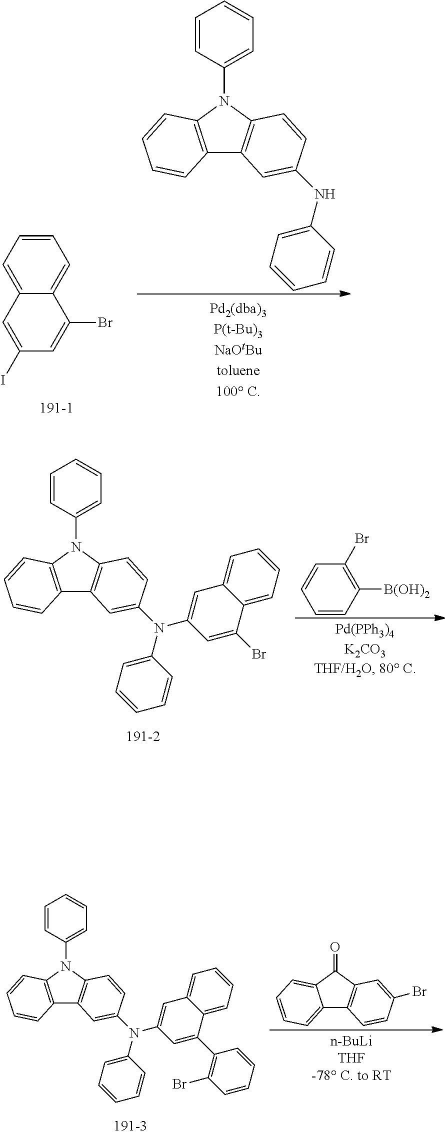

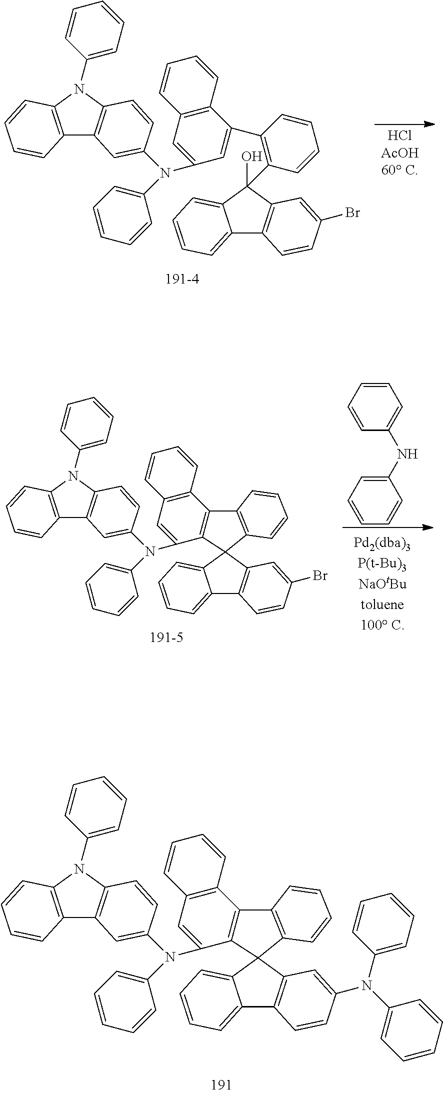 [SMILES: OC1(c2ccccc2-c2cc(N(c3ccccc3)c3ccc4c(c3)c3ccccc3n4-c3ccccc3)cc3ccccc23)c2ccccc2-c2ccc(Br)cc21 ; Brc1ccc2c(c1)C1(c3ccccc3-2)c2ccccc2-c2c1c(N(c1ccccc1)c1ccc3c(c1)c1ccccc1n3-c1ccccc1)cc1ccccc21 ; c1ccc(Nc2ccccc2)cc1 ; c1ccc(N(c2ccccc2)c2ccc3c(c2)C2(c4ccccc4-3)c3ccccc3-c3c2c(N(c2ccccc2)c2ccc4c(c2)c2ccccc2n4-c2ccccc2)cc2ccccc32)cc1]

Synthesis of Intermediate 191-2

Intermediate 191-1 (3.32 g), N,9-diphenyl-9H-carbazol-3-amine (3.34 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was coaled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 191-2 (3.89 g, yield: 730%%)

Synthesis of Intermediate 191-3

Intermediate 191-2 (5.38 g), Pd(PPh₃)₄ (0.56 g), K₂CO₃ (3.45 g), and (2-bromophenyl)boronic acid (2.00 g) were dissolved in THF/H₂O (100 mV/25 ml) and stirred for 12 hours at 80° C. After the reaction solution was coaled to roam temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 191-3 (3.69 g, yield: 60%)

Synthesis of Intermediate 191-4

A solution obtained by dissolving Intermediate 191-3 (6.15 g) in THF (50 ml) was stirred at −78° C. 4 ml of n-BuLi (2.5M in hexane) was slowly added dropwise to the reaction solution at −78° C. and stirred for 30 minutes. 2-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 191-4 (5.00 g, yield: 63%).

Synthesis of Intermediate 191-5

Intermediate 191-4 (7.95 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 191-5 (5.98 g, yield: 77%).

Synthesis of Compound 191

Intermediate 191-5 (7.77 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 191 (7.53 g, yield: 87%).

### Synthesis Example 11: Synthesis of Compound 195

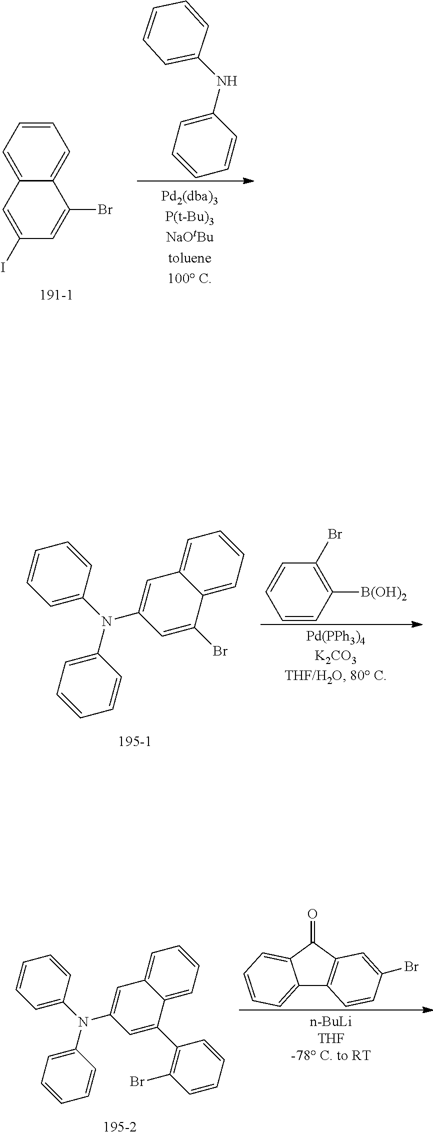

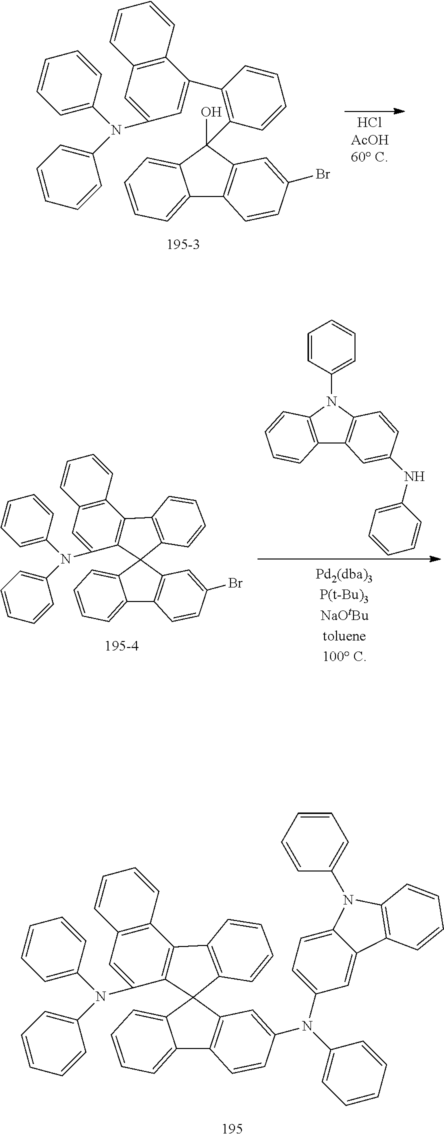 [SMILES: OC1(c2ccccc2-c2cc(N(c3ccccc3)c3ccccc3)cc3ccccc23)c2ccccc2-c2ccc(Br)cc21 ; Brc1ccc2c(c1)C1(c3ccccc3-2)c2ccccc2-c2c1c(N(c1ccccc1)c1ccccc1)cc1ccccc21 ; c1ccc(Nc2ccc3c(c2)c2ccccc2n3-c2ccccc2)cc1 ; c1ccc(N(c2ccc3c(c2)C2(c4ccccc4-3)c3ccccc3-c3c2c(N(c2ccccc2)c2ccccc2)cc2ccccc32)c2ccc3c(c2)c2ccccc2n3-c2ccccc2)cc1]

Synthesis of Intermediate 195-1

Intermediate 191-1 (3.32 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 195-1 (2.99 g, yield: 80%).

Synthesis of Intermediate 195-2

Intermediate 195-1 (3.74 g), Pd(PPh₃)₄ (0.56 g), K₂CO₃ (3.45 g), and (2-bromophenyl)boronic acid (2.00 g) were dissolved in THF/H₂O (100 ml/25 ml) and stirred for 12 hours at 80° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 195-2 (2.52 g, yield: 56%).

Synthesis of Intermediate 195-3

A solution obtained by dissolving Intermediate 195-2 (4.50 g) in THF (50 ml) was stirred at −78° C. 4 ml of n-BuLi (2.5M in hexane) was slowly added dropwise to the reaction solution at −78° C. and stirred for 30 minutes. 2-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 195-3 (3.45 g, yield: 55%).

Synthesis of Intermediate 195-4

Intermediate 1951-3 (6.30 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 195-4 (4.46 g, yield: 73%).

Synthesis of Compound 195

Intermediate 195-4 (6.12 g), N,9-diphenyl-9H-carbazol-3-amine (3.34 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 195 (7.79 g, yield: 90%).

### Synthesis Example 12: Synthesis of Compound 200

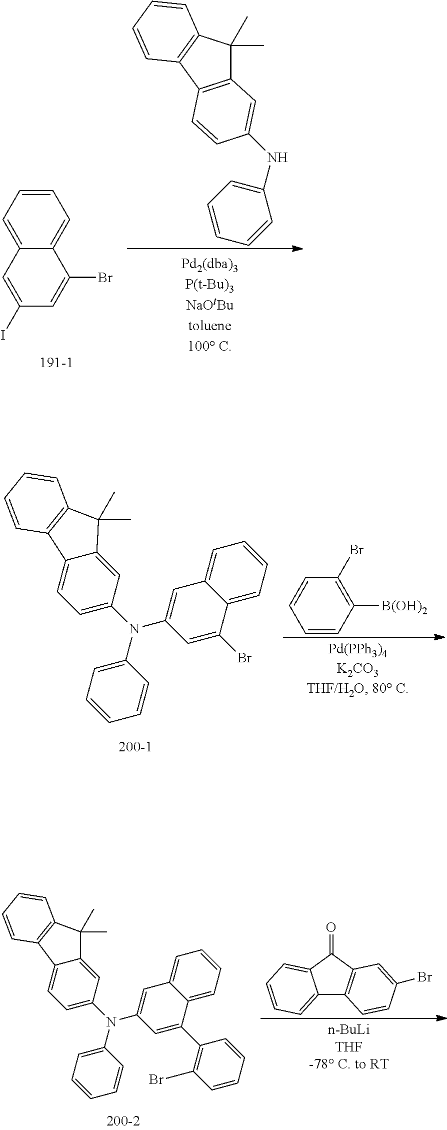

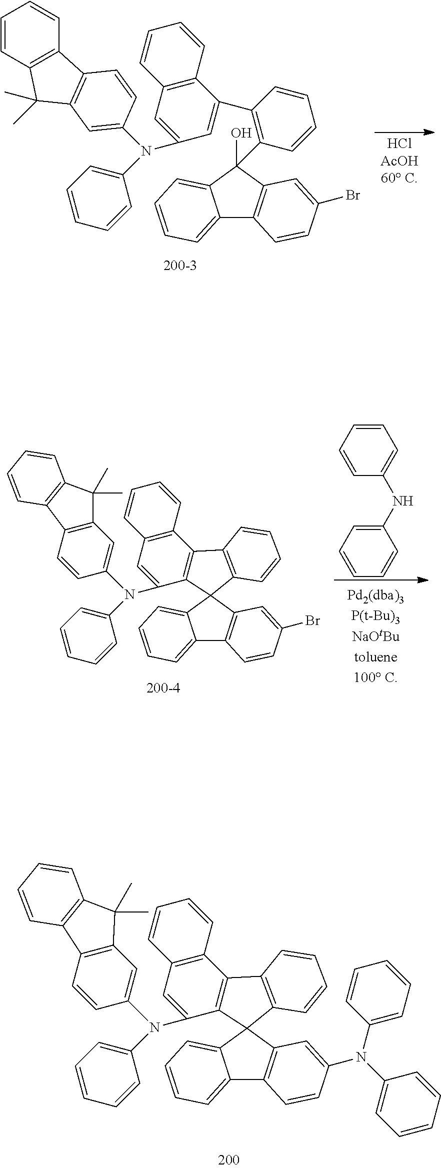 [SMILES: CC1(C)c2ccccc2-c2ccc(N(c3ccccc3)c3cc(-c4ccccc4C4(O)c5ccccc5-c5ccc(Br)cc54)c4ccccc4c3)cc21 ; CC1(C)c2ccccc2-c2ccc(N(c3ccccc3)c3cc4ccccc4c4c3C3(c5ccccc5-c5ccc(Br)cc53)c3ccccc3-4)cc21 ; c1ccc(Nc2ccccc2)cc1 ; CC1(C)c2ccccc2-c2ccc(N(c3ccccc3)c3cc4ccccc4c4c3C3(c5ccccc5-c5ccc(N(c6ccccc6)c6ccccc6)cc53)c3ccccc3-4)cc21]

Synthesis of Intermediate 200-1

Intermediate 191-1 (3.32 g), 9,9-dimethyl-N-phenyl-9H-fluoren-2-amine (2.85 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 200-1 (3.48 g, yield: 71%).

Synthesis of Intermediate 200-2

Intermediate 200-1 (4.90 g), Pd(PPh₃)₄ (0.56 g), K₂CO₃ (3.45 g), and (2-bromophenyl)boronic acid (2.00 g) were dissolved in THF/H₂O (100 ml/25 ml) and stirred for 12 hours at 80° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 200-2 (3.45 g, yield: 61%).

Synthesis of Intermediate 200-3

A solution obtained by dissolving Intermediate 200-2 (5.66 g) in THF (50 ml) was stirred at −78° C. 4 ml of n-BuLi (2.5M in hexane) was slowly added dropwise to the reaction solution at −78° C. and stirred for 30 minutes. 2-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 200-3 (4.17 g, yield: 56%).

Synthesis of Intermediate 200-4

Intermediate 200-3 (7.46 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 200-4 (5.60 g, yield: 77%).

Synthesis of Compound 200

Intermediate 200-4 (7.28 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 200 (7.18 g, yield: 88%).

### Synthesis Example 13: Synthesis of Compound 240

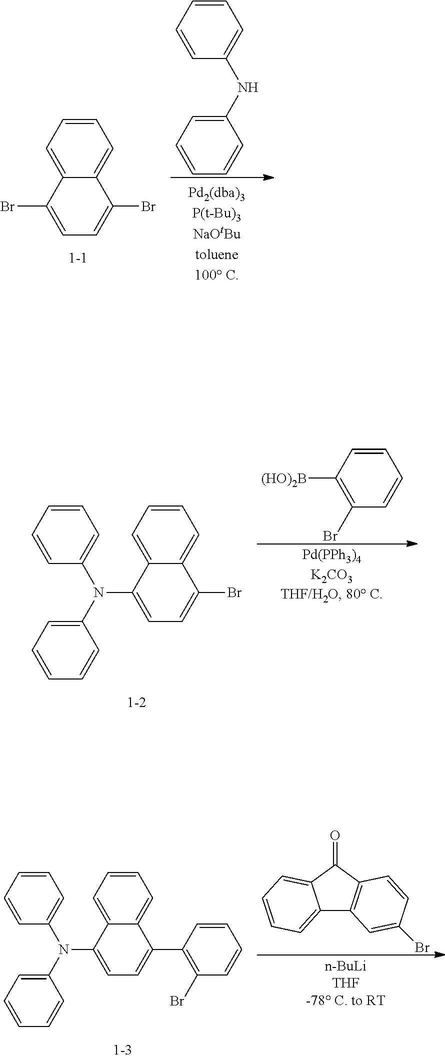

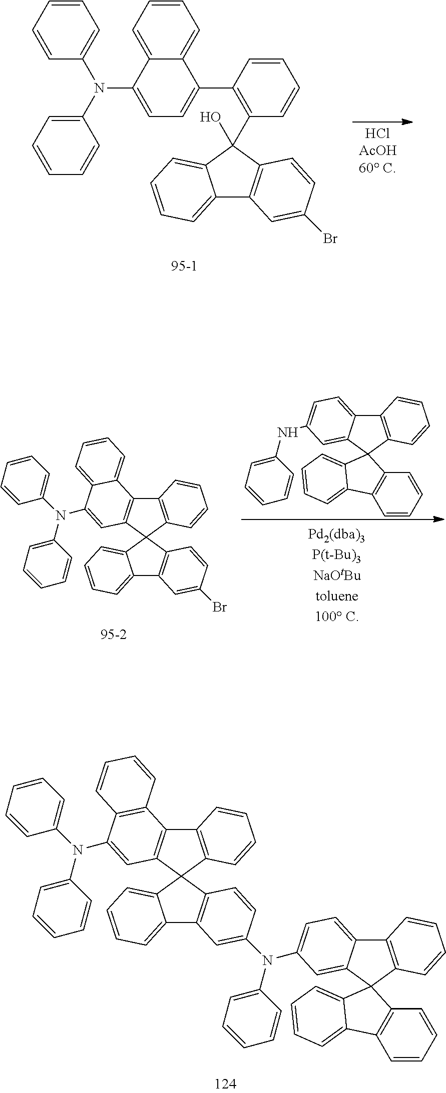 [SMILES: OC1(c2ccccc2-c2ccc(N(c3ccccc3)c3ccccc3)c3ccccc23)c2ccccc2-c2cc(Br)ccc21 ; Brc1ccc2c(c1)-c1ccccc1C21c2ccccc2-c2c1cc(N(c1ccccc1)c1ccccc1)c1ccccc21 ; c1ccc(Nc2ccc3c(c2)C2(c4ccccc4-c4ccccc42)c2ccccc2-3)cc1 ; c1ccc(N(c2ccc3c(c2)-c2ccccc2C32c3ccccc3-c3c2cc(N(c2ccccc2)c2ccccc2)c2ccccc32)c2ccc3c(c2)C2(c4ccccc4-c4ccccc42)c2ccccc2-3)cc1]

Synthesis of Compound 240

Intermediate 95-2 (6.12 g), N-phenyl-9,9′-spirobi[fluoren]-2-amine (4.07 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was coaled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 240 (7.88 g, yield: 84%).

### Synthesis Example 14: Synthesis of Compound 275

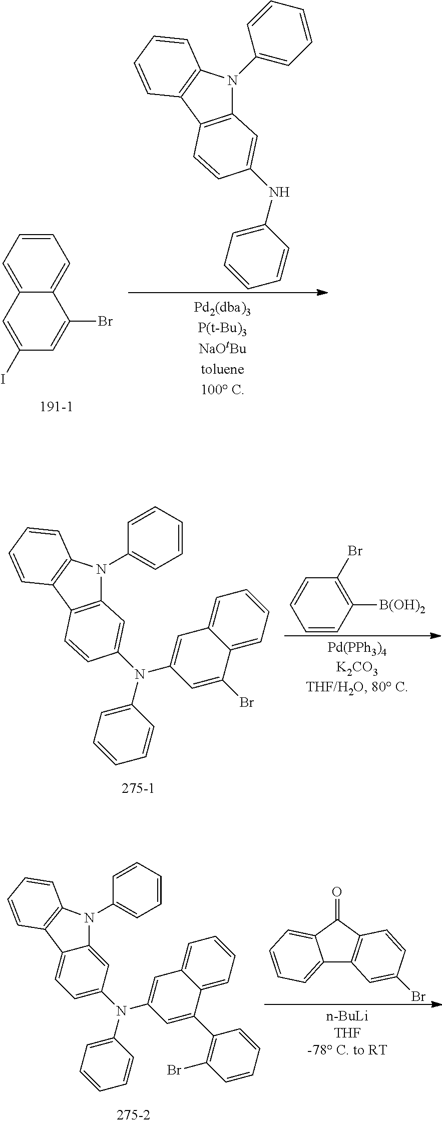

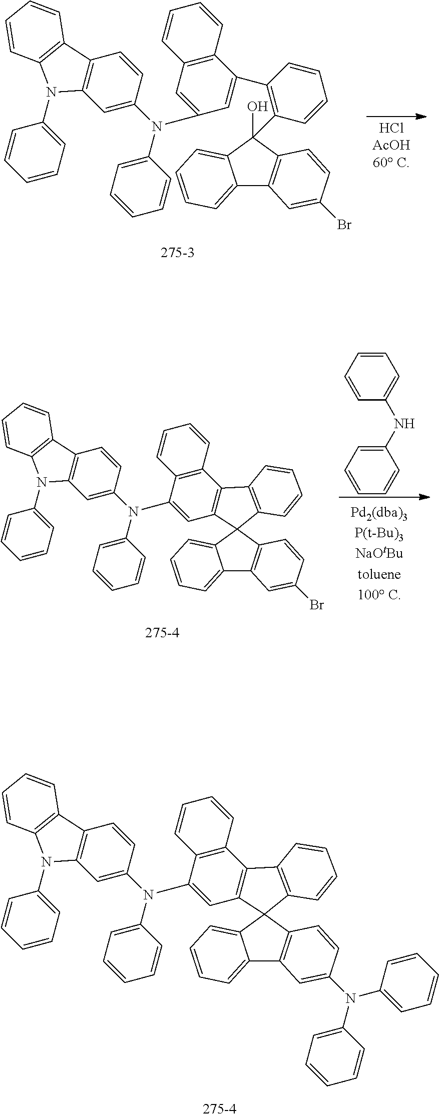 [SMILES: OC1(c2ccccc2-c2cc(N(c3ccccc3)c3ccc4c5ccccc5n(-c5ccccc5)c4c3)cc3ccccc23)c2ccccc2-c2cc(Br)ccc21 ; Brc1ccc2c(c1)-c1ccccc1C21c2ccccc2-c2c1cc(N(c1ccccc1)c1ccc3c4ccccc4n(-c4ccccc4)c3c1)c1ccccc21 ; c1ccc(Nc2ccccc2)cc1 ; c1ccc(N(c2ccccc2)c2ccc3c(c2)-c2ccccc2C32c3ccccc3-c3c2cc(N(c2ccccc2)c2ccc4c5ccccc5n(-c5ccccc5)c4c2)c2ccccc32)cc1]

Synthesis of Intermediate 275-1

Intermediate 191-1 (3.32 g), N,9-diphenyl-9H-carbazol-2-amine (3.34 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 275-1 (3.88 g, yield: 72%).

Synthesis of Intermediate 275-2

Intermediate 275-1 (5.39 g), Pd(PPh₃)₄ (0.56 g), K₂CO₃ (3.45 g), and (2-bromophenyl)boronic acid (2.00 g) were dissolved in THF/H₂O (100 ml/25 ml) and stirred for 12 hours at 80° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 275-2 (3.62 g, yield: 59%).

Synthesis of Intermediate 275-3

A solution obtained by dissolving Intermediate 275-2 (6.15 g) in THF (50 ml) was stirred at −78° C. 4 ml of n-BuLi (2.5M in hexane) was slowly added dropwise to the reaction solution at −78° C. and stirred for 30 minutes. 3-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 275-3 (5.00 g, yield: 63%).

Synthesis of Intermediate 275-4

Intermediate 275-3 (7.95 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 275-4 (6.29 g, yield: 81%).

Synthesis of Compound 275

Intermediate 275-4 (7.77 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 275 (7.71 g, yield: 89%).

### Synthesis Example 15: Synthesis of Compound 282

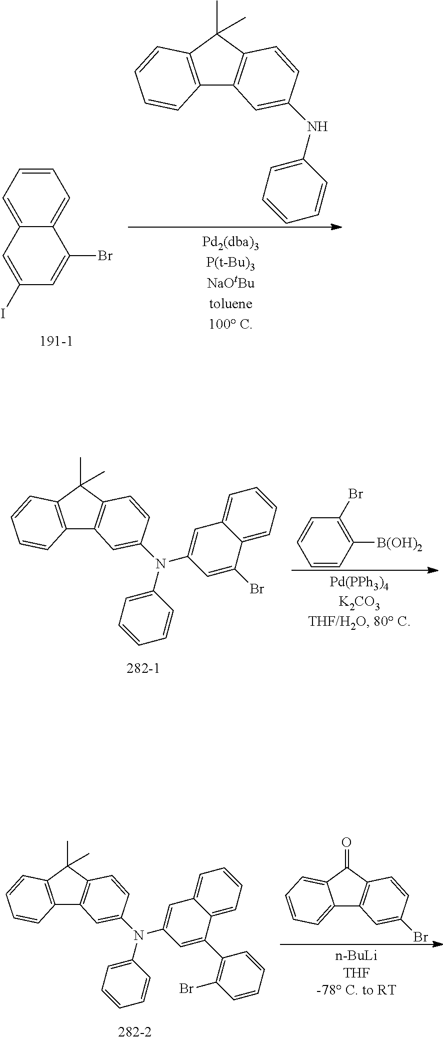

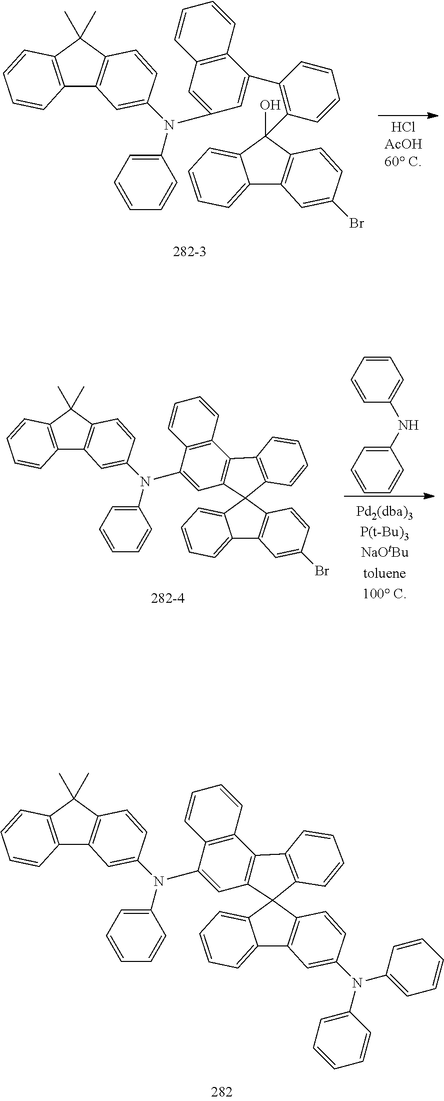 [SMILES: CC1(C)c2ccccc2-c2cc(N(c3ccccc3)c3cc(-c4ccccc4C4(O)c5ccccc5-c5cc(Br)ccc54)c4ccccc4c3)ccc21 ; CC1(C)c2ccccc2-c2cc(N(c3ccccc3)c3cc4c(c5ccccc35)-c3ccccc3C43c4ccccc4-c4cc(Br)ccc43)ccc21 ; c1ccc(Nc2ccccc2)cc1 ; CC1(C)c2ccccc2-c2cc(N(c3ccccc3)c3cc4c(c5ccccc35)-c3ccccc3C43c4ccccc4-c4cc(N(c5ccccc5)c5ccccc5)ccc43)ccc21]

Synthesis of Intermediate 282-1

Intermediate 191-1 (3.32 g), 9,9-dimethyl-N-phenyl-9H-fluoren-3-amine (2.85 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 282-1 (3.77 g, yield: 77%).

Synthesis of Intermediate 282-2

Intermediate 282-1 (4.90 g), Pd(PPh₃)₄ (0.56 g), K₂CO₃ (3.45 g), and (2-bromophenyl)boronic acid (2.00 g) were dissolved in THF/H₂O (100 ml/25 ml) and stirred for 12 hours at 80° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 282-2 (3.56 g, yield: 63%).

Synthesis of Intermediate 282-3

A solution obtained by dissolving Intermediate 282-2 (5.66 g) in THF (50 ml) was stirred at −78° C. 4 ml of n-BuLi (2.5M in hexane) was slowly added dropwise to the reaction solution at −78° C. and stirred for 30 minutes. 3-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 282-3 (4.02 g, yield: 54%).

Synthesis of Intermediate 282-4

Intermediate 282-3 (7.46 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 282-4 (6.62 g, yield: 91%).

Synthesis of Compound 282

Intermediate 282-4 (7.28 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 282 (6.20 g, yield: 76%).

### Synthesis Example 16: Synthesis of Compound 315

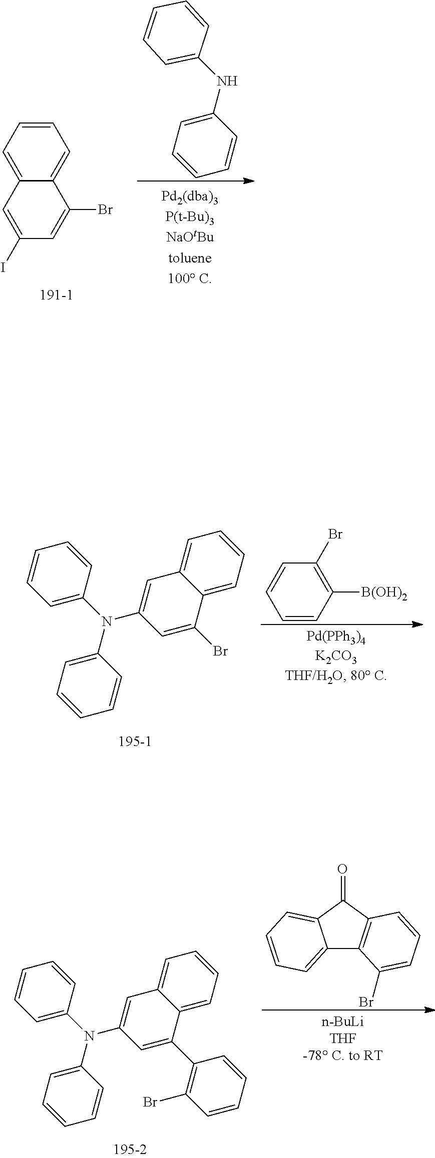

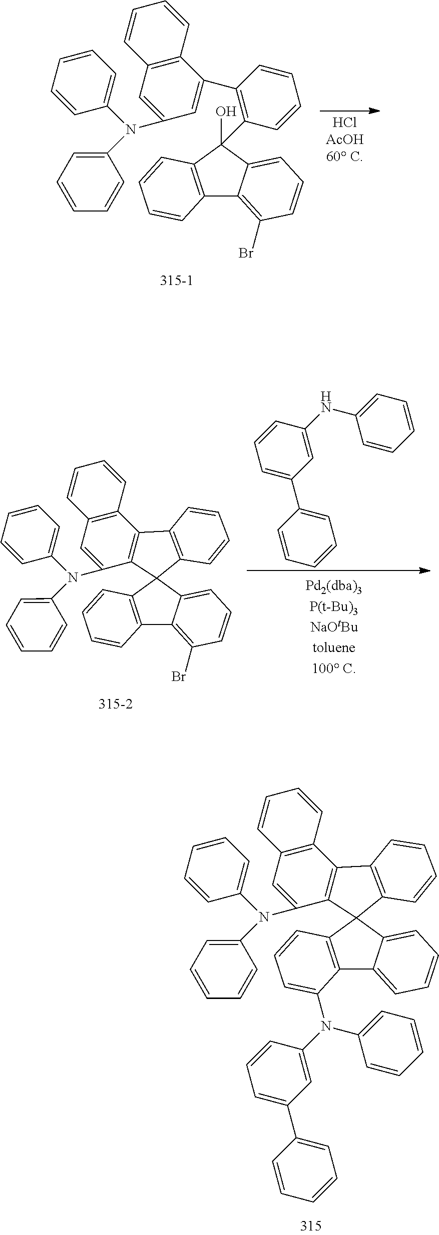 [SMILES: OC1(c2ccccc2-c2cc(N(c3ccccc3)c3ccccc3)cc3ccccc23)c2ccccc2-c2c(Br)cccc21 ; Brc1cccc2c1-c1ccccc1C21c2ccccc2-c2c1c(N(c1ccccc1)c1ccccc1)cc1ccccc21 ; c1ccc(Nc2cccc(-c3ccccc3)c2)cc1 ; c1ccc(-c2cccc(N(c3ccccc3)c3cccc4c3-c3ccccc3C43c4ccccc4-c4c3c(N(c3ccccc3)c3ccccc3)cc3ccccc43)c2)cc1]

Synthesis of Intermediate 315-1

A solution obtained by dissolving Intermediate 195-2 (4.50 g) in THF (50 ml) was stirred at −78° C. 4 ml of n-BuLi (2.5M in hexane) was slowly added dropwise to the reaction solution at −78° C. and stirred for 30 minutes. 4-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 315-1 (3.15 g, yield: 50%).

Synthesis of Intermediate 315-2

Intermediate 315-1 (6.30 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 315-2 (5.32 g, yield: 87%).

Synthesis of Compound 315

Intermediate 315-2 (6.12 g), N-phenyl-[1,1′-biphenyl]-3-amine (2.45 g), Pd₂(dba)₃ (0.46 g), P(t-Bu)₃ (0.21 g), and NaOtBu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 315 (6.28 g, yield: 80%).

### Synthesis Example 17: Synthesis of Compound 334

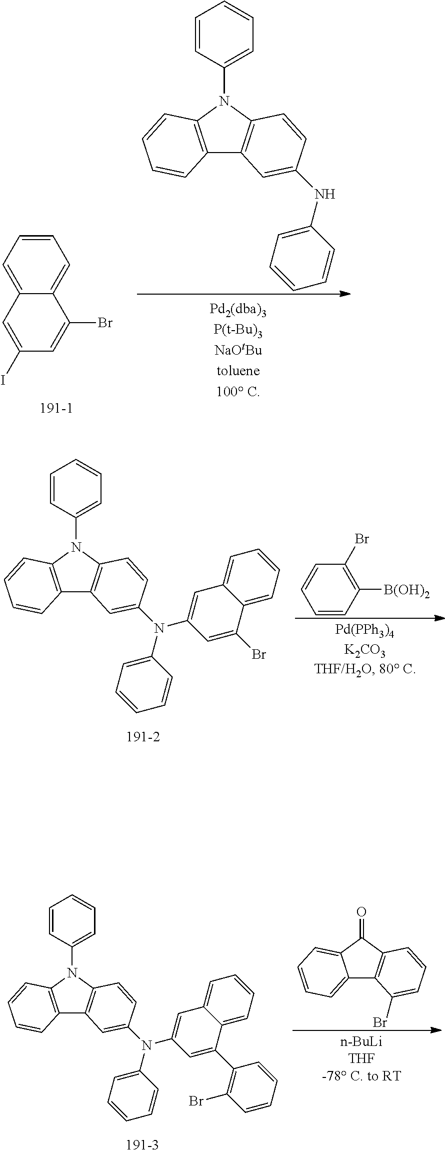

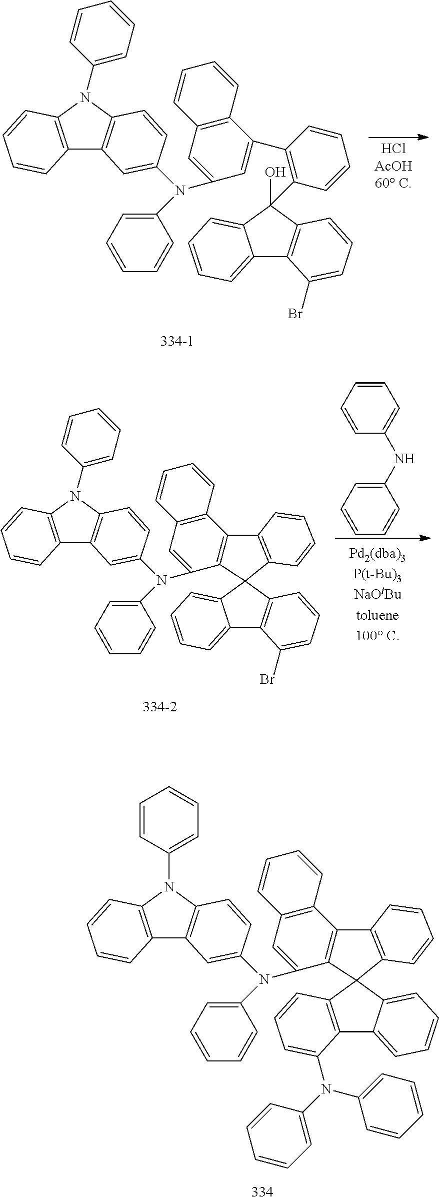

Synthesis of Intermediate 334-1

A solution obtained by dissolving Intermediate 191-3 (6.15 g) in THF (50 ml) was stirred at −78° C. 4 ml of n-BuLi (2.5M in hexane) was slowly added dropwise to the reaction solution at −78° C. and stirred for 30 minutes. 4-bromo-9H-fluoren-9-one (2.59 g) was added dropwise to the reaction solution at −78° C. and stirred for 12 hours at room temperature. The reaction solution was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 334-1 (4.84 g, yield: 61%).

Synthesis of Intermediate 334-2

Intermediate 334-1 (7.95 g) was dissolved in HCl (6 ml) and acetic acid (30 ml) and stirred for 6 hours at 60° C. After the reaction solution was cooled to room temperature, the reaction was terminated by using water, and an extraction process was performed thereon three times by using ethylether. An organic layer extracted therefrom was dried by using anhydrous magnesium sulfate and distilled under reduced pressure, and a residue thus obtained was separated and purified by column chromatography, thereby obtaining Compound 334-2 (6.99 g, yield: 90%).

Synthesis of Compound 334

Intermediate 334-2 (7.77 g), diphenylamine (1.69 g), Pd₂(dba)₃ (0.46 g), P(t-Bu)₃ (0.21 g), and NaO^{t}Bu (2.44 g) were dissolved in toluene (50 ml) and stirred for an hour at 100° C. After the reaction solution was cooled to room temperature, the reaction was terminated by using water, and an extraction process was performed thereon three times by using ethylether. An organic layer separated therefrom was dried by using anhydrous magnesium sulfate and distilled under reduced pressure, and a residue thus obtained was separated and purified by column chromatography, thereby obtaining Compound 334 (6.40 g, yield: 74%).

### Comparative Example 1

As an anode, a 15 Ω/cm² (1,200 Å) ITO glass substrate (available from Corning Co., Ltd) was cut to a size of 50 mm×50 mm×0.7 mm, sonicated in isopropyl alcohol and pure water for 5 minutes in each solvent, and cleaned by irradiation of ultraviolet rays and exposure of ozone thereto for 30 minutes, and the glass substrate was loaded onto a vacuum deposition apparatus.

2-TNATA was vacuum-deposited on the substrate to form a hole injection layer having a thickness of 600 Å, and 4,4′-bis[N-(1-naphthyl)-N-phenylamino]biphenyl (hereinafter, NPB), which is a hole transport material as a hole transport compound, was vacuum-deposited thereon to form a hole transport layer having a thickness of 300 Å.

9,10-di(naphthalen-2-yl)anthracene (hereinafter, DNA) as a host and 4,4′-bis[2-(4-(N,N-diphenylamino)phenyl)vinyl]biphenyl (hereinafter, DPAVBi) as a dopant were co-deposited on the hole transport layer at a weight ratio of 98:2 to form an emission layer having a thickness of 300 Å.

Alq₃ was deposited on the emission layer to form an electron transport layer having a thickness of 300 Å, and LiF, which is a halogenated alkali metal, was deposited on the electron transport layer to form an electron injection layer having a thickness of 10 Å and A1 was vacuum-deposited thereon to form a cathode electrode having a thickness of 3,000 Å, to form a LiF/Al electrode, thereby completing manufacture of an organic electroluminescent device.

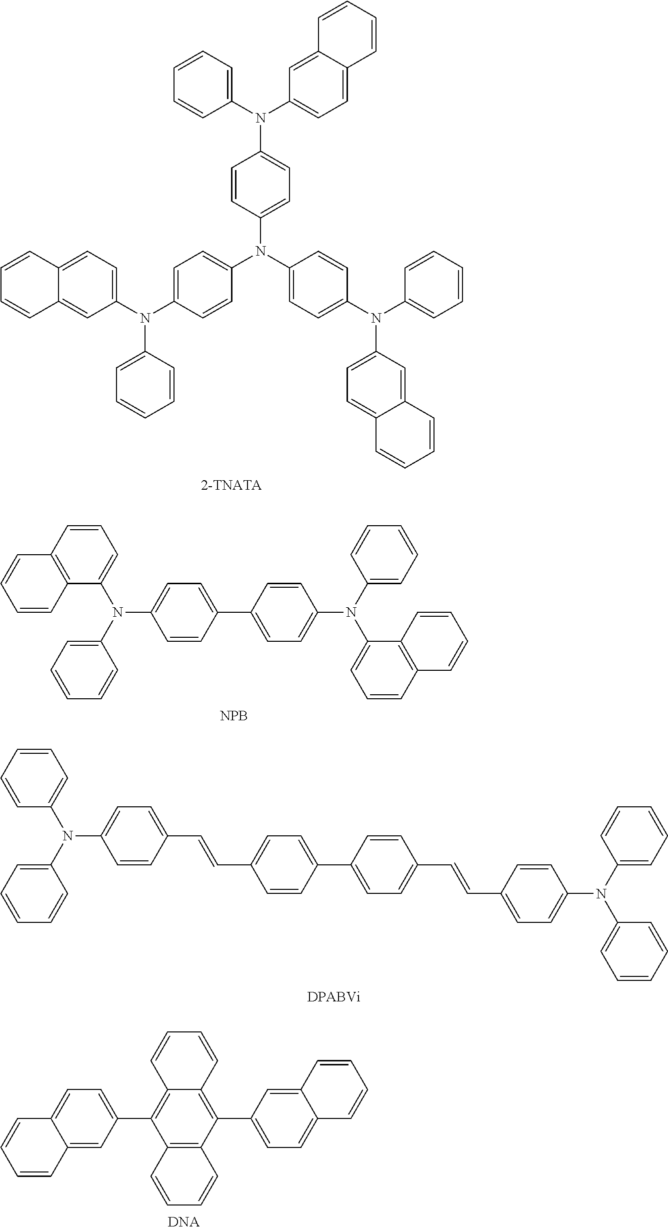

### Comparative Examples 2 to 4 and Examples 1 to 17

Organic electroluminescent devices were manufactured in the same manner as in Comparative Example 1, except that compounds shown in Table 1 were used instead of NPB in forming the hole transport layer.

### Evaluation Example 1

To evaluate characteristics of the organic electroluminescent devices manufactured according to Comparative Examples 1 to 4 and Examples 1 to 17, the driving voltage at the current density of 50 mA/cm², luminance, and luminescence efficiency thereof were measured. The driving voltage and current density of the light-emitting devices were measured by using V7000 OLED IVL Test System (Polaronix). Table 1 shows the evaluation results of the characteristics of the organic electroluminescent devices.

TABLE 1

| | | | | | | | Half |
|---|---|---|---|---|---|---|---|
| | Hole | Driving | Current | | | | lifespan |
| | transport | voltage | density | Luminance | Efficiency | Emission | (hr @100 |
| | material | (V) | (mA/cm²) | (cd/m²) | (cd/A) | color | mA/cm²) |
| Comparative | NPB | 7.01 | 50 | 2645 | 5.29 | Blue | 258 |
| Example 1 | | | | | | | |
| Comparative | A | 5.63 | 50 | 3260 | 6.52 | Blue | 354 |
| Example 2 | | | | | | | |
| Comparative | B | 5.53 | 50 | 3460 | 6.92 | Blue | 370 |
| Example 3 | | | | | | | |
| Comparative | C | 6.20 | 50 | 3010 | 6.02 | Blue | 287 |
| Example 4 | | | | | | | |
| Example 1 | Compound 1 | 4.52 | 50 | 4160 | 8.32 | Blue | 465 |
| Example 2 | Compound 32 | 4.46 | 50 | 4000 | 8.00 | Blue | 434 |
| Example 3 | Compound 36 | 4.33 | 50 | 3975 | 7.95 | Blue | 411 |
| Example 4 | Compound 40 | 4.16 | 50 | 4175 | 8.35 | Blue | 456 |
| Example 5 | Compound 92 | 4.00 | 50 | 4225 | 8.45 | Blue | 399 |
| Example 6 | Compound 95 | 4.51 | 50 | 4120 | 8.24 | Blue | 492 |
| Example 7 | Compound 124 | 4.65 | 50 | 4115 | 8.23 | Blue | 466 |
| Example 8 | Compound 139 | 4.52 | 50 | 4235 | 8.47 | Blue | 384 |
| Example 9 | Compound 147 | 4.44 | 50 | 4280 | 8.56 | Blue | 444 |
| Example 10 | Compound 191 | 4.32 | 50 | 4390 | 8.78 | Blue | 455 |
| Example 11 | Compound 195 | 4.32 | 50 | 3725 | 7.45 | Blue | 467 |
| Example 12 | Compound 200 | 4.42 | 50 | 4050 | 8.10 | Blue | 413 |
| Example 13 | Compound 240 | 4.41 | 50 | 4125 | 8.25 | Blue | 441 |
| Example 14 | Compound 275 | 4.22 | 50 | 3800 | 7.60 | Blue | 399 |
| Example 15 | Compound 282 | 4.11 | 50 | 4225 | 8.45 | Blue | 435 |
| Example 16 | Compound 315 | 4.33 | 50 | 3775 | 7.55 | Blue | 477 |
| Example 17 | Compound 334 | 4.26 | 50 | 4275 | 8.55 | Blue | 476 |

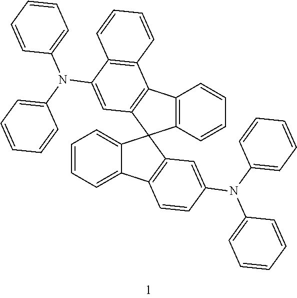 [SMILES: c1ccc(N(c2ccccc2)c2ccc3c(c2)C2(c4ccccc4-3)c3ccccc3-c3c2cc(N(c2ccccc2)c2ccccc2)c2ccccc32)cc1]

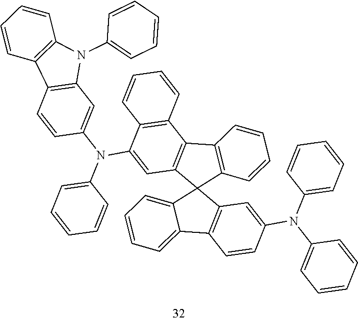 [SMILES: c1ccc(N(c2ccccc2)c2ccc3c(c2)C2(c4ccccc4-3)c3ccccc3-c3c2cc(N(c2ccccc2)c2ccc4c5ccccc5n(-c5ccccc5)c4c2)c2ccccc32)cc1]

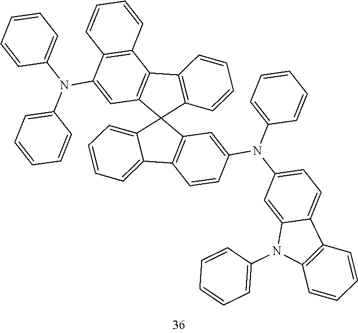 [SMILES: c1ccc(N(c2ccc3c(c2)C2(c4ccccc4-3)c3ccccc3-c3c2cc(N(c2ccccc2)c2ccccc2)c2ccccc32)c2ccc3c4ccccc4n(-c4ccccc4)c3c2)cc1]

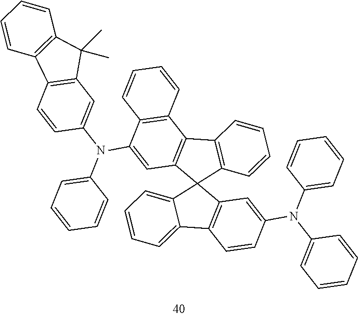 [SMILES: CC1(C)c2ccccc2-c2ccc(N(c3ccccc3)c3cc4c(c5ccccc35)-c3ccccc3C43c4ccccc4-c4ccc(N(c5ccccc5)c5ccccc5)cc43)cc21]

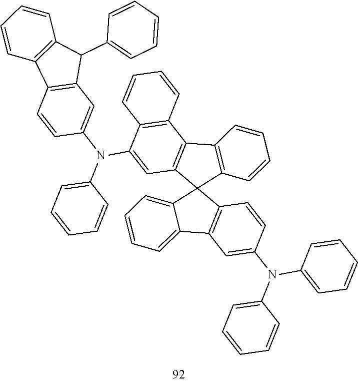 [SMILES: c1ccc(C2c3ccccc3-c3ccc(N(c4ccccc4)c4cc5c(c6ccccc46)-c4ccccc4C54c5ccccc5-c5cc(N(c6ccccc6)c6ccccc6)ccc54)cc32)cc1]

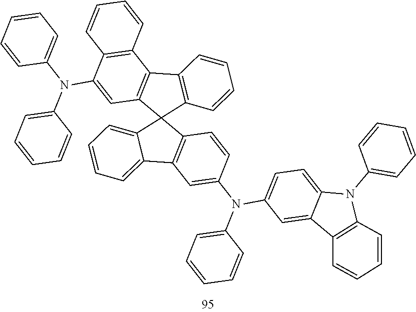 [SMILES: c1ccc(N(c2ccc3c(c2)-c2ccccc2C32c3ccccc3-c3c2cc(N(c2ccccc2)c2ccccc2)c2ccccc32)c2ccc3c(c2)c2ccccc2n3-c2ccccc2)cc1]

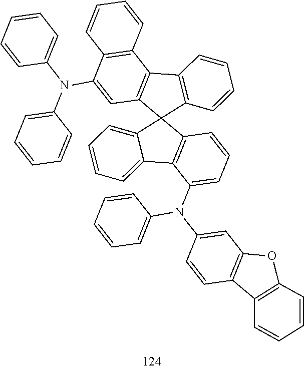 [SMILES: c1ccc(N(c2ccc3c(c2)oc2ccccc23)c2cccc3c2-c2ccccc2C32c3ccccc3-c3c2cc(N(c2ccccc2)c2ccccc2)c2ccccc32)cc1]

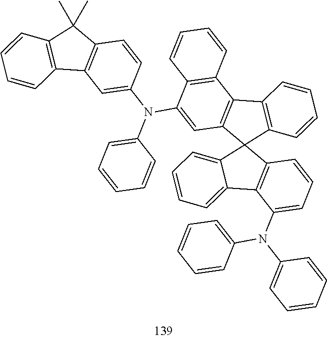 [SMILES: CC1(C)c2ccccc2-c2cc(N(c3ccccc3)c3cc4c(c5ccccc35)-c3ccccc3C43c4ccccc4-c4c(N(c5ccccc5)c5ccccc5)cccc43)ccc21]

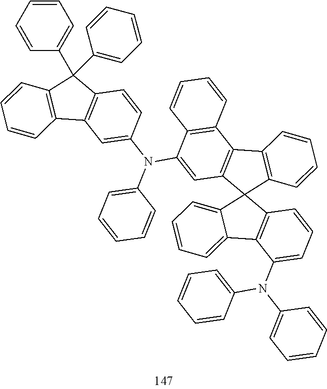 [SMILES: c1ccc(N(c2ccccc2)c2cccc3c2-c2ccccc2C32c3ccccc3-c3c2cc(N(c2ccccc2)c2ccc4c(c2)-c2ccccc2C4(c2ccccc2)c2ccccc2)c2ccccc32)cc1]

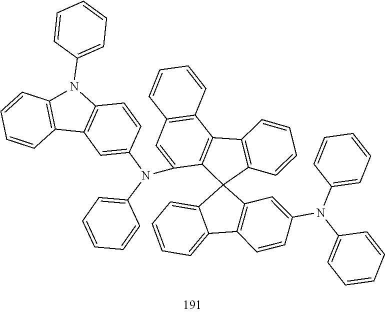 [SMILES: c1ccc(N(c2ccccc2)c2ccc3c(c2)C2(c4ccccc4-3)c3ccccc3-c3c2c(N(c2ccccc2)c2ccc4c(c2)c2ccccc2n4-c2ccccc2)cc2ccccc32)cc1]

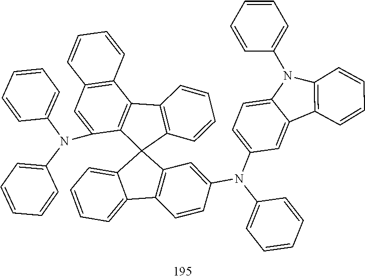 [SMILES: c1ccc(N(c2ccc3c(c2)C2(c4ccccc4-3)c3ccccc3-c3c2c(N(c2ccccc2)c2ccccc2)cc2ccccc32)c2ccc3c(c2)c2ccccc2n3-c2ccccc2)cc1]

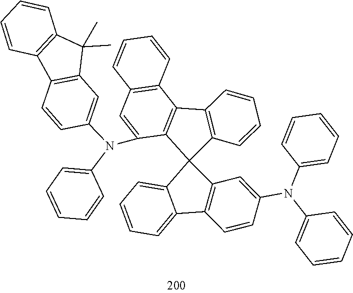 [SMILES: CC1(C)c2ccccc2-c2ccc(N(c3ccccc3)c3cc4ccccc4c4c3C3(c5ccccc5-c5ccc(N(c6ccccc6)c6ccccc6)cc53)c3ccccc3-4)cc21]

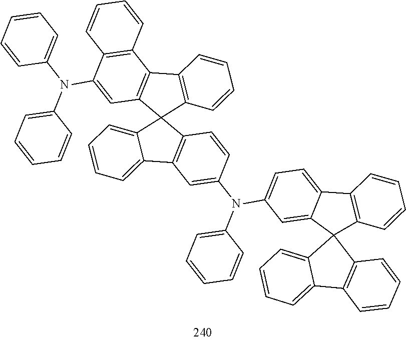 [SMILES: c1ccc(N(c2ccc3c(c2)-c2ccccc2C32c3ccccc3-c3c2cc(N(c2ccccc2)c2ccccc2)c2ccccc32)c2ccc3c(c2)C2(c4ccccc4-c4ccccc42)c2ccccc2-3)cc1]

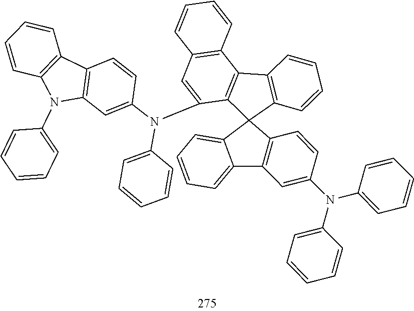 [SMILES: c1ccc(N(c2ccccc2)c2ccc3c(c2)-c2ccccc2C32c3ccccc3-c3c2c(N(c2ccccc2)c2ccc4c5ccccc5n(-c5ccccc5)c4c2)cc2ccccc32)cc1]

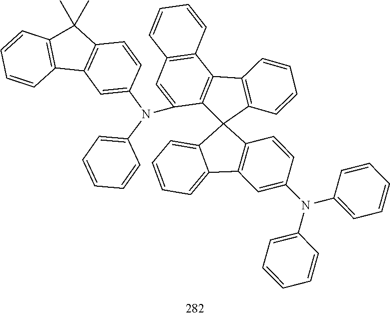 [SMILES: CC1(C)c2ccccc2-c2cc(N(c3ccccc3)c3cc4ccccc4c4c3C3(c5ccccc5-c5cc(N(c6ccccc6)c6ccccc6)ccc53)c3ccccc3-4)ccc21]

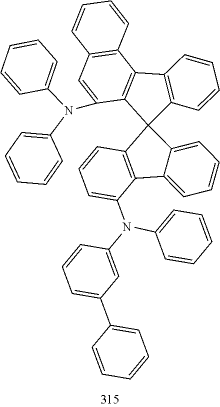 [SMILES: c1ccc(-c2cccc(N(c3ccccc3)c3cccc4c3-c3ccccc3C43c4ccccc4-c4c3c(N(c3ccccc3)c3ccccc3)cc3ccccc43)c2)cc1]

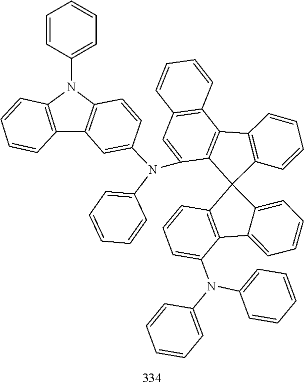 [SMILES: c1ccc(N(c2ccccc2)c2cccc3c2-c2ccccc2C32c3ccccc3-c3c2c(N(c2ccccc2)c2ccc4c(c2)c2ccccc2n4-c2ccccc2)cc2ccccc32)cc1]

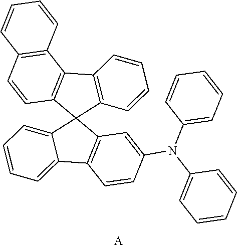

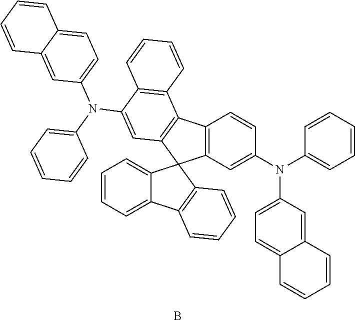

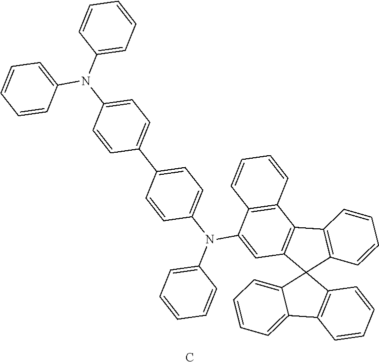

From Table 1, it was confirmed that the organic electroluminescent devices according to Examples 1 to 17 had characteristics of lower driving voltage, higher luminance, higher efficiency, and longer lifespan, as compared to those of the organic electroluminescent devices according to Comparative Examples 1 to 4.

By including the amine-based compound represented by Formula 1, the light-emitting device may have excellent luminescence efficiency and long lifespan. Thus, a high-quality electronic apparatus may be manufactured by using the light-emitting device.

Embodiments have been disclosed herein, and although terms are employed, they are used and are to be interpreted in a generic and descriptive sense only and not for purpose of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. A light-emitting device comprising:
a first electrode;
a second electrode facing the first electrode;
an interlayer between the first electrode and the second electrode and comprising an emission layer; and
an amine-based compound represented by Formula 1: 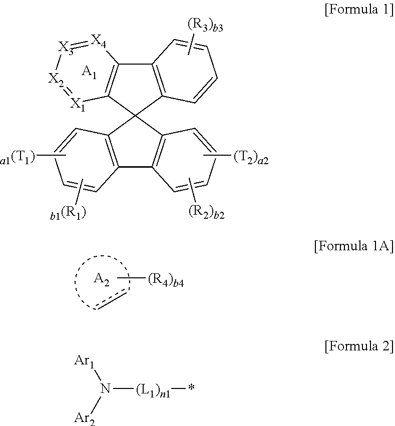
wherein in Formulae 1, 1A, and 2,
X₁ is C(Z₁),
X₂ is C(Z₂),
X₃ is C(Z₃),
X₄ is C(Z₄),
wherein in Z₁ to Z₄:
Z₁ and Z₂ are bonded together to form ring A₂ represented by Formula 1A;
Z₂ and Z₃ are bonded together to form ring A₂ represented by Formula 1A; or
Z₃ and Z₄ are bonded together to form ring A₂ represented by Formula 1A,
ring A₁ and ring A₂ represented by Formula 1A are condensed with each other, [symbol] in Formula 1A indicates a single bond or a double bond,
ring A₂ is a C₃-C₃₀ carbocyclic group,
Z₁ to Z₄ are each independently a group represented by Formula 2, hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
at least one of Z₁ to Z₄ is a group represented by Formula 2,
T₁ and T₂ are each independently a group represented by Formula 2,
a1 and a2 are each independently an integer from 0 to 4,
the sum of a1 and a2 is 1 or more,
R₁ to R₄ are each independently hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),
b1 to b3 are each independently an integer from 0 to 4,
b4 is an integer from 0 to 10,
L₁ is a single bond, a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
n1 is an integer from 1 to 5,
Ar₁ and Ar₂ are each independently a C₃-C₆₀ carbocyclic group that is unsubstituted or substituted with at least one R₁₀ₐ or a C₁-C₆₀ heterocyclic group that is unsubstituted or substituted with at least one R₁₀ₐ,
indicates a binding site to a neighboring atom,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁—CoO heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, a pyridinyl group, a pyrimidinyl group, a pyridazinyl group, a pyrazinyl group, a triazinyl group, or a combination thereof.

2. The light-emitting device of claim 1, wherein the interlayer comprises the amine-based compound.

3. The light-emitting device of claim 1, wherein
the first electrode is an anode,
the second electrode is a cathode,
the interlayer further comprises a hole transport region between the emission layer and the first electrode, and
the hole transport region comprises the amine-based compound.

4. The light-emitting device of claim 3, wherein
the hole transport region comprises at least one of a hole injection layer and a hole transport layer, and
at least one of the hole injection layer and the hole transport layer comprises the amine-based compound.

5. The light-emitting device of claim 1, further comprising a capping layer located outside the first electrode or located outside the second electrode, wherein
the capping layer comprises the amine-based compound.

6. The light-emitting device of claim 1, further comprising:
a first capping layer located outside the first electrode and comprising the amine-based compound;
a second capping layer located outside the second electrode and comprising the amine-based compound; or
the first capping layer and the second capping layer.

7. An electronic apparatus comprising the light-emitting device of claim 1.

8. The electronic apparatus of claim 7, further comprising a thin-film transistor, wherein
the thin-film transistor comprises a source electrode and a drain electrode, and
the first electrode of the light-emitting device is electrically connected to the source electrode or the drain electrode.

9. The electronic apparatus of claim 7, further comprising a color filter, a color conversion layer, a touch screen layer, a polarizing layer, or a combination thereof.

10. An amine-based compound represented by Formula 1: 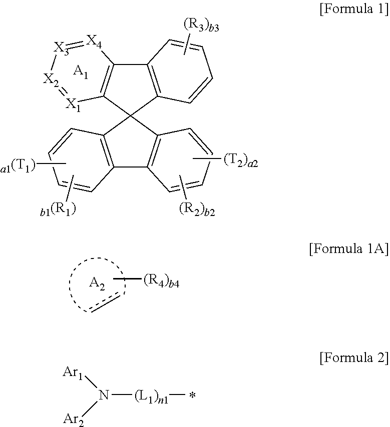
wherein in Formulae 1, 1A, and 2,
X₁ is C(Z₁),
X₂ is C(Z₂),
X₃ is C(Z₃),
X₄ is C(Z₄),
wherein in Z₁ to Z₄:
Z₁ and Z₂ are bonded together to form ring A₂ represented by Formula 1A;
Z₂ and Z₃ are bonded together to form ring A₂ represented by Formula 1A; or
Z₃ and Z₄ are bonded together to form ring A₂ represented by Formula 1A,
ring A₁ and ring A₂ represented by Formula 1A are condensed with each other, [symbol] in Formula 1A indicates a single bond or a double bond,
ring A₂ is a C₃-C₃₀ carbocyclic group,
Z₁ to Z₄ are each independently a group represented by Formula 2, hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
at least one of Z₁ to Z₄ is a group represented by Formula 2,
T₁ and T₂ are each independently a group represented by Formula 2,
a1 and a2 are each independently an integer from 0 to 4,
the sum of a1 and a2 is 1 or more,
R₁ to R₄ are each independently hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),
b1 to b3 are each independently an integer from 0 to 4,
b4 is an integer from 0 to 10,
L₁ is a single bond, a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
n1 is an integer from 1 to 5,
Ar₁ and Ar₂ are each independently a C₃-C₆₀ carbocyclic group that is unsubstituted or substituted with at least one R₁₀ₐ or a C₁-C₆₀ heterocyclic group that is unsubstituted or substituted with at least one R₁₀ₐ,
indicates a binding site to a neighboring atom,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁—CoO heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, a pyridinyl group, a pyrimidinyl group, a pyridazinyl group, a pyrazinyl group, a triazinyl group, or a combination thereof.

11. The amine-based compound of claim 10, wherein ring A₂ is a benzene group or a naphthalene group.

12. The amine-based compound of claim 10, wherein the amine-based compound represented by Formula 1 is represented by one of Formulae 1-1 to 1-3: 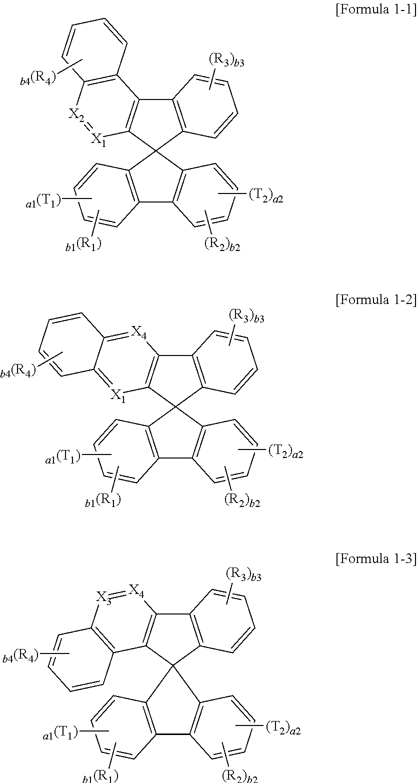
wherein in Formulae 1-1 to 1-3,
b4 is an integer from 0 to 4, and
X₁ to X₄, T1, T₂, a1, a2, R₁ to R₄, and b1 to b3 are respectively the same as described in Formula 1.

13. The amine-based compound of claim 10, wherein Z₁ to Z₄ are each independently:
a group represented by Formula 2, hydrogen, or deuterium;
a C₁-C₁₀ alkyl group that is unsubstituted or substituted with deuterium, a phenyl group, or a combination thereof; or
a phenyl group or a naphthyl group, each unsubstituted or substituted with deuterium, a C₁-C₁₀ alkyl group, a phenyl group, or a combination thereof.

14. The amine-based compound of claim 10, wherein one of Z₁ to Z₄ is a group represented by Formula 2.

15. The amine-based compound of claim 10, wherein the sum of a1 and a2 is 1.

16. The amine-based compound of claim 10, wherein R₁ to R₄ are each independently:
hydrogen, deuterium, —F, —Cl, —Br, —I, a hydroxyl group, a cyano group, a nitro group, a C₁-C₂₀ alkyl group, a C₁-C₂₀ alkoxy group, or a C₁-C₂₀ alkylthio group;
a C₁-C₂₀ alkyl group, a C₁-C₂₀ alkoxy group, or a C₁-C₂₀ alkylthio group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₁₀ alkyl group, a cyclopentyl group, a cyclohexyl group, a cycloheptyl group, a cyclooctyl group, an adamantanyl group, a norbomanyl group, a norbornenyl group, a cyclopentenyl group, a cyclohexenyl group, a cycloheptenyl group, a phenyl group, a biphenyl group, a naphthyl group, a pyridinyl group, a pyrimidinyl group, or a combination thereof;
a cyclopentyl group, a cyclohexyl group, a cycloheptyl group, a cyclooctyl group, an adamantanyl group, a norbornanyl group, a norbomenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a C₁-C₂₀ alkylthio group, a cyclopentyl group, a cyclohexyl group, a cycloheptyl group, a cyclooctyl group, an adamantanyl group, a norbornanyl group, a norbomenyl group, a cyclopentenyl group, a cyclohexenyl group, a cycloheptenyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, an azadibenzosilolyl group, —Si(Q₃₁)(Q₃₂)(Q₃₃), —B(Q₃₁)(Q₃₂), —P(Q₃₁)(Q₃₂), —C(═O)(Q₃₁), —S(═O)₂(Q₃₁), —P(═O)(Q₃₁)(Q₃₂), or a combination thereof; or
—Si(Q₁)(Q₂)(Q₃), —B(Q₁)(Q₂), —C(═O)(Q₁), —S(═O)₂(Q₁), or —P(═O)(Q₁)(Q₂), and
Q₁ to Q₃ and Q₃₁ to Q₃₃ are each independently:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an iso-propyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with at least one of deuterium, a C₁-C₁₀ alkyl group, a phenyl group, a biphenyl group, a pyridinyl group, a pyrimidinyl group, a pyridazinyl group, a pyrazinyl group, and a triazinyl group.

17. The amine-based compound of claim 10, wherein L₁ is:
a single bond; or
a benzene group or a naphthalene group, each unsubstituted or substituted with at least one R₁₀ₐ, and
R₁₀ₐ is the same as defined in Formula 1.

18. The amine-based compound of claim 10, wherein
Ar₁ and Ar₂ are each independently a C₆-C₆₀ aryl group that is unsubstituted or substituted with at least one R₁₀ₐ, a C₁-C₆₀ heteroaryl group that is unsubstituted or substituted with at least one R₁₀ₐ, a monovalent non-aromatic condensed polycyclic group that is unsubstituted or substituted with at least one R₁₀ₐ, or a monovalent non-aromatic condensed heteropolycyclic group that is unsubstituted or substituted with at least one R₁₀ₐ, and
R₁₀ₐ is the same as defined in Formula 1.

19. The amine-based compound of claim 10, wherein Ar₁ and Ar₂ are each independently a group represented by one of Formulae 2A-1 to 2A-14: 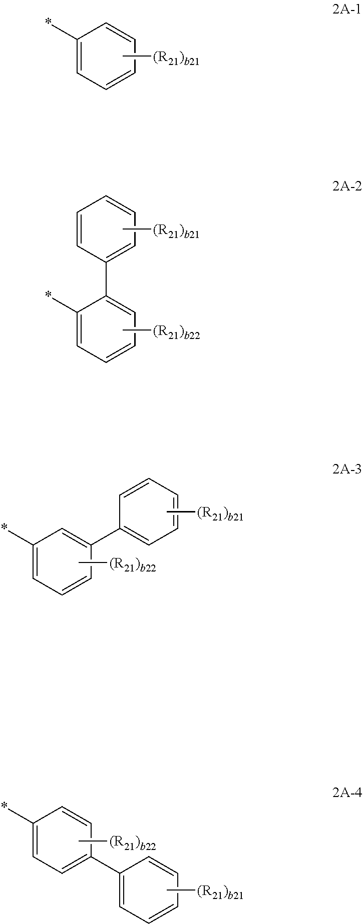 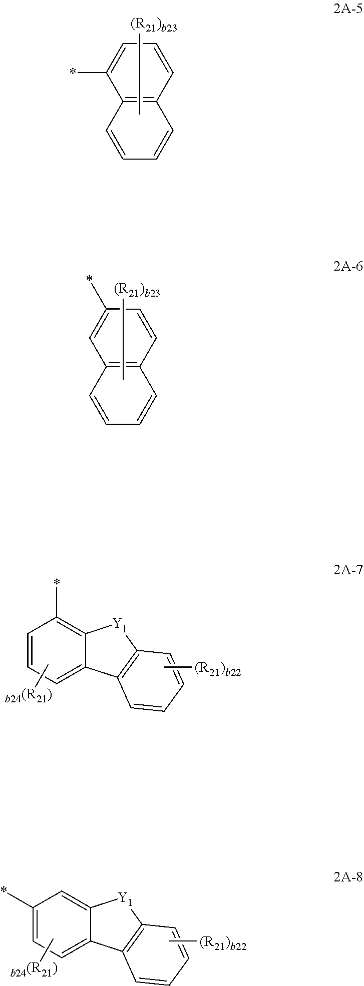 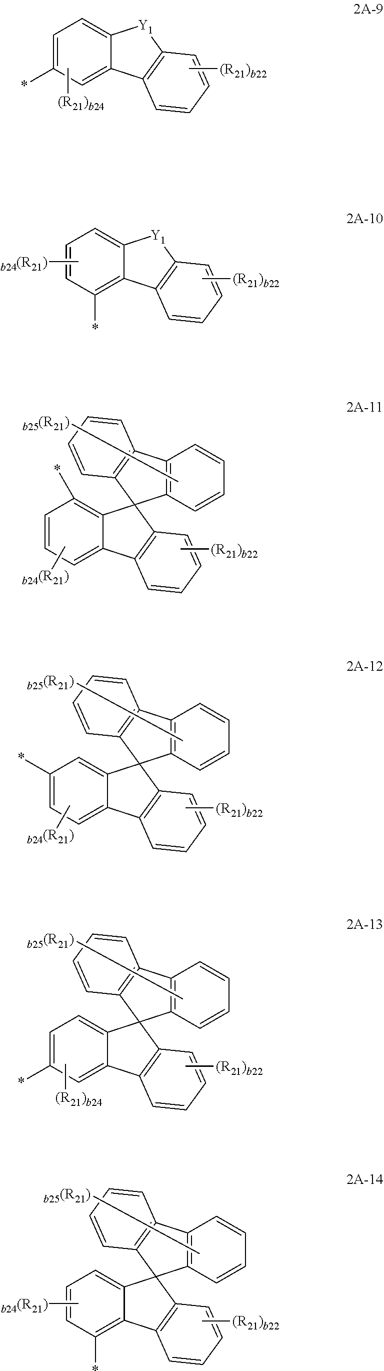
wherein in Formulae 2A-1 to 2A-14,
Y₁ is O, S, N(R₁ₐ), C(R₁ₐ)(R_{1b}), or Si(R₁ₐ)(R_{1b}), R₂₁ is hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂), R₁ₐ and R_{1b} are each independently hydrogen, deuterium, a C₁-C₆₀ alkyl group that is unsubstituted or substituted with at least one R₁₀ₐ, a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
b21 is an integer from 0 to 5,
b22 is an integer from 0 to 4,
b23 is an integer from 0 to 7,
b24 is an integer from 0 to 3,
b25 is an integer from 0 to 8,
indicates a binding site to a neighboring atom, and
R₁₀ₐ and Q₁ to Q₃ are respectively the same as described in Formula 1.

20. The amine-based compound of claim 10, wherein the amine-based compound represented by Formula 1 is represented by one of Formulae 1-1-1 to 1-1-16, 1-2-1 to 1-2-16, and 1-3-1 to 1-3-16: 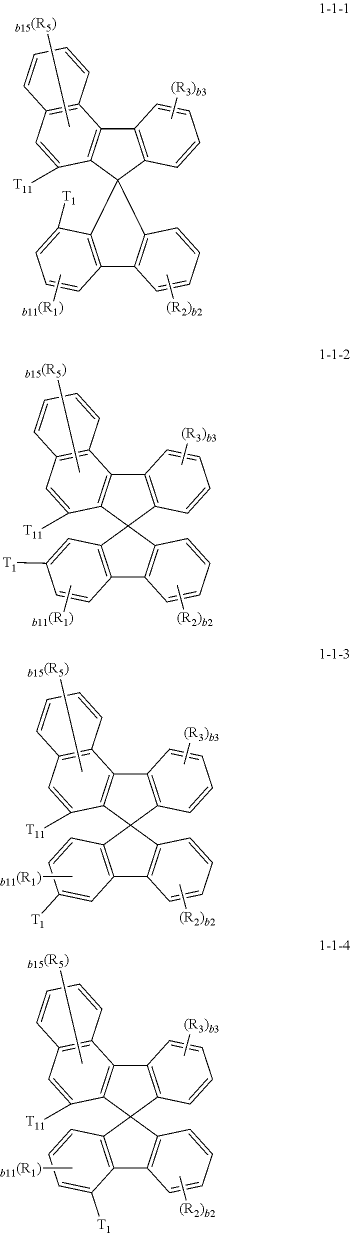 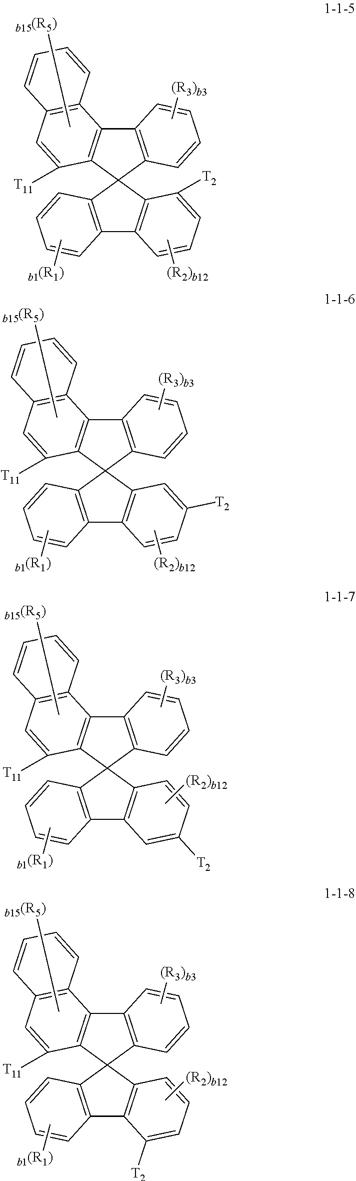 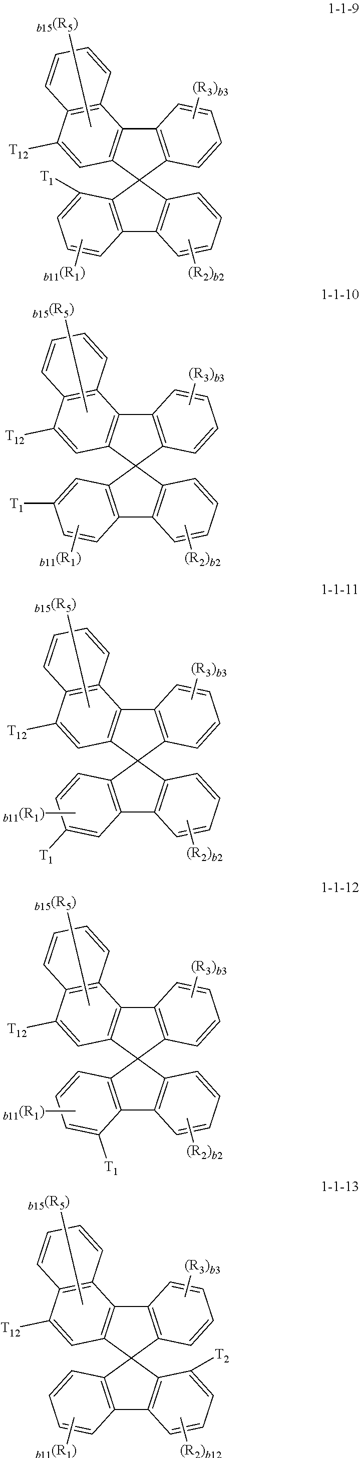 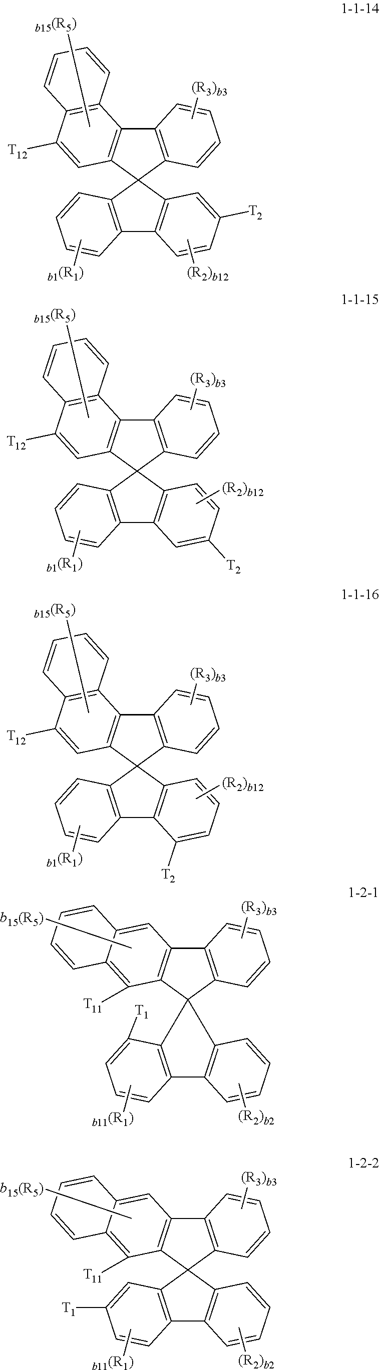 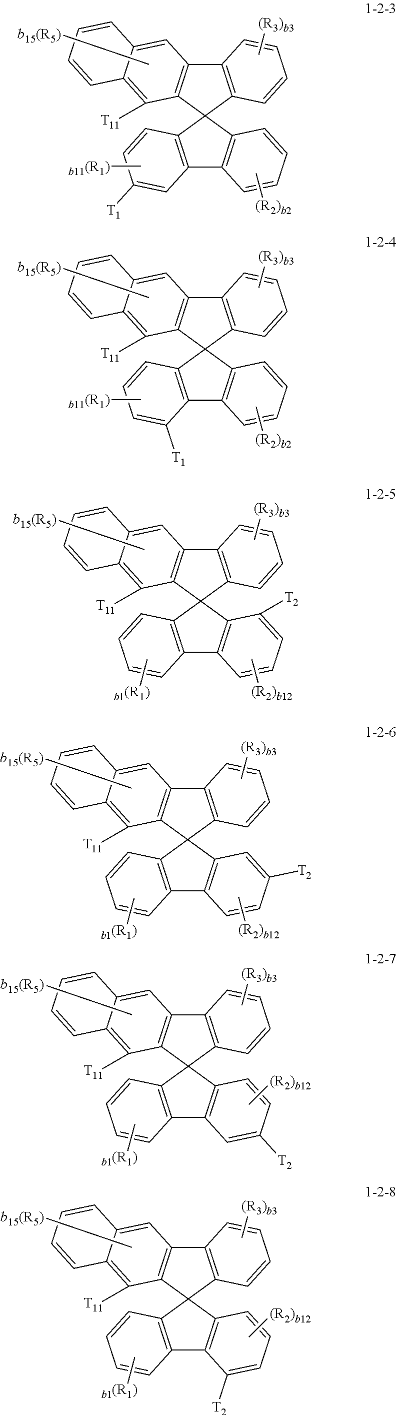 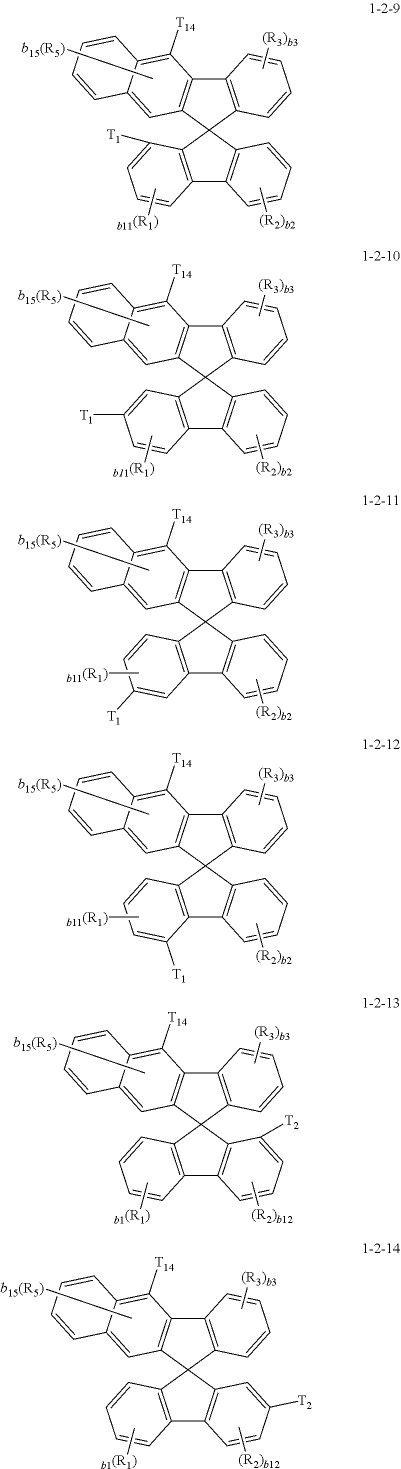 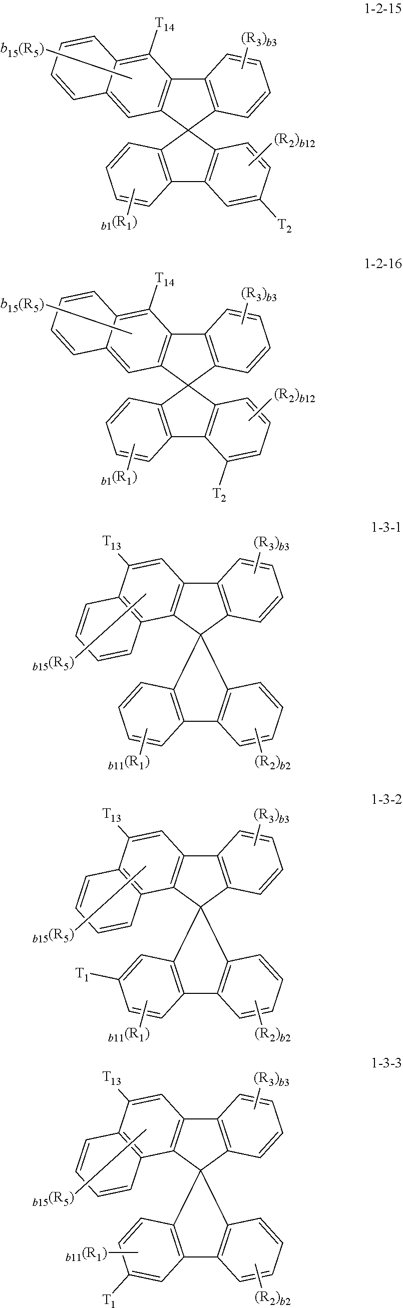 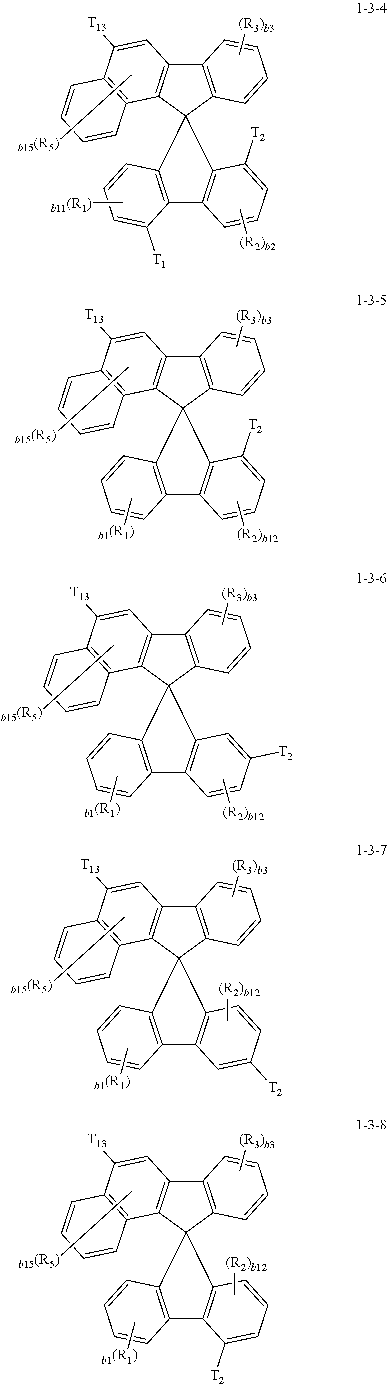 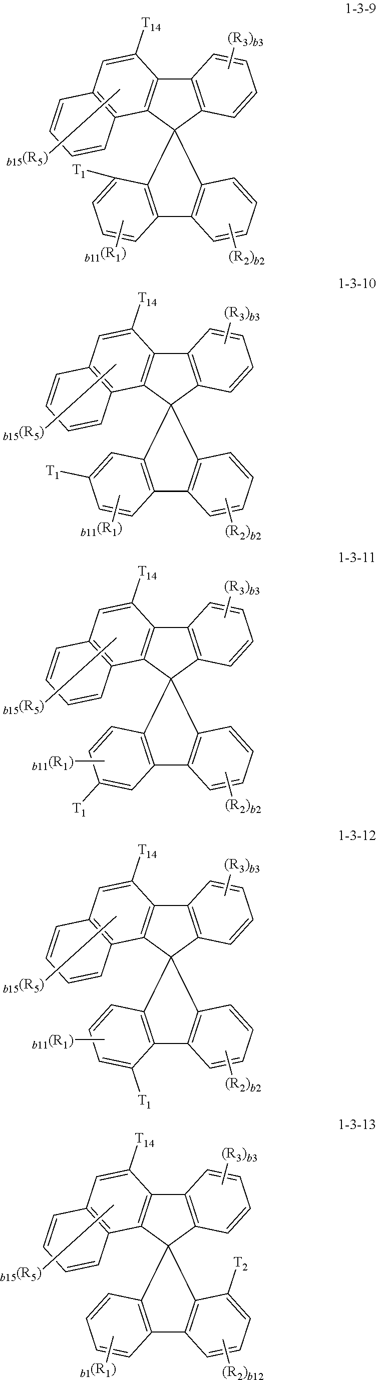 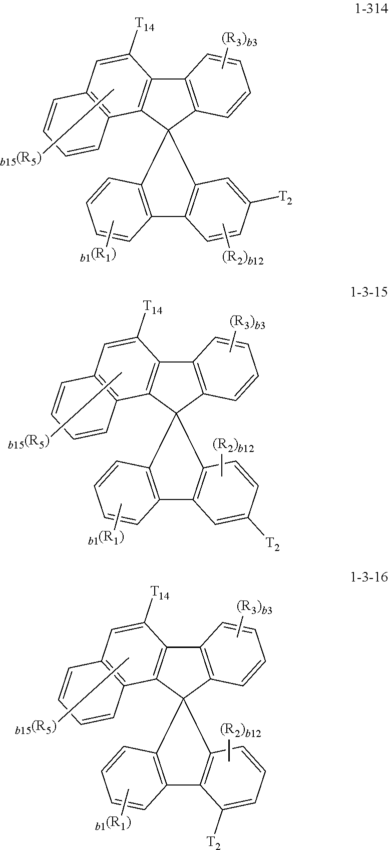
wherein in Formulae 1-1-1 to 1-1-16, 1-2-1 to 1-2-16, and 1-3-1 to 1-3-16,
T₁, T₂, and T₁₁ to T₁₄ are each independently a group represented by Formula 2,
R₅ is hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),
b1 to b3 are each independently an integer from 0 to 4,
b11 and b12 are each independently an integer from 0 to 3,
b15 is an integer from 0 to 5, and
R₁ to R₃, R₁₀ₐ, and Q₁ to Q₃ are respectively the same as described in Formula 1.
